(19) United States
(12) Patent Application Publication
(10) Pub. No.: US 2010/0105700 A1
(43) Pub. Date: Apr. 29, 2010

(54) DECAHYDRO-1H-INDENOQUINOLINONE AND DECAHYDRO-3H-CYCLOPENTAPHENANTHRIDINONE CYP17 INHIBITORS

(75) Inventors: Daniel Chu, Santa Clara, CA (US); Peter L. Myers, Orinda, CA (US); Bing Wang, San Jose, CA (US)

Correspondence Address: WILSON, SONSINI, GOODRICH & ROSATI, 650 PAGE MILL ROAD, PALO ALTO, CA, 94304-1050, US

(73) Assignee: LEAD THERAPEUTICS, INC., San Bruno, CA (US)

(21) Appl. No.: 12/603,377

(22) Filed: Oct. 21, 2009

Related U.S. Application Data
(60) Provisional application No. 61/108,966, filed on Oct. 28, 2008.

(51) Int. Cl. A61K 31/473; C07D 221/18; C07D 311/06; C07D 401/02; A61K 31/497; A61K 31/506; A61K 31/352; A61P 35/00

(52) U.S. Cl. 514/255.05; 546/42; 549/275; 549/382; 544/333; 544/405; 514/280; 514/453

(57) ABSTRACT

Provided herein are inhibitors of CYP17 enzyme. Also described herein are pharmaceutical compositions that include at least one compound described herein and the use of a compound or pharmaceutical composition described herein to treat androgen-dependent diseases, disorders and conditions.

### CROSS REFERENCE

This application claims the benefit of U.S. provisional application Ser. No. 61/108,966, filed Oct. 28, 2008 which is incorporated by reference in its entirety.

### FIELD OF THE INVENTION

Described herein are compounds, methods of making such compounds, pharmaceutical compositions and medicaments containing such compounds, and methods of using such compounds to treat androgen-dependent diseases or conditions.

### BACKGROUND OF THE INVENTION

The 17α-hydroxylase/C₁₇₋₂₀ lyase enzyme complex is essential for the biosynthesis of androgens. CYP17 is a bifunctional enzyme which possess both a C_{17,20}-lyase activity and a C17-hydroxylase activity. These two alternative enzymatic activities of CYP17 result in the formation of critically different intermediates in steroid biosynthesis and each activity appear to be differentially and developmentally regulated.

### SUMMARY OF THE INVENTION

Provided herein are compounds, compositions and methods for inhibiting the CYP17 enzyme. Also described herein is the use of such compounds and compositions for the treatment of cancer and/or androgen-dependent diseases, disorders or conditions.

In one aspect, compounds provided herein have the structure of Formula (I), (II) or (III) and pharmaceutically acceptable salts, solvates, esters, acids and prodrugs thereof.

In some embodiments, isomers and chemically protected forms of compounds having a structure represented by Formula (I), (II), and (III) are also provided.

In one aspect is a compound having the structure of Formula (I):

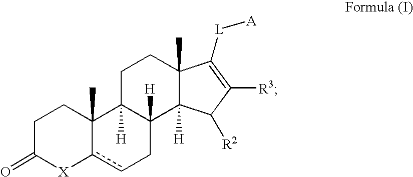

wherein:

X is O or NR¹;

L is a direct bond or

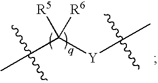

Y is a direct bond, O, C═O, C(O)O, S(O)ᵤ, NR¹ or NR⁷C(O);

q is an integer from 0 to 4;

u is an integer from 0 to 2;

A is a heteroaryl optionally substituted with 1, 2, 3, or 4 R⁸;

[symbol] is a single or double bond;

R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or

R_{A} and R_{B} taken together with the nitrogen atom to which they are attached form an optionally substituted 4 to 7 membered heterocyclic ring having one or two heteroatoms;

R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy;

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl; and

R⁵ and R⁶ are each independently hydrogen, halogen, nitro, cyano, hydroxyl, optionally substituted alkyl, optionally substituted cycloalkyl, perfluoroalkyl, optionally substituted heteroalkyl, optionally substituted heterocycloalkyl, optionally substituted aryl, optionally substituted heteroaryl;

R⁷ is hydrogen or an optionally substituted alkyl;

R⁸ is each independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, or NR_{A}R_{B};

or a pharmaceutically acceptable salt or solvate thereof.

In one embodiment is a compound having the structure of Formula (II):

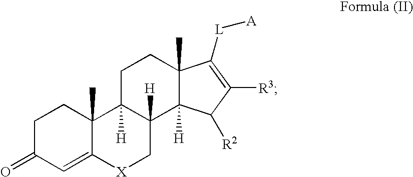

wherein:

X is O or NR¹;

L is a direct bond or

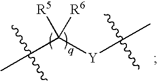

Y is a direct bond, O, C═O, C(O)O, S(O)ᵤ, NR¹, NR⁷C(O);

q is an integer from 0 to 4;

u is an integer from 0 to 2;

A is a heteroaryl optionally substituted with 1, 2, 3, or 4 R⁸;

R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or

R_{A} and R_{B} taken together with the nitrogen atom to which they are attached form an optionally substituted 4 to 7 membered heterocyclic ring having one or two heteroatoms;

R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy;

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl; and

R⁵ and R⁶ are each independently hydrogen, halogen, nitro, cyano, hydroxyl, optionally substituted alkyl, optionally substituted cycloalkyl, perfluoroalkyl, optionally substituted heteroalkyl, optionally substituted heterocycloalkyl, optionally substituted aryl, optionally substituted heteroaryl;

R⁷ is hydrogen or an optionally substituted alkyl;

R⁸ is each independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, or NR_{A}R_{B};

or a pharmaceutically acceptable salt or solvate thereof.

In another embodiment is a compound having the structure of Formula (III):

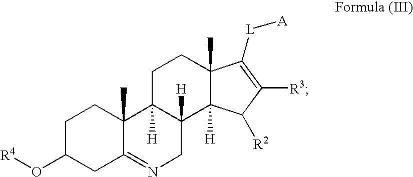

wherein:

L is a direct bond or

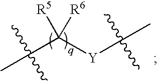

Y is a direct bond, O, C═O, C(O)O, S(O)ᵤ, NR¹, NR⁷C(O);

q is an integer from 0 to 4;

u is an integer from 0 to 2;

A is a heteroaryl optionally substituted with 1, 2, 3, or 4 R⁸;

R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or

R_{A} and R_{B} taken together with the nitrogen atom to which they are attached form an optionally substituted 4 to 7 membered heterocyclic ring having one or two heteroatoms;

R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy;

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl;

R⁴ is selected from the group consisting of hydrogen, optionally substituted alkyl, optionally substituted cycloalkyl, optionally substituted haloalkyl, optionally substituted hydroxyalkyl, R_{A}carbonyl, (NR_{A}R_{B})alkyl, and (NR_{A}R_{B})carbonyl; and

R⁵ and R⁶ are each independently hydrogen, halogen, nitro, cyano, hydroxyl, optionally substituted alkyl, optionally substituted cycloalkyl, perfluoroalkyl, optionally substituted heteroalkyl, optionally substituted heterocycloalkyl, optionally substituted aryl, optionally substituted heteroaryl;

R⁷ is hydrogen or an optionally substituted alkyl;

R⁸ is each independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, or NR_{A}R_{B};

or a pharmaceutically acceptable salt or solvate thereof.

In one embodiment, is a compound having the structure of Formula (IA):

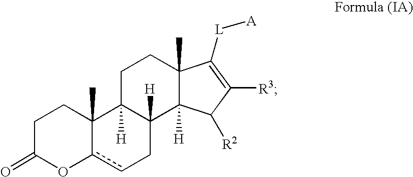

or a pharmaceutically acceptable salt or solvate thereof.

In another embodiment is a compound having the structure of Formula (IB):

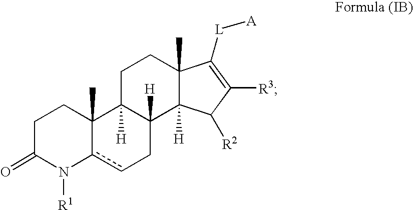

or a pharmaceutically acceptable salt or solvate thereof.

In a further embodiment is a compound having the structure of Formula (IIA):

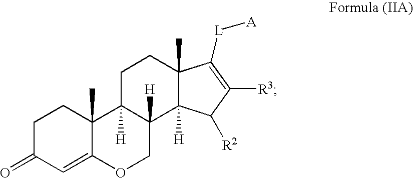

or a pharmaceutically acceptable salt or solvate thereof.

In yet a further embodiment is a compound having the structure of Formula (IIB):

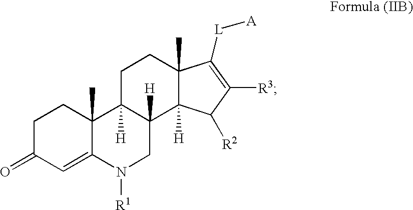

or a pharmaceutically acceptable salt or solvate thereof.

In one embodiment is a compound having the structure of Formula (I), (II) or (III) wherein A is an optionally substituted heteroaryl.

In another embodiment is a compound having the structure of Formula (I), (II) or (III) wherein the heteroaryl is selected from the group consisting of pyridine, pyrimidine, pyrazine, pyrazole, oxazole, thiazole, isoxazole, isothiazole, 1,3,4-oxadiazole, pyridazine, 1,3,5-trazine, 1,2,4-triazine, quinoxaline, benzimidazole, benzotriazole, purine, 1H-[1,2,3]triazolo[4,5-d]pyrimidine, triazole, imidazole, thiophene, furan, isobenzofuran, pyrrole, indolizine, isoindole, indole, indazole, isoquinoline, quinoline, phthalazine, naphthyridine, quinazoline, cinnoline, and pteridine.

In a further embodiment is a compound having the structure of Formula (I), (II) or (III) wherein the heteroaryl is selected from pyridine, imidazole, benzimidazole, pyrrole, pyrazole, pyrimidine, pyrazine, and pyridazine.

In yet another embodiment is a compound having the structure of Formula (I), (II) or (III) wherein the heteroaryl is pyridine.

In one embodiment is a compound having the structure of Formula (I), (II) or (III) wherein the heteroaryl is benzimidazole.

In another embodiment is a compound having the structure of Formula (I), (II) or (III) wherein the heteroaryl is imidazole.

In yet another embodiment is a compound having the structure of Formula (I), (II) or (III) wherein the heteroaryl is selected from the group consisting of pyrazine and pyrimidine.

In yet a further embodiment is a compound having the structure of Formula (I), (II) or (III) wherein R¹ is hydrogen, alkyl, cycloalkyl and wherein the alkyl and cycloalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, and (NR_{A}R_{B})carbonyl.

In another embodiment is a compound having the structure of Formula (I), (II), or (III) wherein R¹ is hydrogen or C₁-C₆ alkyl.

In one embodiment is a compound having the structure of Formula (I), (II) or (III) wherein R² is selected from the group consisting of hydrogen, halogen, hydroxyl, optionally substituted alkyl, optionally substituted cycloalkyl, cyano, or nitro.

In another embodiment is a compound having the structure of Formula (I), (II) or (III) wherein R² is hydrogen or C₁-C₆ alkyl.

In yet another embodiment is a compound having the structure of Formula (I), (II) or (III) wherein R³ is selected from the group consisting of hydrogen, halogen, optionally substituted alkyl, optionally substituted cycloalkyl, cyano, hydroxyl, or nitro.

In a further embodiment is a compound having the structure of Formula (I), (II) or (III) wherein R³ is hydrogen or C₁-C₆ alkyl.

In yet a further embodiment is a compound having the structure of Formula (III) wherein R⁴ is selected from the group consisting of hydrogen, optionally substituted alkyl, optionally substituted cycloalkyl or R_{A}carbonyl.

In another embodiment is a compound having the structure of Formula (III) wherein R⁴ is hydrogen or R_{A}carbonyl.

In yet another embodiment is a compound having the structure of Formula (III) wherein R_{A} is an optionally substituted alkyl.

In one embodiment is a compound having the structure of Formula (I), (II) or (III) wherein L is a direct bond.

In another embodiment is a compound having the structure of Formula (I), (II) or (III) wherein L is

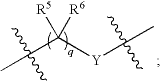

### Y is C═O; and q is 0.

In one embodiment is a compound having the structure of Formula (I), (II) or (III) wherein L is

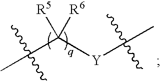

Y is a direct bond; R⁵ and R⁶ are independently hydrogen, and q is 0-4.

In another embodiment is a compound having the structure of Formula (I), (II) or (III) wherein q is 1.

In another embodiment is a compound having the structure of Formula (I) wherein [symbol] is a double bond.

In another embodiment is a compound having the structure of Formula (I) wherein [symbol] is a single bond.

Also described herein is a pharmaceutical composition comprising a compound having a structure of Formula (I), (II) or (III) and a pharmaceutically acceptable carrier, excipient or binder thereof.

In one aspect is a method for treating cancer in a subject comprising administering to a subject in need a therapeutically acceptable amount of a compound having the structure of Formula (I), (II) or (III) or a pharmaceutically acceptable salt or solvate thereof.

In one embodiment is a method for treating cancer in a subject comprising administering to a subject in need a therapeutically acceptable amount of a compound having the structure of Formula (I), (II) or (III) or a pharmaceutically acceptable salt or solvate thereof wherein the cancer is selected from the group consisting of bladder cancer, brain cancer, breast cancer, cervical cancer, colorectal cancer, endometrial cancer, gastric cancer, glioblastoma, head and neck cancer, Kaposi's sarcoma, kidney cancer, leiomyosarcoma, leukemia, liver cancer, lung cancer, melanoma, multiple myeloma, Non-Hodgkin lymphoma, ovarian cancer, pancreatic cancer, papillary renal cell carcinoma, prostate cancer, renal cancer, squamous cell cancer, and thoracic cancer.

In another embodiment is a method for treating cancer in a subject comprising administering to a subject in need a therapeutically acceptable amount of a compound having the structure of Formula (I), (II) or (III) or a pharmaceutically acceptable salt or solvate thereof wherein the cancer is prostate cancer.

In another embodiment is a method for treating cancer in a subject comprising administering to a subject in need a therapeutically acceptable amount of a compound having the structure of Formula (I), (II) or (III) or a pharmaceutically acceptable salt or solvate thereof wherein the cancer is breast cancer.

In a further embodiment the method of treating cancer further comprises providing to the subject in need an additional therapy selected from the group consisting of surgery, radiation therapy, chemotherapy, gene therapy, immunotherapy, or a combination thereof.

In yet a further embodiment, the additional therapy is surgery.

In one embodiment, providing chemotherapy to the subject in need comprises administering a therapeutically effective amount of at least one anti-androgenic agent.

In another embodiment, the at least one anti-androgenic agent is selected from the group consisting of flutamide, nicalutamide, bicalutamide, inhibitors of 17α-hydroxylase/C17-20 lyase, luteinizing hormone-releasing hormone agonists, luteinizing hormone-releasing hormone antagonists, and 5α-reductase type 1 and/or type 2 and combinations thereof.

Also disclosed herein is a method of inhibiting CYP17 enzyme comprising contacting a compound having the structure of Formula (I), (II) or (III) or a pharmaceutically acceptable salt or solvate thereof with a CYP17 enzyme.

In one embodiment, the contacting step is in vivo.

Also described herein is a method of treating an androgen-dependent disorder in a subject comprising administering to a subject in need a therapeutically acceptable amount of a compound having the structure of Formula (I), (II) or (III) or a pharmaceutically acceptable salt or solvate thereof.

In one embodiment, the androgen-dependent disorder is selected from the group consisting of prostate cancer, benign prostatic hyperplasia, prostatic intraepithelial neoplasia, hirsutism, acne, androgenic alopecia, and polycystic ovary syndrome.

In another embodiment, the androgen-dependent disorder is prostate cancer.

Presented herein is a method of treating a proliferative disease comprising administering to a subject in need a therapeutically effective amount of a compound having the structure of Formula (I), (II) or (III) or a pharmaceutically acceptable salt or solvate thereof.

In one embodiment, the method further comprises administering a therapeutically effective amount of at least one agent or therapy selected from the group consisting of a chemotherapeutic agent, a biological agent, surgery, and radiation therapy.

In another embodiment, the administration is performed concurrently or sequentially.

In one aspect is an article of manufacture, comprising packaging material, a compound having the structure of Formula (I), (II) or (III), and a label, wherein the compound is effective for the treatment of an androgen dependent disorder, wherein the compound is packaged within the packaging material, and wherein the label indicates that the compound, or pharmaceutically acceptable salt or solvate thereof is used for the treatment of an androgen dependent disorder.

In one aspect, is a use of a compound having the structure of Formula (I), (II) or (III) or a pharmaceutically acceptable salt or solvate thereof for the manufacture of a medicament for the treatment of prostate cancer.

### DETAILED DESCRIPTION OF THE INVENTION

In the testes and adrenal glands, the last step in the biosynthesis of testosterone involves two key reactions, which act sequentially and are both catalyzed by a single enzyme, the cytochrome P450 monooxygenase 17α-hydroxylase/C_{17,20}-lyase (P450₁₇ or CYP17). CYP17 is a key enzyme in the biosynthesis of androgens, and converts the C₂₁ steroids (pregnenolone and progesterone) to the C₁₉ androgens, dehydroepiandrosterone (DHEA), androstenediol (A-diol), testosterone, and androstenedione in the testes and adrenals. Both DHEA and androstenedione lyase products are key intermediates in the synthesis of not only the androgens testosterone and dihydrotestosterone (DHT), but also the estrogens 17β-estradiol and estrone. Adrenal and ovarian estrogens are the main sources of estrogens in postmenopausal women. The C₁₇-hydroxylase activity of CYP17 catalyzes the conversion of the common intermediate progesterone to 17-hydroxyprogesterone, a precursor of cortisol. Thus, the C₁₇-hydroxylase activity promotes the formation of glucocorticoids while the C_{17,20}-lyase activity promotes the formation of sex hormones—particularly androgens including testosterone as well as estrogens.

Prostate cancer is the most common malignancy and age-related cause of cancer death worldwide. Apart from lung cancer, prostate cancer is the most common form of cancer in men and the second leading cause of death in American men. During the period of 1992 to 1999, the average annual incidence of prostate cancer among African American men was 59% higher than among Caucasian men, and the average annual death rate was more than twice that of Caucasian men (American Cancer Society—Cancer Facts and Figures 2003).

Androgens play an important role in the development, growth, and progression of prostate cancer. Two important androgens in this regard are testosterone and dihydrotestosterone (DHT). The testes synthesize about 90% of testosterone and the rest (10%) is synthesized by the adrenal glands. Testosterone is further converted to the more potent androgen DHT by the enzyme steroid 5α-reductase that is localized primarily in the prostate.

Since prostate cancer is typically androgen-dependent, the reduction of androgen production via surgical or pharmacological castration is the major treatment option for this indication. Androgen deprivation has been used as therapy for advanced and metastatic prostate cancer. Androgen ablation therapy has been shown to produce the most beneficial responses in multiple settings in prostate cancer patients. However, orchidectomy remains the standard treatment option for most prostate cancer patients.

Medical and surgical orchidectomy reduces or eliminates androgen production by the testes but does not affect androgen synthesis in the adrenal glands. Several studies have reported that orchidectomy therapy and treatment with anti-androgens to inhibit the action of adrenal androgens significantly prolongs the survival of prostate cancer patients. Further, it has been shown that testosterone and DHT occur in recurrent prostate cancer tissues at levels sufficient to activate androgen receptor. In addition, the use of microarray-based profiling of isogenic prostate cancer xenograft models showed that a modest increase in androgen receptor mRNA was the only change consistently associated with the development of resistance to anti-androgen therapy. Since CYP17 is implicated in the synthesis of key intermediates of androgens, the pharmacological inhibition of CYP17 is a promising treatment in that testicular, adrenal, and peripheral androgen biosynthesis would be reduced rather than only testicular androgen production. (Njar, V. et al., J. Med. Chem. 1998, 41, 902).

Inhibitors of CYP17 have been previously described. For example, ketoconazole, an active imidazole fungicide has been used to reduce testosterone biosynthesis in the treatment of patients with advanced prostatic cancer. However, there are side-effects including liver damage, inhibition of several other cytochrome P₄₅₀ steroidogenic enzymes, and reduction of cortisol production.

Potent and selective inhibitors of CYP17 as potential prostate cancer treatments have been the subject of previous studies. Finasteride, a 5α-reductatse inhibitor, is an approved treatment for benign prostatic hyperplasia (BPH), although it is only effective with patients exhibiting minimal disease. While finasteride reduces serum DHT levels, it increases testosterone levels and may therefore be insufficient for prostate cancer treatment.

In addition to the use of CYP17 inhibitors in the treatment of prostate cancer, CYP17 inhibitors will find utility for the indication of breast cancer, more particularly, estrogen-dependent breast cancer. In post-menopausal patients with advanced breast cancer, treatment with high doses of ketoconazole resulted in suppression of both testosterone and estradiol levels, implicating CYP17 as a potential target for hormone therapy. (Harris, A. L. et al., Br. J. Cancer 1988, 58, 493).

Provided herein are compounds having the structure of Formulas (I), (II), (III) or pharmaceutically acceptable salts or solvates thereof, in the treatment of cancer, in the inhibition of CYP17, and in the treatment of androgen-dependent diseases.

In one aspect is a compound having the structure of Formula (I):

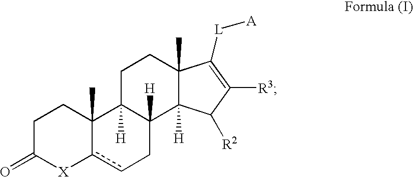

wherein:

X is O or NR¹;

L is a direct bond or

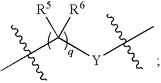

Y is a direct bond, O, C═O, C(O)O, S(O)ᵤ, NR¹, or NR⁷C(O);

q is an integer from 0 to 4;

u is an integer from 0 to 2;

A is a heteroaryl optionally substituted with 1, 2, 3, or 4 R⁸;

[symbol] is a single or double bond;

R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or

R_{A} and R_{B} taken together with the nitrogen atom to which they are attached form an optionally substituted 4 to 7 membered heterocyclic ring having one or two heteroatoms;

R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy;

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl; and

R⁵ and R⁶ are each independently hydrogen, halogen, nitro, cyano, hydroxyl, optionally substituted alkyl, optionally substituted cycloalkyl, perfluoroalkyl, optionally substituted heteroalkyl, optionally substituted heterocycloalkyl, optionally substituted aryl, optionally substituted heteroaryl;

R⁷ is hydrogen or an optionally substituted alkyl;

R⁸ is each independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, or NR_{A}R_{B};

or a pharmaceutically acceptable salt or solvate thereof.

In one embodiment is a compound having the structure of Formula (I) wherein X is O. In another embodiment, is a compound having the structure of Formula (IA):

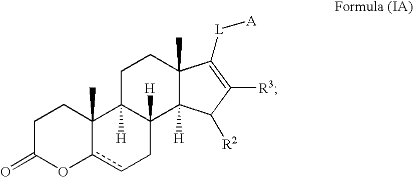

or a pharmaceutically acceptable salt or solvate thereof.

In a further embodiment, is a compound having the structure of Formula (I) wherein X is NR¹. In yet another embodiment, is a compound having the structure of Formula (IB):

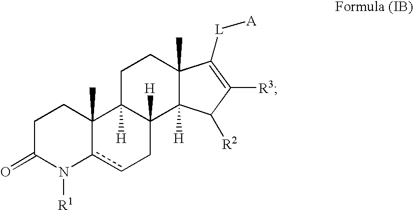

or a pharmaceutically acceptable salt or solvate thereof.

In one embodiment, is a compound having the structure of Formula (I), (IA), or (IB) wherein

A is an optionally substituted heteroaryl. In another embodiment, A is an optionally substituted heteroaryl group. In another embodiment, the heteroaryl group consists of one, two, three, or four heteroatoms selected from N, S, and O. In one embodiment, is a compound having the structure of Formula (I), (IA), or (IB) wherein the heteroaryl group is selected from the group consisting of pyridine, pyrimidine, pyrazine, pyrazole, oxazole, thiazole, isoxazole, isothiazole, 1,3,4-oxadiazole, pyridazine, 1,3,5-trazine, 1,2,4-triazine, quinoxaline, benzimidazole, benzotriazole, purine, 1H-[1,2,3]triazolo[4,5-d]pyrimidine, triazole, imidazole, thiophene, furan, isobenzofuran, pyrrole, indolizine, isoindole, indole, indazole, isoquinoline, quinoline, phthalazine, naphthyridine, quinazoline, cinnoline, and pteridine. In another embodiment, the heteroaryl group is selected from pyridine, imidazole, benzimidazole, pyrrole, pyrazole, pyrimidine, pyrazine, and pyridazine. In a further embodiment, the heteroaryl group is pyridine. In another embodiment, the heteroaryl group is benzimidazole. In yet another embodiment, the heteroaryl group is imidazole. In yet another embodiment, the heteroaryl group is pyrazine.

In one embodiment, is a compound of Formula (I), (IA), or (IB) wherein A is an optionally substituted heteroaryl attached to L at a heteroatom of the heteroaryl group. By way of example only, A is an optionally substituted benzoimidazole group,

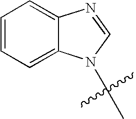

wherein the benzoimidazole group is attached to L at a nitrogen atom,

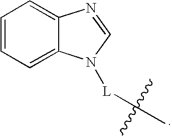

In one embodiment, L is a direct bond such that the benzoimidazole group is attached directly to the steroid scaffold,

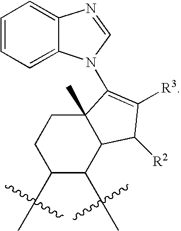

In another embodiment, is a compound of Formula (I), (IA), or (IB) wherein A is an optionally substituted heteroaryl attached to L at a carbon atom of the heteroaryl group. Also by way of example only, A is an optionally substituted pyridine group,

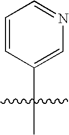

wherein the pyridine group is attached to L at a carbon atom,

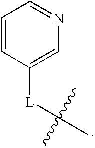

In one embodiment, L is a direct bond such that the pyridine group is attached directly to the steroid scaffold,

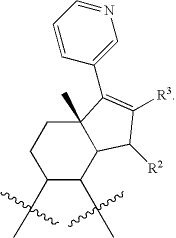

In one embodiment, L is a direct bond. In one embodiment, L is

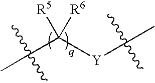

wherein Y is a direct bond, O, C═O, C(O)O, S(O)ᵤ, NR¹, or NR⁷C(O); q is an integer from 0 to 4; u is an integer from 0 to 2; R⁵ and R⁶ are each independently hydrogen, halogen, nitro, cyano, hydroxyl, optionally substituted alkyl, optionally substituted cycloalkyl, perfluoroalkyl, optionally substituted heteroalkyl, optionally substituted heterocycloalkyl, optionally substituted aryl, optionally substituted heteroaryl; and R⁷ is hydrogen or an optionally substituted alkyl. In another embodiment, L is

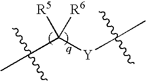

wherein Y is a direct bond and q is 0. In yet another embodiment, Y is a direct bond; q is 1-4; and R⁵ and R⁶ are both hydrogen. In a further embodiment, L is —CH₂—. In another embodiment, L is —CH₂ CH₂—. In another embodiment, Y is —O— and q is 0-4. In another embodiment, L is

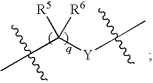

Y is C═O; and q is 0. In yet another embodiment, Y is C═O, C(O)O, NR¹ or NR⁷C(O). In yet another embodiment, Y is NH. In another embodiment, Y is —N(C₁-C₆alkyl)-.

In one embodiment, is a compound having the structure of Formula (I), (IA) or (IB), wherein R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy. In another embodiment, R² is selected from a group consisting of hydrogen, optionally substituted C₁-C₆ alkyl; optionally substituted C₁-C₈ cycloalkyl, cyano, halogen, or nitro. In a further embodiment, R² is hydrogen or C₁-C₆ alkyl. In yet a further embodiment, R² is hydrogen.

In one embodiment, is a compound having the structure of Formula (I), (IA), or (IB), wherein

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl. In another embodiment, R³ is R_{A}carbonyl, wherein R_{A} is hydrogen. In another embodiment, R³ is selected from a group consisting of hydrogen, optionally substituted C₁-C₆ alkyl; optionally substituted C₁-C₈ cycloalkyl, cyano, halogen, or nitro. In a further embodiment, R³ is hydrogen or C₁-C₆ alkyl. In yet a further embodiment, R³ is hydrogen.

In one embodiment, is a compound having the structure of Formula (I), (IA), (IB) wherein R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or

R_{A} and R_{B} taken together with the nitrogen atom to which they are attached form an optionally substituted 4 to 7 membered heterocyclic ring having one or two heteroatoms. In one embodiment, R¹ is selected from the group consisting of hydrogen, optionally substituted alkyl or optionally substituted cycloalkyl. In another embodiment, R¹ is hydrogen or C₁-C₆ alkyl. In a further embodiment, R¹ is hydrogen. In yet a further embodiment, R¹ is —CH₃.

In another aspect is a compound having the structure of Formula (II):

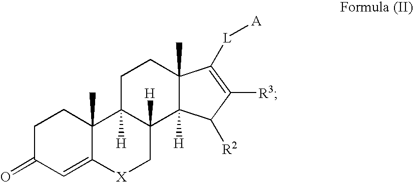

wherein:

X is O or NR¹;

L is a direct bond or

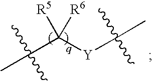

Y is a direct bond, O, C═O, C(O)O, S(O)ᵤ, NR¹, or NR⁷C(O);

q is an integer from 0 to 4;

u is an integer from 0 to 2;

A is a heteroaryl optionally substituted with 1, 2, 3, or 4 R⁸;

R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or

R_{A} and R_{B} taken together with the nitrogen atom to which they are attached form an optionally substituted 4 to 7 membered heterocyclic ring having one or two heteroatoms;

R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy;

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl; and

R⁵ and R⁶ are each independently hydrogen, halogen, nitro, cyano, hydroxyl, optionally substituted alkyl, optionally substituted cycloalkyl, perfluoroalkyl, optionally substituted heteroalkyl, optionally substituted heterocycloalkyl, optionally substituted aryl, optionally substituted heteroaryl;

R⁷ is hydrogen or an optionally substituted alkyl;

R⁸ is each independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, or NR_{A}R_{B};

or a pharmaceutically acceptable salt or solvate thereof.

In one embodiment is a compound having the structure of Formula (II) wherein X is O. In another embodiment, is a compound having the structure of Formula (IIA):

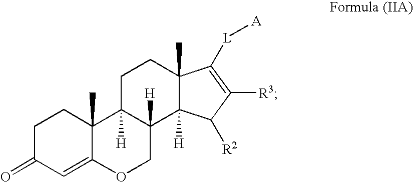

or a pharmaceutically acceptable salt or solvate thereof.

In a further embodiment, is a compound having the structure of Formula (II) wherein X is NR¹. In another embodiment, is a compound having the structure of Formula (IIB):

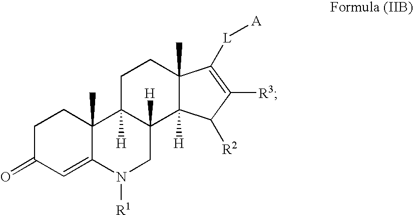

or a pharmaceutically acceptable salt or solvate thereof.

In one embodiment, is a compound having the structure of Formula (II), (IIA), or (IIB) wherein A is an optionally substituted heteroaryl. In another embodiment, A is an optionally substituted heteroaryl group. In another embodiment, the heteroaryl group consists of one, two, three, or four heteroatoms selected from N, S, and O. In one embodiment, is a compound having the structure of Formula (II), (IIA), or (IIB) wherein the heteroaryl group is selected from the group consisting of pyridine, pyrimidine, pyrazine, pyrazole, oxazole, thiazole, isoxazole, isothiazole, 1,3,4-oxadiazole, pyridazine, 1,3,5-trazine, 1,2,4-triazine, quinoxaline, benzimidazole, benzotriazole, purine, 1H-[1,2,3]triazolo[4,5-d]pyrimidine, triazole, imidazole, thiophene, furan, isobenzofuran, pyrrole, indolizine, isoindole, indole, indazole, isoquinoline, quinoline, phthalazine, naphthyridine, quinazoline, cinnoline, and pteridine. In another embodiment, the heteroaryl group is selected from pyridine, imidazole, benzimidazole, pyrrole, pyrazole, pyrimidine, pyrazine, and pyridazine. In a further embodiment, the heteroaryl group is pyridine. In another embodiment, the heteroaryl group is benzimidazole. In yet another embodiment, the heteroaryl group is imidazole. In yet another embodiment, the heteroaryl group is pyrazine.

In one embodiment, is a compound of Formula (II), (IIA), or (IIB) wherein A is an optionally substituted heteroaryl attached to L at a heteroatom of the heteroaryl group. By way of example only, A is an optionally substituted benzoimidazole group,

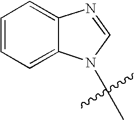

wherein the benzoimidazole group is attached to L at a nitrogen atom,

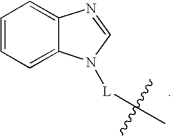

In one embodiment, L is a direct bond such that the benzoimidazole group is attached directly to the steroid scaffold

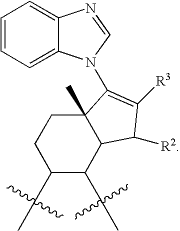

In another embodiment, is a compound of Formula (II), (IIA), or (IIB) wherein A is an optionally substituted heteroaryl attached to L at a carbon atom of the heteroaryl group. Also by way of example only, A is an optionally substituted pyridine group,

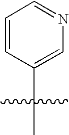

wherein the pyridine group is attached to L at a carbon atom,

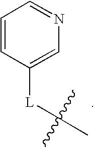

In one embodiment, L is a direct bond such that the pyridine group is attached directly to the steroid scaffold,

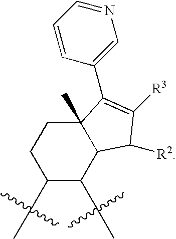

In one embodiment, L is a direct bond. In one embodiment, L is

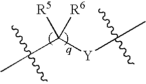

wherein Y is a direct bond, O, C═O, C(O)O, S(O)ᵤ, NR¹, or NR⁷C(O); q is an integer from 0 to 4; u is an integer from 0 to 2; R⁵ and R⁶ are each independently hydrogen, halogen, nitro, cyano, hydroxyl, optionally substituted alkyl, optionally substituted cycloalkyl, perfluoroalkyl, optionally substituted heteroalkyl, optionally substituted heterocycloalkyl, optionally substituted aryl, optionally substituted heteroaryl; and R⁷ is hydrogen or an optionally substituted alkyl. In another embodiment, L is

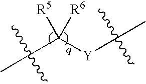

wherein Y is a direct bond and q is 0. In yet another embodiment, Y is a direct bond; q is 1-4; and R⁵ and R⁶ are both hydrogen. In a further embodiment, L is —CH₂—. In another embodiment, L is —CH₂ CH₂—. In another embodiment, Y is —O— and q is 0-4. In yet another embodiment, Y is C═O, C(O)O, NR¹ or NR⁷C(O). In another embodiment, L is

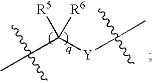

Y is C═O; and q is 0. In yet another embodiment, Y is NH. In another embodiment, Y is —N(C₁-C₆alkyl)-.

In one embodiment, is a compound having the structure of Formula (II), (IIA) or (IIB), wherein R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy. In another embodiment, R² is selected from a group consisting of hydrogen, optionally substituted C₁-C₆ alkyl; optionally substituted C₁-C₈ cycloalkyl, cyano, halogen, or nitro. In a further embodiment, R² is hydrogen or C₁-C₆ alkyl. In yet a further embodiment, R² is hydrogen.

In one embodiment, is a compound having the structure of Formula (II), (IIA), or (IIB), wherein R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl. In another embodiment, R³ is R_{A}carbonyl, wherein R_{A} is hydrogen. In another embodiment, R³ is selected from a group consisting of hydrogen, optionally substituted C₁-C₆ alkyl; optionally substituted C₁-C₈ cycloalkyl, cyano, halogen, or nitro. In a further embodiment, R³ is hydrogen or C₁-C₆ alkyl. In yet a further embodiment, R³ is hydrogen.

In one embodiment, is a compound having the structure of Formula (II), (IIA), (IIB) wherein R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or

R_{A} and R_{B} taken together with the nitrogen atom to which they are attached form an optionally substituted 4 to 7 membered heterocyclic ring having one or two heteroatoms. In one embodiment, R¹ is selected from the group consisting of hydrogen, optionally substituted alkyl or optionally substituted cycloalkyl. In another embodiment, R¹ is hydrogen or C₁-C₆ alkyl. In a further embodiment, R¹ is hydrogen.

In a further aspect is a compound having the structure of Formula (III):

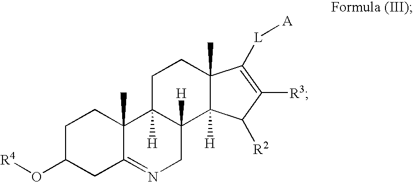

wherein:

L is a direct bond or

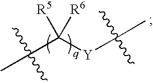

Y is a direct bond, O, C═O, C(O)O, S(O)ᵤ, NR¹, or NR⁷C(O);

q is an integer from 0 to 4;

u is an integer from 0 to 2;

A is a heteroaryl optionally substituted with 1, 2, 3, or 4 R⁸;

R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or

R_{A} and R_{B} taken together with the nitrogen atom form an optionally substituted 4 to 7 membered heterocyclic ring having one or two heteroatoms;

R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy;

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl;

R⁴ is selected from the group consisting of hydrogen, optionally substituted alkyl, optionally substituted cycloalkyl, optionally substituted haloalkyl, optionally substituted hydroxyalkyl, R_{A}carbonyl, (NR_{A}R_{B})alkyl, and (NR_{A}R_{B})carbonyl; and

R⁵ and R⁶ are each independently hydrogen, halogen, nitro, cyano, hydroxyl, optionally substituted alkyl, optionally substituted cycloalkyl, perfluoroalkyl, optionally substituted heteroalkyl, optionally substituted heterocycloalkyl, optionally substituted aryl, optionally substituted heteroaryl;

R⁷ is hydrogen or an optionally substituted alkyl;

R⁸ is each independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, or NR_{A}R_{B};

or a pharmaceutically acceptable salt or solvate thereof.

In one embodiment, is a compound having the structure of Formula (III) wherein A is an optionally substituted heteroaryl. In another embodiment, A is an optionally substituted heteroaryl group. In another embodiment, the heteroaryl group consists of one, two, three, or four heteroatoms selected from N, S, and O. In one embodiment, is a compound having the structure of Formula (III) wherein the heteroaryl group is selected from the group consisting of pyridine, pyrimidine, pyrazine, pyrazole, oxazole, thiazole, isoxazole, isothiazole, 1,3,4-oxadiazole, pyridazine, 1,3,5-trazine, 1,2,4-triazine, quinoxaline, benzimidazole, benzotriazole, purine, 1H-[1,2,3]triazolo[4,5-d]pyrimidine, triazole, imidazole, thiophene, furan, isobenzofuran, pyrrole, indolizine, isoindole, indole, indazole, isoquinoline, quinoline, phthalazine, naphthyridine, quinazoline, cinnoline, and pteridine. In another embodiment, the heteroaryl group is selected from pyridine, imidazole, benzimidazole, pyrrole, pyrazole, pyrimidine, pyrazine, and pyridazine. In a further embodiment, the heteroaryl group is pyridine. In another embodiment, the heteroaryl group is benzimidazole. In yet another embodiment, the heteroaryl group is imidazole. In yet another embodiment, the heteroaryl group is pyrazine.

In one embodiment, is a compound of Formula (III) wherein A is an optionally substituted heteroaryl attached to L at a heteroatom of the heteroaryl group. By way of example only, A is an optionally substituted benzoimidazole group,

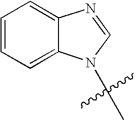

wherein the benzoimidazole group is attached to L at a nitrogen atom,

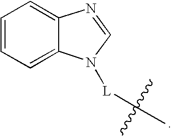

In one embodiment, L is a direct bond such that the benzoimidazole group is attached directly to the steroid scaffold,

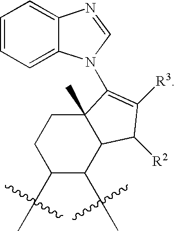

In another embodiment, is a compound of Formula (III) wherein A is an optionally substituted heteroaryl attached to L at a carbon atom of the heteroaryl group. Also by way of example only, A is an optionally substituted pyridine group,

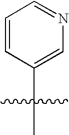

wherein the pyridine group is attached to L at a carbon atom,

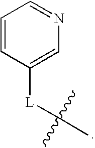

In one embodiment, L is a direct bond such that the pyridine group is attached directly to the steroid scaffold,

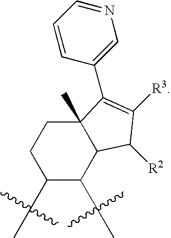

In another embodiment is a compound having the structure of Formula (III) wherein A is an optionally substituted heterocycloalkyl. In one embodiment, the heterocycloalkyl group is selected from the group consisting of pyrrolidine, imidazolidine, piperidine, piperazine, pyrazolidine, tetrahydrofuran, tetrahydrothiophene, 1,3-oxathiolane, indoline, isoindoline, morpholine, and pyrazoline.

In one embodiment, L is a direct bond. In one embodiment, L is

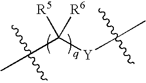

wherein Y is a direct bond, O, C═O, C(O)O, S(O)ᵤ, NR¹, or NR⁷C(O); q is an integer from 0 to 4; u is an integer from 0 to 2; R⁵ and R⁶ are each independently hydrogen, halogen, nitro, cyano, hydroxyl, optionally substituted alkyl, optionally substituted cycloalkyl, perfluoroalkyl, optionally substituted heteroalkyl, optionally substituted heterocycloalkyl, optionally substituted aryl, optionally substituted heteroaryl; and R⁷ is hydrogen or an optionally substituted alkyl. In another embodiment, L is

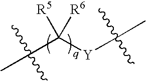

wherein Y is a direct bond and q is 0. In yet another embodiment, Y is a direct bond; q is 1-4; and R⁵ and R⁶ are both hydrogen. In a further embodiment, L is —CH₂—. In another embodiment, L is —CH₂ CH₂—. In another embodiment, Y is —O— and q is 0-4. In another embodiment, L is

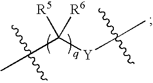

Y is C═O; and q is 0. In yet another embodiment, Y is C═O, C(O)O, NR¹ or NR⁷C(O). In yet another embodiment, Y is NH. In another embodiment, Y is —N(C₁-C₆alkyl)-.

In one embodiment, is a compound having the structure of Formula (III), wherein R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy. In another embodiment, R² is selected from a group consisting of hydrogen, optionally substituted C₁-C₆ alkyl; optionally substituted C₁-C₈ cycloalkyl, cyano, halogen, or nitro. In a further embodiment, R² is hydrogen or C₁-C₆ alkyl. In yet a further embodiment, R² is hydrogen.

In one embodiment, is a compound having the structure of Formula (III), wherein R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl. In another embodiment, R³ is R_{A}carbonyl. In another embodiment, R³ is R_{A}carbonyl wherein R_{A} is hydrogen or C₁-C₆ alkyl. In another embodiment, R³ is CHO. In another embodiment, R³ is selected from a group consisting of hydrogen, optionally substituted C₁-C₆ alkyl; optionally substituted C₁-C₈ cycloalkyl, cyano, halogen, or nitro. In a further embodiment, R³ is hydrogen or C₁-C₆ alkyl. In yet a further embodiment, R³ is hydrogen.

In one embodiment, is a compound having the structure of Formula (III) wherein R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or

R_{A} and R_{B} taken together with the nitrogen atom form an optionally substituted 4 to 7 membered heterocyclic ring having one or two heteroatoms. In one embodiment, R¹ is selected from the group consisting of hydrogen, optionally substituted alkyl or optionally substituted cycloalkyl. In another embodiment, R¹ is hydrogen or C₁-C₆ alkyl. In a further embodiment, R¹ is hydrogen. In yet a further embodiment, R¹ is —CH₃.

In another embodiment, is a compound having the structure of Formula (III) wherein R⁴ is selected from the group consisting of hydrogen, optionally substituted alkyl, optionally substituted cycloalkyl, optionally substituted haloalkyl, optionally substituted hydroxyalkyl, R_{A}carbonyl, (NR_{A}R_{B})alkyl, and (NR_{A}R_{B})carbonyl. In another embodiment, R⁴ is selected from a group consisting of hydrogen, optionally substituted alkyl, optionally substituted cycloalkyl and R_{A}carbonyl. In one embodiment, R⁴ is hydrogen. In another embodiment, R⁴ is R_{A}carbonyl wherein R_{A} is selected from a group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl. In a further embodiment, R_{A} is C₁-C₆ alkyl or hydrogen. In a further embodiment, R_{A} is —CH₃.

In one embodiment, is a compound having the structure of Formula (IC), Formula (IIC), or Formula (IIIC):

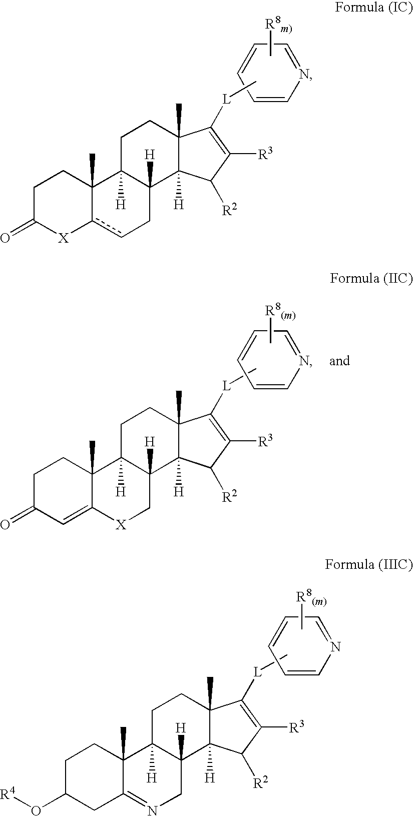

wherein:

L is a direct bond or

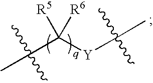

Y is a direct bond, O, C═O, C(O)O, S(O)ᵤ, NR¹, or NR⁷C(O);

q is an integer from 0 to 4;

u is an integer from 0 to 2;

[symbol] is a single or double bond;

R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or

R_{A} and R_{B} taken together with the nitrogen atom to which they are attached form an optionally substituted 4 to 7 membered heterocyclic ring having one or two heteroatoms;

R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy;

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl;

R⁴ is selected from the group consisting of hydrogen, optionally substituted alkyl, optionally substituted cycloalkyl, optionally substituted haloalkyl, optionally substituted hydroxyalkyl, COR_{A}, (NR_{A}R_{B})alkyl, and (NR_{A}R_{B})carbonyl;

R⁵ and R⁶ are each independently hydrogen, halogen, nitro, cyano, hydroxyl, optionally substituted alkyl, optionally substituted cycloalkyl, perfluoroalkyl, optionally substituted heteroalkyl, optionally substituted heterocycloalkyl, optionally substituted aryl, optionally substituted heteroaryl;

R⁷ is hydrogen or an optionally substituted alkyl;

R⁸ is each independently selected from the group consisting of hydrogen, halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, or NR_{A}R_{B}; and m is an integer from 1-4; or a pharmaceutically acceptable salt or solvate thereof.

In another embodiment is a compound having the structure of Formula (IC), (IIC), or (IIIC) wherein L is a direct bond such that the pyridine group is directly attached to the steroid. In a further embodiment, L is attached to the optionally substituted pyridine group at the 1, 2, or 3-position. In another embodiment, L is attached to the optionally substituted pyridine group at the 3-position. In yet another embodiment, L is a bond such that the steroid is directly attached to the optionally ring at the 3-position.

In one embodiment, is a compound having the structure of Formula (IC), (IIC), or (IIIC), wherein R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy. In another embodiment, R² is selected from a group consisting of hydrogen, optionally substituted C₁-C₆ alkyl; optionally substituted C₁-C₈ cycloalkyl, cyano, halogen, or nitro. In a further embodiment, R² is hydrogen or C₁-C₆ alkyl. In yet a further embodiment, R² is hydrogen.

In one embodiment, is a compound having the structure of Formula (IC), (IIC), or (IIIC), wherein R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl. In another embodiment, R³ is R_{A}carbonyl. In another embodiment, R³ is R_{A}carbonyl wherein R_{A} is hydrogen or C₁-C₆ alkyl. In another embodiment, R³ is CHO. In another embodiment, R³ is selected from a group consisting of hydrogen, optionally substituted C₁-C₆ alkyl; optionally substituted C₁-C₈ cycloalkyl, cyano, halogen, or nitro. In a further embodiment, R³ is hydrogen or C₁-C₆ alkyl. In yet a further embodiment, R³ is hydrogen.

In one embodiment, is a compound having the structure of Formula (IC), (IIC), or (IIIC), wherein R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or

R_{A} and R_{B} taken together with the nitrogen atom to which they are attached form an optionally substituted 4 to 7 membered heterocyclic ring having one or two heteroatoms. In one embodiment, R¹ is selected from the group consisting of hydrogen, optionally substituted alkyl or optionally substituted cycloalkyl. In another embodiment, R¹ is hydrogen or C₁-C₆ alkyl. In a further embodiment, R¹ is hydrogen. In yet a further embodiment, R¹ is —CH₃.

In another embodiment, is a compound having the structure of Formula (IIIC) wherein R⁴ is selected from the group consisting of hydrogen, optionally substituted alkyl, optionally substituted cycloalkyl, optionally substituted haloalkyl, optionally substituted hydroxyalkyl, R_{A}carbonyl, (NR_{A}R_{B})alkyl, and (NR_{A}R_{B})carbonyl. In another embodiment, R⁴ is selected from a group consisting of hydrogen, optionally substituted alkyl, optionally substituted cycloalkyl and R_{A}carbonyl. In one embodiment, R⁴ is hydrogen. In another embodiment, R⁴ is R_{A}carbonyl wherein R_{A} is selected from a group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl. In a further embodiment, R_{A} is C₁-C₆ alkyl or hydrogen. In a further embodiment, R_{A} is —CH₃.

In one embodiment is a compound having the structure of Formula (IC), (IIC), or (IIIC), wherein R⁸ is each independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, or NR_{A}R_{B}; and m is an integer from 1-4. In one embodiment, each R⁸ is independently hydrogen. In another embodiment, at least one R⁸ is halogen. In another embodiment, at least one R⁸ is selected from Cl, Br, or F. In a further embodiment, at least one R⁸ is C₁-C₆ alkoxy. In a further embodiment, at least one R⁸ is C₁-C₆ alkyl. In another embodiment, m is an integer from 1-4.

In one embodiment, is a compound having the structure of Formula (ID), Formula (IID), or Formula (IIID):

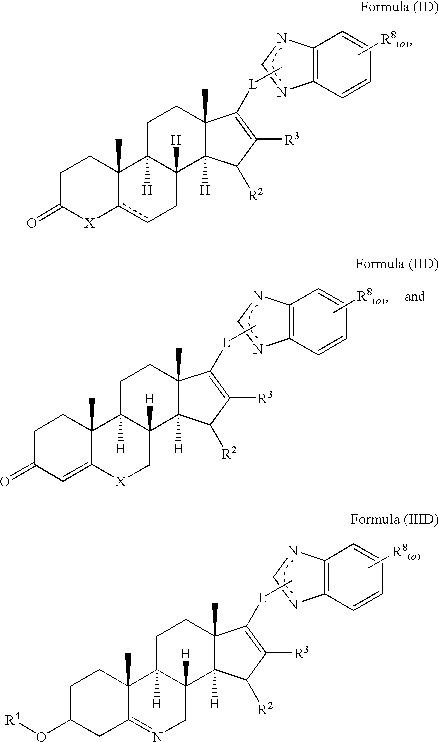

wherein:

L is a direct bond or

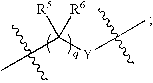

Y is a direct bond, O, C═O, C(O)O, S(O)ᵤ, NR¹, or NR⁷C(O);

q is an integer from 0 to 4;

u is an integer from 0 to 2;

[symbol] is a single or double bond;

R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or

R_{A} and R_{B} taken together with the nitrogen atom to which they are attached form an optionally substituted 4 to 7 membered heterocyclic ring having one or two heteroatoms;

R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy;

R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl;

R⁴ is selected from the group consisting of hydrogen, optionally substituted alkyl, optionally substituted cycloalkyl, optionally substituted haloalkyl, optionally substituted hydroxyalkyl, R_{A}carbonyl, (NR_{A}R_{B})alkyl, and (NR_{A}R_{B})carbonyl;

R⁵ and R⁶ are each independently hydrogen, halogen, nitro, cyano, hydroxyl, optionally substituted alkyl, optionally substituted cycloalkyl, perfluoroalkyl, optionally substituted heteroalkyl, optionally substituted heterocycloalkyl, optionally substituted aryl, optionally substituted heteroaryl;

R⁷ is hydrogen or an optionally substituted alkyl;

R⁸ is each independently selected from hydrogen, halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, or NR_{A}R_{B}; and o is an integer from 1-5; or a pharmaceutically acceptable salt or solvate thereof.

In another embodiment is a compound having the structure of Formula (ID), (IID), or (IIID) wherein L is a direct bond such that the benzimidazole group is directly attached to the steroid. In a further embodiment, L is attached to the optionally substituted benzimidazole group at a nitrogen atom of the benzimidazole group. In another embodiment, L is attached to the optionally substituted benzimidazole group at a nitrogen atom. In yet another embodiment, L is a bond such that the steroid is attached to the optionally substituted benzimidazole at a nitrogen atom.

In one embodiment, is a compound having the structure of Formula (ID), (IID), or (IIID), wherein R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy. In another embodiment, R² is selected from a group consisting of hydrogen, optionally substituted C₁-C₆ alkyl; optionally substituted C₁-C₈ cycloalkyl, cyano, halogen, or nitro. In a further embodiment, R² is hydrogen or C₁-C₆ alkyl. In yet a further embodiment, R² is hydrogen.

In one embodiment, is a compound having the structure of Formula (ID), (IID), or (IIID), wherein R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl. In another embodiment, R³ is R_{A}carbonyl. In another embodiment, R³ is R_{A}carbonyl wherein R_{A} is hydrogen or C₁-C₆ alkyl. In another embodiment, R³ is CHO. In another embodiment, R³ is selected from a group consisting of hydrogen, optionally substituted C₁-C₆ alkyl; optionally substituted C₁-C₈ cycloalkyl, cyano, halogen, or nitro. In a further embodiment, R³ is hydrogen or C₁-C₆ alkyl. In yet a further embodiment, R³ is hydrogen.

In one embodiment, is a compound having the structure of Formula (ID), (IID), or (IIID), wherein R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;

R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or

R_{A} and R_{B} taken together with the nitrogen atom form an optionally substituted 4 to 7 membered heterocyclic ring having one or two heteroatoms. In one embodiment, R¹ is selected from the group consisting of hydrogen, optionally substituted alkyl or optionally substituted cycloalkyl. In another embodiment, R¹ is hydrogen or C₁-C₆ alkyl. In a further embodiment, R¹ is hydrogen. In yet a further embodiment, R¹ is —CH₃.

In another embodiment, is a compound having the structure of Formula (IIID) wherein R⁴ is selected from the group consisting of hydrogen, optionally substituted alkyl, optionally substituted cycloalkyl, optionally substituted haloalkyl, optionally substituted hydroxyalkyl, R_{A}carbonyl, (NR_{A}R_{B})alkyl, and (NR_{A}R_{B})carbonyl. In another embodiment, R⁴ is selected from a group consisting of hydrogen, optionally substituted alkyl, optionally substituted cycloalkyl and R_{A}carbonyl. In one embodiment, R⁴ is hydrogen. In another embodiment, R⁴ is R_{A}carbonyl wherein R_{A} is selected from a group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl. In a further embodiment, R_{A} is C₁-C₆ alkyl or hydrogen. In a further embodiment, R_{A} is —CH₃.

In one embodiment is a compound having the structure of Formula (ID), (IID), or (IIID), wherein R⁸ is each independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B} carbonyl, or NR_{A}R_{B}; and o is an integer from 1-5. In one embodiment, each R⁸ is independently hydrogen. In another embodiment, at least one R⁸ is halogen. In another embodiment, at least one R⁸ is selected from Cl, Br, or F. In a further embodiment, at least one R⁸ is C₁-C₆ alkoxy. In a further embodiment, at least one R⁸ is C₁-C₆ alkyl. In another embodiment, o is an integer from 1-5.

Also described herein is a compound selected from the group consisting of:
(4aR,4bS,6aS,9aS,9bS)-1,4a,6a-trimethyl-7-(pyridin-3-yl)-4,4a,4b,5,6,6a,9,9a,9b,10-decahydro-1H-indeno[5,4-f]quinolin-2(3H)-one,
(4aR,4bS,6aS,9aS)-4a,6a-dimethyl-7-(pyridin-3-yl)-4,4a,4b,5,6,6a,9,9a,9b,10-decahydro-1H-indeno[5,4-f]quinolin-2(3H)-one,
(3aS,3bS,9aR,9bS,11aS)-5,9a,11a-trimethyl-1-(pyridin-3-yl)-3b,4,5,8,9,9a,9b,10,11,11a-dodecahydro-3H-cyclopenta[i]phenanthridin-7(3 aH)-one,
(3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3 aH)-one,
(4aR,4bS,6aS,9aS,9bS)-7-(1H-benzo[d]imidazol-2-yl)-1,4a,6a-trimethyl-4,4a,5,6,6a,9,9a,9b,10-decahydro-1H-indeno[5,4-f]quinolin-2(3H)-one,
(4aR,4bS,6aS,9aS,9bR)-7-(1H-benzo[d]imidazol-1-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-7-(6-methoxypyridin-3-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-7-(5-methoxypyridin-3-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-1,4a,6a-trimethyl-7-(4-methylpyridin-3-yl)-4,4a,4b,5,6,6a,9,9a,9 b,10-decahydro-1H-indeno[5,4-f]quinolin-2-one,
(4aR,4bS,6aS,9aS,9bS)-1,4a,6a-trimethyl-7-(pyrimidin-5-yl)-4,4a,4b,5,6,6a,9,9a,9b,10-decahydro-1H-indeno[5,4-f]quinolin-2(3H)-one,
(4aR,4bS,6aS,9aS,9bS)-1,4a,6a-trimethyl-7-(pyrazin-2-yl)-4,4a,4b,5,6,6a,9,9a,9b,10-decahydro-1H-indeno[5,4-f]quinolin-2(3H)-one,
(4aR,4bS,6aS,9aS,9bS)-1,4a,6a-trimethyl-7-(quinolin-3-yl)-4,4a,4b,5,6,6a,9,9a,9b,10-decahydro-1H-indeno[5,4-f]quinolin-2(3H)-one,
(4aR,4bS,6aS,9aS,9bS)-7-(5-chloropyridin-3-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-7-(4-chloropyridin-3-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-1-ethyl-4a,6a-dimethyl-7-(pyridin-3-yl)-4,4a,4b,5,6,6a,9,9a,9b,10-decahydro-1H-indeno[5,4-f]quinolin-2(3H)-one,
(4aR,4bS,6aS,9aS,9bS)-1-ethyl-4a,6a-dimethyl-7-(pyrimidin-5-yl)-4,4a,4b,5,6,6a,9,9a,9b,10-decahydro-1H-indeno[5,4-f]quinolin-2(3H)-one,
(4aR,4bS,6aS,9aS,9bS)-1-ethyl-4a,6a-dimethyl-7-(pyrazin-2-yl)-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-4a,6a-dimethyl-7-(pyridin-3-yl)-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-4a,6a-dimethyl-7-(pyrimidin-5-yl)-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-4a,6a-dimethyl-7-(pyrazin-2-yl)-4,4a,4b,5,6,6a,9,9a,9b,10-decahydro-1H-indeno[5,4-f]quinolin-2(3H)-one,
(4aR,4bS,6aS,9aS,9bS)-7-(5-chloropyridin-3-yl)-1-ethyl-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-ethyl-7-(6-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-ethyl-7-(4-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-7-(5-chloropyridin-3-yl)-1-cyclopropyl-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(6-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-7-(5-chloropyridin-3-yl)-4a,6a-dimethyl-1-propyl-4,4a,4b,5,6,6a,9,9a,9b,10-decahydro-1H-indeno[5,4-f]quinolin-2(3H)-one,
(4aR,4bS,6aS,9aS,9bS)-4a,6a-dimethyl-7-(pyridin-3-yl)-4,4a,4b,5,6,6a,9,9a,9b,10,11,11a-dodecahydroindeno[5,4-f]chromen-2(3H)-one,
(4aR,4bS,6aS,9aS,9bR)-4a,6a-dimethyl-7-(pyridin-3-yl)-4,4a,4b,5,6,6a,9,9a,9b,10-decahydroindeno[5,4-f]chromen-2(3H)-one,
(3aS,3bS,9aR,9bS,11aS)-9a,11a-dimethyl-1-(pyridin-3-yl)-3a,3b,4,6,7,8,9,9a,9b,10,11,11a-dodecahydro-3H-cyclopenta[i]phenanthridin-7-ol,
(3aS,3bS,9aR,9bS,11aS)-9a,11a-dimethyl-1-(pyridin-3-yl)-3a,3b,4,6,7,8,9,9a,9b,10,11,11a-dodecahydro-3H-cyclopenta[i]phenanthridin-7-yl acetate,
(3aS,3bS,9aR,9bS,11aS)-1-(5-methoxypyridin-3-yl)-9a,11a-dimethyl-3a,3b,4,6,7,8,9,9a,9b,10,11,11a-dodecahydro-3H-cyclopenta[i]phenanthridin-7-ol,
(3aS,3bS,9aR,9bS,11aS)-1-(5-methoxypyridin-3-yl)-9a,11a-dimethyl-3a,3b,4,6,7,8,9,9a,9b,10,11,11a-dodecahydro-3H-cyclopenta[i]phenanthridin-7-yl acetate,
(3aS,3bS,9aR,9bS,11aS)-9a,11a-dimethyl-1-(pyridin-3-yl)-3,3a,3b,4,9,9a,9b,10,11,11a-decahydroindeno[4,5-c]chromen-7(8H)-one,
(3aS,3bS,9aR,9bS,11aS)-1-(5-methoxypyridin-3-yl)-9a,11a-dimethyl-3,3a,3b,4,9,9a,9b,10,11,11a-decahydroindeno[4,5-c]chromen-7(8H)-one,
(3aS,3bS,9aR,9bS,11aS)-1-(4-methoxypyridin-3-yl)-9a,11a-dimethyl-3,3a,3b,4,9,9a,9b,10,11,11a-decahydroindeno[4,5-c]chromen-7(8H)-one,
4aR,4bS,6aS,9aS,9bS)-4a,6a-dimethyl-7-(pyrazin-2-yl)-4,4a,4b,5,6,6a,9,9a,9b,10,11,11a-dodecahydroindeno[5,4-f]chromen-2(3H)-one,
(4aR,4bS,6aS,9aS,9bS)-7-(6-methoxypyrazin-2-yl)-4a,6a-dimethyl-4,4a,4b,5,6,6a,9,9a,9b,10,11,11a-dodecahydroindeno[5,4-f]chromen-2(3H)-one,
(4aR,4bS,6aS,9aS,9bS)-4a,6a-dimethyl-7-(pyrazin-2-yl)-4,4a,4b,5,6,6a,9,9a,9b,10-decahydroindeno[5,4-f]chromen-2(3H)-one,
(4aR,4bS,6aS,9aS,9bS)-7-(6-methoxypyrazin-2-yl)-4a,6a-dimethyl-4,4a,4b,5,6,6a,9,9a,9b,10-decahydroindeno[5,4-f]chromen-2(3H)-one,
(4aR,4bS,6aS,9aS,9bS)-4a,6a-dimethyl-7-(pyridin-3-yl)-4,4a,4b,5,6,6a,9,9a,9b,10,11,11a-dodecahydro-1H-indeno[5,4-f]quinolin-2(3H)-one,
(4aR,4bS,6aS,9aS,9bS)-7-(5-methoxypyridin-3-yl)-4a,6a-dimethyl-4,4a,4b,5,6,6a,9,9a,9b,10,11,11a-dodecahydro-1H-indeno[5,4-f]quinolin-2(3H)-one,
(4aR,4bS,6aS,9aS,9bS)-7-(4-methoxypyridin-3-yl)-4a,6a-dimethyl-4,4a,4b,5,6,6a,9,9a,9b,10,11,11a-dodecahydro-1H-indeno[5,4-f]quinolin-2(3H)-one,
(4aR,4bS,6aS,9aS,9bS)-7-(5-fluoropyridin-3-yl)-1,4a,6a-trimethyl-1,3,4,4a,4b,5,6,6a,9,9a,9b,10-dodecahydro-indeno[5,4-f]quinolin-2(3H)-one,
(4aR,4bS,6aS,9aS,9bS)-7-(5-methylpyridin-3-yl)-1,4a,6a-trimethyl-1,3,4,4a,4b,5,6,6a,9,9a,9b,10-dodecahydro-indeno[5,4-f]quinolin-2(3H)-one,
(4aR,4bS,6aS,9aS,9bS)-7-(4-methoxy-pyridin-3-yl)-1,4a,6a-trimethyl-1,3,4,4a,4b,5,6,6a,9,9a,9b,10-dodecahydro-indeno[5,4-f]quinolin-2(3H)-one,
(4aR,4bS,6aS,9aS,9bS)-7-(5-Ethoxy-pyridin-3-yl)-1,4a,6a-trimethyl-1,3,4,4a,4b,5,6,6a,9,9a,9b,10-dodecahydro-indeno[5,4-f]quinolin-2(3H)-one,
(4aR,4bS,6aS,9aS,9bS)-1-ethyl-7-(5-methoxypyridin-3-yl)-4a,6a-dimethyl 4,4a,4b,5,6,6a,9,9a,9b,10-decahydro-1H-indeno[5,4-f]quinolin-2(3H)-one,
(4aR,4bS,6aS,9aS,9bS)-1-ethyl-7-(5-ethoxypyridin-3-yl)-4a,6a-dimethyl 4,4a,4b,5,6,6a,9,9a,9b,10-decahydro-1H-indeno[5,4-f]quinolin-2(3H)-one,
(4aR,4bS,6aS,9aS,9bS)-1-ethyl-7-(5-fluoropyridin-3-yl)-4a,6a-dimethyl 4,4a,4b,5,6,6a,9,9a,9b,10-decahydro-1H-indeno[5,4-f]quinolin-2(3H)-one,
(4aR,4bS,6aS,9aS,9bS)-1-ethyl-7-(5-methylpyridin-3-yl)-4a,6a-dimethyl 4,4a,4b,5,6,6a,9,9a,9b,10-decahydro-1H-indeno[5,4-f]quinolin-2(3H)-one,
(4aR,4bS,6aS,9aS,9bS)-1-ethyl-7-(5-chloropyridin-3-yl)-4a,6a-dimethyl 4,4a,4b,5,6,6a,9,9a,9b,10-decahydro-1H-indeno[5,4-f]quinolin-2(3H)-one,
4aR,4bS,6aS,9aS,9bS)-1-ethyl-7-(5-methylpyridin-3-yl)-4a,6a-dimethyl 4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(5-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(5-ethoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(5-fluoropyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(5-methylpyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(4-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(4-chloropyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(4-methylpyridin-3-yl)-4a,6a-dimethyl-4,4a,4b,5,6,6a,9,9a,9b,10-decahydro-1H-indeno[5,4-f]quinolin-2(3H)-one,
(3aS,3bS,9aR,9bS,11aS)-1-(5-methoxypyridin-3-yl)-5,9a,11a-trimethyl-3b,4,5,8,9,9a,9b,10,11,11a-decahydro-3H-cyclopenta[i]phenanthridin-7(3aH)-one,
(3aS,3bS,9aR,9bS,11aS)-1-(5-ethoxypyridin-3-yl)-5,9a,11a-trimethyl-3b,4,5,8,9,9a,9b,10,11,11a-decahydro-3H-cyclopenta[i]phenanthridin-7(3aH)-one,
(3aS,3bS,9aR,9bS,11aS)-1-(5-fluoropyridin-3-yl)-5,9a,11a-trimethyl-3b,4,5,8,9,9a,9b,10,11,11a-decahydro-3H-cyclopenta[i]phenanthridin-7(3 aH)-one,
(3aS,3bS,9aR,9bS,11aS)-1-(5-chloropyridin-3-yl)-5,9a,11a-trimethyl-3b,4,5,8,9,9a,9b,10,11,11a-decahydro-3H-cyclopenta[i]phenanthridin-7(3 aH)-one,
(3aS,3bS,9aR,9bS,11aS)-1-(5-methylpyridin-3-yl)-5,9a,11a-trimethyl-3b,4,5,8,9,9a,9b,10,11,11a-decahydro-3H-cyclopenta[i]phenanthridin-7(3aH)-one,
(3aS,3bS,9aR,9bS,11aS)-1-(4-methoxypyridin-3-yl)-5,9a,11a-trimethyl-3b,4,5,8,9,9a,9b,10,11,11a-decahydro-3H-cyclopenta[i]phenanthridin-7(3aH)-one,
(3aS,3bS,9aR,9bS,11aS)-1-(4-chloropyridin-3-yl)-5,9a,11a-trimethyl-3b,4,5,8,9,9a,9b,10,11,11a-decahydro-3H-cyclopenta[i]phenanthridin-7(3 aH)-one,
(3aS,3bS,9aR,9bS,11aS)-1-(4-methylpyridin-3-yl)-5,9a,11a-trimethyl-3b,4,5,8,9,9a,9b,10,11,11a-decahydro-3H-cyclopenta[i]phenanthridin-7(3aH)-one,
(3aS,3bS,9aR,9bS,11aS)-1-(5-methoxypyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-1-(5-ethoxypyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-1-(5-fluoropyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-1-(5-chloropyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-1-(5-methylpyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-1-(4-methoxypyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-1-(4-chloropyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,1aS)-1-(4-methylpyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-5-ethyl-9a,11a-dimethyl-1-(pyridin-3-yl)-3b,4,5,8,9,9a,9b,10,11,11a-decahydro-3H-cyclopenta[i]phenanthridin-7(3 aH)-one,
(3aS,3bS,9aR,9bS,11aS)-5-ethyl-1-(5-methoxypyridin-3-yl)-9a,11a-dimethyl-3b,4,5,8,9,9a,9b,10,11,11a-decahydro-3H-cyclopenta[i]phenanthridin-7(3aH)-one,
(3aS,3bS,9aR,9bS,11aS)-5-ethyl-1-(5-ethoxypyridin-3-yl)-9a,11a-dimethyl-3b,4,5,8,9,9a,9b,10,11,11a-decahydro-3H-cyclopenta[i]phenanthridin-7(3aH)-one,
(3aS,3bS,9aR,9bS,11aS)-5-ethyl-1-(5-fluoropyridin-3-yl)-9a,11a-dimethyl-3b,4,5,8,9,9a,9b,10,11,11a-decahydro-3H-cyclopenta[i]phenanthridin-7(3aH)-one,
(3aS,3bS,9aR,9bS,11aS)-5-ethyl-1-(5-chloropyridin-3-yl)-9a,11a-dimethyl-3b,4,5,8,9,9a,9b,10,11,11a-decahydro-3H-cyclopenta[i]phenanthridin-7(3aH)-one,
(3aS,3bS,9aR,9bS,11aS)-5-ethyl-1-(5-methylpyridin-3-yl)-9a,11a-dimethyl-3b,4,5,8,9,9a,9b,10,11,11a-decahydro-3H-cyclopenta[i]phenanthridin-7(3aH)-one,
(3aS,3bS,9aR,9bS,11aS)-5-ethyl-1-(4-methoxypyridin-3-yl)-9a,11a-dimethyl-3b,4,5,8,9,9a,9b,10,11,11a-decahydro-3H-cyclopenta[i]phenanthridin-7(3aH)-one,
(3aS,3bS,9aR,9bS,11aS)-5-ethyl-1-(4-chloropyridin-3-yl)-9a,11a-dimethyl-3b,4,5,8,9,9a,9b,10,11,11a-decahydro-3H-cyclopenta[i]phenanthridin-7(3aH)-one,
(3aS,3bS,9aR,9bS,11aS)-5-ethyl-1-(4-methylpyridin-3-yl)-9a,11a-dimethyl-3b,4,5,8,9,9a,9b,10,11,11a-decahydro-3H-cyclopenta[i]phenanthridin-7(3aH)-one,
(4aR,4bS,6aS,9aS,9bS)-7-(6-methoxypyrazin-2-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-1,4a,6a-trimethyl-7-(6-methylpyrazin-2-yl)-4,4a,4b,5,6,6a,9,9a,9b,10-decahydro-1H-indeno[5,4-f]quinolin-2(3H)-one,
(4aR,4bS,6aS,9aS,9bS)-7-(6-ethylpyrazin-2-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-7-(6-ethoxypyrazin-2-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-1-ethyl-7-(6-methoxypyrazin-2-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-ethyl-7-(6-methoxypyrazin-2-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-ethyl-7-(6-ethylpyrazin-2-yl)-4a,6a-dimethyl 4,4a,4b,5,6,6a,9,9a,9b,10-decahydro-1H-indeno[5,4-f]quinolin-2(3H)-one,
(4aR,4bS,6aS,9aS,9bS)-1-ethyl-7-(6-ethoxylpyrazin-2-yl)-4a,6a-dimethyl 4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(6-methoxypyrazin-2-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(6-methylpyrazin-2-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(6-ethylpyrazin-2-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(6-ethoxypyrazin-2-yl)-4a,6a-dimethyl-4,4a,4b,5,6,6a,9,9a,9b,10-decahydro-1H-indeno[5,4-f]quinolin-2(3H)-one,
(3aS,3bS,9aR,9bS,11aS)-5,9a,11a-trimethyl-1-(pyrazin-2-yl)-3b,4,5,8,9,9a,9b,10,11,11a-decahydro-3H-cyclopenta[i]phenanthridin-7(3 aH)-one,
(3aS,3bS,9aR,9bS,11aS)-1-(6-methoxypyrazin-2-yl)-5,9a,11a-trimethyl-3b,4,5,8,9,9a,9b,10,11,11a-decahydro-3H-cyclopenta[i]phenanthridin-7(3aH)-one,
(3aS,3bS,9aR,9bS,11aS)-1-(6-ethoxypyrazin-2-yl)-5,9a,11a-trimethyl-3b,4,5,8,9,9a,9b,10,11,11a-decahydro-3H-cyclopenta[i]phenanthridin-7(3aH)-one,
(3aS,3bS,9aR,9bS,11aS)-1-(6-ethylpyrazin-2-yl)-5,9a,11a-trimethyl-3b,4,5,8,9,9a,9b,10,11,11a-decahydro-3H-cyclopenta[i]phenanthridin-7(3 aH)-one,
(4aR,4bS,6aS,9aS,9bS)-7-(5-methoxypyridin-3-yl)-4a,6a-dimethyl-4,4a,4b,5,6,6a,9,9a,9b,10,11,11a-dodecahydroindeno[5,4-f]chromen-2(3H)-one,
(4aR,4bS,6aS,9aS,9bS)-7-(5-ethoxypyridin-3-yl)-4a,6a-dimethyl-4,4a,4b,5,6,6a,9,9a,9b,10,11,11a-dodecahydroindeno[5,4-f]chromen-2(3H)-one,
(4aR,4bS,6aS,9aS,9bS)-7-(4-methoxypyridin-3-yl)-4a,6a-dimethyl-4,4a,4b,5,6,6a,9,9a,9b,10,11,11a-dodecahydroindeno[5,4-f]chromen-2(3H)-one,
(4aR,4bS,6aS,9aS,9bS)-7-(5-methoxypyridin-3-yl)-4a,6a-dimethyl-4,4a,4b,5,6,6a,9,9a,9b,10-decahydroindeno[5,4-f]chromen-2(3H)-one,
(4aR,4bS,6aS,9aS,9bS)-7-(5-ethoxypyridin-3-yl)-4a,6a-dimethyl-4,4a,4b,5,6,6a,9,9a,9b,10-decahydroindeno[5,4-f]chromen-2(3H)-one,
(4aR,4bS,6aS,9aS,9bS)-7-(4-methoxypyridin-3-yl)-4a,6a-dimethyl-4,4a,4b,5,6,6a,9,9a,9b,10-decahydroindeno[5,4-f]chromen-2(3H)-one,
(4aR,4bS,6aS,9aS,9bS)-7-(4-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-7-(5-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-7-(6-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-4a,6a-dimethyl-7-(pyrazin-2-yl)-4,4a,4b,5,6,6a,9,9a,9b,10-decahydro-1H-indeno[5,4-f]quinolin-2(3H)-one,
(4aR,4bS,6aS,9aS,9bS)-7-(6-methoxypyrazin-2-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-4a,6a-dimethyl-7-(pyrimidin-5-yl)-4,4a,4b,5,6,6a,9,9a,9b,10-decahydro-1H-indeno[5,4-f]quinolin-2(3H)-one,
(3aS,3bS,9aR,9bS,11aS)-1-iodo-9a,11a-dimethyl-3b,4,5,8,9,9a,9b,10,11,11a-decahydro-3H-cyclopenta[i]phenanthridin-7(3 aH)-one,
(3aS,3bS,9aR,9bS,111aS)-9a,11a-dimethyl-1-(pyrazin-2-yl)-3b,4,5,8,9,9a,9b,10,11,11a-decahydro-3H-cyclopenta[i]phenanthridin-7(3 aH)-one,
(3aS,3bS,9aR,9bS,11aS)-1-(6-methoxypyrazin-2-yl)-9a,11a-dimethyl-3b,4,5,8,9,9a,9b,10,11,11a-decahydro-3H-cyclopenta[i]phenanthridin-7(3 aH)-one,
(4aR,4bS,6aS,9aS,9bS)-4a,6a-dimethyl-7-(oxazol-5-yl)-4,4a,4b,5,6,6a,9,9a,9b,10-decahydro-1H-indeno[5,4-f]quinolin-2(3H)-one,
(4aR,4bS,6aS,9aS,9bS)-4a,6a-dimethyl-7-(thiazol-5-yl)-4,4a,4b,5,6,6a,9,9a,9b,10-decahydro-1H-indeno[5,4-f]quinolin-2(3H)-one,
(4aR,4bS,6aS,9aS,9bS)-7-(isoxazol-4-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1,4a,6a-trimethyl-7-(oxazol-5-yl)-4,4a,4b,5,6,6a,9,9a,9b,10-decahydro-1H-indeno[5,4-f]quinolin-2(3H)-one,
(4aR,4bS,6aS,9aS,9bS)-1,4a,6a-trimethyl-7-(thiazol-5-yl)-4,4a,4b,5,6,6a,9,9a,9b,10-decahydro-1H-indeno[5,4-f]quinolin-2(3H)-one,
(4aR,4bS,6aS,9aS,9bS)-7-(isoxazol-4-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-4a,6a-dimethyl-7-(oxazol-5-yl)-4,4a,4b,5,6,6a,9,9a,9b,10,11,11a-dodecahydroindeno[5,4-f]chromen-2(3H)-one,
(4aR,4bS,6aS,9aS,9bS)-4a,6a-dimethyl-7-(thiazol-5-yl)-4,4a,4b,5,6,6a,9,9a,9b,10,11,11a-dodecahydroindeno[5,4-f]chromen-2(3H)-one,
(4aR,4bS,6aS,9aS,9bS)-7-(isoxazol-4-yl)-4a,6a-dimethyl-4,4a,4b,5,6,6a,9,9a,9b,10,11,11a-dodecahydroindeno[5,4-f]chromen-2(3H)-one,
(4aR,4bS,6aS,9aS,9bS)-4a,6a-dimethyl-7-(oxazol-5-yl)-4,4a,4b,5,6,6a,9,9a,9b,10-decahydroindeno[5,4-f]chromen-2(3H)-one,
(4aR,4bS,6aS,9aS,9bS)-4a,6a-dimethyl-7-(thiazol-5-yl)-4,4a,4b,5,6,6a,9,9a,9b,10-decahydroindeno[5,4-f]chromen-2(3H)-one,
(4aR,4bS,6aS,9aS,9bS)-7-(isoxazol-4-yl)-4a,6a-dimethyl-4,4a,4b,5,6,6a,9,9a,9b,10-decahydroindeno[5,4-f]chromen-2(3H)-one,
(3aS,3bS,9aR,9bS,11aS)-9a,11a-dimethyl-1-(oxazol-5-yl)-3b,4,5,8,9,9a,9b,10,11,11a-decahydro-3H-cyclopenta[i]phenanthridin-7(3 aH)-one,
(3aS,3bS,9aR,9bS,11aS)-9a,11a-dimethyl-1-(thiazol-5-yl)-3b,4,5,8,9,9a,9b,10,11,11a-decahydro-3H-cyclopenta[i]phenanthridin-7(3 aH)-one,
(3aS,3bS,9aR,9bS,11aS)-1-(isoxazol-4-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-5,9a,11a-trimethyl-1-(oxazol-5-yl)-3b,4,5,8,9,9a,9b,10,11,11a-decahydro-3H-cyclopenta[i]phenanthridin-7(3 aH)-one,
(3aS,3bS,9aR,9bS,11aS)-5,9a,11a-trimethyl-1-(thiazol-5-yl)-3b,4,5,8,9,9a,9b,10,11,11a-decahydro-3H-cyclopenta[i]phenanthridin-7(3 aH)-one,
(3aS,3bS,9aR,9bS,11aS)-1-(isoxazol-4-yl)-5,9a,11a-trimethyl-3b,4,5,8,9,9a,9b,10,11,11a-decahydro-3H-cyclopenta[i]phenanthridin-7(3 aH)-one,
(4aR,4bS,6aS,9aS,9bS)-4a,6a-dimethyl-7-(1H-1,2,3-triazol-4-yl)-4,4a,4b,5,6,6a,9,9a,9b,10,11,11a-dodecahydroindeno[5,4-f]chromen-2(3H)-one,
(4aR,4bS,6aS,9aS,9bS)-4a,6a-dimethyl-7-(1H-1,2,3-triazol-4-yl)-4,4a,4b,5,6,6a,9,9a,9b,10-decahydroindeno[5,4-f]chromen-2(3H)-one,
(3aS,3bS,9aR,9bS,11aS)-5,9a,11a-trimethyl-1-(1H-1,2,3-triazol-4-yl)-3b,4,5,8,9,9a,9b,10,11,11a-decahydro-3H-cyclopenta[i]phenanthridin-7(3aH)-one,
(3aS,3bS,9aR,9bS,11aS)-9a,11a-dimethyl-1-(1H-1,2,3-triazol-4-yl)-3b,4,5,8,9,9a,9b,10,11,11a-decahydro-3H-cyclopenta[i]phenanthridin-7(3 aH)-one,
(4aR,4bS,6aS,9aS,9bS)-1,4a,6a-trimethyl-7-(1H-1,2,3-triazol-4-yl)-4,4a,4b,5,6,6a,9,9a,9b,10-decahydro-1H-indeno[5,4-f]quinolin-2(3H)-one, and
(4aR,4bS,6aS,9aS,9bS)-4a,6a-dimethyl-7-(1H-1,2,3-triazol-4-yl)-4,4a,4b,5,6,6a,9,9a,9b,10-decahydro-1H-indeno[5,4-f]quinolin-2(3H)-one.

Provided herein are pharmaceutical compositions comprising of a compound having the structure of Formula (I), (II), (III), or a pharmaceutically acceptable salt, a pharmaceutically acceptable solvate, pharmaceutically acceptable prodrug thereof in combination with a pharmaceutically acceptable carrier, excipient, binder or diluent.

Also provided herein are methods of treating an androgen-dependent disease in a subject in need of such treatment comprising administering to the subject a therapeutically acceptable amount of a compound having a structure of Formula (I), (II), (III) or a therapeutically acceptable salt or solvate thereof.

In one aspect is a method for treating cancer in a subject comprising administering to a subject in need a therapeutically acceptable amount of a compound having the structure of Formula (I), (II) or (III) or a pharmaceutically acceptable salt or solvate thereof.

In one embodiment is provided methods and compositions for the treatment of CYP17-associated diseases and disorders. Examples include, but are not limited to, sex steroid hormone dependent cancers, such as androgen-dependent prostate cancer, which in some embodiments is treated by inhibiting CYP17-mediated androgen synthesis, and estrogen-dependent breast cancer or ovarian cancer, which in other embodiments is treated by inhibiting CYP17-mediated estrogen synthesis.

For example, adenocarcinoma of the prostate is a common disease that causes significant morbidity and mortality in the adult male population (see Han and Nelson, Expert Opin. Pharmacother. 2000, 1, 443-9). Hormonal therapy for prostate cancer is considered when a patient fails with initial curative therapy, such as radical prostatectomy or definitive radiation therapy, or if he is found with an advanced disease. Hormonal agents have been developed to exploit the fact that prostate cancer growth is dependent on androgen. Non-steroidal anti-androgens (NSAAs) block androgen at the cellular level. Castration is another, albeit drastic means of decreasing androgens levels in order to treat or prevent prostate cancer. The methods and compositions described herein are useful in inhibiting the C_{17,20}-lyase activity of CYP17 and thereby decreasing levels of androgen production and the associated growth of androgen-dependent cancers such as prostate cancer.

In other embodiments, breast cancer, such as, by way of example only, breast cancer in postmenopausal women, is treated by administration of a CYP17 inhibitor described herein since adrenal and ovarian androgens are the main precursors of the estrogens which stimulate the growth of hormone dependent breast cancer. In further embodiments, breast cancer is treated with CYP17 inhibitors that inhibit interconversion of estrogens and adrenal and ovarian androgens. It has been shown that patients failing to respond to aromatase inhibitors show elevated levels of androgens in response to aromatase inhibitor treatment (see Harris et al., Bi. J. Cancer 1988, 58, 493-6). Accordingly, in other embodiments, sequential blockade to inhibit androgen production as well as inhibit aromatase produces greater estrogen suppression and enhanced therapeutic effects in treating breast and other estrogen hormone-dependent forms of cancer. Therefore, in some embodiments the inhibitors described herein are used alone or in combination with other drugs to treat and/or prevent hormone-dependent cancers such as breast and prostate cancer.

Furthermore, susceptibility to prostate cancer and breast cancer has been associated with particular polymorphic alleles of the CYP17 gene (see e.g. McKean-Cowdin, Cancer Res. 2001, 61, 848-9; Haiman et al., Cancer Epidmeiol. Biomarkers 2001, 10, 743-8; Huang et al., Cancer Res. 2001, 59, 4870-5). Accordingly, in other embodiments, the compositions described herein are suited to treating or preventing hormone-dependent cancers in individuals genetically predisposed to such cancers, particularly those predisposed due to an alteration in the CYP17 gene.

In one embodiment is a method for treating cancer in a subject comprising administering to a subject in need a therapeutically acceptable amount of a compound having the structure of Formula (I), (II) or (III) or a pharmaceutically acceptable salt or solvate thereof wherein the cancer is selected from the group consisting of bladder cancer, brain cancer, breast cancer, cervical cancer, colorectal cancer, endometrial cancer, gastric cancer, glioblastoma, head and neck cancer, Kaposi's sarcoma, kidney cancer, leiomyosarcoma, leukemia, liver cancer, lung cancer, melanoma, multiple myeloma, Non-Hodgkin lymphoma, ovarian cancer, pancreatic cancer, papillary renal cell carcinoma, prostate cancer, renal cancer, squamous cell cancer, and thoracic cancer.

In another embodiment is a method for treating cancer in a subject comprising administering to a subject in need a therapeutically acceptable amount of a compound having the structure of Formula (I), (II) or (III) or a pharmaceutically acceptable salt or solvate thereof wherein the cancer is prostate cancer.

In another embodiment is a method for treating cancer in a subject comprising administering to a subject in need a therapeutically acceptable amount of a compound having the structure of Formula (I), (II) or (III) or a pharmaceutically acceptable salt or solvate thereof wherein the cancer is breast cancer.

In a further embodiment the method of treating cancer further comprises providing to the subject in need an additional therapy selected from the group consisting of surgery, radiation therapy, chemotherapy, gene therapy, immunotherapy, or a combination thereof.

In yet a further embodiment, the additional therapy is surgery.

In one embodiment, providing chemotherapy to the subject in need comprises administering a therapeutically effective amount of at least one anti-androgenic agent.

In another embodiment, the at least one anti-androgenic agent is selected from the group consisting of flutamide, nicalutamide, bicalutamide, inhibitors of 17α-hydroxylase/C17-20 lyase, luteinizing hormone-releasing hormone agonists, luteinizing hormone-releasing hormone antagonists, and 5α-reductase type 1 and/or type 2 and combinations thereof.

Also disclosed herein is a method of inhibiting CYP17 enzyme comprising contacting a compound having the structure of Formula (I), (II) or (III) or a pharmaceutically acceptable salt or solvate thereof with a CYP17 enzyme.

In one embodiment, the contacting step is in vivo.

Also described herein is a method of treating an androgen-dependent disorder in a subject comprising administering to a subject in need a therapeutically acceptable amount of a compound having the structure of Formula (I), (II) or (III) or a pharmaceutically acceptable salt or solvate thereof.

In one embodiment, the androgen-dependent disorder is selected from the group consisting of prostate cancer, benign prostatic hyperplasia, prostatic intraepithelial neoplasia, hirsutism, acne, androgenic alopecia, and polycystic ovary syndrome.

In another embodiment, the androgen-dependent disorder is prostate cancer.

Presented herein is a method of treating a proliferative disease comprising administering to a subject in need a therapeutically effective amount of a compound having the structure of Formula (I), (II) or (III) or a pharmaceutically acceptable salt or solvate thereof.

In one embodiment, the method further comprises administering a therapeutically effective amount of at least one agent or therapy selected from the group consisting of a chemotherapeutic agent, a biological agent, surgery, and radiation therapy.

In another embodiment, the administration is performed concurrently or sequentially.

In one embodiment, is a method of treating a disease associated with cancer ameliorated by the inhibition of CYP17 enzyme comprising administering to a subject in need of treatment a therapeutically-effective amount of a compound having the structure of Formula (I), (II), (III), or a therapeutically acceptable salt or solvate thereof.

In some embodiments, is a method for the treatment of or prevention of a disease such as prostate or breast cancer comprising administering to a subject in need of treatment a therapeutically effective amount of a compound having the structure of Formula (I), (II), (III), or a therapeutically acceptable salt or solvate thereof.

It is generally contemplated that a compound having the structure of Formula (I), (II), (III), or therapeutically acceptable salt or solvate thereof can be employed in the treatment of and in some embodiments inhibits especially the inhibition of the CYP17 enzyme.

Another group of CYP17-associated diseases or disorders amenable to treatment with the compositions and methods of the present disclosure include those associated with mineralocorticoid excess such as hypertension caused by sodium retention at renal tubules. In some embodiments, a decrease in CYP17 activity results in an alteration in mineralocorticoid (e.g. aldosterone) biosynthesis. Accordingly, in some embodiments, the CYP17-associated diseases include those associated with altered levels of aldosterone production (e.g. hypertension, primary adrenal hyperplasia).

Still other examples of CYP17-associated diseases or disorders contemplated for treatment using a compound having the structure of Formula (I), (II) or (III) are Cushing's disease, prostatic hyperplasia, glucocorticoid deficiency, and endometrial cancer.

Certain embodiments provide a use of a compound having the structure of Formula (I), (II), (III), or a therapeutically acceptable salt or solvate thereof in combination with other agents for treatment of various diseases or conditions. Combination therapies according to the present disclosure comprise the administration of at least one compound disclosed herein and at least one other pharmaceutically active ingredient. In some embodiments, second pharmaceutically active agents for combination therapy include anti-cancer agents. In some embodiments, the active ingredient(s) and pharmaceutically active agents are administered separately or together. In further embodiments, separate administration occurs simultaneously or separately in any order. The amounts of the active ingredients(s) and pharmaceutically active agent(s) and the relative timings of administration will be selected in order to achieve the desired combined therapeutic effect.

Certain embodiments provide a use of a compound having the structure of Formula (I), (II), (III), or a therapeutically acceptable salt or solvate thereof, to prepare a medicament for treating diseases associated with the CYP17 enzyme.

### Certain Chemical Terminology

Unless defined otherwise, all technical and scientific terms used herein have the standard meaning pertaining to the claimed subject matter belongs. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Unless otherwise indicated, conventional methods of mass spectroscopy, NMR, HPLC, protein chemistry, biochemistry, recombinant DNA techniques and pharmacology are employed. Unless specific definitions are provided, the standard nomenclature employed in connection with, and the standard laboratory procedures and techniques of, analytical chemistry, synthetic organic chemistry, and medicinal and pharmaceutical chemistry are employed. In certain instances, standard techniques are used for chemical syntheses, chemical analyses, pharmaceutical preparation, formulation, and delivery, and treatment of patients. In certain embodiments, standard techniques are used for recombinant DNA, oligonucleotide synthesis, and tissue culture and transformation (e.g., electroporation, lipofection). In some embodiments, reactions and purification techniques are performed e.g., using kits of manufacturer's specifications or as commonly accomplished or as described herein.

As used throughout this application and the appended claims, the following terms have the following meanings:

The term “alkenyl” as used herein, means a straight, branched chain, or cyclic (in which case, it would also be known as a “cycloalkenyl”) hydrocarbon containing from 2-10 carbons and containing at least one carbon-carbon double bond formed by the removal of two hydrogens. Depending on the structure, an alkenyl group includes a monoradical or a diradical (i.e., an alkenylene group). Alkenyl groups include optionally substituted groups. Illustrative examples of alkenyl are ethenyl, 2-propenyl, 2-methyl-2-propenyl, 3-butenyl, 4-pentenyl, 5-hexenyl, 2-heptenyl, 2-methyl-1-heptenyl, and 3-cecenyl.

The term “alkoxy” as used herein, means an alkyl group, as defined herein, appended to the parent molecular moiety through an oxygen atom. Illustrative examples of alkoxy are methoxy, ethoxy, propoxy, 2-propoxy, butoxy, tert-butoxy, pentyloxy, and hexyloxy.

The term “alkyl” as used herein, means a straight, branched chain, or cyclic (in this case, it would also be known as “cycloalkyl”) hydrocarbon containing from 1-10 carbon atoms. Illustrative examples of alkyl are methyl, ethyl, n-propyl, iso-propyl, n-butyl, sec-butyl, tert-butyl, n-pentyl, isopentyl, neopentyl, n-hexyl, 3-methylhexyl, 2,2-dimethylpentyl, 2,3-dimethylhexyl, n-heptyl, n-octyl, n-nonyl, and n-decyl.

The term “cycloalkyl” as used herein, means a monocyclic or polycyclic radical that contains only carbon and hydrogen, and includes those that are saturated, partially unsaturated, or fully unsaturated. Cycloalkyl groups include groups having from 3 to 10 ring atoms. Illustrative examples of cyclic are the following moieties:

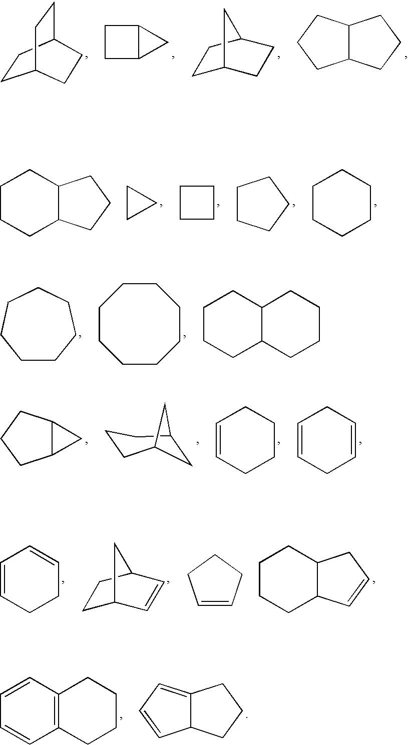

Depending on the structure, a cycloalkyl group includes a monoradical or a diradical (e.g., a cycloalkylene group).

The term “cycloalkyl groups” as used herein refers to groups which are optionally substituted with 1, 2, 3, or 4 substituents selected from alkenyl, alkoxy, alkoxyalkyl, alkoxycarbonyl, alkyl, alkylcarbonyl, alkylcarbonyloxy, alkylthio, alkylthioalkyl, alkynyl, carboxy, cyano, formyl, haloalkoxy, haloalkyl, halogen, hydroxyl, hydroxyalkyl, mercapto, oxo, —NR_{A}R_{A}, and (NR_{A}R_{B})carbonyl.

The term “cycloalkylalkyl” as used herein, means a cycloalkyl group, as defined herein, appended to the parent molecular moiety through an alkyl group, as defined herein. Illustrative examples of cycloalkylalkyl are cyclopropylmethyl, 2-cyclobutylethyl, cyclopentylmethyl, cyclohexylmethyl, and 4-cycloheptylbutyl.

The term “carbocycle” as used herein, refers to a ring, wherein each of the atoms forming the ring is a carbon atom. Carbocyclic rings include those formed by three, four, five, six, seven, eight, nine, or more than nine carbon atoms. Carbocycles are optionally substituted.

The term “alkoxyalkyl” as used herein, means at least one alkoxy group, as defined herein, appended to the parent molecular moiety through an alkyl group, as defined herein. Illustrative examples of alkoxyalkyl are 2-methoxyethyl, 2-ethoxyethyl, tert-butoxyethyl and methoxymethyl.

The term “alkoxycarbonyl” as used herein, means an alkoxy group, as defined herein, appended to the parent molecular moiety through a carbonyl group, as defined herein. Illustrative examples of alkoxycarbonyl are methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through an alkyl group, as defined herein.

The term “alkylcarbonyl” as used herein, means an alkyl group, as defined herein, appended to the parent molecular moiety through a carbonyl group, as defined herein. Illustrative examples of alkylcarbonyl are acetyl, 1-oxopropyl, 2,2-dimethyl-1-oxopropyl, 1-oxobutyl, and 1-oxopentyl.

The term “alkylcarbonyloxy” as used herein, means an alkylcarbonyl group, as defined herein, appended to the parent molecular moiety through an oxygen atom. Illustrative examples of alkylcarbonyloxy are acetyloxy, ethylcarbonyloxy, and tert-butylcarbonyloxy.

The term “alkylthio” or “thioalkoxy” as used herein, means an alkyl group, as defined herein, appended to the parent molecular moiety through a sulfur atom. Illustrative examples of alkylthio are methylthio, ethylthio, butylthio, tert-butylthio, and hexylthio.

The term “alkylthioalkyl” as used herein, means an alkylthio group, as defined herein, appended to the parent molecular moiety through an alkyl group, as defined herein. Illustrative examples of alkylthioalkyl are methylthiomethyl, 2-(ethylthio)ethyl, butylthiomethyl, and hexylthioethyl.

The term “alkynyl” as used herein, means a straight, branched chain hydrocarbon containing from 2-10 carbons and containing at least one carbon-carbon triple bond. Alkynyl groups are optionally substituted. Illustrative examples of alkynyl are acetylenyl, 1-propynyl, 2-propynyl, 3-butynyl, 2-pentynyl, and 1-butynyl.

The term “aromatic” as used herein, refers to a planar ring having a delocalized π-electron system containing 4n+2π electrons, where n is an integer. Aromatic rings include those formed by five, six, seven, eight, nine, or more than nine atoms. Aromatics are be optionally substituted. The term includes monocyclic or fused-ring polycyclic (i.e., rings which share adjacent pairs of carbon atoms) groups.

The term “aryl” as used herein, refers to an aromatic ring wherein each of the atoms forming the ring is a carbon atom. Aryl rings include those formed by five, six, seven, eight, nine, or more than nine carbon atoms. Illustrative examples of aryl groups are phenyl, naphthalenyl, phenanthrenyl, anthracenyl, fluorenyl, and indenyl.

The term “aryl” as used herein means an aryl group that is optionally substituted with one, two, three, four or five substituents independently selected from the group consisting of alkenyl, alkoxy, alkoxyalkyl, alkoxycarbonyl, alkyl, alkylcarbonyl, alkylcarbonyloxy, alkylthio, alkylthioalkyl, alkynyl, carbonyl, cyano, formyl, haloalkoxy, haloalkyl, halogen, hydroxyl, hydroxyalkyl, mercapto, nitro, —NR_{A}R_{A}, and (NR_{A}R_{B})carbonyl.

The term “arylalkyl” as used herein, means an aryl group, as defined herein, appended to the parent molecular moiety through an alkyl group, as defined herein. Illustrative examples of arylalkyl are benzyl, 2-phenylethyl, -phenylpropyl, 1-methyl-3-phenylpropyl, and 2-naphth-2-ylethyl.

The term “carbonyl” as used herein, means a —C(O)— group.

The term “carboxy” as used herein, means a —COOH group.

The term “cyano” as used herein, means a —CN group.

The term “formyl” as used herein, means a —C(O)H group.

The term “halo” or “halogen” as used herein, means a —Cl, —Br, —I or —F.

The term “mercapto” as used herein, means a —SH group.

The term “nitro” as used herein, means a —NO₂ group.

The term “hydroxy” as used herein, means a —OH group.

The term “oxo” as used herein, means a ═O group.

The term “bond” or “single bond” as used herein, refers to a chemical bond between two atoms, or two moieties when the atoms joined by the bond are considered to be part of larger substructure.

The terms “haloalkyl,” “haloalkenyl,” “haloalkynyl” and “haloalkoxy” as used herein, include alkyl, alkenyl, alkynyl and alkoxy structures in which at least one hydrogen is replaced with a halogen atom. In some embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another. The terms “fluoroalkyl” and “fluoroalkoxy” include haloalkyl and haloalkoxy groups, respectively, in which the halo is fluorine. In certain embodiments, haloalkyls are optionally substituted.

The term “alkylamine” refers to the —N(alkyl)ₓH_{y} group, where x and y are selected from among x=11, y=11 and x=2, y=0. When x=2, the alkyl groups, taken together with the N atom to which they are attached, optionally form a cyclic ring system.

The term “amide” as used herein, is a chemical moiety with the formula —C(O)NHR or —NHC(O)R, where R is selected from among hydrogen, alkyl, cycloalkyl, aryl, heteroaryl (bonded through a ring carbon) and heteroalicyclic (bonded through a ring carbon). An amide moiety includes a linkage between an amino acid or a peptide molecule and a compound described herein, e.g., in a prodrug. Any amine, or carboxyl side chain on the compounds described herein is optionally amidified.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein is optionally esterified.

The terms “heteroalkyl” “heteroalkenyl” and “heteroalkynyl” as used herein, include optionally substituted alkyl, alkenyl and alkynyl radicals in which one or more skeletal chain atoms are selected from an atom other than carbon, e.g., oxygen, nitrogen, sulfur, silicon, phosphorus or combinations thereof.

The term “heteroatom” as used herein refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms are the same as one another, or some or all of the two or more heteroatoms are different from the other or others.

The term “ring” as used herein, refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings are optionally substituted. In some instances, rings form part of a ring system.

As used herein, the term “ring system” refers to two or more rings, wherein two or more of the rings are fused. The term “fused” refers to structures in which two or more rings share one or more bonds.

The terms “heteroaryl” or, alternatively, “heteroaromatic” refers to an aromatic group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. The polycyclic heteroaryl group includes both fused and non-fused groups. Illustrative of heteroaryl groups are the following moieties:

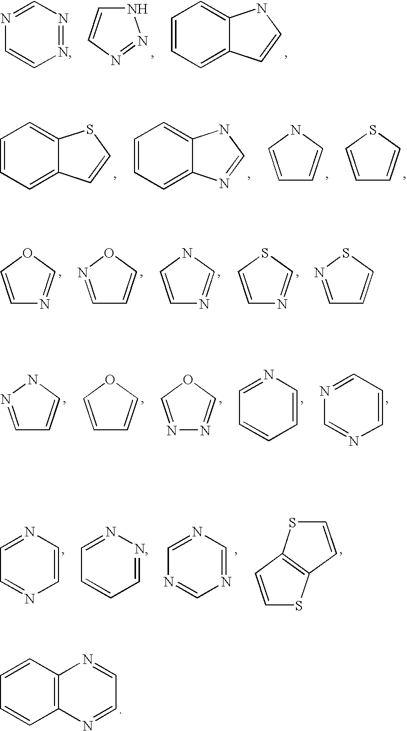

Depending on the structure, a heteroaryl group includes a monoradical or a diradical (i.e., a heteroarylene group).

The term “substituted heteroaryl” (or its equivalent) means heteroaryl groups that are substituted with 0, 1, 2, 3, or 4 substituents independently selected from alkenyl, alkoxy, alkoxyalkyl, alkoxycarbonyl, alkyl, alkylcarbonyl, alkylcarbonyloxy, alkylthio, alkylthioalkyl, alynyl, carboxy, cyano, formyl, haloalkoxy, haloalkyl, halogen, hydroxyl, hydroxyalkyl, mercapto, nitro, —NR_{A}R_{B}, and —(NR_{A}R_{B})carbonyl.

The term “heteroarylalkyl” as used herein, means a heteroaryl, as defined herein, appended to the parent molecular moiety through an alkyl group, as defined herein. An illustrative example of heteroarylalkyl is pyridinylmethyl.

The term “non-aromatic heterocycle”, “non-aromatic heterocyclic”, “heterocycloalkyl” or “heteroalicyclic” as used herein, refers to a non-aromatic ring wherein one or more atoms forming the ring is a heteroatom. A “non-aromatic heterocycle” or “non-aromatic heterocyclic”, “heterocycloalkyl” or “heteroalicyclic” group refers to a cycloalkyl group that includes at least one heteroatom selected from nitrogen, oxygen and sulfur. The radicals include those fused with an aryl or heteroaryl. Non-aromatic heterocycle rings include those formed by three, four, five, six, seven, eight, nine, or more than nine atoms. Heterocycloalkyl rings are optionally substituted. In certain embodiments, non-aromatic heterocycles contain one or more carbonyl or thiocarbonyl groups such as, for example, oxo- and thio-containing groups. Illustrative examples of heterocycloalkyls are lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles are

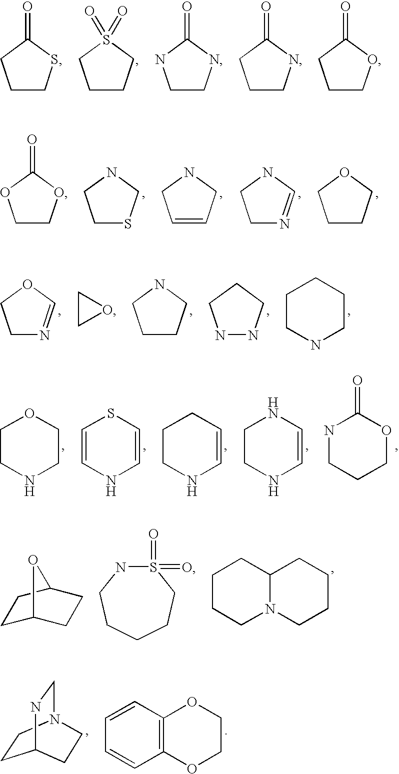

The term heteroalicyclic also includes all ring forms of the carbohydrates, including but not limited to the monosaccharides, the disaccharides and the oligosaccharides.

The term “heterocycle” refers to heteroaromatic and heteroalicyclic used herein, refers to groups containing one to four heteroatoms each selected from O, S and N, wherein each heterocyclic group has from 4 to 10 atoms in its ring system, and with the proviso that the ring of said group does not contain two adjacent O or S atoms. Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocyclic ring optionally has additional heteroatoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In heterocycles that have two or more heteroatoms, those two or more heteroatoms are the same or different from one another. Heterocycles are optionally substituted. Binding to a heterocycle is at a heteroatom or at a carbon atom. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, include those that are C-attached or N-attached where such is possible. For instance, a group derived from pyrrole includes pyrrol-1-yl groups (N-attached) or pyrrol-3-yl groups (C-attached). Further, a group derived from imidazole includes imidazol-1-yl or imidazol-3-yl (both N-attached) or imidazol-2-yl, imidazol-4-yl or imidazol-5-yl (all C-attached) groups. The heterocyclic groups include benzo-fused ring systems and ring systems substituted with one or two oxo (═O) moieties such as pyrrolidin-2-one. Depending on the structure, a heterocycle group includes a monoradical or a diradical (i.e., a heterocyclene group).

The heterocycles described herein are substituted with 0, 1, 2, 3, or 4 substituents independently selected from alkenyl, alkoxy, alkoxyalkyl, alkoxycarbonyl, alkyl, alkylcarbonyl, alkylcarbonyloxy, alkylthio, alkylthioalkyl, alynyl, carboxy, cyano, formyl, haloalkoxy, haloalkyl, halogen, hydroxyl, hydroxyalkyl, mercapto, nitro, —NR_{A}R_{B}, and —(NR_{A}R_{B})carbonyl.

The term “heterocycloalkylalkyl” as used herein, means a heterocycloalkyl, as defined herein, appended to the parent molecular moiety through an alkyl group, as defined herein.

The term “membered ring” embraces any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “non-aromatic 5, 6, 7, 8, 9, 10, 11 or 12-bicyclic heterocycle” as used herein, means a non-aromatic heterocycle, as defined herein, consisting of two carbocyclic rings, fused together at the same carbon atom (forming a spiro structure) or different carbon atoms (in which two rings share one or more bonds), having 5 to 12 atoms in its overall ring system, wherein one or more atoms forming the ring is a heteroatom. Illustrative examples of non-aromatic 5, 6, 7, 8, 9, 10, 11, or 12-bicyclic heterocycle ring are 2-azabicyclo[2.2.1]heptanyl, 7-azabicyclo[2.2.1]heptanyl, 2-azabicyclo[3.2.0]heptanyl, 3-azabicyclo[3.2.0]heptanyl, 4-azaspiro[2.4]heptanyl, 5-azaspiro[2.4]heptanyl, 2-oxa-5-azabicyclo[2.2.1]heptanyl, 4-azaspiro[2.5]octanyl, 5-azaspiro[2.5]octanyl, 5-azaspiro[3.4]octanyl, 6-azaspiro[3.4]octanyl, 4-oxa-7-azaspiro[2.5]octanyl, 2-azabicyclo[2.2.2]octanyl, 1,3-diazabicyclo[2.2.2]octanyl, 5-azaspiro[3.5]nonanyl, 6-azaspiro[3.5]nonanyl, 5-oxo-8-azaspiro[3.5]nonanyl, octahydrocyclopenta[c]pyrrolyl, octahydro-1H-quinolizinyl, 2,3,4,6,7,9a-hexahydro-1H-quinolizinyl, decahydropyrido[1,2-a]azepinyl, decahydro-1H-pyrido[1,2-a]azocinyl, 1-azabicyclo[2.2.1]heptanyl, 1-azabicyclo[3.3.1]nonanyl, quinuclidinyl, and 1-azabicyclo[4.4.0]decanyl.

The term hydroxylalkyl” as used herein, means at least one hydroxyl group, as defined herein, is appended to the parent molecular moiety through an alkyl group, as defined herein. Illustrative examples of hydroxyalkyl are hydroxymethyl, 2-hydroxy-ethyl, 3-hydroxypropyl and 4-hydroxyheptyl.

The term “NR_{A}R_{B}” as used herein, means two group, R_{A} and R_{B}, as defined herein, which are appended to the parent molecular moiety through a nitrogen atom. Illustrative examples of NR_{A}R_{B} are amino, methylamino, acetylamino, and acetylmethylamino.

The term “(NR_{A}R_{B})carbonyl” as used herein, means a NR_{A}R_{B}, group, as defined herein, which are appended to the parent molecular moiety through a carbonyl group, as defined herein. Illustrative examples of (NR_{A}R_{B})carbonyl are aminocarbonyl, (methylamino)carbonyl, (dimethylamino)carbonyl, and (ethylmethylamino)carbonyl.

The term “NR_{C}R_{D}” as used herein, means two group, R_{C} and R_{D}, as defined herein which are appended to the parent molecular moiety through a nitrogen atom. Illustrative examples of NR_{C}R_{D} are amino, methylamino, acetylamino, and acetylmethylamino.

The term “(NR_{C}R_{D})carbonyl” as used herein, means a NR_{C}R_{D}, group, as defined herein, appended to the parent molecular moiety through a carbonyl group, as defined herein. Illustrative examples of (NR_{C}R_{D})carbonyl are aminocarbonyl, (methylamino)carbonyl, (dimethylamino)carbonyl, and (ethylmethylamino)carbonyl.

As used herein, the term “mercaptyl” refers to a (alkyl)S— group.

As used herein, the term “moiety” refers to a specific segment or functional group of a molecule. Chemical moieties are often recognized chemical entities embedded in or appended to a molecule.

As used herein, the term “sulfinyl” refers to a —S(═O)—R, where R is selected from the group consisting of alkyl, cycloalkyl, aryl, heteroaryl (bonded through a ring carbon) and heteroalicyclic (bonded through a ring carbon).

As used herein, the term “sulfonyl” refers to a —S(═O)₂—R, where R is selected from the group consisting of alkyl, cycloalkyl, aryl, heteroaryl (bonded through a ring carbon) and heteroalicyclic (bonded through a ring carbon).

As used herein, the term “O carboxy” refers to a group of formula RC(═O)O—.

As used herein, the term “C carboxy” refers to a group of formula —C(═O)OR.

As used herein, the term “acetyl” refers to a group of formula —C(═O)CH₃.

As used herein, the term “acyl” refers to a group or radical of formula —C(═O)R where R is an organic group (an example of acyl group is the acetyl group).

As used herein, the term “arylacyl” refers to a group or radical of formula —C(═O)R where R is an aryl group wherein aryl is as defined.

As used herein, the term “heteroarylacyl” refers to a group or radical of formula —C(═O)R where R is a heteroaryl group wherein heteroaryl is as defined.

As used herein, the term “substituted arylacyl” refers to a group or radical of formula —C(═O)R where R is a substituted aryl group wherein substituted aryl is as defined.

As used herein, the term “substituted heteroarylacyl” refers to a group or radical of formula —C(═O)R where R is a substituted heteroaryl group wherein substituted heteroaryl is as defined.

As used herein, the term “trihalomethanesulfonyl” refers to a group of formula X₃CS(═O)₂— where X is a halogen.

As used herein, the term “isocyanato” refers to a group of formula —NCO.

As used herein, the term “thiocyanato” refers to a group of formula —CNS.

As used herein, the term “isothiocyanato” refers to a group of formula —NCS.

As used herein, the term “S sulfonamido” refers to a group of formula —S(═O)₂NR₂.

As used herein, the term “N sulfonamido” refers to a group of formula RS(═O)₂NH—.

As used herein, the term “trihalomethanesulfonamido” refers to a group of formula X₃CS(═O)₂NR—.

As used herein, the term “O carbamyl” refers to a group of formula —OC(═O)NR₂.

As used herein, the term “N carbamyl” refers to a group of formula ROC(═O)NH—.

As used herein, the term “O thiocarbamyl” refers to a group of formula —OC(═S)NR₂.

As used herein, the term “N thiocarbamyl” refers to a group of formula ROC(═S)NH—.

As used herein, the term “C amido” refers to a group of formula —C(═O)NR₂.

As used herein, the term “N amido” refers to a group of formula RC(═O)NH—.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

The term “substituted” means that the referenced group is optionally substituted (substituted or unsubstituted) with one or more additional group(s) individually and independently selected from alkyl, cycloalkyl, aryl, heteroaryl, heteroalicyclic, hydroxy, alkoxy, aryloxy, mercapto, alkylthio, arylthio, alkylsulfoxide, arylsulfoxide, alkylsulfone, arylsulfone, cyano, halo, carbonyl, thiocarbonyl, isocyanato, thiocyanato, isothiocyanato, nitro, perhaloalkyl, perfluoroalkyl, silyl, and amino, including mono- and di-substituted amino groups, and the protected derivatives thereof. By way of example an optional substituents is LₛRₛ, wherein each Lₛ is independently selected from a bond, —O—, —C(═O)—, —S—, —S(═O)—, —S(═O)₂—, —NH—, —NHC(O)—, —C(O)NH—, S(═O)₂ NH—, —NHS(═O)₂, —OC(O)NH—, —NHC(O)O—, -(substituted or unsubstituted C₁-C₆ alkyl), or -(substituted or unsubstituted C₂-C₆ alkenyl); and each Rₛ is independently selected from H, (substituted or unsubstituted lower alkyl), (substituted or unsubstituted lower cycloalkyl), heteroaryl, or heteroalkyl.

The term “optionally substituted” as defined herein, means the referenced group is substituted with one or more substituents as defined herein.

The term “protected-hydroxy” refers to a hydroxy group protected with a hydroxy protecting group, as defined above.

In some embodiments, the compounds described herein exist as stereoisomers, wherein asymmetric or chiral centers are present. Stereoisomers are designated (R) or (S) depending on the configuration of substituents around the chiral carbon atom. The term (R) and (S) used herein are configurations as defined in IUPAC 1974 Recommendations for Section E, Fundamental Stereochemistry, Pure Appl. Chem., (1976), 45:13-30, hereby incorporated by reference for this purpose. The embodiments described herein specifically includes the various stereoisomers and mixtures thereof. Stereoisomers include enantiomers, diastereomers, and mixtures of enantiomers or diastereomers. In some embodiments, individual stereoisomers of compounds are prepared synthetically from commercially available starting materials which contain asymmetric or chiral centers or by preparation of racemic mixtures followed by resolution. These methods of resolution are exemplified by (1) attachment of a mixture of enantiomers to a chiral axillary, separation of the resulting mixture of diastereomers by recrystallization or chromatography and liberation of the optically pure product from the auxiliary or (2) direct separation of the mixture of optical enantiomers on chiral chromatographic column.

The methods and formulations described herein include the use of N-oxides, crystalline forms (also known as polymorphs), or pharmaceutically acceptable salts of compounds described herein, as well as active metabolites of these compounds having the same type of activity. In some situations, compounds exist as tautomers. All tautomers are included within the scope of the compounds presented herein. In some embodiments, the compounds described herein exist in unsolvated as well as solvated forms with pharmaceutically acceptable solvents such as water, ethanol, and the like. The solvated forms of the compounds presented herein are also considered to be disclosed herein.

Throughout the specification, groups and substituents thereof are chosen, in certain embodiments, to provide stable moieties and compounds.

### Preparation of Compounds

In certain embodiments, the compounds described herein are synthesized using any synthetic techniques including standard synthetic techniques and the synthetic processes described herein. In specific embodiments, the following synthetic processes are utilized.

Formation of Covalent Linkages by Reaction of an Electrophile with a Nucleophile

Selected examples of covalent linkages and precursor functional groups which yield them are given in the Table entitled “Examples of Covalent Linkages and Precursors Thereof.” Precursor functional groups are shown as electrophilic groups and nucleophilic groups. In certain embodiments, a functional group on an organic substance is attached directly, or attached via any useful spacer or linker as defined below.

TABLE 1
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Suitable carbon nucleophiles include, but are not limited to alkyl, alkenyl, aryl and alkynyl Grignard, organolithium, organozinc, alkyl-, alkenyl, aryl- and alkynyl-tin reagents (organostannanes), alkyl-, alkenyl-, aryl- and alkynyl-borane reagents (organoboranes and organoboronates); these carbon nucleophiles have the advantage of being kinetically stable in water or polar organic solvents. Other carbon nucleophiles include phosphorus ylids, enol and enolate reagents; these carbon nucleophiles have the advantage of being relatively easy to generate from precursors. Carbon nucleophiles, when used in conjunction with carbon electrophiles, engender new carbon-carbon bonds between the carbon nucleophile and carbon electrophile.

Non-carbon nucleophiles suitable for coupling to carbon electrophiles include but are not limited to primary and secondary amines, thiols, thiolates, and thioethers, alcohols, alkoxides, azides, semicarbazides, and the like. These non-carbon nucleophiles, when used in conjunction with carbon electrophiles, typically generate heteroatom linkages (C—X—C), wherein X is a heteroatom, e.g, oxygen or nitrogen.

### Use of Protecting Groups

The term “protecting group” refers to chemical moieties that block some or all reactive moieties and prevent such groups from participating in chemical reactions until the protective group is removed. In specific embodiments, more than one protecting group is utilized. In more specific embodiments, each protective group is removable by a different process. Protective groups that are cleaved under totally disparate reaction conditions fulfill the requirement of differential removal. In various embodiments, protective groups are removed by acid, base, or hydrogenolysis. Groups such as trityl, dimethoxytrityl, acetal and t-butyldimethylsilyl are acid labile and are, in some embodiments, used to protect carboxy and hydroxy reactive moieties in the presence of amino groups protected with Cbz groups, which are removable by hydrogenolysis, and Fmoc groups, which are base labile. In some embodiments, carboxylic acid and hydroxy reactive moieties are blocked with base labile groups such as, without limitation, methyl, ethyl, and acetyl in the presence of amines blocked with acid labile groups such as t-butyl carbamate or with carbamates that are both acid and base stable but hydrolytically removable.

In certain embodiments, carboxylic acid and hydroxy reactive moieties are blocked with hydrolytically removable protective groups such as the benzyl group, while, in some embodiments, amine groups capable of hydrogen bonding with acids are blocked with base labile groups such as Fmoc. In various embodiments, carboxylic acid reactive moieties are protected by conversion to simple ester derivatives as exemplified herein, or they are blocked with oxidatively-removable protective groups such as 2,4-dimethoxybenzyl, while, in some embodiments, co-existing amino groups are blocked with fluoride labile silyl carbamates.

In certain instances, allyl blocking groups are useful in the presence of acid- and base-protecting groups since the former are stable. In some embodiments, such groups are subsequently removed by metal or pi-acid catalysts. For example, in some embodiments, an allyl-blocked carboxylic acid is deprotected with a Pd⁰-catalyzed reaction in the presence of acid labile t-butyl carbamate or base-labile acetate amine protecting groups. In some embodiments, a protecting group is a resin to which a compound or intermediate is attached. As long as the residue is attached to the resin, that functional group is blocked and cannot react. Once released from the resin, the functional group is available to react.

In some embodiments, blocking/protecting groups are selected from, by way of non-limiting example:

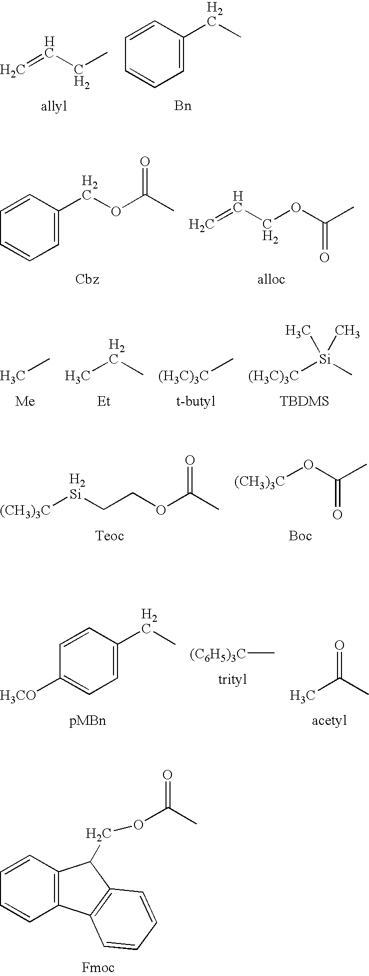

Other protecting groups are described in Greene and Wuts, Protective Groups in Organic Synthesis, 3rd Ed., John Wiley & Sons, New York, N.Y., 1999.

### Compounds of Formula (I), (II), or (III)

In certain embodiments, compounds of Formula (I), (II), and (III) are prepared by various methods, as outlined in Synthetic Schemes I-VI. In each scheme, the variables (e.g., R¹, R², R³, and R⁴) correspond to the same definitions as those recited above. In some embodiments, compounds are synthesized using methodologies analogous to those described below by the use of appropriate alternative starting materials. When R² and R³ are other than hydrogen, the appropriate starting material is obtained before subsequent synthetic steps are performed.

In certain embodiments, compounds of Formula (IE) are synthesized according to Synthetic Scheme I:

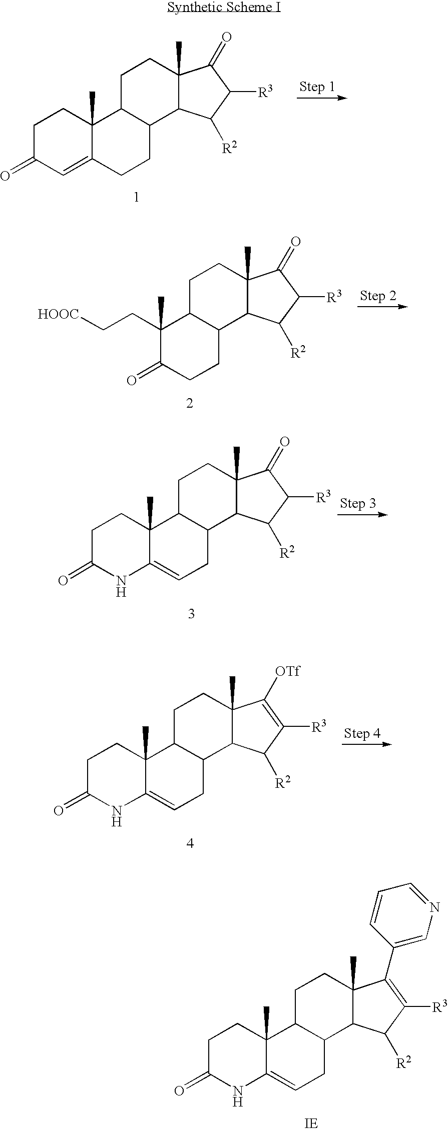

Compounds having the structure of Formula (IE) are synthesized from commercially available starting material 1. Oxidation of compound (1) with sodium periodate, potassium permanganate and potassium carbonate in water and t-butanol solution at refluxing temperature and subsequent treatment with dilute hydrochloric acid gives the ring opening product compound (2) (step 1). Treatment of compound (2) in ethylene glycol or other similar organic solvent with ammonia (or ammonium acetate and acetic acid) in a sealed pressure vessel at high temperature for 1 to 2 hours yields the compound (3) (step 2). Conversion of compound (3) to compound (4) is achieved by using condition as Step 3 which involves the enol triflate formation by the use of triflic anhydride (trifluoromethanesulfonic anhydride) in the presence of base such as triethylamine and the like. Suzuki coupling reaction on compound (4) with 3-(diethylboryl)pyridine, and (Ph₃P)₂PdCl₂ in THF in the presence of a base such as sodium carbonate yields compounds having the structure of Formula (IE) (step 4). Sometimes, it may be necessary to protection the NH in compound (3) with a t-butoxycarbonyl group.

In certain embodiments, compounds of Formula (IF) are synthesized according to Synthetic Scheme II:

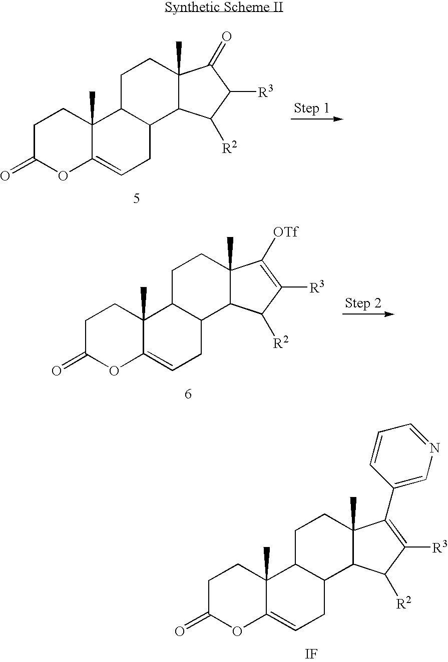

Compounds having the structure of Formula (IF) are synthesized from compound (5). Compound (5) is obtained following the procedure outlined in J. Chem. Soc. (1958), 2311-19. Step 1 of the synthesis requires the triflate formation by the use of triflic anhydride (trifluoromethanesulfonic anhydride) in the presence of base such as triethylamine and the like. Step 2 requires the Suzuki coupling reaction on compound (6) with 3-(diethylboryl)pyridine, and (Ph₃P)₂PdCl₂ in THF in the presence of a base such as sodium carbonate yields compounds having the structure of Formula (IF).

In certain embodiments, compounds of Formula (IIE) are synthesized according to Synthetic Scheme III:

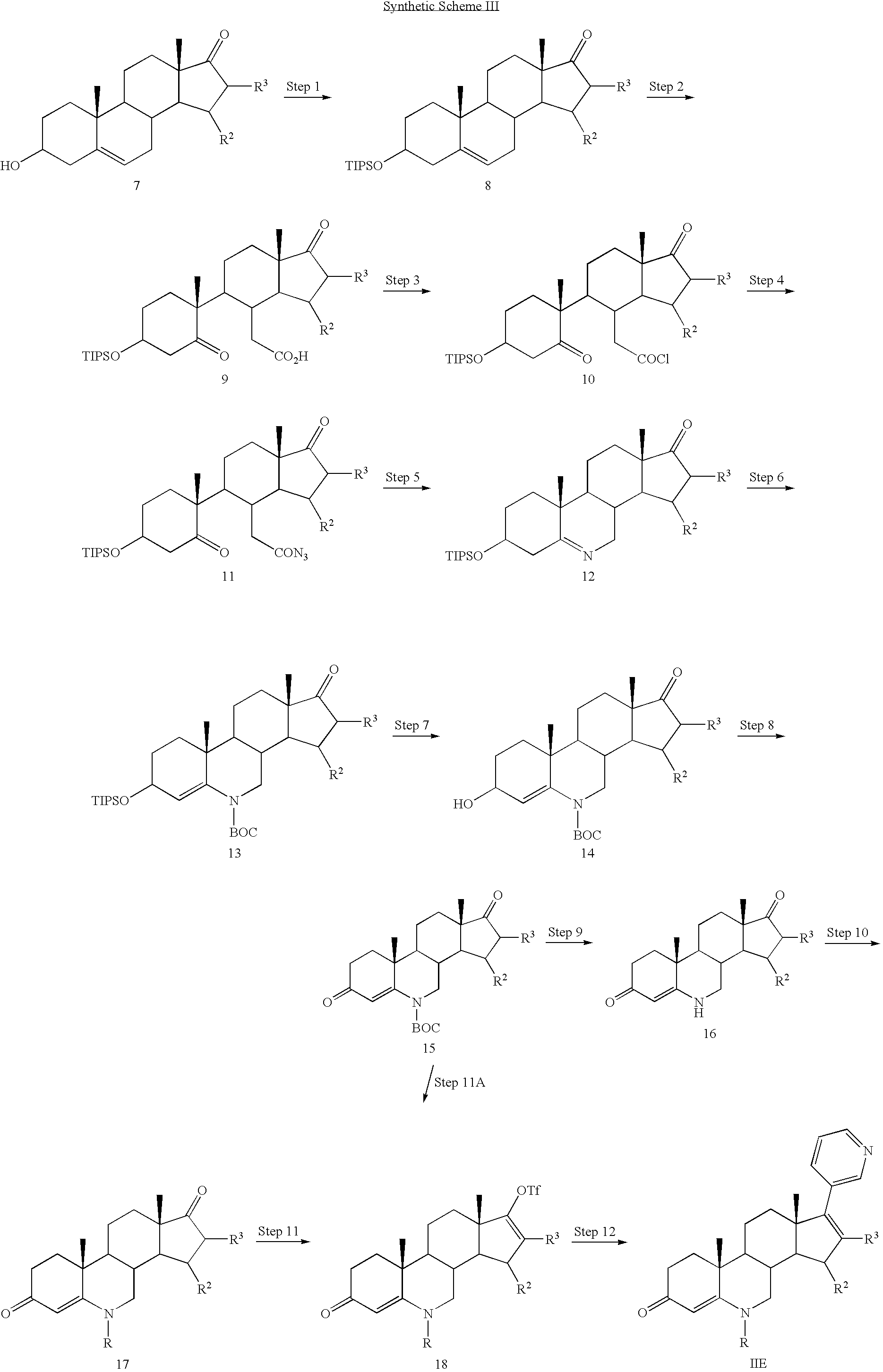

Compounds having the structure of Formula (IIE) are synthesized in 12 steps from intermediate 7. Step 1 of the synthesis requires the hydroxyl protection by the use of triisopropylsilanyl triflate in the presence of an organic base such as 2,6-lutidine and the like to give the 3-triisopropylsilanyloxy derivative compound (8). Step 2 involves the ring opening reaction by the use of ozone followed by the treatment with sodium dihydrogen phosphate and sulfamic acid and then sodium chlorite to give compound (9). Chlorination of compound (9) with thionyl chloride (step 3) gives compound (10). Displacement of the acyl chloride of compound (10) with sodium azide (step 4) provides the compound (11). Step 3 and step 4 can be combined by the use of diphenyl phosphoryl azide. Step 5 of the synthesis requires the heating of compound (11) in dry toluene followed by the addition of neutral alumina to yield compound (12). Treatment of compound (12) with di-tert-butyldicarbonate in pyridine (step 6) gives compound (13). Removal of the hydroxyl protection group of compound (13) (step 7) with tetrabutylammonium fluoride yields compound (14). Oxidation of the hydroxyl group of compound (14) with N-methylmorphine N-oxide and tetrapropylammonium perruthenate (step 8) gives compound (15). Hydrolysis of the t-butoxycarbonyl group with trifluoroacetic acid (step 9) provides compound (16). Treatment of compound (16) with iodomethane or R—Br in dry DMF and sodium hydride (step 10) yields compound (17). Step 11 of the synthesis requires the triflate formation of compound (17) by the use of triflic anhydride (trifluoromethanesulfonic anhydride) in the presence of base such as triethylamine and the like to give the enol triflate compound (18). Step 12 requires the Suzuki coupling reaction on compound (18) with 3-(diethylboryl)pyridine, and (Ph₃P)₂ PdCl₂ in THF in the presence of a base such as sodium carbonate yields compounds having the structure of Formula (IIE). Alternatively, compound (15) can be converted to compound (18) by doing the triflate formation by the use of triflic anhydride (trifluoromethanesulfonic anhydride) in the presence of base such as triethylamine and the like followed by hydrolysis with trifluoroacetic acid (step 11A).

In certain embodiments, compounds of Formula (IIIE) are synthesized according to Synthetic Scheme IV:

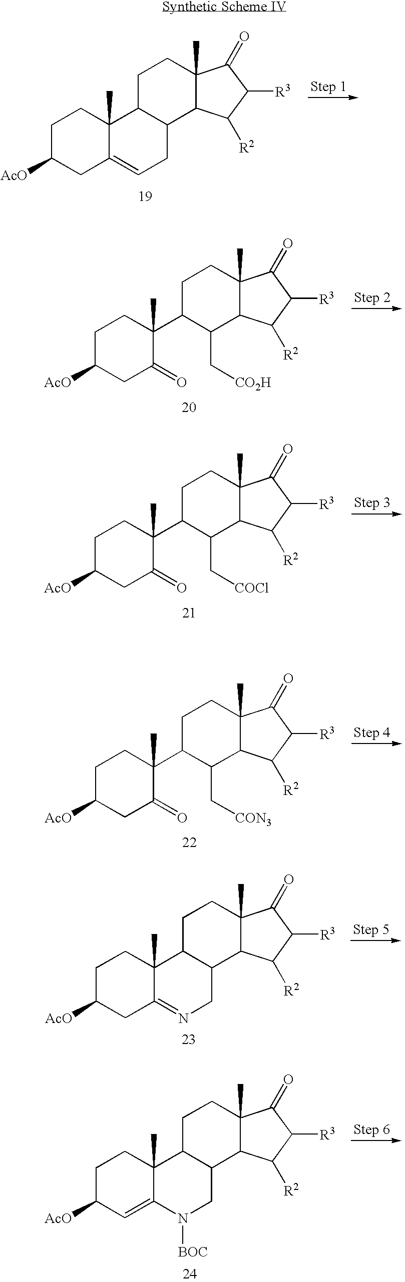

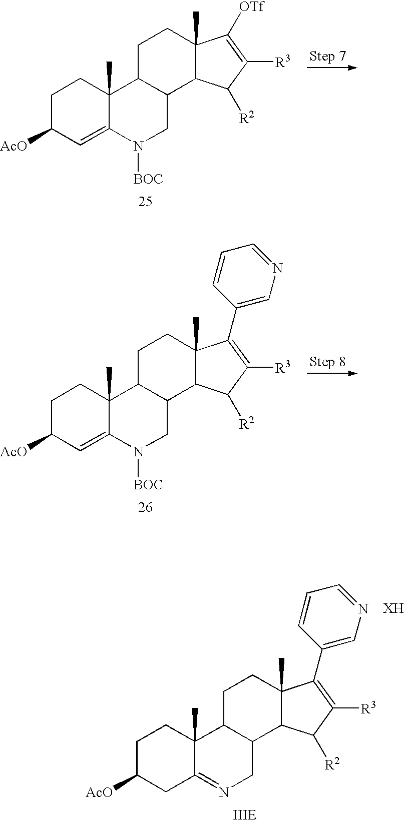

Compounds having the structure of Formula (IIIE) are synthesized using commercially available starting material 19. Step 1 requires the ring opening reaction on compound (19) by the use of ozone followed by the treatment with sodium dihydrogen phosphate and sulfamic acid and then sodium chlorite to give compound (20). Chlorination of compound (20) with thionyl chloride (step 2) gives compound (21). Displacement of the acyl chloride of compound (21) with sodium azide (step 3) provides the compound (22). Step 2 and step 3 can be combined by the use of diphenyl phosphoryl azide. Step 4 of the synthesis requires the heating of compound (22) in dry toluene followed by the addition of neutral alumina to yield compound (23). Treatment of compound (23) with di-tert-butyldicarbonate in pyridine (step 5) gives compound (24). Step 6 of the synthesis requires the triflate formation of compound (24) by the use of triflic anhydride (trifluoromethanesulfonic anhydride) in the presence of base such as triethylamine and the like to give the enol triflate compound (25). Step 7 requires the Suzuki coupling reaction on compound (25) with 3-(diethylboryl)pyridine, and (Ph₃P)₂ PdCl₂ in THF in the presence of a base such as sodium carbonate to yield compound (26). Hydrolysis of the t-butoxycarbonyl group with trifluoroacetic acid at low temperature (step 8) provides compounds having the structure of Formula (IIIE).

In certain embodiments, compounds of Formula (IG) are synthesized according to Synthetic Scheme V:

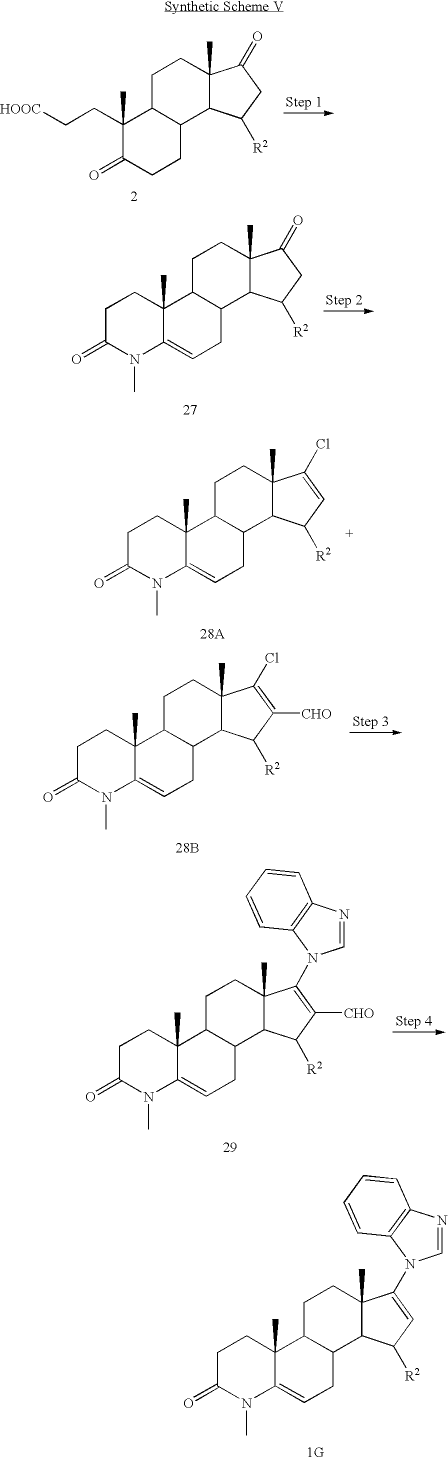

Compounds having the structure of Formula (IG) are synthesized from intermediate 2 in 4 steps. Step 1 requires the heating a mixture of compound (2) with methylamine (33% w/w in ethanol) in a sealed vessel for several hours to yield compound (27). Alternatively, step 1 can involve the treatment of compound (2) with methylamine in ethanol in the presence of sodium ethoxide at refluxing condition. Reaction of compound (27) with phosphoryl trichloride in DMF will yield a mixture of compound (28A) and compound (28B) (step 2). Displacement of the chlorine atom of compound (28) with benzoimidazole in the presence of potassium carbonate in DMF at 80° C. yields compound (29) (step 3). Step 4 of the synthesis involves the reaction of compound (29) with 10% palladium on carbon in refluxing benzonitrile to yield compounds having the structure of Formula (IG).

In certain embodiments, compounds of Formula (IIIF) are synthesized according to Synthetic Scheme VI:

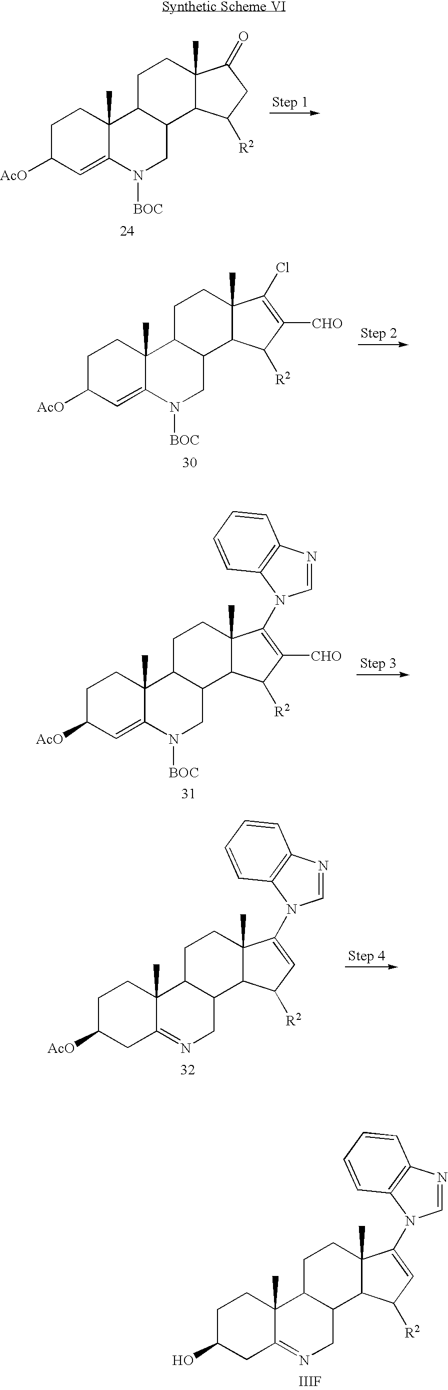

Compounds having the structure of Formula (IIIF) are synthesized from intermediate 24 in 4 steps. Step 1 requires heating compound (24) with phosphoryl trichloride in DMF to yield compound (30). Displacement of the chlorine atom of compound (30) with benzoimidazole in the presence of potassium carbonate in DMF at 80° C. yields compound (31) (step 2). Step 3 of the synthesis involves the reaction of compound (31) with 10% palladium on carbon in refluxing benzonitrile to yield compound (32). Treatment of compound (32) with 10% methanolic potassium hydroxide at room temperature yields compounds having the structure of Formula (IIIF).

### Certain Pharmaceutical Terminology

The term “acceptable” with respect to a formulation, composition or ingredient, as used herein, means having no persistent detrimental effect on the general health of the subject being treated.

As used herein, the term “selective binding compound” refers to a compound that selectively binds to any portion of one or more target proteins.

As used herein, the term “selectively binds” refers to the ability of a selective binding compound to bind to a target protein, such as, for example, CYP17 enzyme, with greater affinity than it binds to a non-target protein. In certain embodiments, specific binding refers to binding to a target with an affinity that is at least about 10, about 50, about 100, about 250, about 500, about 1000 or more times greater than the affinity for a non-target.

As used herein, the term “target protein” refers to a molecule or a portion of a protein capable of being bound by a selective binding compound. In certain embodiments, a target protein is the enzyme CYP17.

As used herein, the terms “treating” or “treatment” encompass either or both responsive and prophylaxis measures, e.g., designed to inhibit, slow or delay the onset of a symptom of a disease or disorder, achieve a full or partial reduction of a symptom or disease state, and/or to alleviate, ameliorate, lessen, or cure a disease or disorder and/or its symptoms.

As used herein, amelioration of the symptoms of a particular disorder by administration of a particular compound or pharmaceutical composition refers to any lessening of severity, delay in onset, slowing of progression, or shortening of duration, whether permanent or temporary, lasting or transient that can be attributed to or associated with administration of the compound or composition.

As used herein, the term inhibitor refers to a compound that decreases in the magnitude of a certain activity of a target protein or molecule compared to the magnitude of the activity in the absence of the inhibitor.

As used herein, the term “selective inhibitor” refers to a compound that selectively inhibits a target activity.

As used herein, the IC₅₀ refers to an amount, concentration or dosage of a particular test compound that achieves a 50% inhibition of a maximal response, such as modulation of CYP17, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

In one embodiment, toxicity and therapeutic efficacy of the compounds is determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and is expressed as the ratio LD₅₀/ED₅₀. Compounds which exhibit large therapeutic indices are contemplated herein. While in some embodiments, compounds that exhibit toxic side effects are used, care should be taken to design a delivery system that targets such reagents to the site of affected tissue in order to minimize potential damage to normal cells and, thereby, reduce side effects.

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The term “CYP17 substrate” includes any of the various steroid hormones acted upon by a CYP17 or a CYP17-like P₄₅₀ enzyme. Examples include pregnenolone, progesterone and their 17α-hydroxylated forms. Pregnenolone is converted to DHEA via a CYP17 C_{17,20}-lyase reaction, but is also subject to C17α-hydroxylation via the C_{17,20}-lyase activity. Progesterone is converted to δ4-androstenedione via a CYP17 C_{17,20}-lyase reaction, but is also subject to C17α-hydroxylation via the C17-hydroxylase activity to form 17-hydroxy-progesterone, a precursor to hydrocortisone (i.e. cortisol).

The term “CYP17 metabolite-associated disease or disorder” refers to a disease or disorder which in some embodiments is treated by alteration of the level of one or more CYP17 metabolites. Examples include a hormone dependent cancer, such as an androgen-dependent prostate cancer, which in other embodiments is treated by inhibiting CYP17-mediated androgen synthesis, and an estrogen-dependent breast cancer or ovarian cancer, which in further embodiments is treated by inhibiting CYP17-mediated estrogen synthesis.

The term “diluent” refers to chemical compounds that are used to dilute the compound of interest prior to delivery. Diluents include chemicals used to stabilize compounds because they provide a more stable environment. Salts dissolved in buffered solutions (which also can provide pH control or maintenance) are utilized as diluents in certain embodiments, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “enzymatically cleavable linker,” as used herein refers to unstable or degradable linkages which are degraded by one or more enzymes.

The terms “kit” and “article of manufacture” are used as synonyms.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, in certain instances, enzymes produce specific structural alterations to a compound. In some embodiments, metabolites of the compounds disclosed herein are identified either by administration of compounds to a host and analysis of tissue samples from the host, or by incubation of compounds with hepatic cells in vitro and analysis of the resulting compounds.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

By “pharmaceutically acceptable” or “therapeutically acceptable”, as used herein, refers a material, such as a carrier or diluent, which does not abrogate the biological activity or properties of the compound, and is relatively nontoxic. In certain instances, nontoxic and non-abrogative materials includes materials that when administered to an individual do not cause substantial, undesirable biological effects and/or do not interact in a deleterious manner with any of the components of the composition in which it is contained.

The term “pharmaceutically acceptable salt” or “therapeutically acceptable salt”, refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods known in the art.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound described herein with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

A “prodrug” refers to an agent that is converted into the parent drug in vivo. Prodrugs are often useful because, in some situations, they are easier to administer than the parent drug. In certain instances, a prodrug is bioavailable by oral administration whereas the parent is not. In some instances, a prodrug has improved solubility in pharmaceutical compositions over the parent drug. An example, without limitation, of a prodrug is a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid or amino group where the peptide is metabolized to reveal the active moiety. In certain embodiments, upon in vivo administration, a prodrug is chemically converted to the biologically, pharmaceutically or therapeutically more active form of the compound. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. In some embodiments, the prodrug is designed to alter the metabolic stability or the transport characteristics of a drug, to mask side effects or toxicity, to improve the flavor of a drug or to alter other characteristics or properties of a drug.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

Pharmaceutical Composition/Formulation

In certain embodiments, pharmaceutical compositions are formulated in any manner, including using one or more physiologically acceptable carriers comprising excipients and/or auxiliaries which facilitate processing of the active compounds into pharmaceutical preparations. In some embodiments, proper formulation is dependent upon the route of administration chosen. In various embodiments, any techniques, carriers, and excipients are used as suitable.

Provided herein are pharmaceutical compositions that include a compound described herein and a pharmaceutically acceptable diluent(s), excipient(s), and/or carrier(s). In addition, in some embodiments, the compounds described herein are administered as pharmaceutical compositions in which compounds described herein are mixed with other active ingredients, as in combination therapy.

A pharmaceutical composition, as used herein, refers to a mixture of a compound described herein with other chemical components, such as carriers, stabilizers, diluents, dispersing agents, suspending agents, thickening agents, and/or excipients. In certain embodiments, a pharmaceutical composition facilitates administration of the compound to an organism. In some embodiments, practicing the methods of treatment or use provided herein, includes administering or using a pharmaceutical composition comprising a therapeutically effective amount of a compound provided herein. In specific embodiments, the methods of treatment provided for herein include administering such a pharmaceutical composition to a mammal having a disease or condition to be treated. In one embodiment, the mammal is a human. In some embodiments, the therapeutically effective amount varies widely depending on the severity of the disease, the age and relative health of the subject, the potency of the compound used and other factors. In various embodiments, the compounds described herein are used singly or in combination with one or more therapeutic agents as components of mixtures.

In certain embodiments, the pharmaceutical compositions provided herein are formulated for intravenous injections. In certain aspects, the intravenous injection formulations provided herein are formulated as aqueous solutions, and, in some embodiments, in physiologically compatible buffers such as Hank's solution, Ringer's solution, or physiological saline buffer. In certain embodiments, the pharmaceutical compositions provided herein are formulated for transmucosal administration. In some aspects, transmucosal formulations include penetrants appropriate to the barrier to be permeated. In certain embodiments, the pharmaceutical compositions provided herein are formulated for other parenteral injections, appropriate formulations include aqueous or nonaqueous solutions, and in one embodiment, with physiologically compatible buffers or excipients.

In certain embodiments, the pharmaceutical compositions provided herein are formulated for oral administration. In certain aspects, the oral formulations provided herein comprise compounds described herein that are formulated with pharmaceutically acceptable carriers or excipients. Such carriers enable the compounds described herein to be formulated as tablets, powders, pills, dragees, capsules, liquids, gels, syrups, elixirs, slurries, suspensions and the like, for oral ingestion by a patient to be treated.

In some embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are optionally added, such as the cross-linked croscarmellose sodium, polyvinylpyrrolidone, agar, or alginic acid or a salt thereof such as sodium alginate.

In certain embodiments, provided herein is a pharmaceutical composition formulated as dragee cores with suitable coatings. In certain embodiments, concentrated sugar solutions are used in forming the suitable coating, and optionally contain gum arabic, talc, polyvinylpyrrolidone, carbopol gel, polyethylene glycol, and/or titanium dioxide, lacquer solutions, and suitable organic solvents or solvent mixtures. In some embodiments, dyestuffs and/or pigments are added to tablets, dragees and/or the coatings thereof for, e.g., identification or to characterize different combinations of active compound doses.

In certain embodiments, pharmaceutical preparations which are used include orally include push-fit capsules made of gelatin, as well as soft, sealed capsules made of gelatin and a plasticizer, such as glycerol or sorbitol. In some embodiments, the push-fit capsules contain the active ingredients in admixture with filler such as lactose, binders such as starches, and/or lubricants such as talc or magnesium stearate and, optionally, stabilizers. In certain embodiments, in soft capsules, the active compounds are dissolved or suspended in suitable liquids, such as fatty oils, liquid paraffin, or liquid polyethylene glycols. In addition, stabilizers are optionally added. In certain embodiments, the formulations for oral administration are in dosages suitable for such administration.

In certain embodiments, the pharmaceutical compositions provided herein are formulated for buccal or sublingual administration. In certain embodiments, buccal or sublingual compositions take the form of tablets, lozenges, or gels formulated in a conventional manner. In certain embodiments, parenteral injections involve bolus injection or continuous infusion. In some embodiments, formulations for injection are presented in unit dosage form, e.g., in ampoules or in multi-dose containers, with an added preservative. In some embodiments, the pharmaceutical composition described herein is in a form suitable for parenteral injection as a sterile suspensions, solutions or emulsions in oily or aqueous vehicles, and optionally contains formulatory agents such as suspending, stabilizing and/or dispersing agents. Pharmaceutical formulations for parenteral administration include aqueous solutions of the active compounds in water-soluble form. In some embodiments, suspensions of the active compounds are prepared as appropriate oily injection suspensions. Suitable lipophilic solvents or vehicles include fatty oils such as sesame oil, or synthetic fatty acid esters, such as ethyl oleate or triglycerides, or liposomes. In certain embodiments, aqueous injection suspensions contain substances which increase the viscosity of the suspension, such as sodium carboxymethyl cellulose, sorbitol, or dextran. Optionally, the suspensions also contain suitable stabilizers or agents which increase the solubility of the compounds to allow for the preparation of highly concentrated solutions. In alternative embodiments, the active ingredient is in powder form for constitution with a suitable vehicle, e.g., sterile pyrogen-free water, before use.

In some embodiments, the compounds described herein are administered topically. In specific embodiments, the compounds described herein are formulated into a variety of topically administrable compositions, such as solutions, suspensions, lotions, gels, pastes, medicated sticks, balms, creams or ointments. Such pharmaceutical compounds optionally contain solubilizers, stabilizers, tonicity enhancing agents, buffers and/or preservatives.

In certain embodiments, the pharmaceutical compositions provided herein are formulated for transdermal administration of compounds described herein. In some embodiments, administration of such compositions employs transdermal delivery devices and transdermal delivery patches. In certain embodiments, the compositions are lipophilic emulsions or buffered, aqueous solutions, dissolved and/or dispersed in a polymer or an adhesive. Such patches include those constructed for continuous, pulsatile, or on demand delivery of pharmaceutical agents. In some embodiments, transdermal delivery of the compounds described herein is accomplished by use of iontophoretic patches and the like. In certain embodiments, transdermal patches provide controlled delivery of the compounds provided herein, such as, for example, compounds of Formula (I), (II), or (III). In certain embodiments, the rate of absorption is slowed by using rate-controlling membranes or by trapping the compound within a polymer matrix or gel. Conversely, absorption enhancers are optionally used to increase absorption. Absorption enhancer and carrier include absorbable pharmaceutically acceptable solvents that assist in passage of the compound through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In certain embodiments, the pharmaceutical compositions provided herein are formulated for administration by inhalation. In certain embodiments, in such pharmaceutical compositions formulated for inhalation, the compounds described herein are in a form as an aerosol, a mist or a powder. In some embodiments, pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In certain aspects of a pressurized aerosol, the dosage unit is determined by providing a valve to deliver a metered amount. In certain embodiments, capsules and cartridges of, such as, by way of example only, gelatin for use in an inhaler or insufflator is formulated containing a powder mix of the compound described herein and a suitable powder base such as lactose or starch.

In some embodiments, the compounds described herein are formulated in rectal compositions such as enemas, rectal gels, rectal foams, rectal aerosols, suppositories, jelly suppositories, or retention enemas. In certain embodiments, rectal compositions optionally contain conventional suppository bases such as cocoa butter or other glycerides, as well as synthetic polymers such as polyvinylpyrrolidone, PEG, and the like. In certain suppository forms of the compositions, a low-melting wax such as, but not limited to, a mixture of fatty acid glycerides, optionally in combination with cocoa butter is first melted.

In various embodiments provided herein, the pharmaceutical compositions are formulated in a conventional manner using one or more physiologically acceptable carriers comprising excipients and auxiliaries which facilitate processing of the active compounds into pharmaceutically acceptable preparations. In certain embodiments, proper formulation is dependent upon the route of administration chosen. In various embodiments, any of the techniques, carriers, and excipients is used as suitable. In some embodiments, pharmaceutical compositions comprising a compound described herein are manufactured in a conventional manner, such as, by way of example only, by means of conventional mixing, dissolving, granulating, dragee-making, levigating, emulsifying, encapsulating, entrapping or compression processes.

In certain embodiments, the pharmaceutical compositions include at least one pharmaceutically acceptable carrier, diluent or excipient and a compound described herein described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds described herein exist as tautomers. All tautomers are included within the scope of the compounds presented herein. Additionally, included herein are the solvated and unsolvated forms of the compounds described herein. Solvated compounds include those that are solvated with pharmaceutically acceptable solvents such as water, ethanol, and the like. The solvated forms of the compounds presented herein are also considered to be disclosed herein. In some embodiments, the pharmaceutical compositions described herein include other medicinal or pharmaceutical agents, carriers, adjuvants, such as preserving, stabilizing, wetting or emulsifying agents, solution promoters, salts for regulating the osmotic pressure, and/or buffers. In additional embodiments, the pharmaceutical compositions described herein also contain other therapeutically valuable substances.

Methods for the preparation of compositions containing the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. In various embodiments, the compositions are in liquid solutions or suspensions, solid forms suitable for solution or suspension in a liquid prior to use, or as emulsions. These compositions optionally contain minor amounts of nontoxic, auxiliary substances, such as wetting or emulsifying agents, pH buffering agents, and so forth.

In some embodiments, a composition comprising a compound described herein takes the form of a liquid where the agents are present in solution, in suspension or both. In some embodiments, when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

Useful aqueous suspension optionally contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Useful compositions optionally comprise an mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful compositions optionally include solubilizing agents to aid in the solubility of a compound described herein. The term “solubilizing agent” generally includes agents that result in formation of a micellar solution or a true solution of the agent. Solubilizing agents include certain acceptable nonionic surfactants, for example polysorbate 80, and ophthalmically acceptable glycols, polyglycols, e.g., polyethylene glycol 400, and glycol ethers.

Useful compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Useful compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Certain useful compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Some useful compositions optionally include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Certain useful compositions optionally one or more antioxidants to enhance chemical stability where required. Suitable antioxidants include, by way of example only, ascorbic acid and sodium metabisulfite.

In some embodiments, aqueous suspension compositions are packaged in single-dose non-reclosable containers. In alternative embodiments, multiple-dose reclosable containers are used, in which case it is typical to include a preservative in the composition.

In various embodiments, any delivery system for hydrophobic pharmaceutical compounds is employed. Liposomes and emulsions are examples of delivery vehicles or carriers for hydrophobic drugs. In certain embodiments, certain organic solvents such as N-methylpyrrolidone are employed. In some embodiments, the compounds are delivered using a sustained-release system, such as semipermeable matrices of solid hydrophobic polymers containing the therapeutic agent. Various sustained-release materials are utilized in the embodiments herein. In certain embodiments, sustained-release capsules release the compounds for a few weeks up to over 100 days. In some embodiments, depending on the chemical nature and the biological stability of the therapeutic reagent, additional strategies for protein stabilization are employed.

In certain embodiments, the formulations or compositions described herein benefit from and/or optionally comprise antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### Methods of Dosing and Treatment Regimens

In certain embodiments, the compounds described herein are used in the preparation or manufacture of medicaments for the treatment of diseases or conditions that are mediated by the CYP17 enzyme. Inhibition of the enzymes ameliorates the disease or condition associated with CYP17. In some embodiments, a method for treating any of the diseases or conditions described herein in a subject in need of such treatment, involves administration of pharmaceutical compositions containing at least one compound described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest the symptoms of the disease or condition. In some embodiments, amounts effective for this use will depend on the severity and course of the disease or condition, previous therapy, the patient's health status, weight, and response to the drugs, and the judgment of the treating physician.

In certain prophylactic applications, compositions containing the compounds described herein are administered to a patient susceptible to or otherwise at risk of a particular disease, disorder or condition. In some embodiments, the amount administered is defined to be a “prophylactically effective amount or dose.” In certain embodiments of this use, the precise amounts of compound administered depend on the patient's state of health, weight, and the like. In certain embodiments, when used in a patient, effective amounts for this use will depend on the severity and course of the disease, disorder or condition, previous therapy, the patient's health status and response to the drugs, and the judgment of the treating physician.

In some embodiments, a patient's condition does not improve or does not significantly improve following administration of a compound or composition described herein and, upon the doctor's discretion the administration of the compounds is optionally administered chronically, that is, for an extended period of time, including throughout the duration of the patient's life in order to ameliorate or otherwise control or limit the symptoms of the patient's disease or condition.

In certain embodiments, once improvement of the patient's conditions has occurred, a maintenance dose is administered if necessary. In some embodiments, the dosage, e.g., of the maintenance dose, or the frequency of administration, or both, are reduced, as a function of the symptoms, to a level at which the improved disease, disorder or condition is retained. In certain embodiments, however, patients are optionally given intermittent treatment on a long-term basis upon any recurrence of symptoms.

In certain embodiments, the amount of a given agent that corresponds to an effective amount varies depending upon factors such as the particular compound, disease or condition and its severity, the identity (e.g., weight) of the subject or host in need of treatment. In some embodiments, the effective amount is, nevertheless, determined according to the particular circumstances surrounding the case, including, e.g., the specific agent that is administered, the route of administration, the condition being treated, and the subject or host being treated. In certain embodiments, however, doses employed for adult human treatment is in the range of about 0.02 to about 5000 mg per day. In one embodiment, dose employment for adult human treatment is about 1 to about 1500 mg per day. In various embodiments, the desired dose is conveniently presented in a single dose or as divided doses administered simultaneously (or over a short period of time) or at appropriate intervals, for example as two, three, four or more sub-doses per day.

In some embodiments, while the dose varies depending on age, body weight, symptom, treatment effect, administration method and the like, the pharmaceutical compositions described herein are given at a dose from about 0.01 mg to about 1 g per administration for an adult given once or several times a day orally or in a dosage form of an injection such as intravenous injection and the like. An anti-cancer agent is generally required to sustain its effect for a long time, so that can be effective not only for temporary suppression but also for prohibition on a long term basis. In one embodiment, the compounds described herein are administered on a long term basis.

In some embodiments, the pharmaceutical compositions described herein are in a unit dosage form suitable for single administration of precise dosages. In some instances, in unit dosage form, the formulation is divided into unit doses containing appropriate quantities of one or more compound. In certain embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. In alternative embodiments, multiple-dose reclosable containers are used, in which case it is typical to include a preservative in the composition. By way of example only, formulations for parenteral injection are, in some embodiments, presented in unit dosage form, which include, but are not limited to ampoules, or in multi-dose containers, with an added preservative.

In certain embodiments, the daily dosages appropriate for the compounds described herein are from about 0.01 to about 5 mg/kg per body weight. In some embodiments, an indicated daily dosage in the larger subject, including, but not limited to, humans, is in the range from about 0.5 mg to about 1000 mg, conveniently administered in divided doses, including, but not limited to, up to four times a day or in extended release form. In certain embodiments, suitable unit dosage forms for oral administration comprise from about 1 to about 500 mg active ingredient. The foregoing ranges are merely suggestive, as the number of variables in regard to an individual treatment regime is large, and considerable excursions from these recommended values are not uncommon. In certain embodiments, the dosages are altered depending on a number of variables, not limited to the activity of the compound used, the disease or condition to be treated, the mode of administration, the requirements of the individual subject, the severity of the disease or condition being treated, and the judgment of the practitioner.

In certain embodiments, toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. In certain embodiments, compounds exhibiting high therapeutic indices are preferred. In some embodiments, the data obtained from cell culture assays and animal studies is used in formulating a range of dosage for use in human. In specific embodiments, the dosage of such compounds lies within a range of circulating concentrations that include the ED₅₀ with minimal toxicity. In certain embodiments, the dosage varies within this range depending upon the dosage form employed and the route of administration utilized.

### Combination Treatments

Presented herein are compounds having the structure of Formula (I), (II) or (III) in combination with a second therapeutic agent for the treatment of an androgen dependent disease, disorder or condition. In one embodiment, the compounds described herein are administered in combination with a second active agent which is effective against cancer.

Suitable compounds used in combination with a compound having the structure of Formula (I), (II) or (III) include anti-cancer agents, such as for example, hormone ablation agents, anti-androgen agents, differentiating agents, anti-neoplastic agents, kinase inhibitors, anti-metabolite agents, alkylating agents, antibiotic agents, immunological agents, interferon-type agents, intercalating agents, growth factor inhibitors, cell cycle inhibitors, enzymes, topoisomerase inhibitors, biological response modifiers, mitotic inhibitors, matrix metalloprotease inhibitors, genetic therapeutics, and anti-androgens. The amount of the additional anti-cancer agent administered to a mammal having cancer is an amount that is sufficient to treat the cancer whether administered alone or in combination with a compound having the structure of Formula (I), (II) or (III). Below are lists of examples of some of the classes of anti-cancer agents. The examples are not all inclusive and are for purposes of illustration and not for purposes of limitation. Many of the examples below are not restricted in any way to the class in which they are listed in and in some embodiments are listed in multiple classes of anti-cancer agents.

Suitable hormonal ablation agents include, but are not limited to, androgen ablation agents and estrogen ablation agents. In some embodiments, a compound having the structure of Formula (I), (II) or (III) is administered with a hormonal ablation agent, such as deslorelin, leuprolide, goserelin or triptorelin. The amount of the hormonal ablation agent administered to a mammal having cancer is an amount that is sufficient to treat the cancer whether administered alone or in combination with a compound having the structure of Formula (I), (II) or (III).

Suitable anti-androgen agents include but are not limited to bicalutamide, flutamide and nilutamide. The amount of the anti-androgen agent administered to a mammal having cancer is an amount that is sufficient to treat the cancer whether administered alone or in combination with a compound having the structure of Formula (I), (II) or (III).

In another embodiment, a compound having the structure of Formula (I), (II) or (III) is administered with a differentiating agent. Suitable differentiating agents include, but are not limited to, polyamine inhibitors; vitamin D and its analogs, such as, calcitriol, doxercalciferol and seocalcitol; metabolites of vitamin A, such as, ATRA, retinoic acid, retinoids; short-chain fatty acids; phenylbutyrate; and nonsteroidal anti-inflammatory agents. The amount of the differentiating agent administered to a mammal having cancer is an amount that is sufficient to treat the cancer whether administered alone or in combination with a compound having the structure of Formula (I), (II) or (III).

In a further embodiment, a compound having the structure of Formula (I), (II) or (III) is administered with an anti-neoplastic agent, including, but not limited to, tubulin interacting agents, topoisomerase inhibitors and agents, acitretin, alstonine, amonafide, amphethinile, amsacrine, ankinomycin, anti-neoplaston, aphidicolin glycinate, asparaginase, baccharin, batracylin, benfluoron, benzotript, bromofosfamide, caracemide, carmethizole hydrochloride, chlorsulfaquinoxalone, clanfenur, claviridenone, crisnatol, curaderm, cytarabine, cytocytin, dacarbazine, datelliptinium, dihaematoporphyrin ether, dihydrolenperone, dinaline, distamycin, docetaxel, elliprabin, elliptinium acetate, epothilones, ergotamine, etoposide, etretinate, fenretinide, gallium nitrate, genkwadaphnin, hexadecylphosphocholine, homoharringtonine, hydroxyurea, ilmofosine, isoglutamine, isotretinoin, leukoregulin, lonidamine, merbarone, merocyanlne derivatives, methylanilinoacridine, minactivin, mitonafide, mitoquidone, mitoxantrone, mopidamol, motretinide, N-(retinoyl)amino acids, N-acylated-dehydroalanines, nafazatrom, nocodazole derivative, ocreotide, oquizanocine, paclitaxel, pancratistatin, pazelliptine, piroxantrone, polyhaematoporphyrin, polypreic acid, probimane, procarbazine, proglumide, razoxane, retelliptine, spatol, spirocyclopropane derivatives, spirogermanium, strypoldinone, superoxide dismutase, teniposide, thaliblastine, tocotrienol, topotecan, ukrain, vinblastine sulfate, vincristine, vindesine, vinestramide, vinorelbine, vintriptol, vinzolidine, and withanolides. The amount of the anti-neoplastic agent administered to a mammal having cancer is an amount that is sufficient to treat the cancer whether administered alone or in combination with a compound having the structure of Formula (I), (II) or (III).

In some embodiments, the compounds described herein, such as for example, a compound having the structure of Formula (I), (II) or (III) is used with a kinase inhibitor including p38 inhibitors and CDK inhibitors, TNF inhibitors, metallomatrix proteases inhibitors (MMP), COX-2 inhibitors including celecoxib, rofecoxib, parecoxib, valdecoxib, and etoricoxib, SOD mimics or αᵥβ₃ inhibitors. The amount of the kinase inhibitor administered to a mammal having cancer is an amount that is sufficient to treat the cancer whether administered alone or in combination with a compound having the structure of Formula (I), (II) or (III).

In another embodiment, a compound having the structure of Formula (I), (II) or (III) is administered with an anti-metabolite agent. In one embodiment, suitable anti-metabolite agents are selected from, but not limited to, 5-FU-fibrinogen, acanthifolic acid, aminothiadiazole, brequinar sodium, carmofur, cyclopentyl cytosine, cytarabine phosphate stearate, cytarabine conjugates, dezaguanine, dideoxycytidine, dideoxyguanosine, didox, doxifluridine, fazarabine, floxuridine, fludarabine phosphate, 5-fluorouracil, N-(2′-furanidyl)-5-fluorouracil, isopropyl pyrrolizine, methobenzaprim, methotrexate, norspermidine, pentostatin, piritrexim, plicamycin, thioguanine, tiazofurin, trimetrexate, tyrosine kinase inhibitors, and uricytin. The amount of the anti-metabolite agent administered to a mammal having cancer is an amount that is sufficient to treat the cancer whether administered alone or in combination with a compound having the structure of Formula (I), (II) or (III).

In another embodiment, a compound having the structure of Formula (I), (II) or (III) is administered with an alkylating agent. In another embodiment, suitable alkylating agents are selected from, but not limited to, aldo-phosphamide analogues, altretamine, anaxirone, bestrabucil, budotitane, carboplatin, carmustine, chlorambucil, cisplatin, cyclophosphamide, cyplatate, diphenylspiromustine, diplatinum cytostatic, elmustine, estramustine phosphate sodium, fotemustine, hepsul-fam, ifosfamide, iproplatin, lomustine, mafosfamide, mitolactol, oxaliplatin, prednimustine, ranimustine, semustine, spiromustine, tauromustine, temozolomide, teroxirone, tetraplatin and trimelamol. The amount of the alkylating agent administered to a mammal having cancer is an amount that is sufficient to treat the cancer whether administered alone or in combination with a compound having the structure of Formula (I), (II) or (III).

In yet another embodiment, a compound having the structure of Formula (I), (II) or (III) is administered with an antibiotic agent. In another embodiment, suitable antibiotic agents are selected from, but not limited to, aclarubicin, actinomycin D, actinoplanone, adriamycin, aeroplysinin derivative, amrubicin, anthracycline, azino-mycin-A, bisucaberin, bleomycin sulfate, bryostatin-1, calichemycin, chromoximycin, dactinomycin, daunorubicin, ditrisarubicin B, dexamethasone, doxorubicin, doxorubicin-fibrinogen, elsamicin-A, epirubicin, erbstatin, esorubicin, esperamicin-Al, esperamicin-Alb, fostriecin, glidobactin, gregatin-A, grincamycin, herbimycin, corticosteroids such as hydrocortisone, idarubicin, illudins, kazusamycin, kesarirhodins, menogaril, mitomycin, neoenactin, oxalysine, oxaunomycin, peplomycin, pilatin, pirarubicin, porothramycin, prednisone, prednisolone, pyrindanycin A, rapamycin, rhizoxin, rodorubicin, sibanomicin, siwenimycin, sorangicin-A, sparsomycin, talisomycin, terpentecin, thrazine, tricrozarin A, and zorubicin. The amount of the antibiotic agent administered to a mammal having cancer is an amount that is sufficient to treat the cancer whether administered alone or in combination with a compound having the structure of Formula (I), (II) or (III).

In a further embodiment, a compound having the structure of Formula (I), (II) or (III) is used with other anti-cancer agents, including but not limited to, acemannan, aclarubicin, aldesleukin, alemtuzumab, alitretinoin, altretamine, amifostine, amsacrine, anagrelide, anastrozole, ancestim, bexarotene, broxuridine, capecitabine, celmoleukin, cetrorelix, cladribine, clotrimazole, daclizumab, dexrazoxane, dilazep, docosanol, doxifluridine, bromocriptine, carmustine, cytarabine, diclofenac, edelfosine, edrecolomab, eflomithine, emitefur, exemestane, exisulind, fadrozole, filgrastim, finasteride, fludarabine phosphate, formestane, fotemustine, gallium nitrate, gemcitabine, glycopine, heptaplatin, ibandronic acid, imiquimod, iobenguane, irinotecan, irsogladine, lanreotide, leflunomide, lenograstim, lentinan sulfate, letrozole, liarozole, lobaplatin, lonidamine, masoprocol, melarsoprol, metoclopramide, mifepristone, miltefosine, mirimostim, mitoguazone, mitolactol, molgramostim, nafarelin, nartograstim, nedaplatin, nilutamide, noscapine, oprelvekin, osaterone, oxaliplatin, pamidronic acid, pegaspargase, pentosan polysulfate sodium, pentostatin, picibanil, pirarubicin, porfimer sodium, raloxifene, raltitrexed, rasburicase, rituximab, romurtide, sargramostim, sizofuran, sobuzoxane, sonermin, suramin, tasonermin, tazarotene, tegafur, temoporfin, temozolomide, teniposide, tetrachlorodecaoxide, thalidomide, thymalfasin, thyrotropin alfa, topotecan, toremifene, trastuzumab, treosulfan, tretinoin, trilostane, trimetrexate, ubenimex, valrubicin, verteporfin, vinorelbine. The amount of the anti-cancer agent administered to a mammal having cancer is an amount that is sufficient to treat the cancer whether administered alone or in combination with a compound having the structure of Formula (I), (II) or (III).

In yet another embodiment, a compound having the structure of Formula (I), (II) or (III) is administered or combined with steroids, such as corticosteroids or glucocorticoids. In a further embodiment, a compound having the structure of Formula (I), (II) or (III) and the steroid are administered in the same or in different compositions. Non-limiting examples of suitable steroids include hydrocortisone, prednisone, or dexamethasone. The amount of the steroid administered to a mammal having cancer is an amount that is sufficient to treat the cancer whether administered alone or in combination with a compound having the structure of Formula (I), (II) or (III).

In some embodiments, if one of the side effects experienced by a patient upon receiving one of the compounds herein is inflammation, then, in some embodiments, it is appropriate to administer an anti-inflammatory agent in combination with the initial therapeutic agent. In some embodiments, the therapeutic effectiveness of one of the compounds described herein is enhanced by administration of an adjuvant (i.e., by itself the adjuvant may have minimal therapeutic benefit, but in combination with another therapeutic agent, the overall therapeutic benefit to the patient is enhanced). In certain embodiments, the benefit experienced by a patient is increased by administering one of the compounds described herein with another therapeutic agent (which also includes a therapeutic regimen that also has same therapeutic benefit (e.g. anti-cancer agent against the same enzyme as the compound described herein but of different mode of action) so as to reduce the chance of enzyme resistant development. In some embodiments, regardless of the disease, disorder or condition being treated, the overall benefit experienced by the patient as a result of a combination treatment is additive or synergistic.

In certain embodiments, therapeutically-effective dosages vary when the drugs are used in treatment combinations. In some embodiments, therapeutically-effective dosages of drugs and other agents for use in combination treatment regimens is determined in any suitable manner, e.g., through the use of metronomic dosing, i.e., providing more frequent, lower doses in order to minimize toxic side effects. In some embodiments, combination treatment regimen described herein encompass treatment regimens in which administration of a compound having the structure of Formula (I), (II) or (III) described herein is initiated prior to, during, or after treatment with a second agent described above, and continues until any time during treatment with the second agent or after termination of treatment with the second agent. It also includes treatments in which a compound having the structure of Formula (I), (II) or (III) described herein and the second agent being used in combination are administered simultaneously or at different times and/or at decreasing or increasing intervals during the treatment period.

In certain embodiments, compositions and methods for combination therapy are provided herein. In accordance with one aspect, the pharmaceutical compositions disclosed herein are used to in a method of treating a CYP17 mediated condition or a disease or condition that is ameliorated by inhibition of these enzymes.

In certain embodiments, combination therapies described herein are used as part of a specific treatment regimen intended to provide a beneficial effect from the co-action of a compound having the structure of Formula (I), (II) or (III) described herein and a concurrent treatment. It is understood that the dosage regimen to treat, prevent, or ameliorate the condition(s) for which relief is sought, is optionally modified in accordance with a variety of factors.

In certain combination therapies described herein, dosages of the co-administered compounds vary depending on the type of co-drug employed, on the specific drug employed, on the disease or condition being treated and so forth. In some embodiments, when co-administered with one or more biologically active agents, the compound provided herein is administered either simultaneously with the biologically active agent(s), or sequentially. In certain aspects wherein the agents are administered sequentially, the attending physician will decide on the appropriate sequence of administering protein in combination with the biologically active agent(s).

In various embodiments, the multiple therapeutic agents (one of which is one of the compounds described herein) are administered in any order or even simultaneously. In certain instances, administration is simultaneous and the multiple therapeutic agents are, optionally, provided in a single, unified form, or in multiple forms (by way of example only, either as a single pill or as two separate pills). In some embodiments, one of the therapeutic agents is given in multiple doses, or both are given as multiple doses. In some instances, administration is not simultaneous and the timing between the multiple doses varies, by way of non-limiting example, from more than zero weeks to less than four weeks. In addition, the combination methods, compositions and formulations are not to be limited to the use of only two agents; the use of multiple therapeutic combinations is also contemplated herein.

In certain embodiments, the compounds described herein and combination therapies are administered before, during or after the occurrence of a disease or condition. In certain embodiments, the timing of administering the composition containing a compound varies. Thus, for example, in some embodiments, the compounds are used as a prophylactic and are administered continuously to subjects with a propensity to develop conditions or diseases in order to prevent the occurrence of the disease or condition. In some embodiments, the compounds and compositions are administered to a subject during or as soon as possible after the onset of the symptoms. The initial administration is achieved via any route practical, such as, for example, an intravenous injection, a bolus injection, infusion over 5 minutes to about 5 hours, a pill, a capsule, transdermal patch, buccal delivery, and the like, or combination thereof.

### Kits/Articles of Manufacture

For use in the therapeutic applications described herein, kits and articles of manufacture are also described herein. In various embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some embodiments, the containers are formed from a variety of materials such as glass or plastic.

In some embodiments, the articles of manufacture provided herein contain packaging materials. Packaging materials for use in packaging pharmaceutical products include, but are not limited to, blister packs, bottles, tubes, inhalers, pumps, bags, vials, containers, syringes, bottles, and any packaging material suitable for a selected formulation and intended mode of administration and treatment.

In some embodiments, the container(s) described herein comprise one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound with an identifying description or label or instructions relating to its use in the methods described herein.

In some embodiments, a kit will comprises one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions is optionally included.

In certain embodiments, a label is on or associated with the container. In some embodiments, a label is on a container when letters, numbers or other characters forming the label are attached, molded or etched into the container itself; a label can be associated with a container when it is present within a receptacle or carrier that also holds the container, e.g., as a package insert. In certain embodiments, a label indicates that the contents are to be used for a specific therapeutic application. In some embodiments, the label indicates directions for use of the contents, such as in the methods described herein.

In certain embodiments, the pharmaceutical compositions are presented in a pack or dispenser device which contains one or more unit dosage forms containing a compound provided herein. In some embodiments, the pack contains a metal or plastic foil, such as a blister pack. The pack or dispenser device is optionally accompanied by instructions for administration. In some embodiments, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. In certain embodiments, such notice is, for example, the labeling approved by the U.S. Food and Drug Administration for prescription drugs, or the approved product insert. In some embodiments, compositions containing a compound provided herein are formulated in a compatible pharmaceutical carrier and are placed in an appropriate container labeled for treatment of an indicated condition.

### EXAMPLES

The following Examples are intended as an illustration of the various embodiments as defined in appended claims. In some embodiments, the compounds are prepared by a variety of synthetic routes.

### Example 1

### Example 1a

### Parenteral Composition

To prepare a parenteral pharmaceutical composition suitable for administration by injection, 100 mg of a water-soluble salt of a compound having the structure of Formula (I), (II) or (III) is mixed with 2-hydroxypropyl-β-cyclodextrin and then dissolved in 10 mL of 0.9% sterile saline. The mixture is incorporated into a dosage unit form suitable for administration by injection.

### Example 1b

### Oral Composition

To prepare a capsule suitable for oral administration, a water-soluble salt of a compound having the structure of Formula (I), (II) or (III) (20 mg) is mixed with lactose (180 mg), microcrystalline cellulose (140 mg) and magnesium stearate (20 mg). The mixture is granulated and the remaining 10 mg of magnesium stearate is added. The content is then sealed in a gelation capsule.

To prepare a tablet suitable for oral administration, a water-soluble salt of a compound having the structure of Formula (I), (II) or (III) (20 mg) is mixed with lactose (70 mg), corn starch (300 mg), microcrystalline cellulose (60 mg) and magnesium stearate (10 mg). The mixture is granulated and the remaining 10 mg of microcrystalline cellulose and 2.5 mg of magnesium stearate is added. The mixture is compression formed to give a suitable tablet.

To prepare a syrup suitable for oral administration, a compound having the structure of Formula (I), (II), or (III) (15 mg per 5 ml of syrup) is added to a solution of 0.1% benzoic acid, 5% alcohol, citric acid, edetate disodium, ethyl maltol, flavors, glycerin, ammoniated glycyrrhizin, propylene glycol, purified water, sodium saccharin, sucrose, FD&C blue #1 and FD&C red #40.

### Example 1c

### Sublingual (Hard Lozenge) Composition

To prepare a pharmaceutical composition for buccal delivery, such as a hard lozenge, mix 100 mg of a compound having the structure of Formula (I), (II), or (III), with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 1d

### Inhalation Composition

To prepare a pharmaceutical composition for inhalation delivery, 20 mg of a compound having the structure of Formula (I), (II), or (III) is mixed with 50 mg of anhydrous citric acid and 100 mL of 0.9% sodium chloride solution. The mixture is incorporated into an inhalation delivery unit, such as a nebulizer, which is suitable for inhalation administration.

### Example 1e

### Rectal Gel Composition

To prepare a pharmaceutical composition for rectal delivery, 100 mg of a compound having the structure of Formula (I), (II), or (III) is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 1f

### Topical Gel Composition

To prepare a pharmaceutical topical gel composition, 100 mg of a compound having the structure of Formula (I), (II), or (III) is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Example 2

### (4aR,4bS,6aS,9aS,9bS)-1,4a,6a-trimethyl-7-(pyridin-3-yl)-4,4a,4b,5,6,6a,9,9a,9b,10-decahydro-1H-indeno[5,4-f]quinolin-2(3H)-one

### Example 2A

### 3-((3aS,5aS,6R,9aR,9bS)-3a,6-dimethyl-3,7-dioxododecahydro-1H-cyclopenta[a]naphthalen-6-yl)propanoic acid

To a mixture of (8R,9S,10R,13S,14S)-10,13-dimethyl-7,8,9,10,11,12,13,14,15,16-decahydro-1H-cyclopenta[a]phenanthrene-3,17(2H,6H)-dione (androstenedione, 5 g, 17.5 mmol) suspended in t-BuOH (200 mL) was added K₂CO₃ (2.9 g, 20.9 mmol, 1.2 equiv) in water (15 mL). After the mixture was heated to 80° C., a solution of KMnO₄ (166 mg, 1.05 mmol, 0.06 equiv) and NaIO₄ (21 g, 99.8 mmol, 5.7 equiv) in water (150 mL) was added dropwise over 1.5 hours. The mixture was heated to 80-90° C. for 5 hours, cooled to room temperature, and filtered. The solid was washed with water (3×). The filtrate was concentrated to remove most of t-BuOH, adjusted pH to 1.5 with 1N HCl, extracted with DCM (3×), dried (Na₂SO₄), concentrated to dryness to give 3-((3aS,5aS,6R,9aR,9bS)-3a,6-dimethyl-3,7-dioxododecahydro-1H-cyclopenta[a]naphthalen-6-yl)propanoic acid as a colorless gum. MS calcd for (C₁₈H₂₆O₄)⁺: 306.2; MS found (electrospray): (M−H)⁻=305.0; ¹H NMR (CDCl₃, 300 MHz) major characteristic peaks: δ 1.15 (s, 3H), 0.90 (s, 3H).

### Example 2B

### (4aR,4bS,6aS,9aS,9bR)-1,4a,6a-trimethyl-4,4a,4b,5,6,6a,9,9a,9b,10-decahydro-1H-indeno[5,4-f]quinoline-2,7(3H,8H)-dione

To 3-((3aS,5aS,6R,9aR,9bS)-3a,6-dimethyl-3,7-dioxododecahydro-1H-cyclopenta[a]naphthalen-6-yl)propanoic acid (7.0 g, 22.8 mmol) in sealed bottle was added methylamine (33% w/w in ethanol, 28 mL, 228 mmol, 10 equiv). The mixture was heated at 140° C. overnight. After being cooled to room temperature, the residue was washed with water, acidified to pH 1.5 with 1N HCl, extracted with ethyl acetate (3×), dried (Na₂ SO₄), concentrated to give (4aR,4bS,6aS,9aS,9bR)-1,4a,6a-trimethyl-4,4a,4b,5,6,6a,9,9a,9b,10-decahydro-1H-indeno[5,4-f]quinoline-2,7(3H,8H)-dione (5 g, 73%). MS calcd for (C₁₉H₂₇NO₂+H)⁺: 302.2; MS found (electrospray): (M+H)⁺=302.2; ¹H NMR (CDCl₃, 300 MHz) major characteristic peaks: δ 4.80 (brs, 1H), 2.85 (s, 3H), 0.80 (s, 3H), 0.60 (s, 3H).

### Example 2C

### (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate

To a solution of (4aR,4bS,6aS,9aS,9bR)-1,4a,6a-trimethyl-4,4a,4b,5,6,6a,9,9a,9b,10-decahydro-1H-indeno[5,4-f]quinoline-2,7(3H,8H)-dione (1.0 g, 3.3 mmol) in DCM (5 mL) was added trifluoromethane sulfonic anhydride (0.61 mL, 3.63 mmol, 1.1 equiv) at room temperature, stirred for 10 min. To the solution was added TEA (0.46 mL, mmol) in DCM (2 mL, 1.0 equiv) dropwise within 20 min. The mixture was stirred 4 hours. TLC indicated SM remained. Additional 0.5 equiv of reagents was added. The mixture was stirred overnight and water (5 mL) was added. The mixture was extracted with DCM (3×). The organic layers were combined, washed with 1N HCl, brine, dried (Na₂ SO₄), concentrated, and purified by chromatography on silica gel (hexanes/ethyl acetate, 1:1) to give (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate (460 mg, 32%). MS calcd for (C₂₀H₂₆F₃NO₄S+H)⁺: 434.1; MS found (electrospray): (M+H)⁺=434.2; ¹H NMR (CDCl₃, 300 MHz) major characteristic peaks: δ 5.60 (s, 1H), 5.05 (brs, 1H), 3.15 (s, 3H), 1.10 (s, 3H), 1.05 (s, 3H).

### Example 2

### (4aR,4bS,6aS,9aS,9bS)-1,4a,6a-trimethyl-7-(pyridin-3-yl)-4,4a,4b,5,6,6a,9,9a,9b,10-decahydro-1H-indeno[5,4-f]quinolin-2(3H)-one

To a solution of (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate (4.3 g, mmol) in THF (100 mL) was added 3-(diethylboryl)pyridine (2.94 g, 20 mmol, 2.0 equiv), (Ph₃P)₂ PdCl₂ (70 mg, 0.1 mmol, 0.01 equiv) and sodium carbonate (4.77 g, 45 mmol, in 40 mL of water). The mixture was degassed and refilled with nitrogen (3×), sealed and heated at 80° C. overnight. The reaction mixture was cooled to room temperature, extracted with DCM (2×), combined, dried (Na₂ SO₄), concentrated and purified by column chromatography on silica gel (DCM/MeOH, 9.5:0.5) to give (4aR,4bS,6aS,9aS,9bS)-1,4a,6a-trimethyl-7-(pyridin-3-yl)-4,4a,4b,5,6,6a,9,9a,9b,10-decahydro-1H-indeno[5,4-f]quinolin-2(3H)-one (1.8 g, 50%) as a off-white solid. MS calcd for (C₂₄H₃₀N₂O+H)⁺: 363.2; MS found (electrospray): (M+H)⁺=363.2; ¹H NMR (CDCl₃, 300 MHz) major characteristic peaks: δ 8.62 (s, 1H), 8.46 (brs, 1H), 7.64 (d, 1H), 7.23 (m, 1H), 6.02 (s, 1H), 5.06 (brs, 1H), 3.13 (s, 3H), 1.10 (s, 3H), 1.06 (s, 3H).

### Example 3

### (4aR,4bS,6aS,9aS,9bS)-4a,6a-dimethyl-7-(pyridin-3-yl)-4,4a,4b,5,6,6a,9,9a,9b,10-decahydro-1H-indeno[5,4-f]quinolin-2(3H)-one

### Example 3A

### (4aR,4bS,6aS,9aS,9bR)-4a,6a-dimethyl-4,4a,4b,5,6,6a,9,9a,9b,10-decahydro-1H-indeno[5,4-f]quinoline-2,7(3H,8H)-dione

To a solution of 3-((3aS,5aS,6R,9aR,9bS)-3a,6-dimethyl-3,7-dioxododecahydro-1H-cyclopenta[a]naphthalen-6-yl)propanoic acid (5.5 g, 17.9 mmol) in ethylene glycol (15 mL) in pressure vessel at −10° C. was added ammonia (2.2 g, 129 mmol). The vessel was sealed and heated to 80° C. for 40 min, later to 120° C. for 30 min, 140° C. for 30 min and 160° C. for min. The reaction mixture was cooled to room temperature, diluted with water (50 mL), acidified to pH 1-1.5 with 1N HCl. The yellow precipitate was filtered, washed with water (3×), dried in vacuo to give (4aR,4bS,6aS,9aS,9bR)-4a,6a-dimethyl-4,4a,4b,5,6,6a,9,9a,9b,10-decahydro-1H-indeno[5,4-f]quinoline-2,7(3H,8H)-dione (3.0 g, 58%) as a yellow solid. ¹H NMR (CDCl₃, 300 MHz) major characteristic peaks: δ 8.20 (s, 1H), 4.90 (brs, 1H), 1.07 (s, 3H), 0.90 (s, 3H).

### Example 3B

### (4aR,4bS,6aS,9aS,9bR)-tert-butyl 4a,6a-dimethyl-2,7-dioxo-2,3,4,4a,4b,5,6,6a,7,8,9,9a,9b,10-tetradecahydro-1H-indeno[5,4-f]quinoline-1-carboxylate

To a solution of (4aR,4bS,6aS,9aS,9bR)-4a,6a-dimethyl-4,4a,4b,5,6,6a,9,9a,9b,10-decahydro-1H-indeno[5,4-f]quinoline-2,7(3H,8H)-dione (8.5 g, 29.6 mmol) in DCM (100 mL) was added TEA (4.94 mL, 35.5 mmol, 1.2 equiv), DMAP (181 mg, 1.48 mmol) and 2.4 equiv of (Boc)₂O (7.74 g, 35.5 mmol). The mixture was heated to reflux overnight. More DMAP (543 mg, 4.44 mmol) and (Boc)₂O (7.74 g, 35.5 mmol) was added. The mixture was refluxed for additional 2 hours. Water was added. The reaction mixture was extracted with DCM (2×), combined, washed with NaH₂ PO₄ (0.5 N aqueous solution), saturated NaHCO3, brine and dried over Na₂ SO₄, concentrated to dryness. The residue was passed through a short silica gel column (DCM/MeOH, 9.5:0.5) to give (4aR,4bS,6aS,9aS,9bR)-tert-butyl 4a,6a-dimethyl-2,7-dioxo-2,3,4,4a,4b,5,6,6a,7,8,9,9a,9b,10-tetradecahydro-1H-indeno[5,4-f]quinoline-1-carboxylate (11 g, 100%) as a brown gum, which was used for next reaction without further purification.

### Example 3C

### 3-((3aS,5aS,6R,9aS,9bS)-3a,6-dimethyl-7-oxo-3-(pyridin-3-yl)-3a,4,5,5a,6,7,8,9,9a,9b-decahydro-1H-cyclopenta[a]naphthalen-6-yl)propanoic acid

To a solution of (4aR,4bS,6aS,9aS,9bR)-tert-butyl 4a,6a-dimethyl-2,7-dioxo-2,3,4,4a,4b,5,6,6a,7,8,9,9a,9b,10-tetradecahydro-1H-indeno[5,4-f]quinoline-1-carboxylate (12 g, 31 mmol) in DCM (150 mL) at 0° C. was added Tf₂O (5.7 mL, 34 mmol). The mixture was stirred at 0° C. for 30 min. To the solution was added a solution of triethylamine (4.3 mL, 31 mmol) in DCM (50 mL) dropwise over 30 min. The mixture was slowly warm up to room temperature, and stirred overnight. After water (25 mL) was added, the reaction mixture was extracted with DCM (2×). The organic layers were combined, washed with NaHCO₃, dried (Na₂ SO₄), concentrated and passed through a short silica gel column, quickly washed with DCM-MeOH (9.5:0.5), concentrated to dry to give ((4aR,4bS,6aS,9aS,9bR)-tert-butyl 4a,6a-dimethyl-2-oxo-7-(trifluoromethylsulfonyloxy)-2,3,4,4a,4b,5,6,6a,9,9a,9b,10-dodecahydro-1H-indeno[5,4-f]quinoline-1-carboxylate (8 g, 50%), which was used for next reaction without further purification. A mixture of ((4aR,4bS,6aS,9aS,9bR)-tert-butyl 4a,6a-dimethyl-2-oxo-7-(trifluoromethylsulfonyloxy)-2,3,4,4a,4b,5,6,6a,9,9a,9b,10-dodecahydro-1H-indeno[5,4-f]quinoline-1-carboxylate (930 mg, 1.79 mmol), 3-(diethylboryl)pyridine (527 mg, 3.58 mmol), (Ph₃P)₂ PdCl₂ (63 mg, 0.11 mmol, 0.05 equiv) and Na₂ CO₃ (854 mg, 8.06 mmol, in 2 mL of water) was heated under nitrogen at 80° C. overnight. After being cooled to room temperature, water was added, extracted with ethyl acetate (3×). The aqueous layers were acidified with 1N HCl to pH 1.5 extracted with ethyl acetate (3×), dried (Na₂ SO₄), concentrated to dry to give 3-((3aS,5aS,6R,9aS9bS)-3a,6-dimethyl-7-oxo-3-(pyridin-3-yl)-3a,4,5,5a,6,7,8,9,9a,9b-decahydro-1H-cyclopenta[a]naphthalen-6-yl)propanoic acid (400 mg, 64%). MS calcd for (C₂₃H₂₉NO₃+H)⁺: 368.2; MS found (electrospray): (M+H)⁺: 368.2; ¹H NMR (CDCl₃, 300 MHz) major characteristic peaks: δ 8.60 (s, 1H), 8.45 (d, 1H), 7.65 (d, 1H), 7.26 (d, 1H), 6.00 (s, 1H), 1.15 (s, 3H), 1.08 (s, 3H).

### Example 3

### (4aR,4bS,6aS,9aS,9bS)-4a,6a-dimethyl-7-(pyridin-3-yl)-4,4a,4b,5,6,6a,9,9a,9b,10-decahydro-1H-indeno[5,4-f]quinolin-2(3H)-one

A mixture of 3-((3aS,5aS,6R,9bS)-3a,6-dimethyl-7-oxo-3-(pyridin-3-yl)-3a,4,5,5a,6,7,8,9,9a,9b-decahydro-1H-cyclopenta[a]naphthalen-6-yl)propanoic acid (1 g, 2.72 mmol) in ethylene glycol (10 mL) and NH₃ (2 g, 118 mmol) in a sealed tube was heated to 80° C. for min, at 120° C. for 30 min, 140° C. for 30 min, and cooled to room temperature. After water was added, the mixture was extracted with ethyl acetate (3×), dried (Na₂ SO₄), concentrated to dryness. The residue was purified on silica gel eluting with (DCM/MeOH, 9.5:0.5) to give (4aR,4bS,6aS,9aS,9bS)-4a,6a-dimethyl-7-(pyridin-3-yl)-4,4a,4b,5,6,6a,9,9a,9b,10-decahydro-1H-indeno[5,4-f]quinolin-2(3H)-one (130 mg, 14%). MS calcd for (C₂₃H₂₈N₂O+H)⁺: 349.2; MS found (electrospray): (M+H)⁺: 349.2; ¹H NMR (CDCl₃, 300 MHz) major characteristic peaks: δ 8.60 (s, 1H), 8.45 (brs, 1H), 7.65 (d, 1H), 7.35 (s, 1H), 7.26 (d, 1H), 6.00 (s, 1H), 4.85 (brs, 1H), 1.15 (s, 3H), 1.05 (s, 3H).

### Example 4

### (3aS,3bS,9aR,9bS,11aS)-5,9a,11a-Trimethyl-1-(pyridin-3-yl)-3b,4,5,8,9,9a,9b,10,11,11a-decahydro-3H-cyclopenta[i]phenanthridin-7(3aH)-one

### Example 4A

### (3S,8R,9S,10R,13S,14S)-3-isopropyl-10,13-dimethyl-3,4,7,8,9,10,11,12,13,14,15,16-dodecahydro-1H-cyclopenta[a]phenanthren-17(2H)-one compound with diisopropylsilanone (1:1)

To a suspension of dehydroisoandrosterone (39.2 g, 136 mmol) in dichloromethane (600 mL) was added 2,6-lutidine (23.8 mL, 1.5 eq), followed by triisopropylsilanyl triflate (50 g, 163 mmol, 1.2 equiv) within 15 min. The mixture was stirred at 25° C. for 0.5 hour. The mixture was then washed twice with 2N HCl, once with saturated NaHCO₃, water and brine, dried over Na₂ SO₄. After removal of solvent a white solid of (3S,8R,9S,10R,13S,14S)-3-isopropyl-10,13-dimethyl-3,4,7,8,9,10,11,12,13,14,15,16-dodecahydro-1H-cyclopenta[a]phenanthren-17(2H)-one compound with diisopropylsilanone (1:1) (65.4 g,) was obtained, which was used directly for the next step. ¹H NMR (CDCl₃, 300 MHz) major characteristic peaks: δ 5.32 (s, 1H), 3.52 (m, 1H), 1.04 (s, 12H), 1.02 (s, 3H), 0.86 (s, 3H).

### Example 4B

### 2-((3aS,4R,5S,7aS)-5-((1R,4S)-4-isopropyl-1-methyl-2oxocyclohexyl)-7a-methyl-1-oxooctahydro-1H-inden-4-yl]-acetic acid compound with diisopropylsilanone (1:1)

To a cold solution of (3S,8R,9S,10R,13S,14S)-3-isopropyl-10,13-dimethyl-3,4,7,8,9,10,11,12,13,14,15,16-dodecahydro-1H-cyclopenta[a]phenanthren-17(2H)-one compound with diisopropylsilanone (1:1) (136 mmol) in dichloromethane-methanol (1 L, 3:1, v/v) at −78° C. was bubbled with ozone until blue color persisted. Excess ozone was purged with nitrogen before dimethyl sulfide (80 mL, 7.5 eq) was added. The mixture was warmed up to room temperature and stirred overnight. After removal of solvent the residue was dissolved in tetrahydrofuran (500 mL), a solution of sodium dihydrogen phosphate (81.6 g, 5 eq) and sulfamic acid (66 g, 5 eq) in water (300 mL) was added. With cooling at 0° C. and vigorous stirring a solution of sodium chlorite (61.5 g, 5 eq) in water (400 mL) was added. After stirring at 0° C. for another hour the mixture was extracted three times with ethyl acetate. The combined organic layers were washed with water, dried over Na₂ SO₄. After removal of solvent a batch of 2-((3aS,4R,5S,7aS)-5-((1R,4S)-4-isopropyl-1-methyl-2oxocyclohexyl)-7a-methyl-1-oxooctahydro-1H-inden-4-yl]-acetic acid compound with diisopropylsilanone (1:1) (18 g) was crystallized out, the rest was purified by silica gel chromatography (2:1 hexane-ethyl acetate) to afford another batch of 36.5 g (total 54.5 g, 81% yield). ¹H NMR (CDCl₃, 300 MHz) major characteristic peaks: δ 4.53 (s, 1H), 3.01 (m, 1H), 1.05 (s, 3H), 1.03 (s, 12H), 0.86 (s, 3H).

### Example 4C

### 2-((3aS,4R,5S,7aS)-5-((1R,4S)-4-isopropyl-1-methyl-2oxocyclohexyl)-7a-methyl-1-oxooctahydro-1H-inden-4-yl]-acetyl azide compound with diisopropylsilanone (1:1)

To a solution of 2-((3aS,4R,5S,7aS)-5-((1R,4S)-4-isopropyl-1-methyl-2oxocyclohexyl)-7a-methyl-1-oxooctahydro-1H-inden-4-yl]-acetic acid compound with diisopropylsilanone (1:1) (54.5 g, 110.6 mmol) in dry toluene (500 mL) was added diphenyl phosphoryl azide (23.9 mL, 1.0 eq) and triethylamine (28.1 mL, 1.5 eq). The mixture was stirred at room temperature for 1 hour. After removal of solvent the residue was purified with silica gel (3:1 to 1:1 hexane-ethyl acetate) to give 2-((3aS,4R,5S,7aS)-5-((1R,4S)-4-isopropyl-1-methyl-2oxocyclohexyl)-7a-methyl-1-oxooctahydro-1H-inden-4-yl]-acetyl azide compound with diisopropylsilanone (1:1) as a colorless syrup (48.5 g, 85% yield). ¹H NMR (CDCl₃, 300 MHz) major characteristic peaks: δ 4.48 (s, 1H), 2.96 (m, 1H), 1.02 (s, 12H), 0.84 (s, 3H).

### Example 4D

### (3aS,3bR,7S,9aR,9bS,11aS)-7-isopropyl-9a,11a-dimethyl-2,3,3a,3b,4,6,7,8,9,9a,9b,10,11,11a-tetradecahydro-1H-cyclopenta[i]phenanthridin-1-one compound with diisopropylsilanone (1:1)

A solution of 2-((3aS,4R,5S,7aS)-5-((1R,4S)-4-isopropyl-1-methyl-2oxocyclohexyl)-7a-methyl-1-oxooctahydro-1H-inden-4-yl]-acetyl azide compound with diisopropylsilanone (1:1) (48.5 g, 93.8 mmol) in dry toluene (500 mL) was heated under nitrogen at 80° C. for 0.5 hour. After cooling neutral alumina (60.0 g) was added, and the mixture was heated again at 70° C. for 2 hours. After filtration and removal of solvent a white solid of (3aS,3bR,7S,9aR,9bS,11aS)-7-isopropyl-9a,11a-dimethyl-2,3,3a,3b,4,6,7,8,9,9a,9b,10,11,11a-tetradecahydro-1H-cyclopenta[i]phenanthridin-1-one compound with diisopropylsilanone (1:1) (35.2 g) was obtained, which was used for the next step without purification. ¹H NMR (CDCl₃, 300 MHz) major characteristic peaks: δ 3.75 (s, 1H), 3.02 (m, 1H), 1.09 (s, 3H), 1.00 (s, 12H), 0.89 (s, 3H).

### Example 4E

### (3aS,3bR,7S,9aR,9bS,11aS)-tert-butyl-9a,11a-dimethyl-1-oxo-7-(triisopropylsilyloxy)-3,3a,3b,4,7,8,9,9a,9b,10,11,11a-dodecahydro-1H-cyclopenta[i]phenanthridine-5(2H)-carboxylate

A mixture of (3aS,3bR,7S,9aR,9bS,11aS)-7-isopropyl-9a,11a-dimethyl-2,3,3a,3b,4,6,7,8,9,9a,9b,10,11,11a-tetradecahydro-1H-cyclopenta[i]phenanthridin-1-one compound with diisopropylsilanone (1:1) (35.2 g, 79 mmol) and Boc anhydride (86 g, 5 eq) in dry pyridine (200 mL) was stirred at room temperature overnight. The solvent was removed in vacuo, and the residue was purified with silica gel chromatography (5:1 to 2:1 hexane-ethyl acetate) to give a white solid of (3aS,3bR,7S,9aR,9bS,11aS)-tert-butyl-9a,11a-dimethyl-1-oxo-7-(triisopropylsilyloxy)-3,3a,3b,4,7,8,9,9a,9b,10,11,11a-dodecahydro-1H-cyclopenta[i]phenanthridine-5(2H)-carboxylate (30.2 g, 59% overall yield for the last two steps). ¹H NMR (CDCl₃, 300 MHz) major characteristic peaks: δ 5.38 (s, 1H), 4.36 (m, 1H), 1.46 (s, 9H), 1.00 (s, 12H), 0.99 (s, 3H), 0.82 (s, 3H).

### Example 4F

### (3aS,3bR,9aR,9bS,11aS)-tert-butyl-9a,11a-dimethyl-1,7-dioxo-3,3a,3b,4,7,8,9,9a,9b,10,11,11a-dodecahydro-1H-cyclopenta[i]phenanthridine-5(2H)-carboxylate

A solution of (3aS,3bR,7S,9aR,9bS,11aS)-tert-butyl-9a,11a-dimethyl-1-oxo-7-(triisopropylsilyloxy)-3,3a,3b,4,7,8,9,9a,9b,10,11,11a-dodecahydro-1H-cyclopenta[i]phenanthridine-5(2H)-carboxylate (30.2 g, 55.3 mmol) in tetrahydrofuran (200 mL) was treated with tetrabutylammonium fluoride (1M in THF, 221 mL, 4 eq). The mixture was stirred at room temperature for 1 hour, and was then diluted with dichloromethane and washed water and brine, dried over Na₂ SO₄. After filtration the filtrate was concentrated to ca. 400 mL, was then added molecular sieves (50 g), followed by N-methylmorpholine N-oxide (9.7 g, 1.5 eq) and tetrapropylammonium perruthenate (1.0 g, 0.05 eq). The mixture was stirred at room temperature for 2 hours. After filtration and concentration the residue was purified with silica gel chromatography (2:1 to 1:1 hexane-ethyl acetate to give pure product (3aS,3bR,9aR,9bS,11aS)-tert-butyl-9a,11a-dimethyl-1,7-dioxo-3,3a,3b,4,7,8,9,9a,9b,10,11,11a-dodecahydro-1H-cyclopenta[i]phenanthridine-5(2H)-carboxylate (21.1 g, 98% yield). MS calcd for (C₂₃H₃₄NO₄)⁺: 387.2; MS found (electrospray): 388.2; ¹H NMR (CDCl₃, 300 MHz) major characteristic peaks: δ 5.79 (s, 1H), 4.39 (dd, 1H), 1.44 (s, 9H), 1.25 (s, 3H), 0.91 (s, 3H).

### Example 4G

### (3aS,3bR,9aR,9bS,11aS)-9a,11a-dimethyl-3,3a,3b,4,5,8,9,9a,9b,10,11,11a-dodecahydro-1H-cyclopenta[i]phenanthridine-1,7(2H)-dione

To a solution of 3aS,3bR,9aR,9bS,11aS)-tert-butyl-9a,11a-dimethyl-1,7-dioxo-3,3a,3b,4,7,8,9,9a,9b,10,11,11a-dodecahydro-1H-cyclopenta[i]phenanthridine-5(2H)-carboxylate (21.1 g, 54.5 mmol) in dichloromethane (400 mL) was added trifluoroacetic acid (90 mL). The mixture was stirred at room temperature for 1 hour. With ice bath cooling the reaction solution was carefully neutralized with 2 N NaOH, was then extracted with dichloromethane (3×) and dried over Na₂ SO₄ to give (3aS,3bR,9aR,9bS,11aS)-9a,11a-dimethyl-3,3a,3b,4,5,8,9,9a,9b,10,11,11a-dodecahydro-1H-cyclopenta[i]phenanthridine-1,7(2H)-dione (17.4 g) to be used for next step without purification. MS calcd for (C₁₈H₂₆NO₂)⁺: 287.2; MS found (electrospray): 288.2; ¹H NMR (CDCl₃, 300 MHz) major characteristic peaks: δ 5.54 (br. s, 1H), 5.17 (s, 1H), 3.48 (m, 1H), 2.95 (m, 1H), 1.26 (s, 3H), 0.92 (s, 3H).

### Example 4H

### (3aS,3bR,9aR,9bS,11aS)-5,9a,11a-trimethyl-3,3a,3b,4,5,8,9,9a,9b,10,11,11a-dodecahydro-1H-cyclopenta[i]phenanthridine-1,7(2H)-dione

To a solution of (3aS,3bR,9aR,9bS,11aS)-9a,11a-dimethyl-3,3a,3b,4,5,8,9,9a,9b,10,11,11a-dodecahydro-1H-cyclopenta[i]phenanthridine-1,7(2H)-dione (5.0 g, 17.4 mmol) in dry N,N′-dimethylformamide (100 mL) was added sodium hydride (1.56 g, 60% in mineral oil, 39 mmol). The mixture was stirred at room temperature for 15 min before iodomethane (1.04 mL, 17.4 mmol) was added. After 30 min further stirring at room temperature the reaction was quenched with saturated NH₄Cl, extracted with dichloromethane (3×). The combined organic layers were washed with water and brine, dried over Na₂ SO₄. After the removal of solvent the crude product was purified with silica gel chromatography (5% methanol in dichloromethane) to give pure product (3aS,3bR,9aR,9bS,11aS)-5,9a,11a-trimethyl-3,3a,3b,4,5,8,9,9a,9b,10,11,11a-dodecahydro-1H-cyclopenta[i]phenanthridine-1,7(2H)-dione (4.22 g, 80% yield). MS calcd for (C₁₉H₂₈NO₂)⁺: 301.2; MS found (electrospray): 302.2; ¹H NMR (CDCl₃, 300 MHz) major characteristic peaks: δ 5.08 (s, 1H), 3.36 (m, 1H), 2.77 (s, 3H), 1.26 (s, 3H), 0.89 (s, 3H).

### Example 41

### (3aS,3bR,9aR,9bS,11aS)-5,9a,11a-trimethyl-7-oxo-3a,3b,4,5,7,8,9,9a,9b,10,11,11a-dodecahydro-3H-cyclopenta[i]phenanthridin-1-yl trifluoromethanesulfonate

To a solution of (3aS,3bR,9aR,9bS,11aS)-5,9a,11a-trimethyl-3,3a,3b,4,5,8,9,9a,9b,10,11,11a-dodecahydro-1H-cyclopenta[i]phenanthridine-1,7(2H)-dione (4.2 g, 14.0 mmol) in dry THF (150 mL) cooling at 0° C. was added KHMDS (0.5M solution in toluene, 33.5 mL, 1.2 eq). After 15 min PhNTf₂ was added as a solution in THF (30 mL). The mixture was stirred at 0° C. for 1 h before quenching with saturated NH₄Cl, extracted with dichloromethane (3×), dried over Na₂ SO₄. After the removal of solvent the crude product was purified with silica gel chromatography (5% methanol in dichloromethane) to give (3aS,3bR,9aR,9bS,11aS)-5,9a,11a-trimethyl-7-oxo-3a,3b,4,5,7,8,9,9a,9b,10,11,11a-dodecahydro-3H-cyclopenta[i]phenanthridin-1-yl trifluoromethanesulfonate (4.6 g, 76% yield). MS calcd for (C₂₀H₂₇F₃NO₄S)⁺: 433.2; MS found (electrospray): 434.2; ¹H NMR (CDCl₃, 300 MHz) major characteristic peaks: δ 5.60 (s, 1H), 5.18 (s, 1H), 3.28 (m, 1H), 2.82 (s, 3H), 1.28 (s, 3H), 1.01 (s, 3H).

### Example 4

### (3aS,3bS,9aR,9bS,11aS)-5,9a,11a-Trimethyl-1-(pyridin-3-yl)-3b,4,5,8,9,9a,9b,10,11,11a-decahydro-3H-cyclopenta[i]phenanthridin-7(3aH)-one

To a solution of (3aS,3bR,9aR,9bS,11aS)-5,9a,11a-trimethyl-7-oxo-3a,3b,4,5,7,8,9,9a,9b,10,11,11a-dodecahydro-3H-cyclopenta[i]phenanthridin-1-yl trifluoromethanesulfonate (4.6 g, 10.6 mmol) in tetrahydrofuran (150 mL) was added diethyl 3-pyridoborane (3.12 g, 2 eq), sodium carbonate (5.06 g, 4.5 eq) in water (30 mL), and bis(triphenylphosphine) palladium chloride (0.75 g, 0.1 eq). The mixture was thoroughly degassed, and heated under nitrogen at 80° C. for overnight. After being filtered through a pad of Celite, the crude product was purified with silica gel column (5% to 10% methanol in dichloromethane) to give (3aS,3bS,9aR,9bS,11aS)-5,9a,11a-trimethyl-1-(pyridin-3-yl)-3b,4,5,8,9,9a,9b,10,11,11a-decahydro-3H-cyclopenta[i]phenanthridin-7(3aH)-one (3.1 g, 81%). MS calcd for (C₂₄H₃₁N₂O)⁺: 362.2; MS found (electrospray): 363.2; ¹H NMR (CDCl₃, 300 MHz) major characteristic peaks: δ 8.61 (brs, 1H), 8.48 (d, 1H), 7.64 (d, 1H), 7.24 (m, 1H), 5.98 (s, 1H), 5.09 (s, 1H), 2.82 (s, 3H), 1.30 (s, 3H), 1.07 (s, 3H).

### Example 5

### (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one

### Example 5A

### 9a,11a-dimethyl-5-((2-(trimethylsilyl)ethoxy)methyl)-3,3a,3b,4,5,8,9,9a,9b,10,11,11a-dodecahydro-1H-cyclopenta[i]phenanthridine-1,7(2H)-dione

To a solution of 9a,11a-dimethyl-2,3,3a,4,5,8,9,9a,9b,10,11,11a-dodecahydro-3bH-cyclopenta[i]phenanthridine-1,7-dione (7.0 g, 24.4 mmol) in DCM (250 mL) was added SEM-Cl (4.88 g, 29.3 mmol) and DIEA (6.05 mL, 36.6 mmol). The solution was stirred at room temperature overnight. The solvent was removed to give a residue, which was purified on silica gel eluting with methanol (5%) in DCM to afford 9a,11a-dimethyl-5-((2-(trimethylsilyl)ethoxy)methyl)-3,3a,3b,4,5,8,9,9a,9b,10,11,11a-dodecahydro-1H-cyclopenta[i]phenanthridine-1,7(2H)-dione (8.75 g, 86%). ¹H NMR (CDCl₃, 300 MHz) major characteristic peaks: δ 5.38 (s, 1H), 4.60 (d, 1H), 4.38 (d, 1H), 1.21 (s, 3H), 0.90 (s, 3H), 0.0 (s, 9H).

### Example 5B

### (3aS,3bR,9aR,9bS,11aS)-9a,11a-dimethyl-7-oxo-5-((2-(trimethylsilyl)ethoxy)methyl)-3a,3b,4,5,7,8,9,9a,9b,10,11,11a-dodecahydro-3H-cyclopenta[i]phenanthridin-1-yl trifluoromethanesulfonate

To a solution of 9a,11a-dimethyl-5-((2-(trimethylsilyl)ethoxy)methyl)-3,3a,3b,4,5,8,9,9a,9b,10,11,11a-dodecahydro-1H-cyclopenta[i]phenanthridine-1,7(2H)-dione (8.75 g, 21.0 mmol) in dry THF (300 mL) cooling at 0° C. was added KHMDS (0.5 M solution in toluene, 50.4 mL, 1.2 eq). After 15 min solid PhNTf₂ (10.5 g, 29.4 mmol) was added. The mixture was stirred at 0° C. for 1 hour before quenching with saturated NH₄Cl, extracted with dichloromethane (3×), dried over Na₂ SO₄. After the removal of solvent the crude product was purified with silica gel chromatography (5% methanol in dichloromethane) to give (3aS,3bR,9aR,9bS,11aS)-9a,11a-dimethyl-7-oxo-5-((2-(trimethylsilyl)ethoxy)methyl)-3a,3b,4,5,7,8,9,9a,9b,10,11,11a-dodecahydro-3H-cyclopenta[i]phenanthridin-1-yl trifluoromethanesulfonate (13.8 g), which contained some PhNTf₂ and PhNHTf. The product was used for next step reaction without further purification. ¹H NMR (CDCl₃, 300 MHz) major characteristic peaks: δ 5.60 (brs, 1H), 5.38 (s, 1H), 4.60 (d, 1H), 4.38 (d, 1H), 1.25 (s, 3H), 1.05 (s, 3H), 0.01 (s, 9H).

### Example 5C

### (3aS,3bS,9aR,9bS,11aS)-9a,1a-dimethyl-1-(pyridin-3-yl)-5-((2-(trimethylsilyl)ethoxy)methyl)-3b,4,5,8,9,9a,9b,10,11,11a-decahydro-3H-cyclopenta[i]phenanthridin-7(3aH)-one

To a solution of (3aS,3bR,9aR,9bS,11aS)-9a,11a-dimethyl-7-oxo-5-((2-(trimethylsilyl)ethoxy)methyl)-3a,3b,4,5,7,8,9,9a,9b,10,11,11a-dodecahydro-3H-cyclopenta[i]phenanthridin-1-yl trifluoromethanesulfonate (13.8 g, 0.021 mmol based previous reaction) in tetrahydrofuran (300 mL) was added diethyl 3-pyridoborane (6.18 g, 2 eq), sodium carbonate (10 g, 4.5 eq) in water (30 mL), and bis(triphenylphosphine) palladium chloride (1.47 g, 0.1 eq). The mixture was thoroughly degassed, and heated under nitrogen at 80° C. for overnight. After being filtered through a pad of Celite, the crude product was purified with silica gel column (5% to 10% methanol in dichloromethane) to give (3aS,3bS,9aR,9bS,11aS)-9a,11a-dimethyl-1-(pyridin-3-yl)-5-((2-(trimethylsilyl)ethoxy)methyl)-3b,4,5,8,9,9a,9b,10,11,11a-decahydro-3H-cyclopenta[i]phenanthridin-7(3aH)-one (7.5 g) as brown sticky solid, which was used for next step reaction without further purification. MS calcd for (C₂₉H₄₂N₂O₂Si)⁺: 478.3; MS found (electrospray): 479.3; ¹H NMR (CDCl₃, 300 MHz) major characteristic peaks: δ 8.60 (brs, 1H), 8.45 (d, 1H), 6.00 (brs, 1H), 5.38 (s, 1H), 4.60 (d, 1H), 4.440 (d, 1H), 1.30 (s, 3H), 0.80 (s, 3H), 0.0 (s, 9H).

### Example 5

### (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one

To a solution of (3aS,3bS,9aR,9bS,11aS)-9a,11a-dimethyl-1-(pyridin-3-yl)-5-((2-(trimethylsilyl)ethoxy)methyl)-3b,4,5,8,9,9a,9b,10,11,11a-decahydro-3H-cyclopenta[i]phenanthridin-7(3aH)-one (7.5 g) in DCM (90 mL) was added trifluoroacetic acid (18 mL). The solution was stirred at room temperature for 1 h. The solution was cooled with an ice-bath and neutralized with NaOH (2 N aqueous solution). The solution was washed with sat. NaHCO3, and dried over sodium sulfate, and concentrated to give a brown solid, which was purified on silica gel eluting with 10% methanol in DCM to give (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one. MS calcd for (C₂₃H₂₈N₂O)⁺: 348.2; MS found (electrospray): M+1, 349.2 ¹H NMR (CDCl₃, 300 MHz) major characteristic peaks: δ 8.59 (brs, 1H), 8.46 (d, 1H), 7.63 (d, 1H), 7.64-7.20 (m, 1H), 5.96 (s, 1H), 5.57 (s, 1H), 1.34 (s, 3H), 1.07 (s, 3H).

### Example 6

### (4aR,4bS,6aS,9aS,9bS)-7-(1H-benzo[d]imidazol-2-yl)-1,4a,6a-trimethyl-4,4a,4b,5,6,6a,9,9a,9b,10-decahydro-1H-indeno[5,4-f]quinolin-2(3H)-one

Dry K₃PO₄ (80 mg, 375 μmol) was transferred into a reaction tube, followed by benzimidazole (88 mg, 750 μmol), XPhos (11.9 mg, 25 μmol) and Pd₂(dba)₃ (11.4 mg, 12.5 μmol). The sealed tube was vacuumed and refilled with N₂ for 6 times, and then dry toluene (1.2 mL) was injected. The resulting deep-red mixture was heated to 60° C. After 30 minutes, a solution of (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate (compound of Example 2C) (108 mg, 250 μmol) in toluene (0.3 mL) was injected via syringe, producing a color change to greenish within approximately 10 minutes. After 1 hour, the temperature was increased to 110° C. and the brown mixture was stirred for 16 hours. The solvent was removed to give a residue, which was purified with HPLC to give (4aR,4bS,6aS,9aS,9bS)-7-(1H-benzo[d]imidazol-2-yl)-1,4a,6a-trimethyl-4,4a,5,6,6a,9,9a,9b,10-decahydro-1H-indeno[5,4-f]quinolin-2(3H)-one (4 mg, 4%). MS calculated for (C₂₆H₃₁N₃O) 401.25; MS found (electrospray, MH⁺): 402.35. ¹HNMR (CDCl₃, 300 MHz) major characteristic peaks: δ 7.60 (2H, m), 7.26 (2H, m), 6.57 (1H, m), 5.09 (1H, d), 3.15 (3H, s), 1.14 (3H, s), 1.12 (3H, s).

### Example 7

### (4aR,4bS,6aS,9aS,9bR)-7-(1H-benzo[d]imidazol-1-yl)-1,4a,6a-trimethyl-4,4a,4b,5,6,6a,9,9a,9b,10-decahydro-1H-indeno[5,4-f]quinolin-2(3H)-one

NaOtBu (34 mg, 340 μmoles) was transferred into a reaction tube, followed by benzimidazole (40 mg, 340 μmoles), XPhos (22 mg, 46 μmoles) and Pd₂(dba)₃ (21 mg, 23 μmoles). The sealed tube was vacuumed and refilled with N₂ for 6 times, and then dry toluene (1 mL) was injected. The resulting deep-red mixture was heated to 60° C. After 30 minutes, a solution of (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate (compound of Example 2C) (100 mg, 230 μmoles) in toluene (0.6 mL) was injected via syringe, producing a color change to greenish within approximately 10 minutes. After 1 hour, the temperature was increased to 110° C. and the brown mixture was stirred for about 16 hours. Afterwards, the solvent was pumped off, and the residue was purified with HPLC to give (4aR,4bS,6aS,9aS,9bR)-7-(1H-benzo[d]imidazol-1-yl)-1,4a,6a-trimethyl-4,4a,4b,5,6,6a,9,9a,9b,10-decahydro-1H-indeno[5,4-f]quinolin-2(3H)-one (4 mg, yield 4%). MS calculated for (C₂₆H₃₁N₃O) 401.25; MS found (electrospray, MH⁺): 402.30. ¹HNMR (CDCl₃, 300 MHz) major characteristic peaks: δ 8.11 (1H, m), 7.86 (1H, m), 7.51 (1H, m), 7.35 (2H, m), 6.06 (1H, s), 5.10 (1H, m), 3.15 (3H, s), 1.12 (3H, s), 1.05 (3H, s).

### Example 8

### (4aR,4bS,6aS,9aS,9bS)-7-(6-Methoxypyridin-3-yl)-1,4a,6a-trimethyl-4,4a,4b,5,6,6a,9,9a,9b,10-decahydro-1H-indeno[5,4-f]quinolin-2(3H)-one

4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate (compound of Example 2C) (87 mg) was mixed with (PPh₃)₂PdCl₂ (11 mg), Na₂ CO₃ (95 mg in 0.4 mL H₂O), and 6-methoxypyridin-3-ylboronic acid (61 mg). The mixture was degassed 3 times and heated to 80° C. for 4 h. The reaction was diluted with ethyl acetate and washed with water. The organic phase was separated, dried, and concentrated. The residue was purified by preparative HPLC to give (4aR,4bS,6aS,9aS,9bS)-7-(6-methoxypyridin-3-yl)-1,4a,6a-trimethyl-4,4a,4b,5,6,6a,9,9a,9b,10-decahydro-1H-indeno[5,4-f]quinolin-2(3H)-one (60 mg) as a white solid. MS calcd for (C₂₅H₃₂N₂O₂+H)⁺: 393.3; MS found: (M+H)⁺=393.3. ¹H NMR (300 MHz, CDCl₃) δ 8.18 (d, 1H), 7.61-7.60 (m, 1H), 6.70 (d, 1H), 5.88-5.87 (m, 1H), 5.09-5.07 (m, 1H), 3.94 (s, 3H), 3.14 (s, 3H), 2.56-2.52 (m, 2H), 2.32-2.24 (m, 2H), 2.23-2.03 (m, 2H), 1.92-1.79 (m, 3H), 1.69-1.46 (m, 5H), 1.26-1.23 (m, 1H), 1.11 (s, 3H), 1.04 (s, 3H).

### Example 9

### (4aR,4bS,6aS,9aS,9bS)-7-(2-Methoxypyrimidin-5-yl)-1,4a,6a-trimethyl-4,4a,4b,5,6,6a,9,9a,9b,10-decahydro-1H-indeno[5,4-f]quinolin-2(3H)-one

Using a synthetic procedure and condition similar to Example 8 in the preparation of (4aR,4bS,6aS,9aS,9bS)-7-(6-methoxypyridin-3-yl)-1,4a,6a-trimethyl-4,4a,4b,5,6,6a,9,9a,9b,10-decahydro-1H-indeno[5,4-f]quinolin-2(3H)-one, replacing 6-methoxypyridin-3-ylboronic acid with 2-methoxypyrimidin-5-ylboronic acid, (4aR,4bS,6aS,9aS,9bS)-7-(2-methoxypyrimidin-5-yl)-1,4a,6a-trimethyl-4,4a,4b,5,6,6a,9,9a,9b,10-decahydro-1H-indeno[5,4-f]quinolin-2(3H)-one was made. MS calcd for (C₂₄H₃₁N₃O₂+H)⁺: 394.3; MS found: (M+H)⁺=394.3. ¹H NMR (300 MHz, CDCl₃) δ 8.50 (s, 2H), 5.97-5.96 (m, 1H), 5.08-5.07 (m, 1H), 4.01 (s, 3H), 3.14 (s, 3H), 2.56-2.52 (m, 2H), 2.34-2.28 (m, 2H), 2.27-1.78 (m, 4H), 1.71-1.46 (m, 5H), 1.25-1.22 (m, 2H), 1.11 (s, 3H), 1.02 (s, 3H).

### Example 10

### (4aR,4bS,6aS,9aS,9bS)-7-(5-Methoxypyridin-3-yl)-1,4a,6a-trimethyl-4,4a,4b,5,6,6a,9,9a,9b,10-decahydro-1H-indeno[5,4-f]quinolin-2(3H)-one

Using a synthetic procedure and condition similar to Example 8 in the preparation of (4aR,4bS,6aS,9aS,9bS)-7-(6-methoxypyridin-3-yl)-1,4a,6a-trimethyl-4,4a,4b,5,6,6a,9,9a,9b,10-decahydro-1H-indeno[5,4-f]quinolin-2(3H)-one, replacing 6-methoxypyridin-3-ylboronic acid with 5-methoxypyridin-3-ylboronic acid, (4aR,4bS,6aS,9aS,9bS)-7-(5-methoxypyridin-3-yl)-1,4a,6a-trimethyl-4,4a,4b,5,6,6a,9,9a,9b,10-decahydro-1H-indeno[5,4-f]quinolin-2(3H)-one was made. MS calcd for (C₂₅H₃₂N₂O₂+H)⁺: 393.3; MS found: (M+H)⁺=393.3. ¹H NMR (300 MHz, CDCl₃) δ 8.25 (s, 1H), 8.18 (s, 1H), 7.17-7.16 (m, 1H), 6.02-6.01 (m, 1H), 5.09-5.07 (m, 1H), 3.89 (s, 3H), 3.14 (s, 3H), 2.56-2.52 (m, 2H), 2.32-2.24 (m, 2H), 2.23-2.03 (m, 2H), 1.92-1.79 (m, 3H), 1.69-1.46 (m, 5H), 1.26-1.23 (m, 1H), 1.11 (s, 3H), 1.04 (s, 3H).

### Example 11

### (4aR,4bS,6aS,9aS,9bS)-1,4a,6a-Trimethyl-7-(4-methylpyridin-3-yl)-4,4a,4b,5,6,6a,9,9a,9b,10-dodecahydro-1H-indeno[5,4-f]quinolin-2(3H)-one

Using a synthetic procedure and condition similar to Example 8 in the preparation of (4aR,4bS,6aS,9aS,9bS)-7-(6-methoxypyridin-3-yl)-1,4a,6a-trimethyl-4,4a,4b,5,6,6a,9,9a,9b,10-decahydro-1H-indeno[5,4-f]quinolin-2(3H)-one, replacing 6-methoxypyridin-3-ylboronic acid with 4-methoxypyridin-3-ylboronic acid, (4aR,4bS,6aS,9aS,9bS)-1,4a,6a-trimethyl-7-(4-methylpyridin-3-yl)-4,4a,4b,5,6,6a,9,9a,9b,10-decahydro-1H-indeno[5,4-f]quinolin-2-one was made. MS calcd for (C₂₅H₃₂N₂O+H)⁺: 377.3; MS found: (M+H)⁺=377.3. ¹H NMR (300 MHz, CD₃OD) δ 8.31-8.30 (m, 1H), 8.23 (s, 1H), 7.36-7.34 (m, 1H), 5.81-5.79 (m, 1H), 5.31-5.29 (m, 1H), 3.17 (s, 3H), 2.56-2.52 (m, 2H), 2.46 (s, 3H), 2.42-2.34 (m, 2H), 2.33-2.08 (m, 2H), 1.92-1.79 (m, 3H), 1.69-1.46 (m, 4H), 1.26-1.23 (m, 2H), 1.11 (s, 3H), 1.04 (s, 3H).

### Example 12

### (4aR,4bS,6aS,9aS,9bS)-7-(2,4-Dimethoxypyrimidin-5-yl)-1,4a,6a-trimethyl-4,4a,4b,5,6,6a,9,9a,9b,10-decahydro-1H-indeno[5,4-f]quinolin-2(3H)-one

Using a synthetic procedure and condition similar to Example 8 in the preparation of (4aR,4bS,6aS,9aS,9bS)-7-(6-methoxypyridin-3-yl)-1,4a,6a-trimethyl-4,4a,4b,5,6,6a,9,9a,9b,10-decahydro-1H-indeno[5,4-f]quinolin-2(3H)-one, replacing 6-methoxypyridin-3-ylboronic acid with 2,4-dimethoxypyrimidin-5-ylboronic acid, 7-(2,4-dimethoxy-pyrimidin-5-yl)-1,4a,6a-trimethyl-4,4a,4b,5,6,6a,9,9a,9b,10-decahydro-1H-indeno[5,4-f]quinolin-2-one was made. MS calcd for (C₂₅H₃₃N₃O₃+H)⁺: 424.3; MS found: (M+H)⁺=424.4. ¹H NMR (300 MHz, CD₃OD) δ 8.09 (s, 1H), 5.99-5.97 (m, 1H), 5.30-5.28 (m, 1H), 4.03 (s, 3H), 4.02 (s, 3H), 3.17 (s, 3H), 2.57-2.52 (m, 2H), 2.51-2.33 (m, 2H), 2.31-2.06 (m, 2H), 2.04-1.75 (m, 3H), 1.73-1.49 (m, 4H), 1.32-1.27 (m, 2H), 1.13 (s, 3H), 1.03 (s, 3H).

### Example 13

### (4aR,4bS,6aS,9aS,9bS)-1,4a,6a-Trimethyl-7-(6-morpholin-4-yl-pyridin-3-yl)-4,4a,4b,5,6,6a,9,9a,9b,10-decahydro-1H-indeno[5,4-f]quinolin-2(3H)-one

Using a synthetic procedure and condition similar to Example 8 in the preparation of (4aR,4bS,6aS,9aS,9bS)-7-(6-methoxypyridin-3-yl)-1,4a,6a-trimethyl-4,4a,4b,5,6,6a,9,9a,9b,10-decahydro-1H-indeno[5,4-f]quinolin-2(3H)-one, replacing 6-methoxypyridin-3-ylboronic acid with 6-morpholinopyridin-3-ylboronic acid, 1,4a,6a-trimethyl-7-(6-morpholin-4-yl-pyridin-3-yl)-4,4a,4b,5,6,6a,9,9a,9b,10-decahydro-1H-indeno[5,4-f]quinolin-2-one was made. MS calcd for (C₂₈H₃₇N₃O₂+H)⁺: 448.3; MS found: (M+H)⁺=448.4. ¹H NMR (300 MHz, CD₃OD) δ 8.18-8.17 (m, 1H), 7.68-7.64 (m, 1H), 6.83-6.80 (m, 1H), 5.92-5.90 (m, 1H), 5.30-5.28 (m, 1H), 4.81-3.81 (m, 4H), 3.50-3.41 (m, 4H), 3.17 (s, 3H), 2.58-2.52 (m, 2H), 2.39-2.30 (m, 2H), 2.29-2.17 (m, 2H), 2.05-1.97 (m, 1H), 1.92-1.50 (m, 8H), 1.15 (s, 3H), 1.10 (s, 3H).

### Example 14

### (4aR,4bS,6aS,9aS,9bS)-7-(2-Aminopyrimidin-5-yl)-1,4a,6a-trimethyl-4,4a,4b,5,6,6a,9,9a,9b,10-decahydro-1H-indeno[5,4-f]quinolin-2(3H)-one

Using a synthetic procedure and condition similar to Example 8 in the preparation of (4aR,4bS,6aS,9aS,9bS)-7-(6-methoxypyridin-3-yl)-1,4a,6a-trimethyl-4,4a,4b,5,6,6a,9,9a,9b,10-decahydro-1H-indeno[5,4-f]quinolin-2(3H)-one, replacing 6-methoxypyridin-3-ylboronic acid with 2-aminopyrimidin-5-ylboronic acid, 7-(2-amino-pyrimidin-5-yl)-1,4a,6a-trimethyl-4,4a,4b,5,6,6a,9,9a,9b,10-decahydro-1H-indeno[5,4-f]quinolin-2-one was made. MS calcd for (C₂₃H₃₀N₄O+H)⁺: 379.2; MS found: (M+H)⁺=379.3. ¹H NMR (300 MHz, CDCl₃) δ 8.33 (br, 2H), 5.88-5.86 (m, 1H), 5.13 (s, 2H), 5.08-5.06 (m, 1H), 3.14 (s, 3H), 2.56-2.51 (m, 2H), 2.33-2.23 (m, 2H), 2.11-1.62 (m, 6H), 1.58-1.42 (m, 3H), 1.42-1.22 (m, 2H), 1.20 (s, 3H), 1.10 (s, 3H).

### Example 15

### (4aR,4bS,6aS,9aS,9bS)-1,4a,6a-Trimethyl-7-(1-methyl-1H-indol-5-yl)-4,4a,4b,5,6,6a,9,9a,9b,10-decahydro-1H-indeno[5,4-f]quinolin-2(3H)-one

Using a synthetic procedure and condition similar to Example 8 in the preparation of (4aR,4bS,6aS,9aS,9bS)-7-(6-methoxypyridin-3-yl)-1,4a,6a-trimethyl-4,4a,4b,5,6,6a,9,9a,9b,10-decahydro-1H-indeno[5,4-f]quinolin-2(3H)-one, replacing 6-methoxypyridin-3-ylboronic acid with 1-methyl-1H-indol-6-ylboronic acid, 1,4a,6a-trimethyl-7-(1-methyl-1H-indol-5-yl)-4,4a,4b,5,6,6a,9,9a,9b,10-decahydro-1H-indeno[5,4-f]quinolin-2-one was made. MS calcd for (C₂₈H₃₄N₂O+H)⁺: 415.3; MS found: (M+H)⁺=415.5. ¹H NMR (300 MHz, CDCl₃) δ 7.62 (s, 1H), 7.27-7.25 (m, 2H), 7.03-7.02 (m, 1H), 6.46-6.45 (m, 1H), 5.87-5.86 (m, 1H), 5.10-5.08 (m, 1H), 3.78 (s, 3H), 3.15 (s, 3H), 2.56-2.52 (m, 2H), 2.31-2.09 (m, 4H), 2.05-1.81 (m, 3H), 1.69-1.50 (m, 5H), 1.27-1.24 (m, 1H), 1.11 (s, 6H).

### Example 16

### Tert-butyl 2-((4aR,4bS,6aS,9aS,9bS)-1,4a,6a-trimethyl-2-oxo-2,3,4,4a,4b,5,6,6a,9,9a,9b,10-dodecahydro-1H-indeno[5,4-f]quinolin-7-yl)-1H-pyrrole-1-carboxylate

Using a synthetic procedure and condition similar to Example 8 in the preparation of (4aR,4bS,6aS,9aS,9bS)-7-(6-methoxypyridin-3-yl)-1,4a,6a-trimethyl-1,3,4,4a,4b,5,6,6a,9,9a,9b,10-decahydro-indeno[5,4-f]quinolin-2(3H)-one, replacing 6-methoxypyridin-3-ylboronic acid with 1 (tert-butoxycarbonyl)-1H-pyrrol-2-ylboronic acid, 2-(1,4a,6a-trimethyl-2-oxo-2,3,4,4a,4b,5,6,6a,9,9a,9b,10-dodecahydro-1H-indeno[5,4-f]quinolin-7-yl)-pyrrole-1-carboxylic acid tert-butyl ester was made. MS calcd for (C₂₈H₃₈N₂O₃+H)⁺: 451.3; MS found: (M+H)⁺=451.3. ¹H NMR (300 MHz, CDCl₃) δ 7.22-7.20 (m, 1H), 6.14-6.12 (m, 1H), 6.01-6.00 (m, 1H), 5.69-5.67 (m, 1H), 5.08-5.06 (m, 1H), 3.13 (s, 3H), 2.55-2.50 (m, 2H), 2.33-2.28 (m, 2H), 2.06-1.92 (m, 2H), 1.88-1.49 (m, 7H), 1.64 (s, 9H), 1.28-1.23 (m, 2H), 1.08 (s, 3H), 0.88 (s, 3H).

### Example 17

### N-5((4aR,4bS,6aS,9aS,9bS)-1,4a,6a-Trimethyl-2-oxo-2,3,4,4a,4b,5,6,6a,9,9a,9b,10-dodecahydro-1H-indeno[5,4-f]quinolin-7-yl)-pyridin-2-yl]-acetamide

Using a synthetic procedure and condition similar to Example 8 in the preparation of (4aR,4bS,6aS,9aS,9bS)-7-(6-methoxypyridin-3-yl)-1,4a,6a-trimethyl-1,3,4,4a,4b,5,6,6a,9,9a,9b,10-decahydro-indeno[5,4-f]quinolin-2(3H)-one, replacing 6-methoxypyridin-3-ylboronic acid with 6-acetamidopyridin-3-ylboronic acid, N-[5-(1,4a,6a-trimethyl-2-oxo-2,3,4,4a,4b,5,6,6a,9,9a,9b,10-dodecahydro-1H-indeno[5,4-f]quinolin-7-yl)-pyridin-2-yl]-acetamide was made. MS calcd for (C₂₆H₃₃N₃O₂+H)⁺: 420.3; MS found: (M+H)⁺=420.4. ¹H NMR (300 MHz, CDCl₃) δ 8.61 (br, 1H), 8.24 (s, 1H), 8.23-8.18 (m, 1H), 7.78-7.74 (m, 1H), 6.00-5.98 (m, 1H), 5.08-5.06 (m, 1H), 3.14 (s, 3H), 2.57-2.52 (m, 2H), 2.34-2.26 (m, 2H), 2.24 (s, 3H), 2.15-2.02 (m, 3H), 1.96-1.46 (m, 6H), 1.43-1.23 (m, 2H), 1.10 (s, 3H), 1.05 (s, 3H).

### Example 18

### (4aR,4bS,6aS,9aS,9bS)-1,4a,6a-trimethyl-7-(pyrimidin-5-yl)-4,4a,4b,5,6,6a,9,9a,9b,10-decahydro-1H-indeno[5,4-f]quinolin-2(3H)-one

Pd(PPh₃)₂Cl₂ (1.2 mg, 0.0017 mmol), 5-pyrimidinylboronic acid (61 mg, 0.49 mmol) and Na₂ CO₃ (2 M, 0.65 mL, 1.3 mmol) were added consecutively to a stirred solution of (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate (125 mg, 0.29 mmol) in THF (15 mL). The reaction was heated to 80° C. under N₂ for 5 hours. Then cooled to room temperature and partitioned between ethyl acetate (100 mL) and water (100 mL). The layers were separated and the aqueous layer extracted with ethyl acetate (25 mL×3). The combined organic layers were dried over Na₂ SO₄. After filtration, the organic phase was concentrated under vacuum and the residue was purified by prep-chromatogram to afford (4aR,4bS,6aS,9aS,9bS)-1,4a,6a-trimethyl-7-(pyrimidin-5-yl)-4,4a,4b,5,6,6a,9,9a,9b,10-decahydro-1H-indeno[5,4-f]quinolin-2(3H)-one as a white solid (80 mg, yield 75%). ¹H NMR (CDCl₃, 400 MHz) major characteristic peaks: δ 9.12 (s, 1H), 8.80 (s, 2H), 6.17 (m, 1H), 5.12 (m, 1H), 3.16 (s, 3H), 1.12 (s, 3H), 1.08 (s, 3H). LC-MS (m/z) 364 [M+H]⁺.

### Example 19

### (4aR,4bS,6aS,9aS,9bS)-1,4a,6a-trimethyl-7-(pyridin-4-yl)-4,4a,4b,5,6,6a,9,9a,9b,10-decahydro-1H-indeno[5,4-f]quinolin-2(3H)-one

Pd(PPh₃)₂Cl₂ (1 mg, 0.001 mmol), pyridine-4-boronic acid (31 mg, 0.26 mmol) and K₂ CO₃ (2 M, 1.25 mL, 0.88 mmol,) were added consecutively to a stirred solution of (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate (65 mg, 0.15 mmol) in THF (10 mL). The reaction was heated to 80° C. and stirred at this temperature for 5 hours. The reaction was cooled to room temperature and partitioned between ethyl acetate (50 mL) and water (50 mL). The layers were separated and the aqueous layer extracted with ethyl acetate (25 mL×3). The combined organic layers were dried over Na₂ SO₄. After filtration, the organic phase was concentrated under vacuum and the residue was purified by prep-HPLC to afford (4aR,4bS,6aS,9aS,9bS)-1,4a,6a-trimethyl-7-(pyridin-4-yl)-4,4a,4b,5,6,6a,9,9a,9b,10-decahydro-1H-indeno[5,4-f]quinolin-2(3H)-one as a white solid (11 mg, yield 20%). ¹H NMR (CDCl₃, 400 MHz) major characteristic peaks: δ 8.54 (s, 2H), 7.29 (s, 2H), 6.21 (s, 1H), 5.06 (m, 1H), 3.14 (s, 3H), 1.12 (s, 3H), 1.10 (s, 3H). LC-MS (m/z) 363 [M+H]⁺.

### Example 20

### (4aR,4bS,6aS,9aS,9bS)-1,4a,6a-trimethyl-7-(pyrazin-2-yl)-4,4a,4b,5,6,6a,9,9a,9b,10-decahydro-1H-indeno[5,4-f]quinolin-2(3H)-one

Pd(PPh₃)₄ (30 mg, 0.027 mmol) and 2-(tributylstannyl)pyrazine (257 mg, 0.693 mmol) were added consecutively to a stirred solution of (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate (200 mg, 0.462 mmol) in DMF (20 mL). The reaction was heated to 120° C. under N₂ for 6 hours. The reaction was cooled to room temperature and partitioned between ethyl acetate (50 mL) and water (50 mL). The layers were separated and the aqueous layer extracted with ethyl acetate (25 mL×3). The combined organic layers were dried over Na₂ SO₄. After filtration, the organic phase was concentrated under vacuum and the residue was purified by prep-HPLC to afford (4aR,4bS,6aS,9aS,9bS)-1,4a,6a-trimethyl-7-(pyrazin-2-yl)-4,4a,4b,5,6,6a,9,9a,9b,10-decahydro-1H-indeno[5,4-f]quinolin-2(3H)-one as a white solid (5 mg, yield 3%). ¹H NMR (CDCl₃, 400 MHz) major characteristic peaks: δ 8.70 (s, 1H), 8.48 (s, 1H), 8.34 (d, J=2 Hz, 1H), 6.49 (m, 1H), 5.09 (t, J=2 Hz, 1H), 3.15 (s, 3H), 1.16 (s, 3H), 1.12 (s, 3H). LC-MS (m/z) 364 [M+H]⁺.

### Example 21

### (4aR,4bS,6aS,9aS,9bS)-1,4a,6a-trimethyl-7-(quinolin-3-yl)-4,4a,4b,5,6,6a,9,9a,9b,10-decahydro-1H-indeno[5,4-f]quinolin-2(3H)-one

Pd(PPh₃)₂Cl₂ (1.5 mg, 0.021 mmol), 3-quinoline boronic acid (103 mg, 0.60 mmol) and K₂ CO₃ (2 M, 2.24 mL, 1.58 mmol) were added consecutively to a stirred solution of (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate (150 mg, 0.35 mmol) in THF (10 mL). The reaction was heated to 80° C. under N₂ for 0.5 hour. The reaction was cooled to room temperature and partitioned between ethyl acetate (50 mL) and water (50 mL). The layers were separated and the aqueous layer extracted with ethyl acetate (25 mL×3). The combined organic layers were dried over Na₂ SO₄. After filtration, the organic phase was concentrated under vacuum and the residue was purified by prep-HPLC to afford (4aR,4bS,6aS,9aS,9bS)-1,4a,6a-trimethyl-7-(quinolin-3-yl)-4,4a,4b,5,6,6a,9,9a,9b,10-decahydro-1H-indeno[5,4-f]quinolin-2(3H)-one as a white solid (14 mg, yield 10%). ¹H NMR (CDCl₃, 400 MHz) major characteristic peaks: δ 9.30 (s, 1H), 8.63 (s, 1H), 8.43 (d, J=8.4 Hz, 1H), 8.05 (d, J=8 Hz, 1H), 7.95 (m, J=7.2 Hz, 1H), 7.82 (m, J=7.2 Hz, 1H), 6.40 (s, 1H), 5.15 (m, 1H), 3.15 (s, 3H), 1.16 (s, 3H), 1.10 (s, 3H). LC-MS (m/z) 413 [M+H]⁺

### Example 22

### (4aR,4bS,6aS,9aS,9bS)-7-(2-chloropyridin-3-yl)-1,4a,6a-trimethyl-4,4a,4b,5,6,6a,9,9a,9b,10-decahydro-1H-indeno[5,4-f]quinolin-2(3H)-one

Pd(PPh₃)₂Cl₂ (1.7 mg, 0.024 mmol), 2-chloropyridin-3-ylboronic acid (105 mg, 0.667 mmol) and 2M K₂ CO₃ (2 M, 2.44 mL, 1.77 mmol) were added consecutively to a stirred solution of (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate (170 mg, 0.393 mmol) in THF (10 mL). The reaction was heated to 80° C. under N₂ for 0.5 hour. The reaction was cooled to room temperature and partitioned between ethyl acetate (50 mL) and water (50 mL). The layers were separated and the aqueous layer extracted with ethyl acetate (25 mL×3). The combined organic layers were dried over Na₂ SO₄. After filtration, the organic phase was concentrated under vacuum and the residue was purified by prep-chromatogram to afford (4aR,4bS,6aS,9aS,9bS)-7-(2-chloropyridin-3-yl)-1,4a,6a-trimethyl-4,4a,4b,5,6,6a,9,9a,9b,10-decahydro-1H-indeno[5,4-f]quinolin-2(3H)-one as a white solid (20 mg, yield 20%). ¹H NMR (CDCl₃, 400 MHz) major characteristic peaks: δ 8.23 (dd, J₁=2.0 Hz, J₂=4.8 Hz, 1H), 7.41 (dd, J₁=2.0 Hz, J₂=4.8 Hz, 1H, J=2.4 Hz), 7.12 (dd, J₁=2.0 Hz, J₂=4.8 Hz, 1H), 5.79 (m, 1H), 5.01 (t, J=2.4 Hz, 1H), 3.08 (s, 3H), 1.03 (s, 3H), 0.91 (s, 3H). LC-MS (m/z) 397 [M+H]⁺.

### Example 23

### (4aR,4bS,6aS,9aS,9bS)-1,4a,6a-trimethyl-7-(pyridin-2-yl)-4,4a,4b,5,6,6a,9,9a,9b,10-decahydro-1H-indeno[5,4-f]quinolin-2(3H)-one

Pd(PPh₃)₄ (30 mg, 0.027 mmol) and 2-(tributylstannyl)pyridine (257 mg, 0.693 mmol) were added consecutively to a stirred solution of (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate (300 mg, 0.693 mmol) in DMF (20 mL). The reaction was heated to 120° C. and stirred at this temperature for 2 hours. The reaction was cooled to room temperature and partitioned between ethyl acetate (50 mL) and water (50 mL). The layers were separated and the aqueous layer extracted with ethyl acetate (25 mL×3). The combined organic layers were dried over Na₂ SO₄. After filtration, the organic phase was concentrated under vacuum and the residue was purified by prep-HPLC to afford (4aR,4bS,6aS,9aS,9bS)-1,4a,6a-trimethyl-7-(pyridin-2-yl)-4,4a,4b,5,6,6a,9,9a,9b,10-decahydro-1H-indeno[5,4-f]quinolin-2(3H)-one as a white solid (23 mg, yield 9%). ¹H NMR (CDCl₃, 400 MHz) major characteristic peaks: δ 9.50 (br, 2H), 8.99 (s, 1H), 8.26 (t, J=7.6 Hz, 1H), 7.74 (d, J=5.2 Hz, 1H), 7.66 (d, J=5.2 Hz, 1H), 7.09 (s, 1H), 5.10 (d, J=4.4 Hz, 1H), 3.14 (s, 3H), 1.17 (s, 3H), 1.12 (s, 3H). LC-MS (m/z) 363 [M+H]⁺.

### Example 24

### (4aR,4bS,6aS,9aS,9bS)-7-(isoquinolin-5-yl)-1,4a,6a-trimethyl-4,4a,4b,5,6,6a,9,9a,9b,10-decahydro-1H-indeno[5,4-f]quinolin-2(3H)-one

Pd(dppf)Cl₂ (10 mg, 5% w/w), 5-isoquinolineboronic acid (136 mg, 0.785 mmol) and K₂ CO₃ (287 mg, in 1 mL water, 2.08 mmol) were added consecutively to a stirred solution of (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate (200 mg, 0.462 mmol) in 1,4-dioxane (15 mL). The reaction was heated to 80° C. under N₂ for 1.5 hours. The reaction was cooled to room temperature and partitioned between ethyl acetate (50 mL) and water (50 mL). The layers were separated and the aqueous layer extracted with ethyl acetate (25 mL×3). The combined organic layers were dried over Na₂ SO₄. After filtration, the organic phase was concentrated under vacuum and the residue was purified by prep-TLC (using PE/EA=1/1) to afford (4aR,4bS,6aS,9aS,9bS)-7-(isoquinolin-5-yl)-1,4a,6a-trimethyl-4,4a,4b,5,6,6a,9,9a,9b,10-decahydro-1H-indeno[5,4-f]quinolin-2(3H)-one as a white solid (39 mg, yield 20%). ¹H NMR (CDCl₃, 400 MHz) major characteristic peaks: δ 9.23 (s, 1H), 8.49 (d, J=4.4 Hz, 1H), 7.89 (d, J=8 Hz, 1H), 7.85 (d, J=6 Hz, 1H), 7.57 (t, J=7.2 Hz, 1H), 7.09 (dd, J₁=7.2 Hz, J₂=1.2 Hz, 1H), 5.82 (m, 1H), 5.10 (m, 1H), 3.15 (s, 3H), 1.09 (s, 3H), 1.02 (s, 3H). LC-MS (m/z) 413 [M+H]⁺.

### Example 25

### (4aR,4bS,6aS,9aS,9bS)-7-(5-chloropyridin-3-yl)-1,4a,6a-trimethyl-4,4a,4b,5,6,6a,9,9a,9b,10-decahydro-1H-indeno[5,4-f]quinolin-2(3H)-one

Pd(PPh₃)₂Cl₂ (10 mg, 5% w/w), 5-chloropyridine-3-boronic acid (124 mg, 0.785 mmol) and K₂ CO₃ (287 mg in 1 mL water, 2.08 mmol) were added consecutively to a stirred solution of (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate (200 mg, 0.462 mmol) in 1,4-dioxane (15 mL). The reaction was heated to 80° C. under N₂ for 3 hours. The reaction was cooled to room temperature and partitioned between ethyl acetate (50 mL) and water (50 mL). The layers were separated and the aqueous layer extracted with ethyl acetate (25 mL×3). The combined organic layers were dried over Na₂ SO₄. After filtration, the organic phase was concentrated under vacuum and the residue was purified by prep-TLC (using PE/EA=1/1) to afford (4aR,4bS,6aS,9aS,9bS)-7-(5-chloropyridin-3-yl)-1,4a,6a-trimethyl-4,4a,4b,5,6,6a,9,9a,9b,10-decahydro-1H-indeno[5,4-f]quinolin-2(3H)-one as a white solid (84 mg, yield 46%). ¹H NMR (CDCl₃, 400 MHz) major characteristic peaks: δ 8.49 (d, J=2 Hz, 1H), 8.43 (d, J=2.4 Hz, 1H), 7.63 (t, J=2 Hz, 1H), 6.06 (m, 1H), 5.07 (m, 1H), 3.14 (s, 3H), 1.11 (s, 3H), 1.06 (s, 3H). LC-MS (m/z) 397 [M+H]⁺.

### Example 26

### (4aR,4bS,6aS,9aS,9bS)-7-(4-chloropyridin-3-yl)-1,4a,6a-trimethyl-4,4a,4b,5,6,6a,9,9a,9b,10-decahydro-1H-indeno[5,4-f]quinolin-2(3H)-one

Pd(PPh₃)₂Cl₂ (10 mg, 5% w/w), 4-chloropyridin-3-ylboronic acid (124 mg, 0.785 mmol) and K₂ CO₃ (287 mg in 1 mL water, 2.08 mmol) is added consecutively to a stirred solution of (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate (200 mg, 0.462 mmol) in 1,4-dioxane (15 mL). The reaction is heated to 80° C. under N₂ for 3 hours. The reaction is cooled to room temperature and partitioned between ethyl acetate (50 mL) and water (50 mL). The layers are separated and the aqueous layer extracted with ethyl acetate (25 mL×3). The combined organic layers are dried over Na₂ SO₄. After filtration, the organic phase is concentrated under vacuum and the residue is purified by prep-TLC (using PE/EA=1/1) to afford (4aR,4bS,6aS,9aS,9bS)-7-(4-chloropyridin-3-yl)-1,4a,6a-trimethyl-4,4a,4b,5,6,6a,9,9a,9b,10-decahydro-1H-indeno[5,4-f]quinolin-2(3H)-one.

### Example 27

### (4aR,4bS,6aS,9aS,9bS)-1-ethyl-4a,6a-dimethyl-7-(pyridin-3-yl)-4,4a,4b,5,6,6a,9,9a,9b,10-decahydro-1H-indeno[5,4-f]quinolin-2(3H)-one

### Example 27A

### (4aR,4bS,6aS,9aS,9bR)-1-ethyl-4a,6a-dimethyl-4,4a,4b,5,6,6a,9,9a,9b,10-decahydro-1H-indeno[5,4-f]quinoline-2, 7 (3H,8H)-dione

A mixture of 3-((3aS,5aS,6R,9aR,9bS)-3a,6-dimethyl-3,7-dioxododecahydro-1H-cyclopenta[a]naphthalen-6-yl) propanoic acid (1, 3.0 g, 9.8 mmol) and ethylamine (4 M in ethanol, 10 mL, 40 mmol) was heated at 140° C. under microwave for 1 hour. After being cooled to room temperature, the residue was washed out with water, acidified to pH 1.5 with 1N HCl, extracted with dichloromethane (3×50 mL), dried over Na₂ SO₄, concentrated to give (4aR,4bS,6aS,9aS,9bR)-1-ethyl-4a,6a-dimethyl-4,4a,4b,5,6,6a,9,9a,9b,10-decahydro-1H-indeno[5,4-f]quinoline-2, 7 (3H,8H)-one (1.85 g, 60%) as a yellow solid. ¹H NMR (CDCl₃, 400 MHz) major characteristic peaks: δ 0.92 (s, 3H), 1.05 (s, 3H), 1.13 (t, J=7.6 Hz, 3H), 3.68 (m, 1H), 3.82 (m, 1H), 5.13 (s, 1H). LCMS (m/z) 316 [M+H]⁺.

### Example 27B

### (4aR,4bS,6aS,9aS,9bR)-1-ethyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate

To a stirred solution of (4aR,4bS,6aS,9aS,9bR)-1-ethyl-4a,6a-dimethyl-4,4a,4b,5,6,6a,9,9a,9b,10-decahydro-1H-indeno[5,4-f]quinoline-2, 7 (3H,8H)-dione (1.85 g, 5.9 mmol) in dry DCM (20 mL) was added Tf₂O (1.10 mL, 6.5 mmol) and the mixture was stirred at room temperature for 30 minutes. Then a solution of triethylamine (0.82 mL, 5.9 mmol) in dry DCM (20 mL) was added over 30 minutes. The resulting mixture was stirred at room temperature for 3.5 hours, and then quenched by addition of water (75 mL) and the layers separated. The aqueous layer was extracted with DCM (3×50 mL). The combined organic layer was washed with 2 N HCl (30 mL) and brine (30 mL), dried over Na₂ SO₄, concentrated. The residue was purified with silica gel column chromatography (Hexane/EtOAc, 10/1) to give (4aR,4bS,6aS,9aS,9bR)-1-ethyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (560 mg, yield 21%). LCMS (m/z) 448 [M+H]⁺.

### Example 27

### (4aR,4bS,6aS,9aS,9bS)-1-ethyl-4a,6a-dimethyl-7-(pyridin-3-yl)-4,4a,4b,5,6,6a,9,9a,9b,10-decahydro-1H-indeno[5,4-f]quinolin-2(3H)-one

To a stirred solution of (4aR,4bS,6aS,9aS,9bR)-1-ethyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (600 mg, 1.34 mmol) in 1,4-dioxane (10 mL) was added pyridin-3-ylboronic acid (280 mg, 2.28 mmol), Pd(PPh₃)₂Cl₂ (48 mg, 0.067 mmol), cesium carbonate (1.96 g, 6.03 mmol) and water (2 mL). The resulting mixture was stirred at 100° C. under N₂ for 0.5 h, then cooled to room temperature and partitioned with EA (50 mL) and water (50 mL). The aqueous layer was extracted with EA (50 mL×3). The combined organic layers were dried over Na₂ SO₄ and concentrated. The residue was purified by prep-HPLC to give (4aR,4bS,6aS,9aS,9bS)-1-ethyl-4a,6a-dimethyl-7-(pyridin-3-yl)-4,4a,4b,5,6,6a,9,9a,9b,10-decahydro-1H-indeno[5,4-f]quinolin-2(3H)-one (120 mg, yield 24%) as a white solid. ¹H NMR (CDCl₃, 400 MHz) major characteristic peaks: δ 1.07 (s, 3H), 1.08 (s, 3H), 1.17 (t, J=5.6 Hz, 3H), 3.68 (m, 1H), 3.82 (m, 1H), 5.14 (d, J=1.6 Hz, 1H), 6.03 (s, 1H), 7.26 (m, 1H), 7.68 (m, 1H), 8.48 (d, J=1.6 Hz, 1H), 8.63 (d, J=1.6 Hz, 1H). LCMS (m/z) 377 [M+H]⁺.

### Example 28

### (4aR,4bS,6aS,9aS,9bS)-1-ethyl-4a,6a-dimethyl-7-(pyridin-4-yl)-4,4a,4b,5,6,6a,9,9a,9b,10-decahydro-1H-indeno[5,4-f]quinolin-2(3H)-one

To a stirred solution of (4aR,4bS,6aS,9aS,9bR)-1-ethyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (500 mg, 1.12 mmol) in dioxane (10 mL) was added pyridin-4-ylboronic acid (275 mg, 2.24 mmol), Pd(PPh₃)Cl₂ (79 mg, 0.11 mmol), Cs₂ CO₃ (1.09 g, 3.36 mmol) and water (3 mL). The mixture was stirred at reflux under N₂ for 2 hrs, and then cooled to room temperature and partitioned with EA (10 mL) and water (10 mL). The aqueous layer was extracted with EA (50 mL×3). The combined organic layers were dried over Na₂ SO₄ and concentrated. The residue was purified by prep-HPLC to give (4aR,4bS,6aS,9aS,9bS)-1-ethyl-4a,6a-dimethyl-7-(pyridin-4-yl)-4,4a,4b,5,6,6a,9,9a,9b,10-decahydro-1H-indeno[5,4-f]quinolin-2(3H)-one (108 mg, yield 26%) as a white solid. ¹HNMR (CDCl₃, 400 MHz) major characteristic peaks: δ 1.09 (s, 3H), 1.11 (s, 3H), 1.14 (t, J=5.6 Hz, 3H), 3.70 (m, 1H), 3.82 (m, 1H), 5.14 (d, J=3.6 Hz, 1H), 6.22 (s, 1H), 7.29 (d, J=6.0 Hz, 2H), 8.52 (d, J=5.6 Hz, 2H).

### Example 29

### (4aR,4bS,6aS,9aS,9bS)-1-ethyl-4a,6a-dimethyl-7-(pyrimidin-5-yl)-4,4a,4b,5,6,6a,9,9a,9b,10-decahydro-1H-indeno[5,4-f]quinolin-2(3H)-one

To a stirred solution of (4aR,4bS,6aS,9aS,9bR)-1-ethyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (100 mg, 0.22 mmol) in dry THF (5 mL) was added pyrimidin-5-ylboronic acid (47 mg, 0.38 mmol), Pd(PPh₃)Cl₂ (8 mg, 0.011 mmol), sodium acetate (82 mg, 0.99 mmol) and water (0.2 mL). The mixture was stirred at 80° C. under N₂ for 2 hr, and then cooled to room temperature and partitioned with EA (10 mL) and water (10 mL). The aqueous layer was extracted with EA (50 mL×3). The combined organic layers were dried over Na₂ SO₄ and concentrated. The residue was purified by prep-HPLC to give (4aR,4bS,6aS,9aS,9bS)-1-ethyl-4a,6a-dimethyl-7-(pyrimidin-5-yl)-4,4a,4b,5,6,6a,9,9a,9b,10-decahydro-1H-indeno[5,4-f]quinolin-2(3H)-one (10 mg, yield 12%) as a white solid. ¹H NMR (CDCl₃, 400 MHz) major characteristic peaks: δ 1.07 (s, 3H), 1.08 (s, 3H), 1.14 (t, J=5.6 Hz, 3H), 3.70 (m, 1H), 3.82 (m, 1H), 5.15 (d, J=4.0 Hz, 1H), 6.13 (s, 1H), 8.75 (s, 2H), 9.09 (s, 1H). LCMS (m/z) 378 [M+H]⁺.

### Example 30

### (4aR,4bS,6aS,9aS,9bS)-1-ethyl-4a,6a-dimethyl-7-(pyrazin-2-yl)-4,4a,4b,5,6,6a,9,9a,9b,10-decahydro-1H-indeno[5,4-f]quinolin-2(3H)-one

To a stirred solution of (4aR,4bS,6aS,9aS,9bR)-1-ethyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (200 mg, 0.45 mmol) in dry DMF (6 mL) wad added 2-(tributylstannyl)pyrazine (248 mg, 0.76 mmol) and Pd(PPh₃)₄ (46 mg, 0.04 mmol). The mixture was stirred at 120° C. for 2 hrs, and then cooled to room temperature and partitioned with EA (10 mL) and water (10 mL). The aqueous layer was extracted with EA (50 mL×3). The combined organic layers were dried over Na₂ SO₄ and concentrated. The residue was purified by prep-TLC to give (4aR,4bS,6aS,9aS,9bS)-1-ethyl-4a,6a-dimethyl-7-(pyrazin-2-yl)-4,4a,4b,5,6,6a,9,9a,9b,10-decahydro-1H-indeno[5,4-f]quinolin-2(3H)-one (20 mg, yield 12%) as a white solid. ¹H NMR (CDCl₃, 400 MHz) major characteristic peaks: δ 5.14 (m, 1H), 6.67 (s, 1H), 8.43 (d, J=2.4 Hz, 1H), 8.55 (s, 1H), 8.87 (s, 1H). LCMS (m/z) 378 [M+H]⁺.

### Example 31

### (4aR,4bS,6aS,9aS,9bS)-1-ethyl-4a,6a-dimethyl-7-(quinolin-3-yl)-4,4a,4b,5,6,6a,9,9a,9b,10-decahydro-1H-indeno[5,4-f]quinolin-2(3H)-one

To a stirred solution of (4aR,4bS,6aS,9aS,9bR)-1-ethyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (200 mg, 0.44 mmol) in 1,4-dioxane (10 mL) was added Pd(PPh₃)₂Cl₂ (2.0 mg, 0.0028 mmol), quinolin-3-ylboronic acid (128 mg, 0.74 mmol) and Cs₂ CO₃ (2 M, 1.0 mL, 1.98 mmol). The mixture was stirred at 100° C. under N₂ for 15 min, and then cooled to room temperature and partitioned with EA (10 mL) and water (10 mL). The aqueous layer was extracted with EA (50 mL×3). The combined organic layers were dried over Na₂ SO₄ and concentrated. The residue was purified by prep-TLC to give (4aR,4bS,6aS,9aS,9bS)-1-ethyl-4a,6a-dimethyl-7-(quinolin-3-yl)-4,4a,4b,5,6,6a,9,9a,9b,10-decahydro-1H-indeno[5,4-f]quinolin-2(3H)-one (28 mg, yield 15%) as a white solid. ¹H NMR (400 MHz, CDCl₃): δ 1.10 (s, 3H), 1.15 (m, 6H), 1.27 (m, 1H), 1.50 (m, 5H), 1.90 (m, 3H), 2.18 (m, 2H), 2.36 (m, 2H), 2.52 (m, 2H), 3.70 (m, 1H), 3.83 (m, 1H), 5.15 (m, 1H), 6.19 (m, 1H), 7.55 (t, J=7.6 Hz, 1H), 7.08 (t, J=7.6 Hz, 1H), 7.81 (d, J=7.6 Hz, 1H), 8.09 (s, 2H), 8.99 (s, 1H). LCMS (m/z) 427 [M+H]⁺.

### Example 32

### (4aR,4bS,6aS,9aS,9bS)-7-(2-chloropyridin-3-yl)-1-ethyl-4a,6a-dimethyl-4,4a,4b,5,6,6a,9,9a,9b,10-decahydro-1H-indeno[5,4-f]quinolin-2(3H)-one

To a stirred solution of (4aR,4bS,6aS,9aS,9bR)-1-ethyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (200 mg, 0.45 mmol) in dioxane (10 mL) was added 2-chloropyridin-3-ylboronic acid (120 mg, 0.76 mmol), Pd(PPh₃)Cl₂ (32 mg, 0.04 mmol), Cs₂ CO₃ (438 mg, 1.34 mmol) and water (1.5 mL). The reaction was stirred at reflux under N₂ for 2 hrs, and then cooled to room temperature and partitioned with EA (10 mL) and water (10 mL). The aqueous layer was extracted with EA (50 mL×3). The combined organic layers were dried over Na₂ SO₄ and concentrated. The residue was purified by prep-TLC to give (4aR,4bS,6aS,9aS,9bS)-7-(2-chloropyridin-3-yl)-1-ethyl-4a,6a-dimethyl-4,4a,4b,5,6,6a,9,9a,9b,10-decahydro-1H-indeno[5,4-f]quinolin-2(3H)-one (78 mg, yield 34%) as a white solid. ¹HNMR (CDCl₃, 400 MHz) major characteristic peaks: δ 0.98 (s, 3H), 1.06 (s, 3H), 1.14 (t, J=7.2 Hz, 3H), 3.70 (m, 1H), 3.82 (m, 1H), 5.14 (m, 1H), 5.87 (s, 1H), 7.21 (d, J=7.6 Hz, 4.8 Hz, 1H), 7.50 (dd, J₁=7.6 Hz, J=2.0 Hz, 1H), 8.31 (d, J=4.8 Hz, 1.6 Hz, 1H). LCMS (m/z) 411 [M+H]⁺.

### Example 33

### (4aR,4bS,6aS,9aS,9bS)-1-ethyl-4a,6a-dimethyl-7-(pyridin-2-yl)-4,4a,4b,5,6,6a,9,9a,9b,10-decahydro-1H-indeno[5,4-f]quinolin-2(3H)-one

To a stirred solution of (4aR,4bS,6aS,9aS,9bR)-1-ethyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (3, 200 mg, 0.447 mmol) in DMF (10 mL) were added 2-(tributylstannyl)pyridine (329 mg, 0.9 mmol), Pd(PPh₃)₄ (20 mg, 0.02 mmol). The mixture was stirred at 120° C. under N₂ for 3 h, and then cooled to room temperature and partitioned with EA (10 mL) and water (10 mL). The aqueous layer was extracted with EA (50 mL×3). The combined organic layers were dried over Na₂ SO₄ and concentrated. The residue was purified by TLC to give (4aR,4bS,6aS,9aS,9bS)-1-ethyl-4a,6a-dimethyl-7-(pyridin-2-yl)-4,4a,4b,5,6,6a,9,9a,9b,10-decahydro-1H-indeno[5,4-f]quinolin-2(3H)-one (5 mg, yield 3%) as a white solid. ¹H NMR (CDCl₃, 400 MHz) major characteristic peaks: δ 1.03 (s, 3H), 1.07 (s, 3H), 1.15 (t, J=5.6 Hz, 3H), 3.75 (m, 2H), 5.17 (d, J=3.6 Hz, 1H), 6.41 (s, 1H), 7.11 (t, J=6.0 Hz, 1H), 7.39 (d, J=8.4 Hz, 1H), 7.60 (t, J=7.2 Hz, 1H), 8.56 (d, J=4.0 Hz, 1H). LCMS (m/z) 377 [M+H]⁺.

### Example 34

### (4aR,4bS,6aS,9aS,9bS)-1-ethyl-7-(isoquinolin-4-yl)-4a,6a-dimethyl-4,4a,4b,5,6,6a,9,9a,9b,10-decahydro-1H-indeno[5,4-f]quinolin-2(3H)-one

To a stirred solution of (4aR,4bS,6aS,9aS,9bR)-1-ethyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (200 mg, 0.447 mmol) in 1,4-dioxane (10 mL) were added isoquinolin-4-ylboronic acid (116 mg, 0.67 mmol), Pd(PPh₃)₂Cl₂ (20 mg, 0.02 mmol), potassium carbonate (185 mg, 1.34 mmol) and water (2 mL). The mixture was stirred at 120° C. under N₂ for 3 h, and then cooled to room temperature and partitioned with EA (10 mL) and water (10 mL). The aqueous layer was extracted with EA (50 mL×3). The combined organic layers were dried over Na₂ SO₄ and concentrated. The residue was purified by prep-TLC (EA/PE=3:1) to give (4aR,4bS,6aS,9aS,9bS)-1-ethyl-7-(isoquinolin-4-yl)-4a,6a-dimethyl-4,4a,4b,5,6,6a,9,9a,9b,10-decahydro-1H-indeno[5,4-f]quinolin-2(3H)-one (15 mg, yield 8%) as a white solid. ¹HNMR (CDCl₃, 400 MHz) major characteristic peaks: δ 1.03 (s, 3H), 1.07 (s, 3H), 1.15 (t, J=5.6 Hz, 3H), 3.75 (m, 2H), 5.17 (s, 1H), 5.89 (s, 1H), 7.60 (m, 1H), 7.68 (m, 1H), 8.00 (q, J=8 Hz, 2H), 8.34 (s, 1H), 9.17 (s, 1H). LCMS (m/z) 427 [M+H]⁺.

### Example 35

### (4aR,4bS,6aS,9aS,9bS)-1-Cyclopropyl-4a,6a-dimethyl-7-(pyridin-3-yl)-4,4a,4b,5,6,6a,9,9a,9b,10-decahydro-1H-indeno[5,4-f]quinolin-2(3H)-one

### Example 35A

### (4aR,4bS,6aS,9aS,9bR)-1-Cyclopropyl-4a,6a-dimethyl-4,4a,4b,5,6,6a,9,9a,9b,10-decahydro-1H-indeno[5,4-f]quinoline-2,7(3H,8H)-dione

A suspension of 3-((3aS,5aS,6R,9aR,9bS)-3a,6-dimethyl-3,7-dioxododecahydro-1H-cyclopenta[a]naphthalen-6-yl) propanoic acid (5.1 g, 17 mmol) in ethylene glycol (30 mL) in ice-bath was treated with cyclopropanamine (6.65 g, 117 mmol,). The mixture was stirred at room temperature for 1 h. Then the solution was slowly (3° C./min) heated to reach 180° C. and keep at this temperature for 30 min. After being cooled to room temperature, the residue was washed out with water, extracted with DCM (30 mL×3), dried, concentrated to give (4aR,4bS,6aS,9aS,9bR)-1-cyclopropyl-4a,6a-dimethyl-4,4a,4b,5,6,6a,9,9a,9b,10-decahydro-1H-indeno[5,4-f]quinoline-2,7(3H,8H)-dione (1.6 g, yield 30%). ¹H NMR (CDCl₃, 400 MHz) major characteristic peaks: δ 5.37 (m, 1H), 0.72 (m, 2H), 0.39 (m, 1H). LC-MS (m/z) 328 [M+H]⁺.

### Example 35B

### (4aR,4bS,6aS,9aS,9bR)-1-Cyclopropyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate

To a stirred solution of (4aR,4bS,6aS,9aS,9bR)-1-cyclopropyl-4a,6a-dimethyl-4,4a,4b,5,6,6a,9,9a,9b,10-decahydro-1H-indeno[5,4-f]quinoline-2, 7 (3H,8H)-dione (1.1 g, 3.5 mmol) in DCM (20 mL) was added trifluoromethanesulfonic anhydride (1.2 g, 4.2 mmol) and the mixture was stirred at room temperature for 10 minutes. A solution of triethylamine (0.35 g, 3.5 mmol) in DCM (3 mL) was added dropwise at 0° C. over 30 min. The resulting purple solution was stirred at room temperature for 0.5 hour, and then quenched by addition of water (25 mL) and the layers separated. The aqueous layer was extracted with DCM (75 mL×3). The combined organic fraction was washed with 2N HCl (75 mL) and brine (75 mL), dried with Na₂ SO₄ and concentrated to afford the crude product. The mixture was purified by column chromatography on silica gel (PE/EA, 3/1) to give (4aR,4bS,6aS,9aS,9bR)-1-cyclopropyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (500 mg, yield 31%). ¹H NMR (DMSO, 400 MHz) major characteristic peaks: δ 5.75 (s, 1H), 5.29 (m, 1H), 2.51 (m, 1H), 0.97 (s, 3H), 0.92 (s, 3H), 1.01 (s, 3H), 0.18 (s, 1H). LC-MS (m/z) 460 [M+H]⁺.

### Example 35

### (4aR,4bS,6aS,9aS,9bS)-1-Cyclopropyl-4a,6a-dimethyl-7-(pyridin-3-yl)-4,4a,4b,5,6,6a,9,9a,9b,10-decahydro-1H-indeno[5,4-f]quinolin-2(3H)-one

Pd(PPh₃)₂Cl₂ (2.0 mg, 0.0028 mmol), pyridin-3-ylboronic acid (92 mg, 0.74 mmol) and K₂ CO₃ (2 M, 1.00 mL, 1.98 mmol) were added consecutively to a stirred solution of (4aR,4bS,6aS,9aS,9bR)-1-cyclopropyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (200 mg, 0.44 mmol) in 1,4-dioxane (10 mL). The reaction was heated to 100° C. under N₂ for 0.5 hour. The reaction was cooled to room temperature and partitioned between ethyl acetate (100 mL) and water (100 mL). The layers were separated and the aqueous layer extracted with ethyl acetate (25 mL×3). The combined organic layers were dried over Na₂ SO₄. After filtration, the organic phase was concentrated under vacuum to afford (4aR,4bS,6aS,9aS,9bS)-1-cyclopropyl-4a,6a-dimethyl-7-(pyridin-3-yl)-4,4a,4b,5,6,6a,9,9a,9b,10-decahydro-1H-indeno[5,4-f]quinolin-2(3H)-one as a white solid (80 mg, yield 47%). ¹H-NMR (400 MHz, CDCl₃): 0.29 (m, 1H), 0.78 (m, 2H), 1.00 (s, 3H), 1.07 (s, 3H), 1.23 (m, 2H), 1.51-1.79 (m, 8H), 2.04-2.16 (m, 2H), 2.29-2.36 (m, 2H), 2.42-2.48 (m, 2H), 2.58 (m, 1H), 5.38 (m, 1H), 6.02 (m, 1H), 7.24 (m, 1H), 7.66 (m, 1H), 8.47 (m, 1H), 8.63 (s, 1H). LC-MS (m/z) 389 [M+H]⁺.

### Example 36

### (4aR,4bS,6aS,9aS,9bS)-1-Cyclopropyl-4a,6a-dimethyl-7-(pyridin-4-yl)-4,4a,4b,5,6,6a,9,9a,9b,10-decahydro-1H-indeno[5,4-f]quinolin-2(3H)-one

Pd(PPh₃)₂Cl₂ (2.0 mg, 0.0028 mmol), pyridin-4-ylboronic acid (92 mg, 0.74 mmol) and K₂ CO₃ (2 M, 1.0 mL, 1.98 mmol) were added consecutively to a stirred solution of (4aR,4bS,6aS,9aS,9bR)-1-cyclopropyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (200 mg, 0.44 mmol) in 1,4-dioxane (10 mL). The mixture was heated to 100° C. under N₂ for 0.5 hour. The reaction was cooled to room temperature and partitioned between ethyl acetate (100 mL) and water (100 mL). The layers were separated and the aqueous layer extracted with ethyl acetate (25 mL×3). The combined organic layers were dried over Na₂ SO₄. After filtration, the organic phase was concentrated under vacuum and the residue was purified by prep-HPLC to afford (4aR,4bS,6aS,9aS,9bS)-1-Cyclopropyl-4a,6a-dimethyl-7-(pyridin-4-yl)-4,4a,4b,5,6,6a,9,9a,9b,10-decahydro-1H-indeno[5,4-f]quinolin-2(3H)-one as a white solid (48 mg, yield 29%). ¹H-NMR (CDCl₃, 400 MHz) major characteristic peaks: δ 8.44-8.46 (d, J=6.0 Hz, 2H), 7.21-7.22 (d, J=6.0 Hz, 2H), 6.14-6.15 (q, J=2.0 Hz, 1H), 5.30-5.32 (d, J=4.4 Hz, 1H), 2.51 (m, 1H), 1.03 (s, 3H), 0.93 (s, 3H), 0.23 (s, 1H). LC-MS (m/z) 389 [M+H]⁺.

### Example 37

### (4aR,4bS,6aS,9aS,9bS)-1-cyclopropyl-4a,6a-dimethyl-7-(pyrimidin-5-yl)-4,4a,4b,5,6,6a,9,9a,9b,10-decahydro-1H-indeno[5,4-f]quinolin-2(3H)-one

Pd(PPh₃)₂Cl₂ (1.2 mg, 0.0017 mmol), 5-pyrimidinylboronic acid (56 mg, 0.44 mmol) and Na₂ CO₃ (2 M, 120 mg, 1.2 mmol) were added consecutively to a stirred solution of (4aR,4bS,6aS,9aS,9bR)-1-cyclopropyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (120 mg, 0.26 mmol) in THF (10 mL). The mixture was heated to 80° C. under N₂ and stirred at this temperature for 5 hours. The reaction was cooled to room temperature and partitioned between ethyl acetate (100 mL) and water (100 mL). The layers were separated and the aqueous layer extracted with ethyl acetate (25 mL×3). The combined organic layers were dried over Na₂ SO₄. After filtration, the organic phase was concentrated under vacuum and the residue was purified by prep-HPLC to afford (4aR,4bS,6aS,9aS,9bS)-1-cyclopropyl-4a,6a-dimethyl-7-(pyrimidin-5-yl)-4,4a,4b,5,6,6a,9,9a,9b,10-decahydro-1H-indeno[5,4-f]quinolin-2(3H)-one as a white solid (13 mg, yield 13%). ¹H NMR (CDCl₃, 400 MHz) major characteristic peaks: δ 9.01 (s, 1H), 8.67 (s, 2H), 6.06 (m, 1H), 5.31 (d, J=3.6 Hz, 1H), 2.51 (s, 1H), 1.00 (s, 3H), 0.93 (s, 3H), 0.23 (s, 1H). LC-MS (m/z) 390 [M+H]⁺.

### Example 38

### (4aR,4bS,6aS,9aS,9bS)-1-Cyclopropyl-4a,6a-dimethyl-7-(pyrazin-2-yl)-4,4a,4b,5,6,6a,9,9a,9b,10-decahydro-1H-indeno[5,4-f]quinolin-2(3H)-one

To a mixture of (4aR,4bS,6aS,9aS,9bR)-1-cyclopropyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (100 mg, 0.218 mmol) and 2-(tributylstannyl)pyrazine (112 mg, 0.3 mmol) in DMF (2 mL) was added Pd(PPh₃)₄ (catalytic amount), The mixture was heated to 120° C. under N₂ for 2 hours. Then cooled to room temperature and partitioned between ethyl acetate (50 mL) and water (50 mL). The layers were separated and the aqueous layer extracted with ethyl acetate (25 mL×3). The combined organic layers were dried over Na₂ SO₄. After filtration, the organic phase was concentrated under vacuum and the residue was purified by prep-HPLC to afford (4aR,4bS,6aS,9aS,9bS)-1-cyclopropyl-4a,6a-dimethyl-7-(pyrazin-2-yl)-4,4a,4b,5,6,6a,9,9a,9b,10-decahydro-1H-indeno[5,4-f]quinolin-2(3H)-one as a white solid (6 mg, yield 6%). ¹H-NMR (CDCl₃, 400 MHz) major characteristic peaks: δ 8.65 (s, 1H), 8.45 (s, 1H), 8.29 (s, 1H), 8.46 (s, 1H), 5.31-5.32 (d, J=5.2 Hz, 1H), 2.53 (s, 1H), 1.15 (s, 3H), 1.10 (s, 3H), 0.25 (s, 1H). LC-MS (m/z) 390 [M+H]⁺.

### Example 39

### (4aR,4bS,6aS,9aS,9bS)-1-Cyclopropyl-4a,6a-dimethyl-7-(quinolin-3-yl)-4,4a,4b,5,6,6a,9,9a,9b,10-decahydro-1H-indeno[5,4-f]quinolin-2(3H)-one

Pd(PPh₃)₂Cl₂ (2.8 mg, 0.0039 mmol), quinolin-4-ylboronic acid (179 mg, 1.04 mmol) and K₂ CO₃ (2 M, 1.4 mL, 2.74 mmol) were added consecutively to a stirred solution of (4aR,4bS,6aS,9aS,9bR)-1-cyclopropyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (280 mg, 0.61 mmol) in 1,4-dioxane (15 mL). The mixture was heated to 90° C. under N₂ for 10 minute. Then cooled to room temperature and partitioned between ethyl acetate (100 mL) and water (100 mL). The layers were separated and the aqueous layer extracted with ethyl acetate (25 mL×3). The combined organic layers were dried over Na₂ SO₄. After filtration, the organic phase was concentrated under vacuum and the residue was purified by prep-HPLC to afford (4aR,4bS,6aS,9aS,9bS)-1-cyclopropyl-4a,6a-dimethyl-7-(quinolin-3-yl)-4,4a,4b,5,6,6a,9,9a,9b,10-decahydro-1H-indeno[5,4-f]quinolin-2(3H)-one as a white solid (80 mg, yield 30%). ¹H-NMR (CDCl₃, 400 MHz) major characteristic peaks: δ 8.90 (d, J=2.0 Hz, 1H), 8.03 (d, J=8.0 Hz, 2H), 7.73 (d, J=8.4 Hz, 1H), 7.62 (t, J=7.2 Hz, 1H), 7.48 (t, J=7.2 Hz, 1H), 6.12 (s, 1H), 5.33 (d, J=4.0 Hz, 1H), 2.52 (m, 1H), 1.1 (s, 3H), 0.95 (s, 3H), 0.24 (s, 1H). LC-MS (m/z) 439 [M+H]⁺.

### Example 40

### (4aR,4bS,6aS,9aS,9bS)-7-(2-Chloropyridin-3-yl)-1-cyclopropyl-4a,6a-dimethyl-4,4a,4b,5,6,6a,9,9a,9b,10-decahydro-1H-indeno[5,4-f]quinolin-2(3H)-one

Pd(PPh₃)₂Cl₂ (1.0 mg, 0.0014 mmol), 2-chloropyridin-3-ylboronic acid (58 mg, 0.37 mmol) and K₂ CO₃ (2 M, 0.5 mL, 0.98 mmol) were added consecutively to a stirred solution of (4aR,4bS,6aS,9aS,9bR)-1-cyclopropyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (100 mg, 0.22 mmol) in 1,4-dioxane (5 mL). The mixture was heated to 100° C. under N₂ for 0.5 hour. Then cooled to room temperature and partitioned between ethyl acetate (50 mL) and water (50 mL). The layers were separated and the aqueous layer extracted with ethyl acetate (25 mL×3). The combined organic layers were dried over Na₂ SO₄. After filtration, the organic phase was concentrated under vacuum to afford (4aR,4bS,6aS,9aS,9bS)-7-(2-chloropyridin-3-yl)-1-cyclopropyl-4a,6a-dimethyl-4,4a,4b,5,6,6a,9,9a,9b,10-decahydro-1H-indeno[5,4-f]quinolin-2(3H)-one as a white solid (18 mg, yield 19%). ¹H-NMR (400 MHz, CDCl₃): 0.31 (m, 1H), 0.80 (m, 3H), 0.98 (s, 6H), 1.20 (m, 3H), 1.48 (m, 3H), 1.80 (m, 6H), 2.17 (m, 1H), 2.40 (m, 4H), 2.50 (m, 1H), 5.39 (m, 1H), 5.86 (m, 1H), 7.19 (m, 1H), 748 (m, 1H), 8.30 (m, 1H). LC-MS (m/z) 423 [M+H]⁺.

### Example 41

### (4aR,4bS,6aS,9aS,9bS)-1-Cyclopropyl-4a,6a-dimethyl-7-(pyridin-2-yl)-4,4a,4b,5,6,6a,9,9a,9b,10-decahydro-1H-indeno[5,4-f]quinolin-2(3H)-one

To a mixture of (4aR,4bS,6aS,9aS,9bR)-1-cyclopropyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (200 mg, 0.436 mmol) and 2-(tributylstannyl)pyridine (225 mg, 0.61 mmol) in DMF (2 mL) was added Pd(PPh₃)₄ (catalytic amount), The mixture were heated to 120° C. under N₂ for 2 hours. The reaction was cooled to room temperature and partitioned between ethyl acetate (100 mL) and water (100 mL). The layers were separated and the aqueous layer extracted with ethyl acetate (25 mL×3). The combined organic layers were dried over Na₂ SO₄. After filtration, the organic phase was concentrated under vacuum and the residue was purified by prep-HPLC to afford (4aR,4bS,6aS,9aS,9bS)-1-cyclopropyl-4a,6a-dimethyl-7-(pyridin-2-yl)-4,4a,4b,5,6,6a,9,9a,9b,10-decahydro-1H-indeno[5,4-f]quinolin-2(3H)-one as a white solid (7 mg, yield 3%). ¹H-NMR (CDCl₃, 400 MHz) major characteristic peaks: δ 8.49-8.50 (d, J=4.4 Hz, 1H), 7.54-7.55 (t, J=6.0 Hz, 1H), 7.32-7.34 (d, J=8.4 Hz, 1H), 7.03-7.06 (t, J=6.0 Hz, 1H), 6.34 (s, 1H), 5.31-5.32 (d, J=4.0 Hz, 1H), 2.51 (m, 1H), 1.10 (s, 3H), 0.93 (s, 3H), 0.24 (s, 1H). LC-MS (m/z) 389 [M+H]⁺.

### Example 42

### (4aR,4bS,6aS,9aS,9bS)-1-Cyclopropyl-7-(isoquinolin-5-yl)-4a,6a-dimethyl-4,4a,4b,5,6,6a,9,9a,9b,10-decahydro-1H-indeno[5,4-f]quinolin-2(3H)-one

Pd(PPh₃)₂Cl₂ (1.0 mg, 0.0014 mmol), isoquinolin-5-ylboronic acid (45 mg, 0.255 mmol) and K₂ CO₃ (2 M, 0.5 mL, 0.87 mmol) were added consecutively to a stirred solution of (4aR,4bS,6aS,9aS,9bR)-1-cyclopropyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (90 mg, 0.20 mmol) in 1,4-dioxane (5 mL). The mixture was heated to 120° C. under N₂ for 0.5 hour. Then cooled to room temperature and partitioned between ethyl acetate (50 mL) and water (50 mL). The layers were separated and the aqueous layer extracted with ethyl acetate (15 mL×3). The combined organic layers were dried over Na₂ SO₄. After filtration, the organic phase was concentrated under vacuum and the residue was purified by prep-chromatogram to afford (4aR,4bS,6aS,9aS,9bS)-1-cyclopropyl-7-(isoquinolin-5-yl)-4a,6a-dimethyl-4,4a,4b,5,6,6a,9,9a,9b,10-decahydro-1H-indeno[5,4-f]quinolin-2(3H)-one as a white solid (29 mg, yield 34%). ¹H-NMR (CDCl₃, 400 MHz) major characteristic peaks: δ 9.25 (s, 1H), 8.50 (s, 1H), 7.90 (d, J=8.4 Hz, 1H), 7.87 (d, J=5.6 Hz, 1H), 7.58 (q, J=7.2 Hz, 1H), 7.50 (d, J=8.0 Hz, 1H), 5.83 (t, J=1.6 Hz, 1H), 5.41-5.42 (dd, J₁=1.6 Hz, J₂=5.2 Hz, 1H), 2.59 (m, 1H), 1.02 (s, 3H), 1.00 (s, 3H), 0.31 (s, 1H). LC-MS (m/z) 439 [M+H]⁺.

### Example 43

### (4aR,4bS,6aS,9aS,9bS)-1-Cyclopropyl-7-(isoquinolin-4-yl)-4a,6a-dimethyl-4,4a,4b,5,6,6a,9,9a,9b,10-decahydro-1H-indeno[5,4-f]quinolin-2(3H)-one

Pd(PPh₃)₂Cl₂ (1.0 mg, 0.0014 mmol), isoquinolin-4-ylboronic acid (64 mg, 0.37 mmol) and K₂ CO₃ (2 M, 0.5 mL, 0.98 mmol) were added consecutively to a stirred solution of (4aR,4bS,6aS,9aS,9bR)-1-cyclopropyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (3, 100 mg, 0.22 mmol) in 1,4-dioxane (5 mL). The mixture was heated to 100° C. under N₂ for 0.5 hour. Then cooled to room temperature and partitioned between ethyl acetate (50 mL) and water (50 mL). The layers were separated and the aqueous layer extracted with ethyl acetate (25 mL×3). The combined organic layers were dried over Na₂ SO₄. After filtration, the organic phase was concentrated under vacuum to afford (4aR,4bS,6aS,9aS,9bS)-1-cyclopropyl-7-(isoquinolin-4-yl)-4a,6a-dimethyl-4,4a,4b,5,6,6a,9,9a,9b,10-decahydro-1H-indeno[5,4-f]quinolin-2(3H)-one as a white solid (40 mg, yield 42%). ¹H-NMR (400 MHz, CDCl₃): 0.33 (m, 1H), 0.80 (m, 2H), 0.98 (s, 3H), 1.03 (s, 3H), 1.25 (m, 2H), 1.53 (m, 5H), 1.85 (m, 4H), 2.30 (m, 1H), 2.46 (m, 4H), 2.60 (m, 1H), 5.41 (m, 1H), 5.88 (m, 1H), 7.60 (m, 1H), 7.68 (m, 1H), 8.00 (m, 2H), 8.34 (s, 1H), 9.17 (s, 1H). LC-MS (m/z) 439 [M+H]⁺.

### Example 44

### (4aR,4bS,6aS,9aS,9bS)-1-(2-(Dimethylamino)ethyl)-4a,6a-dimethyl-7-(pyridin-3-yl)-4,4a,4b,5,6,6a,9,9a,9b,10-decahydro-1H-indeno[5,4-f]quinolin-2(3H)-one

### Example 44A

### (4aR,4bS,6aS,9aS,9bR)-1-(2-(dimethylamino)ethyl)-4a,6a-dimethyl-4,4a,4b,5,6,6a,9,9a,9b,10-decahydro-1H-indeno[5,4-f]quinoline-2,7(3H,8H)-dione

A solution of 3-((3aS,5aS,6R,9aR,9bS)-3a,6-dimethyl-3,7-dioxododecahydro-1H-cyclopenta[a]naphthalen-6-yl) propanoic acid (15.0 g, 49 mmol) and N¹,N¹-dimethylethane-1,2-diamine (30.2 g, 0.3 mol) in methanol (120 mL) was stirred under microwave at 145° C. for 45 min. Then cooled down to room temperature, concentrated, the residue was washed out with water, extracted with DCM (100 mL×3), dried, concentrated to give (4aR,4bS,6aS,9aS,9bR)-1-(2-(dimethylamino)ethyl)-4a,6a-dimethyl-4,4a,4b,5,6,6a,9,9a,9b,10-decahydro-1H-indeno[5,4-f]quinoline-2,7(3H,8H)-dione (8.8 g, yield 60%). LC-MS (m/z) 359 [M+H]⁺.

### Example 44B

### (4aR,4bS,6aS,9aS,9bR-E)-1-(2-(Dimethylamino)ethyl)-7-hydrazono-4a,6a-dimethyl-4,4a,4b,5,6,6a,7,8,9,9a,9b,10-dodecahydro-1H-indeno[5,4-f]quinolin-2(3H)-one

To a suspension containing of (4aR,4bS,6aS,9aS,9bR)-1-(2-(dimethylamino)ethyl)-4a,6a-dimethyl-4,4a,4b,5,6,6a,9,9a,9b,10-decahydro-1H-indeno[5,4-f]quinoline-2, 7 (3H,8H)-dione (30 g, 8.4 mmol) in ethanol (40 mL), hydrazine hydrate (8.0 g, 251 mmol) and triethylamine (2.54 g, 25.1 mmol) were added, and the mixture was stirred under reflux for 2 hours. Then cooled down, concentrated to give (4aR,4bS,6aS,9aS,9bRE)-1-(2-(dimethylamino)ethyl)-7-hydrazono-4a,6a-dimethyl-4,4a,4b,5,6,6a,7,8,9,9a,9b,10-dodecahydro-1H-indeno[5,4-f]quinolin-2(3H)-one (3.1 g). LC-MS (m/z) 373 [M+H]⁺.

### Example 44C

### (4aR,4bS,6aS,9aS,9bR)-1-(2-(Dimethylamino)ethyl)-7-iodo-4a,6a-dimethyl-4,4a,4b,5,6,6a,9,9a,9b,10-decahydro-1H-indeno[5,4-f]quinolin-2(3H)-one

Iodine (11.7 g, 46.2 mmol) was dissolved in dry THF (80 mL) and dry ether (40 mL). The solution was cooled in an ice bath and then treated with 1,1,3,3-tetramethylguanidine (5.8 g, 50.8 mmol). A solution of (4aR,4bS,6aS,9aS,9bR-E)-1-(2-(dimethylamino)ethyl)-7-hydrazono-4a,6a-dimethyl-4,4a,4b,5,6,6a,7,8,9,9a,9b,10-dodecahydro-1H-indeno[5,4-f]quinolin-2(3H)-one (3.21 g, 8.4 mmol) in THF (40 mL) was added dropwise into the iodine solution over 2 hours maintaining the reaction temperature at 0° C. Then solvents were removed in vacuum, and the residue was re-dissolved with methylene chloride and washed with Na₂ SO₃ and brine. The solution was dried over Na₂ SO₄ and then concentrated to give (4aR,4bS,6aS,9aS,9bR)-1-(2-(dimethylamino)ethyl)-7-iodo-4a,6a-dimethyl-4,4a,4b,5,6,6a,9,9a,9b,10-decahydro-1H-indeno[5,4-f]quinolin-2(3H)-one. LC-MS (m/z) 469 [M+H]⁺.

### Example 44

### (4aR,4bS,6aS,9aS,9bS)-1-(2-(Dimethylamino)ethyl)-4a,6a-dimethyl-7-(pyridin-3-yl)-4,4a,4b,5,6,6a,9,9a,9b,10-decahydro-1H-indeno[5,4-f]quinolin-2(3H)-one

Pd(PPh₃)₂Cl₂ (1.7 mg, 0.024 mmol), pyridin-3-ylboronic acid (105 mg, 0.667 mmol) and K₂ CO₃ (2M, 2.5 mL, 1.77 mmol) were added to a stirred solution of (4aR,4bS,6aS,9aS, 9bR)-1-(2-(dimethylamino)ethyl)-7-iodo-4a,6a-dimethyl-4,4a,4b,5,6,6a,9,9a,9b,10-decahydro-1H-indeno[5,4-f]quinolin-2(3H)-one (170 mg, 0.393 mmol) in 1,4-dioxane (10 mL). The mixture was heated to 85° C. under N₂ for 1 hour, then cooled down to room temperature. Water (50 mL) was added, the mixture was extracted with ethyl acetate (25 mL×3). The combined organic layers were dried over Na₂ SO₄, filtered, concentrated. The residue was purified by prep-HPLC to afford (4aR,4bS,6aS,9aS,9bS)-1-(2-(dimethylamino)ethyl)-4a,6a-dimethyl-7-(pyridin-3-yl)-4,4a,4b,5,6,6a,9,9a,9b,10-decahydro-1H-indeno[5,4-f]quinolin-2(3H)-one as a white solid (55 mg, yield 45%). ¹H NMR (CDCl₃, 400 MHz) major characteristic peaks: δ 8.81 (s, 1H), 8.61 (m, 1H), 8.19 (m, 1H), 7.71 (m, 1H), 6.24 (m, 1H), 5.18 (m, 1H), 4.18 (m, 1H), 3.87 (m, 1H), 3.16 (m, 2H), 2.86 (s, 6H), 1.04 (s, 3H), 1.01 (s, 3H). LC-MS (m/z) 420 [M+H]⁺.

### Example 45

### (4aR,4bS,6aS,9aS,9bS)-1-(2-(Dimethylamino)ethyl)-4a,6a-dimethyl-7-(quinolin-3-yl)-4,4a,4b,5,6,6a,9,9a,9b,10-decahydro-1H-indeno[5,4-f]quinolin-2(3H)-one

Pd(PPh₃)₂Cl₂ (1.7 mg, 0.024 mmol), quinolin-3-ylboronic acid (126 mg, 0.73 mmol) and K₂ CO₃ (2M, 2.5 mL, 1.77 mmol) were added to a stirred solution of (4aR,4bS,6aS,9aS,9bR)-1-(2-(dimethylamino)ethyl)-7-iodo-4a,6a-dimethyl-4,4a,4b,5,6,6a,9,9a,9b,10-decahydro-1H-indeno[5,4-f]quinolin-2(3H)-one (200 mg, 0.43 mmol) in 1,4-dioxane (10 mL). The mixture was heated to 85° C. under N₂ for 1 hour, then cooled down to room temperature. Water (50 mL) was added, the mixture was extracted with ethyl acetate (25 mL×3). The combined organic layers were dried over Na₂ SO₄, filtered, concentrated. The residue was purified by prep-HPLC to afford (4aR,4bS,6aS,9aS,9bS)-1-(2-(dimethylamino)ethyl)-4a,6a-dimethyl-7-(quinolin-3-yl)-4,4a,4b,5,6,6a,9,9a,9b,10-decahydro-1H-indeno[5,4-f]quinolin-2(3H)-one as a white solid (19 mg, yield 11%). ¹H NMR (CDCl₃, 400 MHz) major characteristic peaks: δ 8.96 (d, J=2.0 Hz, 1H), 8.06 (m, 2H), 7.78 (d, J=8.0 Hz, 1H), 7.66 (t, J=9.0 Hz, 1H), 7.54 (t, J=9.0 Hz, 1H), 6.17 (m, 1H), 5.20 (t, J=4.4 Hz, 1H), 3.87 (m, 2H), 2.31 (s, 6H), 1.14 (s, 3H), 1.09 (s, 3H). LC-MS (m/z) 470 [M+H]⁺.

### Example 46

### (4aR,4bS,6aS,9aS,9bS)-7-(2-Chloropyridin-3-yl)-1-(2-(dimethylamino)ethyl)-4a,6a-dimethyl-4,4a,4b,5,6,6a,9,9a,9b,10-decahydro-1H-indeno[5,4-f]quinolin-2(3H)-one

Pd(PPh₃)₂Cl₂ (1.7 mg, 0.024 mmol), 2-chloropyridin-3-ylboronic acid (115 mg, 0.73 mmol) and K₂ CO₃ (2M, 2.5 mL, 1.77 mmol) were added to a stirred solution of (4aR,4bS,6aS,9aS,9bR)-1-(2-(dimethylamino)ethyl)-7-iodo-4a,6a-dimethyl-4,4a,4b,5,6,6a,9,9a,9b,10-decahydro-1H-indeno[5,4-f]quinolin-2(3H)-one (200 mg, 0.43 mmol) in 1,4-dioxane (10 mL). The mixture was heated to 85° C. under N₂ for 1 hour, then cooled down to room temperature. Water (50 mL) was added, the mixture was extracted with ethyl acetate (25 mL×3). The combined organic layers were dried over Na₂ SO₄, filtered, concentrated. The residue was purified by prep-HPLC to afford (4aR,4bS,6aS,9aS,9bS)-7-(2-chloropyridin-3-yl)-1-(2-(dimethylamino)ethyl)-4a,6a-dimethyl-4,4a,4b,5,6,6a,9,9a,9b,10-decahydro-1H-indeno[5,4-f]quinolin-2(3H)-one as a white solid (35 mg, yield 38%). ¹H NMR (CDCl₃, 400 MHz) major characteristic peaks: δ 8.29 (dd, J; =2 Hz, J₂=4.8 Hz, 1H), 7.47 (dd, J₁=2 Hz, J₂=7.2 Hz, 1H), 7.19 (dd, J₁=4.8 Hz, J₂=7.2 Hz, 1H), 5.85 (m, 1H), 5.25 (t, J=4.0 Hz, 1H), 4.21 (m, 1H), 3.97 (m, 1H), 3.17 (t, J=8.0 Hz, 2H), 2.89 (s, 6H), 1.05 (s, 3H), 0.97 (s, 3H). LC-MS (m/z) 454 [M+H]⁺.

### Example 47

### (4aR,4bS,6aS,9aS,9bS)-1-(2-(Dimethylamino)ethyl)-7-(isoquinolin-5-yl)-4a,6a-dimethyl-4,4a,4b,5,6,6a,9,9a,9b,10-decahydro-1H-indeno[5,4-f]quinolin-2(3H)-one

Pd(PPh₃)₂Cl₂ (1.7 mg, 0.024 mmol), isoquinolin-5-ylboronic acid (126 mg, 0.73 mmol) and K₂ CO₃ (2M, 2.5 mL, 1.77 mmol) were added to a stirred solution of (4aR,4bS,6aS,9aS,9bR)-1-(2-(dimethylamino)ethyl)-7-iodo-4a,6a-dimethyl-4,4a,4b,5,6,6a,9,9a,9b,10-decahydro-1H-indeno[5,4-f]quinolin-2(3H)-one (200 mg, 0.43 mmol) in 1,4-dioxane (10 mL). The mixture was heated to 85° C. under N₂ for 1 hour, then cooled down to room temperature. Water (50 mL) was added, the mixture was extracted with ethyl acetate (25 mL×3). The combined organic layers were dried over Na₂ SO₄, filtered, concentrated. The residue was purified by prep-HPLC to afford (4aR,4bS,6aS,9aS,9bS)-1-(2-(dimethylamino)ethyl)-7-(isoquinolin-5-yl)-4a,6a-dimethyl-4,4a,4b,5,6,6a,9,9a,9b,10-decahydro-1H-indeno[5,4-f]quinolin-2(3H)-one as a white solid (27 mg, yield 21%). ¹H NMR (CDCl₃, 400 MHz) major characteristic peaks: δ 9.17 (s, 1H), 8.42 (d, J=6.0 Hz, 1H), 7.82 (d, J=8.0 Hz, 1H), 7.78 (d, J=5.6 Hz, 1H), 7.50 (t, J=7.2 Hz, 1H), 7.42 (d, J=7.2 Hz, 1H), 5.83 (s, 1H), 5.23 (m, 1H), 3.89 (m, 2H), 2.37 (s, 6H), 1.07 (s, 3H), 1.02 (s, 3H). LC-MS (m/z) 470 [M+H]⁺.

### Example 48

### 2-((4aR,4bS,6aS,9aS,9bS)-4a,6a-Dimethyl-2-oxo-7-(pyridin-3-yl)-2,3,4,4a,4b,5,6,6a,9,9a,9b,10-dodecahydroindeno[5,4-f]quinolin-1-yl)-N,N-dimethylacetamide

### Example 48A

### (4aR,4bS,6aS,9aS,9bR)-4a,6a-Dimethyl-4,4a,4b,5,6,6a,9,9a,9b,10-decahydro-1H-indeno[5,4-f]quinoline-2,7(3H,8H)-dione

To a solution of 3-((3aS,5aS,6R,9aR,9bS)-3a,6-dimethyl-3,7-dioxo-dodecahydro-1H-cyclopenta[a]naphthalen-6-yl)propanoic acid (6.12 g, 20 mmol) in glacial acetic acid (80 mL) was added ammonium acetate, the mixture was stirred under reflux for 4 h. After removed of glacial acetic acid under reduced pressure, the residue was poured into water. The precipitate was filtered and washed with water to afford (4aR,4bS,6aS,9aS,9bR)-4a,6a-dimethyl-4,4a,4b,5,6,6a,9,9a,9b,10-decahydro-1H-indeno[5,4-f]quinoline-2,7(3H,8H)-dione as a yellow solid (4.8 g, yield 84%). ¹H-NMR (400 MHz, CDCl₃): 0.91 (s, 3H), 1.13 (s, 3H), 1.18-2.00 (m, 12H), 2.06-2.16 (m, 1H), 2.22-2.27 (m, 1H), 2.45-2.52 (m, 3H), 4.92-4.94 (m, 1H), 8.63 (s, 1H). LC-MS (m/z) 288 [M+H]⁺.

### Example 48B

### 2-((4aR,4bS,6aS,9aS,9bR)-4a,6a-Dimethyl-2,7-dioxo-2,3,4,4a,4b,5,6,6a,7,8,9,9a,9b,10-tetradecahydroindeno[5,4-f]quinolin-1-yl)-N,N-dimethylacetamide

To a suspension of (4aR,4bS,6aS,9aS,9bR)-4a,6a-dimethyl-4,4a,4b,5,6,6a,9,9a,9b,10-decahydro-1H-indeno[5,4-f]quinoline-2,7(3H,8H)-dione (794 mg, 2.7 mmol) in anhydrous DMF (30 mL) was added NaH (60%, 324 mg, 8.1 mmol) and 2-chloro-N,N-dimethylacetamide (670 mg, 5.5 mmol) at 0° C., then the reaction was stirred under 50° C. for 2 hour. Then cooled down to room temperature and partitioned between ethyl acetate (20 mL) and water (20 mL). The layers were separated and the aqueous layer extracted with ethyl acetate (25 mL×3). The combined organic layers were dried over Na₂ SO₄. After filtration, the organic phase was concentrated under vacuum to afford 2-((4aR,4bS,6aS,9aS,9bR)-4a,6a-dimethyl-2,7-dioxo-2,3,4,4a,4b,5,6,6a,7,8,9,9a,9b,10-tetradecahydroindeno[5,4-f]quinolin-1-yl)-N,N-dimethylacetamide as a yellow solid (760 mg, yield 74%). ¹H-NMR (400 MHz, CDCl₃): 4.82 (m, 1H), 4.15 (m, 1H), 3.06 (s, 3H), 2.97 (m, 3H), 2.50 (m, 3H), 2.25 (m, 1H), 2.14 (m, 1H), 1.19 (m, 12H), 1.15 (s, 3H), 0.90 (s, 3H). LC-MS (m/z) 373 [M+H]⁺.

### Example 48C

### (4aR,4bS,6aS,9aS,9bR)-1-(2-(Dimethylamino)-2-oxoethyl)-4a,6a-dimethyl-2-oxo-2,3,4,4a,4b,5,6,6a,9,9a,9b,10-dodecahydro-1H-indeno[5,4-f]quinolin-7-yl trifluoromethanesulfonate

To a solution of 2-((4aR,4bS,6aS,9aS,9bR)-4a,6a-dimethyl-2,7-dioxo-2,3,4,4a,4b,5,6,6a,7,8,9,9a,9b,10-tetradecahydroindeno[5,4-f]quinolin-1-yl)-N,N-dimethylacetamide (220 mg, 0.6 mmol) in DCM (5 mL) was added Tf₂O (288 mg, 0.5 mmol). Triethylamine (60 mg, 0.6 mmol) was diluted with DCM (1 mL) and added dropwise into the above solution. The mixture was stirred at room temperature for 2 h. Water (10 mL) was added, and the mixture was extracted with DCM (20 mL×3). The combined organic layers were washed with 2N HCl (20 mL) and brine (20 mL), dried, and concentrated to afford (4aR,4bS,6aS,9aS,9bR)-1-(2-(dimethylamino)-2-oxoethyl)-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (70 mg, yield 23%). LC-MS (m/z) 505 [M+H]⁺.

### Example 48

### 2-((4aR,4bS,6aS,9aS,9bS)-4a,6a-Dimethyl-2-oxo-7-(pyridin-3-yl)-2,3,4,4a,4b,5,6,6a,9,9a,9b,10-dodecahydroindeno[5,4-f]quinolin-1-yl)-N,N-dimethylacetamide

Pd(PPh₃)₂Cl₂ (2.0 mg, 0.0028 mmol), pyridin-3-ylboronic acid (30 mg, 0.24 mmol) and K₂ CO₃ (2M, 0.3 mL, 0.63 mmol) were added to a stirred solution of (4aR,4bS,6aS,9aS,9bR)-1-(2-(dimethylamino)-2-oxoethyl)-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (70 mg, 0.14 mmol) in 1,4-dioxane (3 mL). The mixture was heated to 100° C. under N₂ for 0.5 hour, then cooled down to room temperature. Water (20 mL) was added, the mixture was extracted with ethyl acetate (35 mL×3). The combined organic layers were dried over Na₂ SO₄, filtered, concentrated. The residue was purified by prep-HPLC to afford 2-((4aR,4bS,6aS,9aS,9bS)-4a,6a-dimethyl-2-oxo-7-(pyridin-3-yl)-2,3,4,4a,4b,5,6,6a,9,9a,9b,10-dodecahydroindeno[5,4-f]quinolin-1-yl)-N,N-dimethylacetamide as a yellow solid (13 mg, yield 22%). ¹H-NMR (400 MHz, CDCl₃): 8.62 (s, 1H), 8.50 (m, 1H), 7.65 (m, 1H), 7.24 (m, 1H), 6.00 (m, 1H), 4.85 (m, 2H), 4.13 (m, 1H), 3.07 (s, 3H), 2.98 (s, 3H), 2.60 (m, 2H), 2.28 (m, 2H), 2.07 (m, 2H), 1.95 (m, 1H), 1.83 (m, 2H), 1.70 (m, 1H), 1.62 (m, 3H), 1.79 (m, 1H), 1.26 (m, 2H), 1.19 (s, 3H), 1.06 (s, 3H). LC-MS (m/z) 434 [M+H]⁺.

### Example 49

### 2-((4aR,4bS,6aS,9aS,9bS)-4a,6a-Dimethyl-2-oxo-7-(pyridin-4-yl)-2,3,4,4a,4b,5,6,6a,9,9a,9b,10-dodecahydroindeno[5,4-f]quinolin-1-yl)-N,N-dimethylacetamide

Pd(PPh₃)₂Cl₂ (14 mg, 0.02 mmol), pyridin-4-ylboronic acid (42 mg, 0.34 mmol) and K₂ CO₃ (2M, 0.3 mL, 0.6 mmol) were added to a stirred solution of (4aR,4bS,6aS,9aS,9bR)-1-(2-(dimethylamino)-2-oxoethyl)-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (100 mg, 0.20 mmol) in 1,4-dioxane (5 mL). The mixture was heated to 100° C. under N₂ for 0.5 hour, then cooled down to room temperature. Water (20 mL) was added, the mixture was extracted with ethyl acetate (35 mL×3). The combined organic layers were dried over Na₂ SO₄, filtered, concentrated. The residue was purified by TLC (DCM/CH₃ OH=20/1) to afford 2-((4aR,4bS,6aS,9aS,9bS)-4a,6a-dimethyl-2-oxo-7-(pyridin-4-yl)-2,3,4,4a,4b,5,6,6a,9,9a,9b,10-dodecahydroindeno[5,4-f]quinolin-1-yl)-N,N-dimethylacetamide as a white solid (3 mg, yield 4%). ¹H-NMR 6 (400 MHz, CDCl₃) major characteristic peaks: 8.52 (s, 2H), 7.26 (s, 2H), 6.18 8.52 (s, 1H), 4.84 (m, 2H), 4.13 (d, J=16.4 Hz, 1H), 3.07 (s, 3H), 2.98 (s, 3H), 1.19 (s, 3H), 1.09 (s, 3H). LC-MS (m/z) 434 [M+H]⁺.

### Example 50

### 2-((4aR,4bS,6aS,9aS,9bS)-4a,6a-Dimethyl-2-oxo-7-(pyrimidin-5-yl)-2,3,4,4a,4b,5,6,6a,9,9a,9b,10-dodecahydroindeno[5,4-f]quinolin-1-yl)-N,N-dimethylacetamide

Pd(PPh₃)₂Cl₂ (2.0 mg, 0.003 mmol), pyrimidin-5-ylboronic acid (43 mg, 0.34 mmol) and K₂ CO₃ (2 M, 0.45 mL, 0.9 mmol) were added to a stirred solution of (4aR,4bS,6aS,9aS,9bR)-1-(2-(dimethylamino)-2-oxoethyl)-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (100 mg, 0.20 mmol) in 1,4-dioxane (5 mL). The mixture was heated to 100° C. under N₂ for 0.5 hour, then cooled down to room temperature. Water (20 mL) was added, the mixture was extracted with ethyl acetate (35 mL×3). The combined organic layers were dried over Na₂ SO₄, filtered, concentrated. The residue was purified by TLC (DCM/CH₃ OH=20/1) to afford 2-((4aR,4bS,6aS,9aS,9bS)-4a,6a-dimethyl-2-oxo-7-(pyrimidin-5-yl)-2,3,4,4a,4b,5,6,6a,9,9a,9b,10-dodecahydroindeno[5,4-f]quinolin-1-yl)-N,N-dimethylacetamide as a white solid (10 mg, yield 11%). ¹H-NMR 6 (400 MHz, CDCl₃): 9.06 (s, 1H), 8.74 (s, 2H), 6.11 (s, 1H), 4.85 (m, 2H), 4.13 (m, 1H), 3.07 (s, 3H), 2.98 (s, 3H), 2.58 (m, 2H), 2.34 (m, 1H), 2.23 (s, 1H), 2.12 (m, 2H), 1.95 (m, 1H), 1.83 (m, 3H), 1.60 (m, 3H), 1.26 (m, 2H), 1.19 (s, 3H), 1.06 (s, 3H). LC-MS (m/z) 435 [M+H]⁺.

### Example 51

### 2-((4aR,4bS,6aS,9aS,9bS)-7-(5-Chloropyridin-3-yl)-4a,6a-dimethyl-2-oxo-2,3,4,4a,4b,5,6,6a,9,9a,9b,10-dodecahydro-1H-indeno[5,4-f]quinolin-1-yl)-N,N-dimethylacetamide

Pd(PPh₃)₂Cl₂ (2.0 mg, 0.003 mmol), 5-chloropyridin-3-ylboronic acid (42 mg, 0.27 mmol) and K₂ CO₃ (2 M, 0.36 mL, 0.72 mmol) were added to a stirred solution of (4aR,4bS,6aS,9aS,9bR)-1-(2-(dimethylamino)-2-oxoethyl)-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (80 mg, 0.16 mmol) in 1,4-dioxane (5 mL). The mixture was heated to 100° C. under N₂ for 0.5 hour, then cooled down to room temperature. Water (20 mL) was added, the mixture was extracted with ethyl acetate (35 mL×3). The combined organic layers were dried over Na₂ SO₄, filtered, concentrated. The residue was purified by prep-TLC (DCM/CH₃ OH=20/1) to afford 2-((4aR,4bS,6aS,9aS,9bS)-7-(5-chloropyridin-3-yl)-4a,6a-dimethyl-2-oxo-2,3,4,4a,4b,5,6,6a,9,9a,9b,10-dodecahydro-1H-indeno[5,4-f]quinolin-1-yl)-N,N-dimethylacetamide as a gray solid (20 mg, yield 27%). ¹H-NMR 6 (400 MHz, CDCl₃): 0.90 (m, 2H), 1.05 (s, 3H), 1.14 (s, 3H), 4.10 (m, 1H), 4.72 (s, 1H), 5.05 (s, 1H), 6.06 (s, 1H), 7.63 (s, 1H), 8.45 (m, 2H). LC-MS (m/z) [441+H]⁺.

### Example 52

### 2-((4aR,4bS,6aS,9aS,9bS)-7-(Isoquinolin-4-yl)-4a,6a-dimethyl-2-oxo-2,3,4,4a,4b,5,6,6a,9,9a,9b,10-dodecahydroindeno[5,4-f]quinolin-1-yl)-N,N-dimethylacetamide

Pd(PPh₃)₂Cl₂ (40 mg, 0.05 mmol), isoquinolin-4-ylboronic acid (158 mg, 0.92 mmol) and Cs₂ CO₃ (2 M, 0.80 mL, 1.62 mmol) were added to a stirred solution of (4aR,4bS,6aS,9aS,9bR)-1-(2-(dimethylamino)-2-oxoethyl)-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (270 mg, 0.54 mmol) in 1,4-dioxane (10 mL). The mixture was heated to reflux under N₂ for 1 hour, and then cooled down to room temperature. Water (20 mL) was added, the mixture was extracted with ethyl acetate (35 mL×3). The combined organic layers were dried over Na₂ SO₄, filtered, concentrated. The residue was purified by TLC (DCM/CH₃ OH=20/1) to afford 2-((4aR,4bS,6aS,9aS,9bS)-7-(isoquinolin-4-yl)-4a,6a-dimethyl-2-oxo-2,3,4,4a,4b,5,6,6a,9,9a,9b,10-dodecahydroindeno[5,4-f]quinolin-1-yl)-N,N-dimethylacetamide as a white solid (40 mg, yield 15%). ¹H-NMR 6 (400 MHz, CDCl₃) major characteristic peaks: 9.17 (s, 1H), 8.33 (s, 1H), 8.02 (m, 2H), 7.70 (t, J=7.2 Hz 1H), 7.62 (t, J=7.2 Hz 1H), 5.88 (s, 1H), 4.87 (m, 2H), 4.13 (d, J=16.4 Hz 1H), 3.08 (s, 3H), 2.99 (s, 3H), 1.17 (s, 3H), 1.03 (s, 3H). LC-MS (m/z) 484 [M+H]⁺

### Example 53

### (4aR,4bS,6aS,9aS,9bS)-4a,6a-Dimethyl-1-(2-morpholinoethyl)-7-(pyridin-3-yl)-4,4a,4b,5,6,6a,9,9a,9b,10-decahydro-1H-indeno[5,4-f]quinolin-2(3H)-one

### Example 53A

### (4aR,4bS,6aS,9aS,9bR)-4a,6a-Dimethyl-1-(2-morpholinoethyl)-4,4a,4b,5,6,6a,9,9a,9b,10-decahydro-1H-indeno[5,4-f]quinoline-2,7(3H,8H)-dione

A suspension of 3-((3aS,5aS,6R,9aR,9bS)-3a,6-dimethyl-3,7-dioxododecahydro-1H-cyclopenta[a]naphthalene-6-yl)propanoic acid (2.6 g, 8.48 mmol) in ethanol (40 mL) and ethylene glycol (100 mL) was treated with 2-morpholinoethanamine (3.3 g, 25.45 mmol) in ice-bath. The mixture was stirred at room temperature overnight. Then the solution was slowly (3° C./min) heated to reach 180° C. and keep at this temperature for 30 min. After being cooled to room temperature, the residue was washed out with water, extracted with DCM (100 mL×3), dried, concentrated to give (4aR,4bS,6aS,9aS,9bR)-4a,6a-dimethyl-1-(2-morpholinoethyl)-4,4a,4b,5,6,6a,9,9a,9b,10-decahydro-1H-indeno[5,4-f]quinoline-2,7(3H,8H)-dione (1.9 g). ¹H-NMR (400 MHz, CDCl₃) major characteristic peaks: δ 0.85 (s, 3H), 1.00 (s, 3H), 2.42 (m, 9H), 3.63 (t, J=4.4 Hz, 4H), 3.72 (m, 1H), 3.90 (m, 1H), 5.10 (m, 1H). LC-MS (m/z) 401 [M+H]⁺.

### Example 53B

### (4aR,4bS,6aS,9aS,9bR)-7-Hydrazono-4a,6a-dimethyl-1-(2-morpholinoethyl)-4,4a,4b,5,6,6a,7,8,9,9a,9b,10-dodecahydro-1H-indeno[5,4-f]quinolin-2(3H)-one

(4aR,4bS,6aS,9aS,9bR)-4a,6a-dimethyl-1-(2-morpholinoethyl)-4,4a,4b,5,6,6a,9,9a,9b,10-decahydro-1H-indeno[5,4-f]quinoline-2,7(3H,8H)-dione (3.8 g, 9.49 mmol) was dissolved in ethanol (100 mL), hydrazine hydrate (9.1 g, 284.7 mmol) and Et₃ N (2.9 g, 28.46 mmol) were added to the solution at room temperature, then the mixture was boiled under reflux for 2 h. After the reaction was finished, the mixture was cooled down, the solution was evaporated to one tenth of its original volume, water was added, extracted with DCM three times, the organic phase was separated and combined, washed with brine, dried over anhydrous Na₂ SO₄, concentrated to give (4aR,4bS,6aS,9aS,9bR)-7-hydrazono-4a,6a-dimethyl-1-(2-morpholinoethyl)-4,4a,4b,5,6,6a,7,8,9,9a,9b,10-dodecahydro-1H-indeno[5,4-f]quinolin-2(3H)-one. LC-MS (m/z) 344 [M+H]⁺.

### Example 53C

### (4aR,4bS,6aS,9aS,9bR)-7-Iodo-4a,6a-dimethyl-1-(2-morpholino ethyl)-4,4a,4b,5,6,6a,9,9a,9b,10-decahydro-1H-indeno[5,4-f]quinolin-2(3H)-one

I₂ (1.35 g, 5.31 mmol) was dissolved in THF/ether (V:V=2:1, 15 mL) in ice bath, 1,1,3,3-tetramethylguanidine (0.8 mL, 5.76 mmol) was added dropwise, then (4aR,4bS,6aS,9aS,9bR)-7-hydrazono-4a,6a-dimethyl-1-(2-morpholinoethyl)-4,4a,4b,5,6,6a,7,8,9,9a,9b,10-dodecahydro-1H-indeno[5,4-f]quinolin-2(3H)-one (400 mg) which was dissolved in THF (5 mL) followed. After the drop was finished, the mixture was stirred for 2 h in ice bath, concentrated under vacuum, the residue was re-dissolved in methylene chloride and washed with Na₂ SO₃ and brine. The solution was dried over anhydrous Na₂ SO₄, and then concentrated to give (4aR,4bS,6aS,9aS,9bR)-7-iodo-4a,6a-dimethyl-1-(2-morpholino ethyl)-4,4a,4b,5,6,6a,9,9a,9b,10-decahydro-1H-indeno[5,4-f]quinolin-2(3H)-one (450 mg). LC-MS (m/z): 511 [M+H]⁺.

### Example 53

### (4aR,4bS,6aS,9aS,9bS)-4a,6a-Dimethyl-1-(2-morpholinoethyl)-7-(pyridin-3-yl)-4,4a,4b,5,6,6a,9,9a,9b,10-decahydro-1H-indeno[5,4-f]quinolin-2(3H)-one

Pd(dppf)Cl₂ (17 mg, 0.021 mmol), pyridin-3-ylboronic acid (70 mg, 0.583 mmol) and K₂ CO₃ (2 M, 0.77 mL, 1.54 mmol) were added consecutively to a stirred solution of compound (4aR,4bS,6aS,9aS,9bR)-7-iodo-4a,6a-dimethyl-1-(2-morpholino ethyl)-4,4a,4b,5,6,6a,9,9a,9b,10-decahydro-1H-indeno[5,4-f]quinolin-2(3H)-one (175 mg, 0.34 mmol) in 1,4-dioxane (17 mL). The mixture was stirred at 80° C. under N₂ for 2 h. Then cooled to room temperature and partitioned between ethyl acetate (50 mL) and water (50 mL). The organic phase was separated and the aqueous phase was extracted again for 3 times. The combined organic phase was dried over anhydrous Na₂ SO₄. After filtration, the phase was concentrated under vacuum and the residue was purified by column chromatography to afford (4aR,4bS,6aS,9aS,9bS)-4a,6a-dimethyl-1-(2-morpholinoethyl)-7-(pyridin-3-yl)-4,4a,4b,5,6,6a,9,9a,9b,10-decahydro-1H-indeno[5,4-f]quinolin-2(3H)-one as a white solid (56 mg, yield 35%). ¹H NMR (CD₃OD, 400 MHz) major characteristic peaks: δ 8.89 (s, 1H), 8.72 (m, 1H), 8.64 (d, J=8.4 Hz, 1H), 8.04 (m, 1H), 6.50 (m, 1H), 5.06 (m, 1H), 1.16 (m, 6H). LC-MS (m/z): 463 [M+H]⁺.

### Example 54

### (4aR,4bS,6aS,9aS,9bS)-4a,6a-Dimethyl-1-(2-morpholinoethyl)-7-(pyrimidin-5-yl)-4,4a,4b,5,6,6a,9,9a,9b,10-decahydro-1H-indeno[5,4-f]quinolin-2(3H)-one

Pd(dppf)Cl₂ (16 mg, 0.024 mmol), pyrimidin-5-ylboronic acid (82 mg, 0.67 mmol) and K₂ CO₃ (2 M, 0.88 mL, 1.76 mmol) were added consecutively to a stirred solution of (4aR,4bS,6aS,9aS,9bR)-7-iodo-4a,6a-dimethyl-1-(2-morpholinoethyl)-4,4a,4b,5,6,6a,9,9a,9b,10-decahydro-1H-indeno[5,4-f]quinolin-2(3H)-one (65 mg, 0.15 mmol) in 1,4-dioxane (10 mL). The reaction was heated to 80° C. and stirred at this temperature for 2 hours. The reaction was cooled to room temperature and partitioned between ethyl acetate (50 mL) and water (50 mL). The layers were separated and the aqueous layer extracted with ethyl acetate (25 mL×3). The combined organic layers were dried over Na₂ SO₄. After filtration, the organic phase was concentrated under vacuum and the residue was purified by prep-HPLC to afford (4aR,4bS,6aS,9aS,9bS)-4a,6a-dimethyl-1-(2-morpholinoethyl)-7-(pyrimidin-5-yl)-4,4a,4b,5,6,6a,9,9a,9b,10-decahydro-1H-indeno[5,4-f]quinolin-2(3H)-one as a white solid (24 mg, yield 35%). ¹H NMR (CD₃OD, 400 MHz) major characteristic peaks: δ 9.00 (s, 1H), 8.74 (s, 2H), 6.20 (m, 1H), 5.27 (m, 1H), 1.09 (s, 6H). LC-MS (m/z): 463 [M+H]⁺.

### Example 55

### (4aR,4bS,6aS,9aS,9bS)-7-(5-Chloropyridin-3-yl)-1-ethyl-4a,6a-dimethyl-4,4a,4b,5,6,6a,9,9a,9b,10-decahydro-1H-indeno[5,4-f]quinolin-2(3H)-one

Pd(dppf)Cl₂ (33 mg, 0.04 mmol), 5-chloropyridin-3-ylboronic acid (116 mg, 0.76 mmol) and K₂ CO₃ (2 M, 1.0 mL, 2.03 mmol) were added consecutively to a stirred solution of (4aR,4bS,6aS,9aS,9bR)-1-ethyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (200 mg, 0.45 mmol) in 1,4-dioxane (10 mL). The mixture was heated at 100° C. under N₂ for 1 hour. Then cooled to room temperature and partitioned between ethyl acetate (20 mL) and water (20 mL). The aqueous layer extracted with ethyl acetate (50 mL×3). The combined organic layers were dried over Na₂ SO₄. After filtration, the organic phase was concentrated and purified by prep-TLC (Petroleum/Ethyl acetate, 4/1) to give (4aR,4bS,6aS,9aS,9bS)-7-(5-chloropyridin-3-yl)-1-ethyl-4a,6a-dimethyl-4,4a,4b,5,6,6a,9,9a,9b,10-decahydro-1H-indeno[5,4-f]quinolin-2(3H)-one (33 mg, yield 18%) as a white solid. ¹HNMR (DMSO-d₆, 400 MHz) major characteristic peaks: δ 1.04 (m, 9H), 3.67 (m, 2H), 5.14 (d, J=4.0 Hz, 1H), 6.29 (s, 1H), 7.86 (t, J=2.0 Hz, 1H), 8.50 (d, J=2.0 Hz, 1H), 8.57 (d, J=1.6 Hz, 1H). LC-MS (m/z) 411 [M+H]⁺.

### Example 56

### (4aR,4bS,6aS,9aS,9bS)-1-Ethyl-7-(6-methoxypyridin-3-yl)-4a,6a-dimethyl-4,4a,4b,5,6,6a,9,9a,9b,10-decahydro-1H-indeno[5,4-f]quinolin-2(3H)-one

Pd(PPh₃)₂Cl₂ (20 mg, 0.028 mmol), 4-methoxypyridin-3-ylboronic acid (117 mg, 0.76 mmol) and K₂ CO₃ (2 M, 1.00 mL, 2.00 mmol) were added consecutively to a stirred solution of ((4aR,4bS,6aS,9aS,9bR)-1-ethyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (200 mg, 0.45 mmol) in 1,4-dioxane (15 mL). The mixture was heated at 100° C. under N₂ for 1 h. Then cooled to room temperature and partitioned between ethyl acetate (100 mL) and water (100 mL). The layers were separated and the aqueous layer extracted with ethyl acetate (25 mL×3). The combined organic layers were dried over Na₂ SO₄, filtrated, concentrated under vacuum and the residue was purified by prep-HPLC to afford (4aR,4bS,6aS,9aS,9bS)-1-ethyl-7-(6-methoxypyridin-3-yl)-4a,6a-dimethyl-4,4a,4b,5,6,6a,9,9a,9b,10-decahydro-1H-indeno[5,4-f]quinolin-2(3H)-one as a white solid (45 mg, yield 23%). ¹H-NMR (CDCl₃, 400 MHz) major characteristic peaks: δ 8.18 (d, J=2.4 Hz 1H), 7.58 (dd, J1=8.8 Hz, J2=2.4 Hz 1H), 6.70 (d, J=8.8 Hz 1H), 5.87 (m, 1H), 5.14 (m, 1H), 3.93 (s, 3H), 3.83 (m, 1H), 3.68 (m, 1H), 1.14 (t, J=7.2 Hz, 3H), 1.07 (s, 3H), 1.03 (s, 3H). LC-MS (m/z) 407 [M+H]⁺.

### Example 57

### (4aR,4bS,6aS,9aS,9bS)-1-Ethyl-7-(4-methoxypyridin-3-yl)-4a,6a-dimethyl-4,4a,4b,5,6,6a,9,9a,9b,10-decahydro-1H-indeno[5,4-f]quinolin-2(3H)-one

Pd(PPh₃)₂Cl₂ (20 mg, 0.028 mmol), 4-methoxypyridin-3-ylboronic acid (116 mg, 0.76 mmol) and K₂ CO₃ (2 M, 1.0 mL, 2.0 mmol) were added consecutively to a stirred solution of ((4aR,4bS,6aS,9aS,9bR)-1-ethyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (200 mg, 0.45 mmol) in 1,4-dioxane (15 mL). The mixture was heated at 100° C. under N₂ for 1 hour. Then cooled to room temperature and partitioned between ethyl acetate (100 mL) and water (100 mL). The layers were separated and the aqueous layer extracted with ethyl acetate (25 mL×3). The combined organic layers were dried over Na₂ SO₄. After filtration, the organic phase was concentrated under vacuum and the residue was purified by prep-HPLC to afford (4aR,4bS,6aS,9aS,9bS)-1-ethyl-7-(4-methoxypyridin-3-yl)-4a,6a-dimethyl-4,4a,4b,5,6,6a,9,9a,9b,10-decahydro-1H-indeno[5,4-f]quinolin-2(3H)-one as a white solid (46 mg, yield 25%). ¹H-NMR (CDCl₃, 400 MHz) major characteristic peaks: δ 8.43 (s, 1H), 8.24 (s, 1H), 6.90 (s, 1H), 5.93 (s, 1H), 5.14 (d, J=4.0 Hz, 1H), 3.89 (s, 3H), 3.17 (s, 1H), 1.14 (s, 3H), 1.16 (s, 3H), 0.97 (s, 3H). LC-MS (m/z) 407 [M+H]⁺.

### Example 58

### (4aR,4bS,6aS,9aS,9bS)-7-(5-Chloropyridin-3-yl)-1-cyclopropyl-4a,6a-dimethyl-4,4a,4b,5,6,6a,9,9a,9b,10-decahydro-1H-indeno[5,4-f]quinolin-2(3H)-one

Pd(PPh₃)₂Cl₂ (catalytic amount), 5-chloropyridin-3-ylboronic acid (161 mg, 1.03 mmol) and K₂ CO₃ (2 M, 1.35 mL, 2.71 mmol) were added consecutively to a stirred solution of (4aR,4bS,6aS,9aS,9bR)-1-cyclopropyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (277 mg, 0.60 mmol) in 1,4-dioxane (10 mL). The mixture was heated at 100° C. under N₂ for 2 h. Then cooled to room temperature and partitioned between ethyl acetate (50 mL) and water (50 mL). The layers were separated and the aqueous layer extracted with ethyl acetate (15 mL×3). The combined organic layers were dried over Na₂ SO₄, concentrated under vacuum and the residue was purified by prep-TLC (DCM/CH₃ OH=20/1) to afford (4aR,4bS,6aS,9aS,9bS)-7-(5-chloropyridin-3-yl)-1-cyclopropyl-4a,6a-dimethyl-4,4a,4b,5,6,6a,9,9a,9b,10-decahydro-1H-indeno[5,4-f]quinolin-2(3H)-one as a white solid (38 mg, yield 15%). ¹H-NMR (CDCl₃, 400 MHz) major characteristic peaks: δ 8.43 (s, 1H), 8.37 (s, 1H), 7.58 (s, 1H), 6.00 (m, 1H), 5.31 (m, 1H), 1.00 (s, 3H), 0.93 (s, 3H), 0.73 (m, 1H), 0.67 (m, 1H). LC-MS (m/z) 423 [M+H]⁺.

### Example 59

### (4aR,4bS,6aS,9aS,9bS)-1-Cyclopropyl-7-(6-methoxypyridin-3-yl)-4a,6a-dimethyl-4,4a,4b,5,6,6a,9,9a,9b,10-decahydro-1H-indeno[5,4-f]quinolin-2(3H)-one

Pd(PPh₃)₂Cl₂ (catalytic amount), 6-methoxypyridin-3-ylboronic acid (15 mg, 0.095 mmol) and K₂ CO₃ (2 M, 0.20 mL, 0.39 mmol) were added consecutively to a stirred solution of (4aR,4bS,6aS,9aS,9bR)-1-cyclopropyl-4a,6a-dimethyl-2-oxo-2,3,4,4a,4b,5,6,6a,9,9a,9b,10-dodecahydro-1H-indeno[5,4-f]quinolin-7-yl trifluoromethanesulfonate (40 mg, 0.087) in 1,4-dioxane (3 mL). The mixture was heated to 100° C. under N₂ for 2 h. Then cooled to room temperature and partitioned between ethyl acetate (20 mL) and water (20 mL). The layers were separated and the aqueous layer extracted with ethyl acetate (10 mL×3). The combined organic layers were dried over Na₂ SO₄, concentrated under vacuum and the residue was purified by prep-TLC (DCM/CH₃ OH=20/1) to afford (4aR,4bS,6aS,9aS,9bS)-1-cyclopropyl-7-(6-methoxypyridin-3-yl)-4a,6a-dimethyl-4,4a,4b,5,6,6a,9,9a,9b,10-decahydro-1H-indeno[5,4-f]quinolin-2(3H)-one as a white solid (2 mg, yield 6%). ¹H-NMR (CDCl₃, 400 MHz) major characteristic peaks: δ 8.10 (d, J=1.6 Hz, 1H), 7.51 (dd, J₁=2.0 Hz, J₂=8.8 Hz, 1H), 6.63 (d, J=8.4 Hz, 1H), 5.80 (q, J=1.2 Hz, 1H), 5.31 (d, J=4.0 Hz, 1H), 3.86 (s, 3H), 2.51 (m, 1H), 0.96 (s, 3H), 0.92 (s, 3H), 0.24 (m, 1H). LC-MS (m/z) 419 [M+H]⁺.

### Example 60

### (4aR,4bS,6aS,9aS,9bS)-7-(5-Chloropyridin-3-yl)-4a,6a-dimethyl-1-propyl-4,4a,4b,5,6,6a,9,9a,9b,10-decahydro-1H-indeno[5,4-f]quinolin-2(3H)-one

Pd(PPh₃)₂Cl₂ (20 mg, 0.018 mmol), 5-chloropyridin-3-ylboronic acid (120 mg, 0.336 mmol) and K₂ CO₃ (2 M, 0.93 mL, 1.95 mmol) were added consecutively to a stirred solution of (4aR,4bS,6aS,9aS,9bR)-4a,6a-dimethyl-2-oxo-1-propyl-2,3,4,4a,4b,5,6,6a,9,9a,9b,10-dodecahydro-1H-indeno[5,4-f]quinolin-7-yl trifluoromethanesulfonate (200 mg, 0.433 mmol) in 1,4-dioxane (20 mL). The mixture was heated at 80° C. under N₂ for 2 h, then cooled to room temperature and partitioned between ethyl acetate (20 mL) and water (20 mL). The layers were separated and the aqueous layer extracted with ethyl acetate (10 mL×3). The combined organic layers were dried over Na₂ SO₄, concentrated under vacuum and the residue was purified by prep-TLC (PE/EA=5/1) to afford (4aR,4bS,6aS,9aS,9bS)-7-(5-chloropyridin-3-yl)-4a,6a-dimethyl-1-propyl-4,4a,4b,5,6,6a,9,9a,9b,10-decahydro-1H-indeno[5,4-f]quinolin-2(3H)-one as a white solid (48 mg, yield 34%). ¹H-NMR (400 MHz, CDCl₃) major characteristic peaks: δ 0.89 (t, 3H, J=7.2 Hz), 1.06 (s, 3H), 1.23 (s, 3H), 3.62 (m, 2H), 5.08 (m, 1H), 6.05 (m, 1H), 7.61 (m, 1H), 8.41 (s, 1H), 8.47 (s, 1H). LC-MS (m/z): 425 [M+H]⁺.

### Example 61

### (4aR,4bS,6aS,9aS,9bS)-4a,6a-dimethyl-7-(pyridin-3-yl)-4,4a,4b,5,6,6a,9,9a,9b,10,11,11a-dodecahydroindeno[5,4-f]chromen-2(3H)-one

### Example 61A

### (4aR,4bS,6aS,9aS,9bR)-4a,6a-dimethyl-4,4a,4b,5,6,6a,9,9a,9b,10-decahydroindeno[5,4-f]chromene-2,7(3H,8H)-dione

To a solution of 3-((3aS,5aS,6R,9aR,9bS)-3a,6-dimethyl-3,7-dioxododecahydro-1H-cyclopenta[a]naphthalen-6-yl)propanoic acid (5 g) in Ac₂O (60 ml) was added solid NaOAc (1.34 g). The reaction mixture was refluxed for 5 h. The mixture was cooled to room temperature and filtered. The solid was washed with 25% EtOAc in hexanes. The solution was concentrated under vacuum. The residue was purified by column chromatography on silica gel (hexanes:EtOAc=8:1 then 4:1) to provide (4aR,4bS,6aS,9aS,9bR)-4a,6a-dimethyl-4,4a,4b,5,6,6a,9,9a,9b,10-decahydroindeno[5,4-f]chromene-2,7(3H,8H)-dione (3.8 g, 80%). MS calcd for (C18H24O3) [2M+Na]+ 599.76. Found: 599.9; [2M−H]− 575.76. Found: 576.0. ¹H NMR (CDCl3, 300 MHz): δ 5.35 (1H), 1.11 (3H) 0.88 (3H).

### Example 61B

### (4aR,4bS,6aS,9aS,9bR)-4a,6a-dimethyl-2-oxo-2,3,4,4a,4b,5,6,6a,9,9a,9b,10-decahydroindeno[5,4-f]chromene-7-yl trifluoromethanesulfonate and 3-((3aS,5aS,6R,9aS,9bS)-3a,6-dimethyl-7-oxo-3-(trifluoromethylsulfonyloxy)-3a,4,5,5a,6,7,8,9,9a,9b-decahydro-1H-cyclopenta[a]naphthalen-6-yl)propanoic acid

To a solution of (4aR,4bS,6aS,9aS,9bR)-4a,6a-dimethyl-4,4a,4b,5,6,6a,9,9a,9b,10-decahydroindeno[5,4-f]chromene-2,7(3H,8H)-dione (2.0 g, 6.9 mmol) in DCM (35 mL) was added trifluoromethane sulfonic anhydride (0.75 mL, 10.4 mmol, 1.5 equiv) at room temperature. The solution was stirred over 10 min and TEA (1 mL, 6.9 mmol, 1 eq.) in dichloromethane (DCM) (10 mL) was added dropwise within 30 min. The mixture was stirred for 5 h. The reaction was monitored by TLC (EtOAc:hexanes=1:3) and the starting material was completely consumed. Water (20 mL) was added and the layers were separated. The aqueous layer was extracted with DCM (3×50 mL). The organic layers were combined, washed with 2N HCl, brine, dried (MgSO₄). The solution was concentrated and purified by column chromatography on silica gel (hexanes/EA=1:1, 1% HOAc) to give a mixture of (4aR,4bS,6aS,9aS,9bR)-4a,6a-dimethyl-2-oxo-2,3,4,4a,4b,5,6,6a,9,9a,9b,10-decahydroindeno[5,4-f]chromene-7-yl trifluoromethanesulfonate and 3-((3aS,5aS,6R,9aS,9bS)-3a,6-dimethyl-7-oxo-3-(trifluoromethylsulfonyloxy)-3a,4,5,5a,6,7,8,9,9a,9b-decahydro-1H-cyclopenta[a]naphthalen-6-yl)propanoic acid (2 g, 66%), which was used in the next step without further purification. MS calcd for C0023 (C₁₉H₂₅F₃O₆S), [M+H]+ 439.46. Found: 439.0; [2M−H]− 437.46. Found: 437.1.

### Example 61C

### 3-((3aS,5aS,6R,9aS,9bS)-3a,6-dimethyl-7-oxo-3-(pyridin-3-yl)-3a,4,5,5a,6,7,8,9,9a,9b-decahydro-1H-cyclopenta[a]naphthalen-6-yl)propanoic acid

To a solution of (4aR,4bS,6aS,9aS,9bR)-4a,6a-dimethyl-2-oxo-2,3,4,4a,4b,5,6,6a,9,9a,9b,10-decahydroindeno[5,4-f]chromene-7-yl trifluoromethanesulfonate and 3-((3aS,5aS,6R,9aS,9bS)-3a,6-dimethyl-7-oxo-3-(trifluoromethylsulfonyloxy)-3a,4,5,5a,6,7,8,9,9a,9b-decahydro-1H-cyclopenta[a]naphthalen-6-yl)propanoic acid (2.3 g) in THF (350 mL) was added pyridin-3-yl boronic acid (1.5 g, 2.5 equiv), (Ph₃P)₂ PdCl₂ (160 mg, 0.05 equiv) and 2 N aqueous Na₂ CO₃ (12 mL). The mixture was degassed and refilled with Argon three times. And, the mixture was heated at 80° C. overnight. The reaction was monitored by TLC. The mixture was cool to room temperature and extracted with DCM (2×30 mL). The organic layers were combined, washed with brine (2×20 mL) dried (Na₂ SO₄). The solution was concentrated and purified by column chromatography on silica gel (EtOAc/Hexanes=1:1, 0.5% HOAc) to give 3-((3aS,5aS,6R,9aS,9bS)-3a,6-dimethyl-7-oxo-3-(pyridin-3-yl)-3a,4,5,5a,6,7,8,9,9a,9b-decahydro-1H-cyclopenta[a]naphthalen-6-yl)propanoic acid (1.1 g, 65%) as a pale yellow solid. MS calcd for (C₂₃H₂₉NO₃) [2M+H]+ 735.96. Found: 735.5; [2M−H]− 733.96. Found: 733.6. ¹H NMR (CDCl₃, 300 MHz): δ 8.619 (s, 1H), 8.45 (brs, 1H), 7.68 (d, 1H), 7.29 (m, 1H), 6.00 (s, 1H), 1.163 (s, 3H), 1.062 (s, 3H).

### Example 61

### (4aR,4bS,6aS,9aS,9bS)-4a,6a-dimethyl-7-(pyridin-3-yl)-4,4a,4b,5,6,6a,9,9a,9b,10,11,11a-dodecahydroindeno[5,4-f]chromen-2(3H)-one

NaBH₄ (160 mg, 4 mmol) was added in a solution of 3-((3aS,5aS,6R,9aS,9bS)-3a,6-dimethyl-7-oxo-3-(pyridin-3-yl)-3a,4,5,5a,6,7,8,9,9a,9b-decahydro-1H-cyclopenta[a]naphthalen-6-yl)propanoic acid (370 mg, 1 mmol) in methanol (5 mL) at 0° C. After the solution was kept stirring at room temperature for 1.5 hours, the methanol was remove away under reduce pressure. The residue acidified with 1 N HCl. The crude product was extracted with EtOAc (2×5 mL), dried over Na₂ SO₄. The solution was concentrated to afford (4aR,4bS,6aS,9aS, 9bS)-4a,6a-dimethyl-7-(pyridin-3-yl)-4,4a,4b,5,6,6a,9,9a,9b,10,11,11a-dodecahydroindeno[5,4-f]chromen-2(3H)-one. MS calculated for (C23H29NO2) [M+H]+ 352.48. Found: 352.4. ¹H NMR (CDCl3, 300 MHz): δ 8.61 (s, 1H), 8.46 (brs, 1H), 7.65 (d, 1H), 7.26 (m, 1H), 5.98 (s, 1H), 4.00-4.20 (m, 1H), 1.14 (s, 3H), 1.02 (s, 3H).

### Example 62

### (4aR,4bS,6aS,9aS,9bS)-4a,6a-dimethyl-7-(pyridin-3-yl)-4,4a,4b,5,6,6a,9,9a,9b,10-decahydroindeno[5,4-f]chromen-2(3H)-one

To a solution of 3-((3aS,5aS,6R,9aS,9bS)-3a,6-dimethyl-7-oxo-3-(pyridin-3-yl)-3a,4,5,5a,6,7,8,9,9a,9b-decahydro-1H-cyclopenta[a]naphthalen-6-yl)propanoic acid (50 mg, 0.136 mmol) in Ac₂O (2 ml) was added solid NaOAc (22.2 mg, 0.163 mmol). The reaction mixture was refluxed overnight. The reaction was monitored by TLC and the starting material was completely consumed. The mixture was cooled to room temperature and concentrated. The residue was added DCM (10 ml) and saturated aqueous Na₂ CO₃ (2 ml). The layers were separated and the aqueous was extracted with DCM (3×10 mL). The combined organic layers were washed with brine, dried over Na₂ SO₄. The solution was concentrated under vacuum to afford (4aR,4bS,6aS,9aS,9bS)-4a,6a-dimethyl-7-(pyridin-3-yl)-4,4a,4b,5,6,6a,9,9a,9b,10-decahydroindeno[5,4-f]chromen-2(3H)-one (25 mg). MS calcd for (C23H27NO2) [2M+Na]+ 721.94. Found: 722.1. ¹H NMR (CDCl3, 300 MHz): δ 8.61 (s, 1H), 8.46 (brs, 1H), 7.64 (d, 1H), 7.22 (m, 1H), 6.00 (s, 1H), 5.30 (s, 1H), 2.65 (m, 2H), 2.01-2.35 (m, 4H), 1.4-1.95 (m, 8H), 1.31 (m, 1H), 1.16 (s, 3H), 1.06 (s, 3H).

### Example 63

### (4aR,4bS,6aS,9aS,9bS)-7-(5-Fluoropyridin-3-yl)-1,4a,6a-trimethyl-1,3,4,4a,4b,5,6,6a,9,9a,9b,10-dodecahydro-indeno[5,4-f]quinolin-2(3H)-one

Using a synthetic procedure and condition similar to Example 8 in the preparation of 7-(6-methoxy-pyridin-3-yl)-1,4a,6a-trimethyl-1,3,4,4a,4b,5,6,6a,9,9a,9b,10-dodecahydro-indeno[5,4-f]quinolin-2-one, replacing 6-methoxypyridin-3-ylboronic acid with 5-fluoropyridin-3-ylboronic acid, (4aR,4bS,6aS,9aS,9bS)-7-(5-fluoropyridin-3-yl)-1,4a,6a-trimethyl-1,3,4,4a,4b,5,6,6a,9,9a,9b,10-dodecahydro-indeno[5,4-f]quinolin-2(3H)-one is made.

### Example 64

### (4aR,4bS,6aS,9aS,9bS)-7-(5-Methylpyridin-3-yl)-1,4a,6a-trimethyl-1,3,4,4a,4b,5,6,6a,9,9a,9b,10-dodecahydro-indeno[5,4-f]quinolin-2(3H)-one

Using a synthetic procedure and condition similar to Example 8 in the preparation of 7-(6-methoxy-pyridin-3-yl)-1,4a,6a-trimethyl-1,3,4,4a,4b,5,6,6a,9,9a,9b,10-dodecahydro-indeno[5,4-f]quinolin-2-one, replacing 6-methoxypyridin-3-ylboronic acid with 5-methylpyridin-3-ylboronic acid, (4aR,4bS,6aS,9aS,9bS)-7-(5-methylpyridin-3-yl)-1,4a,6a-trimethyl-1,3,4,4a,4b,5,6,6a,9,9a,9b,10-dodecahydro-indeno[5,4-f]quinolin-2(3H)-one is made.

### Example 65

### (4aR,4bS,6aS,9aS,9bS)-7-(4-Methoxy-pyridin-3-yl)-1,4a,6a-trimethyl-1,3,4,4a,4b,5,6,6a,9,9a,9b,10-dodecahydro-indeno[5,4-f]quinolin-2(3H)-one

Using a synthetic procedure and condition similar to Example 8 in the preparation of 7-(6-methoxy-pyridin-3-yl)-1,4a,6a-trimethyl-1,3,4,4a,4b,5,6,6a,9,9a,9b,10-dodecahydro-indeno[5,4-f]quinolin-2-one, replacing 6-methoxypyridin-3-ylboronic acid with 4-methoxypyridin-3-ylboronic acid, (4aR,4bS,6aS,9aS,9bS)-7-(4-methoxy-pyridin-3-yl)-1,4a,6a-trimethyl-1,3,4,4a,4b,5,6,6a,9,9a,9b,10-dodecahydro-indeno[5,4-f]quinolin-2(3H)-one was made as a white solid. ¹H NMR (CDCl₃, 400 MHz) major characteristic peaks: δ 8.39 (s, 1H), 8.24 (s, 1H), 6.80 (d, J=2.8 Hz, 1H), 5.86 (s, 1H), 5.05 (m, 1H), 3.83 (s, 3H), 3.11 (s, 3H), 1.06 (s, 3H), 0.94 (s, 3H). LC-MS (m/z) 393 [M+H]⁺.

### Example 66

### (4aR,4bS,6aS,9aS,9bS)-7-(5-Ethoxy-pyridin-3-yl)-1,4a,6a-trimethyl-1,3,4,4a,4b,5,6,6a,9,9a,9b,10-dodecahydro-indeno[5,4-f]quinolin-2(3H)-one

Using a synthetic procedure and condition similar to Example 8 in the preparation of 7-(6-methoxy-pyridin-3-yl)-1,4a,6a-trimethyl-1,3,4,4a,4b,5,6,6a,9,9a,9b,10-dodecahydro-indeno[5,4-f]quinolin-2-one, replacing 6-methoxypyridin-3-ylboronic acid with 5-ethoxypyridin-3-ylboronic acid, (4aR,4bS,6aS,9aS,9bS)-7-(5-Ethoxy-pyridin-3-yl)-1,4a,6a-trimethyl-1,3,4,4a,4b,5,6,6a,9,9a,9b,10-dodecahydro-indeno[5,4-f]quinolin-2(3H)-one is made.

### Example 67

### (4aR,4bS,6aS,9aS,9bS)-1-ethyl-7-(5-Methoxypyridin-3-yl)-4a,6a-dimethyl 4,4a,4b,5,6,6a,9,9a,9b,10-decahydro-1H-indeno[5,4-f]quinolin-2(3H)-one

Using a synthetic procedure and condition similar to Example 27 in the preparation of (4aR,4bS,6aS,9aS,9bS)-1-ethyl-4a,6a-dimethyl-7-(pyridin-3-yl)-4,4a,4b,5,6,6a, 9,9a,9b,10-decahydro-1H-indeno[5,4-f]quinolin-2(3H)-one, replacing pyridin-3-ylboronic acid with 5-methoxypyridin-3-ylboronic acid, (4aR,4bS,6aS,9aS,9bS)-1-ethyl-7-(5-methoxypyridin-3-yl)-4a,6a-dimethyl 4,4a,4b,5,6,6a,9,9a,9b,10-decahydro-1H-indeno[5,4-f]quinolin-2(3H)-one is made.

### Example 68

### (4aR,4bS,6aS,9aS,9bS)-1-ethyl-7-(5-Ethoxypyridin-3-yl)-4a,6a-dimethyl 4,4a,4b,5,6,6a,9,9a,9b,10-decahydro-1H-indeno[5,4-f]quinolin-2(3H)-one

Using a synthetic procedure and condition similar to Example 27 in the preparation of (4aR,4bS,6aS,9aS,9bS)-1-ethyl-4a,6a-dimethyl-7-(pyridin-3-yl)-4,4a,4b,5,6,6a,9,9a,9b,10-decahydro-1H-indeno[5,4-f]quinolin-2(3H)-one, replacing pyridin-3-ylboronic acid with 5-ethoxypyridin-3-ylboronic acid, (4aR,4bS,6aS,9aS,9bS)-1-ethyl-7-(5-ethoxypyridin-3-yl)-4a,6a-dimethyl 4,4a,4b,5,6,6a,9,9a,9b,10-decahydro-1H-indeno[5,4-f]quinolin-2(3H)-one is made.

### Example 69

### (4aR,4bS,6aS,9aS,9bS)-1-ethyl-7-(5-Fluoropyridin-3-yl)-4a,6a-dimethyl 4,4a,4b,5,6,6a,9,9a,9b,10-decahydro-1H-indeno[5,4-f]quinolin-2(3H)-one

Using a synthetic procedure and condition similar to Example 27 in the preparation of (4aR,4bS,6aS,9aS,9bS)-1-ethyl-4a,6a-dimethyl-7-(pyridin-3-yl)-4,4a,4b,5,6,6a,9,9a,9b,10-decahydro-1H-indeno[5,4-f]quinolin-2(3H)-one, replacing pyridin-3-ylboronic acid with 5-ethoxyfluoropyridin-3-ylboronic acid, (4aR,4bS,6aS,9aS,9bS)-1-ethyl-7-(5-fluoropyridin-3-yl)-4a,6a-dimethyl 4,4a,4b,5,6,6a,9,9a,9b,10-decahydro-1H-indeno[5,4-f]quinolin-2(3H)-one is made.

### Example 70

### (4aR,4bS,6aS,9aS,9bS)-1-ethyl-7-(5-Methylpyridin-3-yl)-4a,6a-dimethyl 4,4a,4b,5,6,6a,9,9a,9b,10-decahydro-1H-indeno[5,4-f]quinolin-2(3H)-one

Using a synthetic procedure and condition similar to Example 27 in the preparation of (4aR,4bS,6aS,9aS,9bS)-1-ethyl-4a,6a-dimethyl-7-(pyridin-3-yl)-4,4a,4b,5,6,6a,9,9a,9b,10-decahydro-1H-indeno[5,4-f]quinolin-2(3H)-one, replacing pyridin-3-ylboronic acid with 5-methylpyridin-3-ylboronic acid, (4aR,4bS,6aS,9aS,9bS)-1-ethyl-7-(5-methylpyridin-3-yl)-4a,6a-dimethyl 4,4a,4b,5,6,6a,9,9a,9b,10-decahydro-1H-indeno[5,4-f]quinolin-2(3H)-one is made.

### Example 71

### (4aR,4bS,6aS,9aS,9bS)-1-ethyl-7-(5-chloropyridin-3-yl)-4a,6a-dimethyl 4,4a,4b,5,6,6a,9,9a,9b,10-decahydro-1H-indeno[5,4-f]quinolin-2(3H)-one

Using a synthetic procedure and condition similar to Example 27 in the preparation of (4aR,4bS,6aS,9aS,9bS)-1-ethyl-4a,6a-dimethyl-7-(pyridin-3-yl)-4,4a,4b,5,6,6a,9,9a,9b,10-decahydro-1H-indeno[5,4-f]quinolin-2(3H)-one, replacing pyridin-3-ylboronic acid with 5-chlorolpyridin-3-ylboronic acid, (4aR,4bS,6aS,9aS,9bS)-1-ethyl-7-(5-chloropyridin-3-yl)-4a,6a-dimethyl 4,4a,4b,5,6,6a,9,9a,9b,10-decahydro-1H-indeno[5,4-f]quinolin-2(3H)-one is made.

### Example 72

### (4aR,4bS,6aS,9aS,9bS)-1-ethyl-7-(4-Methylpyridin-3-yl)-4a,6a-dimethyl 4,4a,4b,5,6,6a,9,9a,9b,10-decahydro-1H-indeno[5,4-f]quinolin-2(3H)-one

Using a synthetic procedure and condition similar to Example 27 in the preparation of (4aR,4bS,6aS,9aS,9bS)-1-ethyl-4a,6a-dimethyl-7-(pyridin-3-yl)-4,4a,4b,5,6,6a,9,9a,9b,10-decahydro-1H-indeno[5,4-f]quinolin-2(3H)-one, replacing pyridin-3-ylboronic acid with 4-methylpyridin-3-ylboronic acid, (4aR,4bS,6aS,9aS,9bS)-1-ethyl-7-(5-methylpyridin-3-yl)-4a,6a-dimethyl 4,4a,4b,5,6,6a,9,9a,9b,10-decahydro-1H-indeno[5,4-f]quinolin-2(3H)-one is made.

### Example 73

### (4aR,4bS,6aS,9aS,9bS)-1-Cyclopropyl-7-(5-Methoxypyridin-3-yl)-4a,6a-dimethyl-4,4a,4b,5,6,6a,9,9a,9b,10-decahydro-1H-indeno[5,4-f]quinolin-2(3H)-one

Using a synthetic procedure and condition similar to Example 35 in the preparation of (4aR,4bS,6aS,9aS,9bS)-1-cyclopropyl-4a,6a-dimethyl-7-(pyridin-3-yl)-4,4a,4b,5,6,6a,9,9a,9b,10-decahydro-1H-indeno[5,4-f]quinolin-2(3H)-one, replacing pyridin-3-ylboronic acid with 5-methoxypyridin-3-ylboronic acid, (4aR,4bS,6aS,9aS,9bS)-1-cyclopropyl-7-(5-methoxypyridin-3-yl)-4a,6a-dimethyl-4,4a,4b,5,6,6a,9,9a,9b,10-decahydro-1H-indeno[5,4-f]quinolin-2(3H)-one is made.

### Example 74

### (4aR,4bS,6aS,9aS,9bS)-1-Cyclopropyl-7-(5-Ethoxypyridin-3-yl)-4a,6a-dimethyl-4,4a,4b,5,6,6a,9,9a,9b,10-decahydro-1H-indeno[5,4-f]quinolin-2(3H)-one

Using a synthetic procedure and condition similar to Example 35 in the preparation of (4aR,4bS,6aS,9aS,9bS)-1-cyclopropyl-4a,6a-dimethyl-7-(pyridin-3-yl)-4,4a,4b,5,6,6a,9,9a,9b,10-decahydro-1H-indeno[5,4-f]quinolin-2(3H)-one, replacing pyridin-3-ylboronic acid with 5-ethoxypyridin-3-ylboronic acid, (4aR,4bS,6aS,9aS,9bS)-1-cyclopropyl-7-(5-ethoxypyridin-3-yl)-4a,6a-dimethyl-4,4a,4b,5,6,6a,9,9a,9b,10-decahydro-1H-indeno[5,4-f]quinolin-2(3H)-one is made.

### Example 75

### (4aR,4bS,6aS,9aS,9bS)-1-Cyclopropyl-7-(5-Fluoropyridin-3-yl)-4a,6a-dimethyl-4,4a,4b,5,6,6a,9,9a,9b,10-decahydro-1H-indeno[5,4-f]quinolin-2(3H)-one

Using a synthetic procedure and condition similar to Example 35 in the preparation of (4aR,4bS,6aS,9aS,9bS)-1-cyclopropyl-4a,6a-dimethyl-7-(pyridin-3-yl)-4,4a,4b,5,6,6a,9,9a,9b,10-decahydro-1H-indeno[5,4-f]quinolin-2(3H)-one, replacing pyridin-3-ylboronic acid with 5-fluoropyridin-3-ylboronic acid, (4aR,4bS,6aS,9aS,9bS)-1-cyclopropyl-7-(5-fluoropyridin-3-yl)-4a,6a-dimethyl-4,4a,4b,5,6,6a,9,9a,9b,10-decahydro-1H-indeno[5,4-f]quinolin-2(3H)-one is made.

### Example 76

### (4aR,4bS,6aS,9aS,9bS)-1-Cyclopropyl-7-(5-Methylpyridin-3-yl)-4a,6a-dimethyl-4,4a,4b,5,6,6a,9,9a,9b,10-decahydro-1H-indeno[5,4-f]quinolin-2(3H)-one

Using a synthetic procedure and condition similar to Example 35 in the preparation of (4aR,4bS,6aS,9aS,9bS)-1-cyclopropyl-4a,6a-dimethyl-7-(pyridin-3-yl)-4,4a,4b,5,6,6a,9,9a,9b,10-decahydro-1H-indeno[5,4-f]quinolin-2(3H)-one, replacing pyridin-3-ylboronic acid with 5-methylpyridin-3-ylboronic acid, (4aR,4bS,6aS,9aS,9bS)-1-cyclopropyl-7-(5-methylpyridin-3-yl)-4a,6a-dimethyl-4,4a,4b,5,6,6a,9,9a,9b,10-decahydro-1H-indeno[5,4-f]quinolin-2(3H)-one is made.

### Example 77

### (4aR,4bS,6aS,9aS,9bS)-1-Cyclopropyl-7-(4-Methoxypyridin-3-yl)-4a,6a-dimethyl-4,4a,4b,5,6,6a,9,9a,9b,10-decahydro-1H-indeno[5,4-f]quinolin-2(3H)-one

Using a synthetic procedure and condition similar to Example 35 in the preparation of (4aR,4bS,6aS,9aS,9bS)-1-cyclopropyl-4a,6a-dimethyl-7-(pyridin-3-yl)-4,4a,4b,5,6,6a,9,9a,9b,10-decahydro-1H-indeno[5,4-f]quinolin-2(3H)-one, replacing pyridin-3-ylboronic acid with 4-methoxypyridin-3-ylboronic acid, (4aR,4bS,6aS,9aS,9bS)-1-cyclopropyl-7-(4-methoxypyridin-3-yl)-4a,6a-dimethyl-4,4a,4b,5,6,6a,9,9a,9b,10-decahydro-1H-indeno[5,4-f]quinolin-2(3H)-one is made.

### Example 78

### (4aR,4bS,6aS,9aS,9bS)-1-Cyclopropyl-7-(4-Chloropyridin-3-yl)-4a,6a-dimethyl-4,4a,4b,5,6,6a,9,9a,9b,10-decahydro-1H-indeno[5,4-f]quinolin-2(3H)-one

Using a synthetic procedure and condition similar to Example 35 in the preparation of (4aR,4bS,6aS,9aS,9bS)-1-cyclopropyl-4a,6a-dimethyl-7-(pyridin-3-yl)-4,4a,4b,5,6,6a,9,9a,9b,10-decahydro-1H-indeno[5,4-f]quinolin-2(3H)-one, replacing pyridin-3-ylboronic acid with 4-chloropyridin-3-ylboronic acid, (4aR,4bS,6aS,9aS,9bS)-1-cyclopropyl-7-(4-chloropyridin-3-yl)-4a,6a-dimethyl-4,4a,4b,5,6,6a,9,9a,9b,10-decahydro-1H-indeno[5,4-f]quinolin-2(3H)-one is made.

### Example 79

### (4aR,4bS,6aS,9aS,9bS)-1-Cyclopropyl-7-(4-Methylpyridin-3-yl)-4a,6a-dimethyl-4,4a,4b,5,6,6a,9,9a,9b,10-decahydro-1H-indeno[5,4-f]quinolin-2(3H)-one

Using a synthetic procedure and condition similar to Example 35 in the preparation of (4aR,4bS,6aS,9aS,9bS)-1-cyclopropyl-4a,6a-dimethyl-7-(pyridin-3-yl)-4,4a,4b,5,6,6a,9,9a,9b,10-decahydro-1H-indeno[5,4-f]quinolin-2(3H)-one, replacing pyridin-3-ylboronic acid with 4-methylpyridin-3-ylboronic acid, (4aR,4bS,6aS,9aS,9bS)-1-cyclopropyl-7-(4-methylpyridin-3-yl)-4a,6a-dimethyl-4,4a,4b,5,6,6a,9,9a,9b,10-decahydro-1H-indeno[5,4-f]quinolin-2(3H)-one is made.

### Example 80

### (3aS,3bS,9aR,9bS,11aS)-1-(5-Methoxypyridin-3-yl)-5,9a,11a-trimethyl-3b,4,5,8,9,9a,9b,10,11,11a-decahydro-3H-cyclopenta[i]phenanthridin-7(3aH)-one

Using a synthetic procedure and condition similar to Example 4 in the preparation of (3aS,3bS,9aR,9bS,11aS)-5,9a,11a-trimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-methoxypyridin-3-ylboronate, (3aS,3bS,9aR,9bS,11aS)-1-(5-methoxypyridin-3-yl)-5,9a,11a-trimethyl-3b,4,5,8,9,9a,9b,10,11,11a-decahydro-3H-cyclopenta[i]phenanthridin-7(3aH)-one is made.

### Example 81

### (3aS,3bS,9aR,9bS,11aS)-1-(5-Ethoxypyridin-3-yl)-5,9a,11a-trimethyl-3b,4,5,8,9,9a,9b,10,11,11a-decahydro-3H-cyclopenta[i]phenanthridin-7(3aH)-one

Using a synthetic procedure and condition similar to Example 4 in the preparation of (3aS,3bS,9aR,9bS,11aS)-5,9a,11a-trimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-ethoxypyridin-3-ylboronate, (3aS,3bS,9aR,9bS,11aS)-1-(5-ethoxypyridin-3-yl)-5,9a,11a-trimethyl-3b,4,5,8,9,9a,9b,10,11,11a-decahydro-3H-cyclopenta[i]phenanthridin-7(3aH)-one is made.

### Example 82

### (3aS,3bS,9aR,9bS,11aS)-1-(5-Fluoropyridin-3-yl)-5,9a,11a-trimethyl-3b,4,5,8,9,9a,9b,10,11,11a-decahydro-3H-cyclopenta[i]phenanthridin-7(3aH)-one

Using a synthetic procedure and condition similar to Example 4 in the preparation of (3aS,3bS,9aR,9bS,11aS)-5,9a,11a-trimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-fluoropyridin-3-ylboronate, (3aS,3bS,9aR,9bS,11aS)-1-(5-fluoropyridin-3-yl)-5,9a,11a-trimethyl-3b,4,5,8,9,9a,9b,10,11,11a-decahydro-3H-cyclopenta[i]phenanthridin-7(3aH)-one is made.

### Example 83

### (3aS,3bS,9aR,9bS,11aS)-1-(5-Chloropyridin-3-yl)-5,9a,11a-trimethyl-3b,4,5,8,9,9a,9b,10,11,11a-decahydro-3H-cyclopenta[i]phenanthridin-7(3aH)-one

Using a synthetic procedure and condition similar to Example 4 in the preparation of (3aS,3bS,9aR,9bS,11aS)-5,9a,11a-trimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-chloropyridin-3-ylboronate, (3aS,3bS,9aR,9bS,11aS)-1-(5-chloropyridin-3-yl)-5,9a,11a-trimethyl-3b,4,5,8,9,9a,9b,10,11,11a-decahydro-3H-cyclopenta[i]phenanthridin-7(3aH)-one is made.

### Example 84

### (3aS,3bS,9aR,9bS,11aS)-1-(5-Methylpyridin-3-yl)-5,9a,11a-trimethyl-3b,4,5,8,9,9a,9b,10,11,11a-decahydro-3H-cyclopenta[i]phenanthridin-7(3aH)-one

Using a synthetic procedure and condition similar to Example 4 in the preparation of (3aS,3bS,9aR,9bS,11aS)-5,9a,11a-trimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-methylpyridin-3-ylboronate, (3aS,3bS,9aR,9bS,11aS)-1-(5-methylpyridin-3-yl)-5,9a,11a-trimethyl-3b,4,5,8,9,9a,9b,10,11,11a-decahydro-3H-cyclopenta[i]phenanthridin-7(3aH)-one is made.

### Example 85

### (3aS,3bS,9aR,9bS,11aS)-1-(4-Methoxypyridin-3-yl)-5,9a,11a-trimethyl-3b,4,5,8,9,9a,9b,10,11,11a-decahydro-3H-cyclopenta[i]phenanthridin-7(3aH)-one

Using a synthetic procedure and condition similar to Example 4 in the preparation of (3aS,3bS,9aR,9bS,11aS)-5,9a,11a-trimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 4-methoxypyridin-3-ylboronate, (3aS,3bS,9aR,9bS,11aS)-1-(4-methoxypyridin-3-yl)-5,9a,11a-trimethyl-3b,4,5,8,9,9a,9b,10,11,11a-decahydro-3H-cyclopenta[i]phenanthridin-7(3aH)-one is made.

### Example 86

### (3aS,3bS,9aR,9bS,11aS)-1-(4-Chloropyridin-3-yl)-5,9a,11a-trimethyl-3b,4,5,8,9,9a,9b,10,11,11a-decahydro-3H-cyclopenta[i]phenanthridin-7(3aH)-one

Using a synthetic procedure and condition similar to Example 4 in the preparation of (3aS,3bS,9aR,9bS,11aS)-5,9a,11a-trimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 4-chloropyridin-3-ylboronate, (3aS,3bS,9aR,9bS,11aS)-1-(4-chloropyridin-3-yl)-5,9a,11a-trimethyl-3b,4,5,8,9,9a,9b,10,11,11a-decahydro-3H-cyclopenta[i]phenanthridin-7(3aH)-one is made.

### Example 87

### (3aS,3bS,9aR,9bS,11aS)-1-(4-Methylpyridin-3-yl)-5,9a,11a-trimethyl-3b,4,5,8,9,9a,9b,10,11,11a-decahydro-3H-cyclopenta[i]phenanthridin-7(3aH)-one

Using a synthetic procedure and condition similar to Example 4 in the preparation of (3aS,3bS,9aR,9bS,11aS)-5,9a,11a-trimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 4-methylpyridin-3-ylboronate, (3aS,3bS,9aR,9bS,11aS)-1-(4-methylpyridin-3-yl)-5,9a,11a-trimethyl-3b,4,5,8,9,9a,9b,10,11,11a-decahydro-3H-cyclopenta[i]phenanthridin-7(3aH)-one is made.

### Example 88

### (3aS,3bS,9aR,9bS,11aS)-1-(5-Methoxypyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 5 in the preparation of (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-methoxypyridin-3-ylboronate as in Example 5C and following the rest of reaction in Example 5, (3aS,3bS,9aR,9bS,11aS)-1-(5-methoxypyridin-3-yl)-9a,11a-dimethyl-3b,4,5,8,9,9a 9b,10,11,11a-decahydro-3H-cyclopenta[i]phenanthridin-7(3aH)-one is made.

### Example 89

### (3aS,3bS,9aR,9bS,11aS)-1-(5-Ethoxypyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 5 in the preparation of (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-ethoxypyridin-3-ylboronate as in Example 5C and following the rest of reaction in Example 5, (3aS,3bS,9aR,9bS,11aS)-1-(5-ethoxypyridin-3-yl)-9a,11a-dimethyl-3b,4,5,8,9,9a, 9b,10,11,11a-decahydro-3H-cyclopenta[i]phenanthridin-7(3aH)-one is made.

### Example 90

### (3aS,3bS,9aR,9bS,11aS)-1-(5-Fluoropyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 5 in the preparation of (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-fluoropyridin-3-ylboronate as in Example 5C and following the rest of reaction in Example 5, (3aS,3bS,9aR,9bS,11aS)-1-(5-fluoropyridin-3-yl)-9a,11a-dimethyl-3b,4,5,8,9,9a, 9b,10,11,11a-decahydro-3H-cyclopenta[i]phenanthridin-7(3aH)-one is made.

### Example 91

### (3aS,3bS,9aR,9bS,11aS)-1-(5-Chloropyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 5 in the preparation of (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-chloropyridin-3-ylboronate as in Example 5C and following the rest of reaction in Example 5, (3aS,3bS,9aR,9bS,11aS)-1-(5-chloropyridin-3-yl)-9a,11a-dimethyl-3b,4,5,8,9,9a, 9b,10,11,11a-decahydro-3H-cyclopenta[i]phenanthridin-7(3aH)-one is made.

### Example 92

### (3aS,3bS,9aR,9bS,11aS)-1-(5-Methylpyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 5 in the preparation of (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-methylpyridin-3-ylboronate as in Example 5C and following the rest of reaction in Example 5, (3aS,3bS,9aR,9bS,11aS)-1-(5-methylpyridin-3-yl)-9a,11a-dimethyl-3b,4,5,8,9,9a,9b,10,11,11a-decahydro-3H-cyclopenta[i]phenanthridin-7(3aH)-one is made.

### Example 93

### (3aS,3bS,9aR,9bS,11aS)-1-(4-methoxypyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 5 in the preparation of (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 4-methoxypyridin-3-ylboronate as in Example 5C and following the rest of reaction in Example 5, (3aS,3bS,9aR,9bS,11aS)-1-(4-methoxypyridin-3-yl)-9a,11a-dimethyl-3b,4,5,8,9,9a 9b,10,11,11a-decahydro-3H-cyclopenta[i]phenanthridin-7(3aH)-one is made.

### Example 94

### (3aS,3bS,9aR,9bS,11aS)-1-(4-Chloropyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 5 in the preparation of (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 4-chloropyridin-3-ylboronate as in Example 5C and following the rest of reaction in Example 5, (3aS,3bS,9aR,9bS,11aS)-1-(4-chloropyridin-3-yl)-9a,11a-dimethyl-3b,4,5,8,9,9a, 9b,10,11,11a-decahydro-3H-cyclopenta[i]phenanthridin-7(3aH)-one is made.

### Example 95

### (3aS,3bS,9aR,9bS,11aS)-1-(4-Methylpyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 5 in the preparation of (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 4-methylpyridin-3-ylboronate as in Example 5C and following the rest of reaction in Example 5, (3aS,3bS,9aR,9bS,11aS)-1-(4-methylpyridin-3-yl)-9a,11a-dimethyl-3b,4,5,8,9,9a,9b,10,11,11a-decahydro-3H-cyclopenta[i]phenanthridin-7(3aH)-one is made.

### Example 96

### (3aS,3bS,9aR,9bS,11aS)-5-ethyl-9a,11a-dimethyl-1-(pyridin-3-yl)-3b,4,5,8,9,9a,9b,10,11,11a-decahydro-3H-cyclopenta[i]phenanthridin-7(3aH)-one

### Example 96A

### (3aS,3bR,9aR,9bS,11aS)-5-ethyl-9a,11a-dimethyl-3,3a,3b,4,5,8,9,9a,9b,10,11,11a-dodecahydro-1H-cyclopenta[i]phenanthridine-1,7(2H)-dione

Using a synthetic procedure and condition similar to Example 4H in the preparation of (3aS,3bR,9aR,9bS,11aS)-5,9a,11a-trimethyl-3,3a,3b,4,5,8,9,9a,9b,10,11,11a-dodecahydro-1H-cyclopenta[i]phenanthridine-1,7(2H)-dione, replacing iodomethane with ethyl bromide, (3aS,3bR,9aR,9bS,11aS)-5-ethyl-9a,11a-dimethyl-3,3a,3b,4,5,8,9,9a,9b,10,11,11a-dodecahydro-1H-cyclopenta[i]phenanthridine-1,7(2H)-dione was made.

### Example 96B

### (3aS,3bR,9aR,9bS,11aS)-5-ethyl-9a,11a-dimethyl-7-oxo-3a,3b,4,5,7,8,9,9a,9b,10,11,11a-dodecahydro-3H-cyclopenta[i]phenanthridin-1-yl trifluoromethanesulfonate

Using a synthetic procedure and condition similar to Example 41 in the preparation of 3aS,3bR,9aR,9bS,11aS)-5,9a,11a-trimethyl-7-oxo-3a,3b,4,5,7,8,9,9a,9b,10,11,11a-dodecahydro-3H-cyclopenta[i]phenanthridin-1-yl trifluoromethanesulfonate, replacing (3aS,3bR,9aR,9bS,11aS)-5,9a,11a-trimethyl-3,3a,3b,4,5,8,9,9a,9b,10,11,11a-dodecahydro-1H-cyclopenta[i]phenanthridine-1,7(2H)-dione with (3aS,3bR,9aR,9bS,11aS)-5-ethyl-9a,11a-dimethyl-3,3a,3b,4,5,8,9,9a,9b,10,11,11a-dodecahydro-1H-cyclopenta[i]phenanthridine-1,7(2H)-dione, (3aS,3bR,9aR,9bS,11aS)-5-ethyl-9a,11a-dimethyl-7-oxo-3a,3b,4,5,7,8,9,9a,9b,10,11,11a-dodecahydro-3H-cyclopenta[i]phenanthridin-1-yl trifluoromethanesulfonate is made.

### Example 96

### (3aS,3bS,9aR,9bS,11aS)-5-Ethyl-9a,11a-dimethyl-1-(pyridin-3-yl)-3b,4,5,8,9,9a,9b,10,11,11a-decahydro-3H-cyclopenta[i]phenanthridin-7(3aH)-one

To a solution of (3aS,3bR,9aR,9bS,11aS)-5-ethyl-9a,11a-dimethyl-7-oxo-3a,3b,4,5,7,8,9,9a,9b,10,11,11a-dodecahydro-3H-cyclopenta[i]phenanthridin-1-yl trifluoromethanesulfonate (10.6 mmol) in tetrahydrofuran (150 mL) is added diethyl 3-pyridoborane (3.12 g, 21.2 mmol), sodium carbonate (5.06 g,) in water (30 mL), and bis(triphenylphosphine) palladium chloride (0.75 g,). The mixture is thoroughly degassed, and heated under nitrogen at 80° C. for overnight. After being filtered through a pad of Celite, the crude product is purified with silica gel column (5% to 10% methanol in dichloromethane) to give (3aS,3bS,9aR,9bS,11aS)-5-ethyl-9a,11a-dimethyl-1-(pyridin-3-yl)-3b,4,5,8,9,9a,9b,10,11,11a-decahydro-3H-cyclopenta[i]phenanthridin-7(3 aH)-one.

### Example 97

### (3aS,3bS,9aR,9bS,11aS)-5-Ethyl-1-(5-methoxypyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 96 in the preparation (3aS,3bS,9aR,9bS,11aS)-5-ethyl-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-methoxypyridin-3-ylboronate, (3aS,3bS,9aR,9bS,1aS)-5-ethyl-1-(5-methoxypyridin-3-yl)-9a,11a-dimethyl-3b,4,5,8,9,9a,9b,10,11,11a-decahydro-3H-cyclopenta[i]phenanthridin-7(3aH)-one is made.

### Example 98

### (3aS,3bS,9aR,9bS,11aS)-5-Ethyl-1-(5-ethoxypyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 96 in the preparation (3aS,3bS,9aR,9bS,11aS)-5-ethyl-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-ethoxypyridin-3-ylboronate, (3aS,3bS,9aR,9bS,111aS)-5-ethyl-1-(5-ethoxypyridin-3-yl)-9a,11a-dimethyl-3b,4,5,8,9,9a,9b,10,11,11a-decahydro-3H-cyclopenta[i]phenanthridin-7(3aH)-one is made.

### Example 99

### (3aS,3bS,9aR,9bS,11aS)-5-Ethyl-1-(5-fluoroypyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 96 in the preparation (3aS,3bS,9aR,9bS,11aS)-5-ethyl-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-fluoropyridin-3-ylboronate, (3aS,3bS,9aR,9bS,11aS)-5-ethyl-1-(5-fluoropyridin-3-yl)-9a,11a-dimethyl-3b,4,5,8,9,9a,9b,10,11,11a-decahydro-3H-cyclopenta[i]phenanthridin-7(3aH)-one is made.

### Example 100

### (3aS,3bS,9aR,9bS,11aS)-5-Ethyl-1-(5-chloropyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 96 in the preparation (3aS,3bS,9aR,9bS,11aS)-5-ethyl-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-chloropyridin-3-ylboronate, (3aS,3bS,9aR,9bS,111aS)-5-ethyl-1-(5-chloropyridin-3-yl)-9a,11a-dimethyl-3b,4,5,8,9,9a,9b,10,11,11a-decahydro-3H-cyclopenta[i]phenanthridin-7(3aH)-one is made.

### Example 101

### (3aS,3bS,9aR,9bS,11aS)-5-Ethyl-1-(5-methylpyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 96 in the preparation (3aS,3bS,9aR,9bS,11aS)-5-ethyl-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 5-chloropyridin-3-ylboronate, (3aS,3bS,9aR,9bS,111aS)-5-ethyl-1-(5-methylpyridin-3-yl)-9a,11a-dimethyl-3b,4,5,8,9,9a,9b,10,11,11a-decahydro-3H-cyclopenta[i]phenanthridin-7(3aH)-one is made.

### Example 102

### (3aS,3bS,9aR,9bS,11aS)-5-Ethyl-1-(4-methoxypyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 96 in the preparation (3aS,3bS,9aR,9bS,11aS)-5-ethyl-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 4-methoxypyridin-3-ylboronate, (3aS,3bS,9aR,9bS,11aS)-5-ethyl-1-(4-methoxypyridin-3-yl)-9a,11a-dimethyl-3b,4,5,8,9,9a,9b,10,11,11a-decahydro-3H-cyclopenta[i]phenanthridin-7(3aH)-one is made.

### Example 103

### (3aS,3bS,9aR,9bS,11aS)-5-Ethyl-1-(4-chloropyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 96 in the preparation (3aS,3bS,9aR,9bS,11aS)-5-ethyl-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 4-chloropyridin-3-ylboronate, (3aS,3bS,9aR,9bS,11aS)-5-ethyl-1-(4-chloropyridin-3-yl)-9a,11a-dimethyl-3b,4,5,8,9,9a,9b,10,11,11a-decahydro-3H-cyclopenta[i]phenanthridin-7(3aH)-one is made.

### Example 104

### (3aS,3bS,9aR,9bS,11aS)-5-Ethyl-1-(4-methylpyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 96 in the preparation (3aS,3bS,9aR,9bS,11aS)-5-ethyl-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 4-methylpyridin-3-ylboronate, (3aS,3bS,9aR,9bS,11aS)-5-ethyl-1-(4-methylpyridin-3-yl)-9a,11a-dimethyl-3b,4,5,8,9,9a,9b,10,11,11a-decahydro-3H-cyclopenta[i]phenanthridin-7(3aH)-one is made.

### Example 105

### (4aR,4bS,6aS,9aS,9bS)-7-(6-Methoxypyrazin-2-yl)-1,4a,6a-trimethyl-4,4a,4b,5,6,6a,9,9a,9b,10-decahydro-1H-indeno[5,4-f]quinolin-2(3H)-one

Using a synthetic procedure and condition similar to Example 20 in the preparation of (4aR,4bS,6aS,9aS,9bS)-1,4a,6a-trimethyl-7-(pyrazin-2-yl)-4,4a,4b,5,6,6a,9,9a,9b,10-decahydro-1H-indeno[5,4-f]quinolin-2(3H)-one, replacing 2-(tributylstannyl)pyrazine with 2-methoxy-6-(tributylstannyl)pyrazine, (4aR,4bS,6aS,9aS,9bS)-7-(6-methoxypyrazin-2-yl)-1,4a,6a-trimethyl-4,4a,4b,5,6,6a,9,9a,9b,10-decahydro-1H-indeno[5,4-f]quinolin-2(3H)-one is made.

### Example 106

### (4aR,4bS,6aS,9aS,9bS)-1,4a,6a-Trimethyl-7-(6-methylpyrazin-2-yl)-4,4a,4b,5,6,6a,9,9a,9b,10-decahydro-1H-indeno[5,4-f]quinolin-2(3H)-one

Using a synthetic procedure and condition similar to Example 20 in the preparation of (4aR,4bS,6aS,9aS,9bS)-1,4a,6a-trimethyl-7-(pyrazin-2-yl)-4,4a,4b,5,6,6a,9,9a,9b,10-decahydro-1H-indeno[5,4-f]quinolin-2(3H)-one, replacing 2-(tributylstannyl)pyrazine with 2-methyl-6-(tributylstannyl)pyrazine, (4aR,4bS,6aS,9aS,9bS)-1,4a,6a-trimethyl-7-(6-methylpyrazin-2-yl)-4,4a,4b,5,6,6a,9,9a,9b,10-decahydro-1H-indeno[5,4-f]quinolin-2(3H)-one is made.

### Example 107

### (4aR,4bS,6aS,9aS,9bS)-7-(6-Ethylpyrazin-2-yl)-1,4a,6a-trimethyl-4,4a,4b,5,6,6a,9,9a,9b,10-decahydro-1H-indeno[5,4-f]quinolin-2(3H)-one

Using a synthetic procedure and condition similar to Example 20 in the preparation of (4aR,4bS,6aS,9aS,9bS)-1,4a,6a-trimethyl-7-(pyrazin-2-yl)-4,4a,4b,5,6,6a,9,9a,9b,10-decahydro-1H-indeno[5,4-f]quinolin-2(3H)-one, replacing 2-(tributylstannyl)pyrazine with 2-ethyl-6-(tributylstannyl)pyrazine, (4aR,4bS,6aS,9aS,9bS)-7-(6-ethylpyrazin-2-yl)-1,4a,6a-trimethyl-4,4a,4b,5,6,6a,9,9a,9b,10-decahydro-1H-indeno[5,4-f]quinolin-2(3H)-one is made.

### Example 108

### (4aR,4bS,6aS,9aS,9bS)-7-(6-Ethoxypyrazin-2-yl)-1,4a,6a-trimethyl-4,4a,4b,5,6,6a,9,9a,9b,10-decahydro-1H-indeno[5,4-f]quinolin-2(3H)-one

Using a synthetic procedure and condition similar to Example 20 in the preparation of (4aR,4bS,6aS,9aS,9bS)-1,4a,6a-trimethyl-7-(pyrazin-2-yl)-4,4a,4b,5,6,6a,9,9a,9b,10-decahydro-1H-indeno[5,4-f]quinolin-2(3H)-one, replacing 2-(tributylstannyl)pyrazine with 2-ethoxy-6-(tributylstannyl)pyrazine, (4aR,4bS,6aS,9aS,9bS)-7-(6-ethoxypyrazin-2-yl)-1,4a,6a-trimethyl-4,4a,4b,5,6,6a,9,9a,9b,10-decahydro-1H-indeno[5,4-f]quinolin-2(3H)-one is made.

### Example 109

### (4aR,4bS,6aS,9aS,9bS)-1-Ethyl-7-(6-methoxypyrazin-2-yl)-4a,6a-dimethyl-4,4a,4b,5,6,6a,9,9a,9b,10-decahydro-1H-indeno[5,4-f]quinolin-2(3H)-one

Using a synthetic procedure and condition similar to Example 30 in the preparation of (4aR,4bS,6aS,9aS,9bS)-1-ethyl-4a,6a-dimethyl-7-(pyrazin-2-yl)-4,4a,4b,5,6,6a,9,9a,9b,10-decahydro-1H-indeno[5,4-f]quinolin-2(3H)-one, replacing 2-(tributylstannyl)pyrazine with 2-methoxy-6-(tributylstannyl)pyrazine, (4aR,4bS,6aS,9aS,9bS)-1-ethyl-7-(6-methoxypyrazin-2-yl)-4a,6a-dimethyl-4,4a,4b,5,6,6a,9,9a,9b,10-decahydro-1H-indeno[5,4-f]quinolin-2(3H)-one is made.

### Example 110

### (4aR,4bS,6aS,9aS,9bS)-1-Ethyl-4a,6a-dimethyl-7-(6-methylpyrazin-2-yl)-4,4a,4b,5,6,6a,9,9a,9b,10-decahydro-1H-indeno[5,4-f]quinolin-2(3H)-one

Using a synthetic procedure and condition similar to Example 30 in the preparation of (4aR,4bS,6aS,9aS,9bS)-1-ethyl-4a,6a-dimethyl-7-(pyrazin-2-yl)-4,4a,4b,5,6,6a,9,9a,9b,10-decahydro-1H-indeno[5,4-f]quinolin-2(3H)-one, replacing 2-(tributylstannyl)pyrazine with 2-methyl-6-(tributylstannyl)pyrazine, (4aR,4bS,6aS,9aS,9bS)-1-ethyl-7-(6-methoxypyrazin-2-yl)-4a,6a-dimethyl-4,4a,4b,5,6,6a,9,9a,9b,10-decahydro-1H-indeno[5,4-f]quinolin-2(3H)-one is made.

### Example 111

### (4aR,4bS,6aS,9aS,9bS)-1-Ethyl-7-(6-ethylpyrazin-2-yl)-4a,6a-dimethyl 4,4a,4b,5,6,6a,9,9a,9b,10-decahydro-1H-indeno[5,4-f]quinolin-2(3H)-one

Using a synthetic procedure and condition similar to Example 30 in the preparation of (4aR,4bS,6aS,9aS,9bS)-1-ethyl-4a,6a-dimethyl-7-(pyrazin-2-yl)-4,4a,4b,5,6,6a,9,9a,9b,10-decahydro-1H-indeno[5,4-f]quinolin-2(3H)-one, replacing 2-(tributylstannyl)pyrazine with 2-ethyl-6-(tributylstannyl)pyrazine, (4aR,4bS,6aS,9aS,9bS)-1-ethyl-7-(6-ethylpyrazin-2-yl)-4a,6a-dimethyl 4,4a,4b,5,6,6a,9,9a,9b,10-decahydro-1H-indeno[5,4-f]quinolin-2(3H)-one is made.

### Example 112

### (4aR,4bS,6aS,9aS,9bS)-1-Ethyl-7-(6-ethoxylpyrazin-2-yl)-4a,6a-dimethyl 4,4a,4b,5,6,6a,9,9a,9b,10-decahydro-1H-indeno[5,4-f]quinolin-2(3H)-one

Using a synthetic procedure and condition similar to Example 30 in the preparation of (4aR,4bS,6aS,9aS,9bS)-1-ethyl-4a,6a-dimethyl-7-(pyrazin-2-yl)-4,4a,4b,5,6,6a,9,9a,9b,10-decahydro-1H-indeno[5,4-f]quinolin-2(3H)-one, replacing 2-(tributylstannyl)pyrazine with 2-ethoxy-6-(tributylstannyl)pyrazine, (4aR,4bS,6aS,9aS,9bS)-1-ethyl-7-(6-ethoxylpyrazin-2-yl)-4a,6a-dimethyl 4,4a,4b,5,6,6a,9,9a,9b,10-decahydro-1H-indeno[5,4-f]quinolin-2(3H)-one is made.

### Example 113

### (4aR,4bS,6aS,9aS,9bS)-1-Cyclopropyl-7-(6-methoxypyrazin-2-yl)-4a,6a-dimethyl-4,4a,4b,5,6,6a,9,9a,9b,10-decahydro-1H-indeno[5,4-f]quinolin-2(3H)-one

Using a synthetic procedure and condition similar to Example 38 in the preparation of (4aR,4bS,6aS,9aS,9bS)-1-cyclopropyl-4a,6a-dimethyl-7-(pyrazin-2-yl)-4,4a,4b, 5,6,6a,9,9a,9b,10-decahydro-1H-indeno[5,4-f]quinolin-2(3H)-one, replacing 2-(tributylstannyl)pyrazine with 2-methoxy-6-(tributylstannyl)pyrazine, (4aR,4bS,6aS,9aS,9bS)-1-cyclopropyl-7-(6-methoxypyrazin-2-yl)-4a,6a-dimethyl-4,4a,4b,5,6,6a,9,9a,9b,10-decahydro-1H-indeno[5,4-f]quinolin-2(3H)-one is made.

### Example 114

### (4aR,4bS,6aS,9aS,9bS)-1-Cyclopropyl-7-(6-methylpyrazin-2-yl)-4a,6a-dimethyl-4,4a,4b,5,6,6a,9,9a,9b,10-decahydro-1H-indeno[5,4-f]quinolin-2(3H)-one

Using a synthetic procedure and condition similar to Example 38 in the preparation of (4aR,4bS,6aS,9aS,9bS)-1-cyclopropyl-4a,6a-dimethyl-7-(pyrazin-2-yl)-4,4a,4b,5,6,6a,9,9a,9b,10-decahydro-1H-indeno[5,4-f]quinolin-2(3H)-one, replacing 2-(tributylstannyl)pyrazine with 2-methyl-6-(tributylstannyl)pyrazine, (4aR,4bS,6aS,9aS,9bS)-1-cyclopropyl-7-(6-methylpyrazin-2-yl)-4a,6a-dimethyl-4,4a,4b,5,6,6a,9,9a,9b,10-decahydro-1H-indeno[5,4-f]quinolin-2(3H)-one is made.

### Example 115

### (4aR,4bS,6aS,9aS,9bS)-1-Cyclopropyl-7-(6-ethylpyrazin-2-yl)-4a,6a-dimethyl-4,4a,4b,5,6,6a,9,9a,9b,10-decahydro-1H-indeno[5,4-f]quinolin-2(3H)-one

Using a synthetic procedure and condition similar to Example 38 in the preparation of (4aR,4bS,6aS,9aS,9bS)-1-cyclopropyl-4a,6a-dimethyl-7-(pyrazin-2-yl)-4,4a,4b,5,6,6a,9,9a,9b,10-decahydro-1H-indeno[5,4-f]quinolin-2(3H)-one, replacing 2-(tributylstannyl)pyrazine with 2-ethyl-6-(tributylstannyl)pyrazine, (4aR,4bS,6aS,9aS,9bS)-1-cyclopropyl-7-(6-ethylpyrazin-2-yl)-4a,6a-dimethyl-4,4a,4b,5,6,6a,9,9a,9b,10-decahydro-1H-indeno[5,4-f]quinolin-2(3H)-one is made.

### Example 116

### (4aR,4bS,6aS,9aS,9bS)-1-Cyclopropyl-7-(6-ethoxypyrazin-2-yl)-4a,6a-dimethyl-4,4a,4b,5,6,6a,9,9a,9b,10-decahydro-1H-indeno[5,4-f]quinolin-2(3H)-one

Using a synthetic procedure and condition similar to Example 38 in the preparation of (4aR,4bS,6aS,9aS,9bS)-1-cyclopropyl-4a,6a-dimethyl-7-(pyrazin-2-yl)-4,4a,4b,5,6,6a,9,9a,9b,10-decahydro-1H-indeno[5,4-f]quinolin-2(3H)-one, replacing 2-(tributylstannyl)pyrazine with 2-ethoxy-6-(tributylstannyl)pyrazine, (4aR,4bS,6aS,9aS,9bS)-1-cyclopropyl-7-(6-ethoxypyrazin-2-yl)-4a,6a-dimethyl-4,4a,4b,5,6,6a,9,9a,9b,10-decahydro-1H-indeno[5,4-f]quinolin-2(3H)-one is made.

### Example 117

### (3aS,3bS,9aR,9bS,11aS)-5,9a,11a-trimethyl-1-(pyrazin-2-yl)-3b,4,5,8,9,9a,9b,10,11,11a-decahydro-3H-cyclopenta[i]phenanthridin-7(3aH)-one

Using a synthetic procedure and condition similar to Example 20 in the preparation of (4aR,4bS,6aS,9aS,9bS)-1,4a,6a-trimethyl-7-(pyrazin-2-yl)-4,4a,4b,5,6,6a,9,9a,9b,10-decahydro-1H-indeno[5,4-f]quinolin-2(3H)-one, replacing (4aR,4bS,6aS,9aS,9bR)-1,4a,6a-trimethyl-2-oxo-2,3,4,4a,4b,5,6,6a,9,9a,9b,10-dodecahydro-1H-indeno[5,4-f]quinolin-7-yl trifluoromethanesulfonate with (3aS,3bR,9aR,9bS,11aS)-5,9a,11a-trimethyl-7-oxo-3a,3b,4,5,7,8,9,9a,9b,10,11,11a-dodecahydro-3H-cyclopenta[i]phenanthridin-1-yl trifluoromethanesulfonate and keeping the use of Pd(PPh₃)₄ and 2-(tributylstannyl)pyrazine, (3aS,3bS,9aR,9bS,11aS)-5,9a,11a-trimethyl-1-(pyrazin-2-yl)-3b,4,5,8,9,9a,9b,10,11,11a-decahydro-3H-cyclopenta[i]phenanthridin-7(3aH)-one is made.

### Example 118

### (3aS,3bS,9aR,9bS,11aS)-1-(6-methoxypyrazin-2-yl)-5,9a,11a-trimethyl-3b,4,5,8,9,9a,9b,10,11,11a-decahydro-3H-cyclopenta[i]phenanthridin-7(3aH)-one

Using a synthetic procedure and condition similar to Example 117 in the preparation of (3aS,3bS,9aR,9bS,11aS)-5,9a,11a-trimethyl-1-(pyrazin-2-yl)-3b,4,5,8,9,9a,9b,10,11,11a-decahydro-3H-cyclopenta[i]phenanthridin-7(3aH)-one, replacing 2-(tributylstannyl)pyrazine with 2-methoxy-6-(tributylstannyl)pyrazine, (3aS,3bS,9aR,9bS,11aS)-1-(6-methoxypyrazin-2-yl)-5,9a,11a-trimethyl-3b,4,5,8,9,9a,9b,10,11,11a-decahydro-3H-cyclopenta[i]phenanthridin-7(3aH)-one is made.

### Example 119

### (3aS,3bS,9aR,9bS,11aS)-1-(6-ethoxypyrazin-2-yl)-5,9a,11a-trimethyl-3b,4,5,8,9,9a,9b,10,11,11a-decahydro-3H-cyclopenta[i]phenanthridin-7(3aH)-one

Using a synthetic procedure and condition similar to Example 117 in the preparation of (3aS,3bS,9aR,9bS,11aS)-5,9a,11a-trimethyl-1-(pyrazin-2-yl)-3b,4,5,8,9,9a,9b,10,11,11a-decahydro-3H-cyclopenta[i]phenanthridin-7(3aH)-one, replacing 2-(tributylstannyl)pyrazine with 2-ethoxy-6-(tributylstannyl)pyrazine, (3aS,3bS,9aR,9bS,11aS)-1-(6-ethoxypyrazin-2-yl)-5,9a,11a-trimethyl-3b,4,5,8,9,9a,9b,10,11,11a-decahydro-3H-cyclopenta[i]phenanthridin-7(3aH)-one is made.

### Example 120

### (3aS,3bS,9aR,9bS,11aS)-1-(6-ethylpyrazin-2-yl)-5,9a,11a-trimethyl-3b,4,5,8,9,9a,9b,10,11,11a-decahydro-3H-cyclopenta[i]phenanthridin-7(3aH)-one

Using a synthetic procedure and condition similar to Example 117 in the preparation of (3aS,3bS,9aR,9bS,11aS)-5,9a,11a-trimethyl-1-(pyrazin-2-yl)-3b,4,5,8,9,9a,9b,10,11,11a-decahydro-3H-cyclopenta[i]phenanthridin-7(3aH)-one, replacing 2-(tributylstannyl)pyrazine with 2-ethyl-6-(tributylstannyl)pyrazine, (3aS,3bS,9aR,9bS,11aS)-1-(6-ethylpyrazin-2-yl)-5,9a,11a-trimethyl-3b,4,5,8,9,9a,9b,10,11,11a-decahydro-3H-cyclopenta[i]phenanthridin-7(3aH)-one is made.

### Example 121

### (4aR,4bS,6aS,9aS,9bS)-7-(5-Methoxypyridin-3-yl)-4a,6a-dimethyl-4,4a,4b,5,6,6a,9,9a,9b,10,11,11a-dodecahydroindeno[5,4-f]chromen-2(3H)-one

Using a synthetic procedure and condition similar to Example 61 in the preparation of (4aR,4bS,6aS,9aS,9bS)-4a,6a-dimethyl-7-(pyridin-3-yl)-4,4a,4b,5,6,6a,9,9a,9b,10,11,11a-dodecahydroindeno[5,4-f]chromen-2(3H)-one, replacing pyridin-3-ylboronic acid with 5-methoxypyridin-3-ylboronic acid in Example 61C followed by reacting the product with NaBH₄ in methanol in similar condition as in Example 61, (4aR,4bS,6aS,9aS,9bS)-7-(5-methoxypyridin-3-yl)-4a,6a-dimethyl-4,4a,4b,5,6,6a,9,9a,9b,10,11,11a-dodecahydroindeno[5,4-f]chromen-2(3H)-one is made.

### Example 122

### (4aR,4bS,6aS,9aS,9bS)-7-(5-Ethoxypyridin-3-yl)-4a,6a-dimethyl-4,4a,4b,5,6,6a,9,9a,9b,10,11,11a-dodecahydroindeno[5,4-f]chromen-2(3H)-one

Using a synthetic procedure and condition similar to Example 61 in the preparation of (4aR,4bS,6aS,9aS,9bS)-4a,6a-dimethyl-7-(pyridin-3-yl)-4,4a,4b,5,6,6a,9,9a,9b,10,11,11a-dodecahydroindeno[5,4-f]chromen-2(3H)-one, replacing pyridin-3-ylboronic acid with 5-ethoxypyridin-3-ylboronic acid in Example 61C followed by reacting the product with NaBH₄ in methanol in similar condition as in Example 61, (4aR,4bS,6aS,9aS,9bS)-7-(5-ethoxypyridin-3-yl)-4a,6a-dimethyl-4,4a,4b,5,6,6a,9,9a,9b,10,11,11a-dodecahydroindeno[5,4-f]chromen-2(3H)-one is made.

### Example 123

### (4aR,4bS,6aS,9aS,9bS)-7-(4-Methoxypyridin-3-yl)-4a,6a-dimethyl-4,4a,4b,5,6,6a,9,9a,9b,10,11,11a-dodecahydroindeno[5,4-f]chromen-2(3H)-one

Using a synthetic procedure and condition similar to Example 61 in the preparation of (4aR,4bS,6aS,9aS,9bS)-4a,6a-dimethyl-7-(pyridin-3-yl)-4,4a,4b,5,6,6a,9,9a,9b,10,11,11a-dodecahydroindeno[5,4-f]chromen-2(3H)-one, replacing pyridin-3-ylboronic acid with 4-methoxypyridin-3-ylboronic acid in Example 61C followed by reacting the product with NaBH₄ in methanol in similar condition as in Example 61, (4aR,4bS,6aS,9aS,9bS)-7-(4-methoxypyridin-3-yl)-4a,6a-dimethyl-4,4a,4b,5,6,6a,9,9a,9b,10,11,11a-dodecahydroindeno[5,4-f]chromen-2(3H)-one is made.

### Example 124

### (4aR,4bS,6aS,9aS,9bS)-7-(5-Methoxypyridin-3-yl)-4a,6a-dimethyl-4,4a,4b,5,6,6a,9,9a,9b,10-decahydroindeno[5,4-f]chromen-2(3H)-one

Using a synthetic procedure and condition similar to Example 62 in the preparation of (4aR,4bS,6aS,9aS,9bS)-4a,6a-dimethyl-7-(pyridin-3-yl)-4,4a,4b,5,6,6a,9,9a,9b,10-decahydroindeno[5,4-f]chromen-2(3H)-one, replacing pyridin-3-ylboronic acid with 5-methoxypyridin-3-ylboronic acid in Example 61C followed by reacting the product with NaOAc in acetic anhydride, in similar experimental condition as in Example 62, (4aR,4bS,6aS,9aS,9bS)-7-(5-methoxypyridin-3-yl)-4a,6a-dimethyl-4,4a,4b,5,6,6a,9,9a,9b,10-decahydroindeno[5,4-f]chromen-2(3H)-one is made.

### Example 125

### (4aR,4bS,6aS,9aS,9bS)-7-(5-Ethoxypyridin-3-yl)-4a,6a-dimethyl-4,4a,4b,5,6,6a,9,9a,9b,10-decahydroindeno[5,4-f]chromen-2(3H)-one

Using a synthetic procedure and condition similar to Example 62 in the preparation of (4aR,4bS,6aS,9aS,9bS)-4a,6a-dimethyl-7-(pyridin-3-yl)-4,4a,4b,5,6,6a,9,9a,9b,10-decahydroindeno[5,4-f]chromen-2(3H)-one, replacing pyridin-3-ylboronic acid with 5-ethoxypyridin-3-ylboronic acid in Example 61C followed by reacting the product with NaOAc in acetic anhydride, in similar experimental condition as in Example 62, (4aR,4bS,6aS,9aS,9bS)-7-(5-ethoxypyridin-3-yl)-4a,6a-dimethyl-4,4a,4b,5,6,6a,9,9a,9b,10-decahydroindeno[5,4-f]chromen-2(3H)-one is made.

### Example 126

### (4aR,4bS,6aS,9aS,9bS)-7-(4-Methoxypyridin-3-yl)-4a,6a-dimethyl-4,4a,4b,5,6,6a,9,9a,9b,10-decahydroindeno[5,4-f]chromen-2(3H)-one

Using a synthetic procedure and condition similar to Example 62 in the preparation of (4aR,4bS,6aS,9aS,9bS)-4a,6a-dimethyl-7-(pyridin-3-yl)-4,4a,4b,5,6,6a,9,9a,9b,10-decahydroindeno[5,4-f]chromen-2(3H)-one, replacing pyridin-3-ylboronic acid with 4-methoxypyridin-3-ylboronic acid in Example 61C followed by reacting the product with NaOAc in acetic anhydride, in similar experimental condition as in Example 62, (4aR,4bS,6aS,9aS,9bS)-7-(4-methoxypyridin-3-yl)-4a,6a-dimethyl-4,4a,4b,5,6,6a,9,9a,9b,10-decahydroindeno[5,4-f]chromen-2(3H)-one is made.

### Example 127

### (4aR,4bS,6aS,9aS,9bS)-7-(4-Methoxypyridin-3-yl)-4a,6a-dimethyl-4,4a,4b,5,6,6a,9,9a,9b,10-decahydro-1H-indeno[5,4-f]quinolin-2(3H)-one

### Example 127A

### (4aR,4bS,6aS,9aS,9bS, E)-7-Hydrazono-4a,6a-dimethyl-4,4a,4b,5,6,6a,7,8,9,9a,9b,10-dodecahydro-1H-indeno[5,4-f]quinolin-2(3H)-one

To a solution of (4aR,4bS,6aS,9aS,9bR)-4a,6a-dimethyl-4,4a,4b,5,6,6a,9,9a,9b,10-decahydro-1H-indeno[5,4-f]quinoline-2,7(3H,8H)-dione (1.0 g, 3.5 mmol) in ethanol (40 mL) was added hydrazine hydrate (2.06 g, 35 mmol) and TEA (1.06 g, 10.5 mmol). The resulting mixture was stirred at reflux for 2 h. Then cooled down to room temperature and concentrated to give (4aR,4bS,6aS,9aS,9bS, E)-7-hydrazono-4a,6a-dimethyl-4,4a,4b,5,6,6a,7,8,9,9a,9b,10-dodecahydro-1H-indeno[5,4-f]quinolin-2(3H)-one (1.0 g, yield 95%) as a white solid. ¹H-NMR (400 MHz, CDCl₃) major characteristic peaks: δ 0.77 (s, 3H), 1.01 (s, 3H), 4.84 (s, 1H), 5.31 (s, 2H), 9.28 (s, 1H). LC-MS (m/z) 302 [M+H]⁺.

### Example 127B

### (4aR,4bS,6aS,9aS,9bR)-7-Iodo-4a,6a-dimethyl-4,4a,4b,5,6,6a,9,9a,9b,10-decahydro-1H-indeno[5,4-f]quinolin-2(3H)-one

To a mixture of (4aR,4bS,6aS,9aS,9bS, E)-7-hydrazono-4a,6a-dimethyl-4,4a,4b,5,6,6a,7,8,9,9a,9b,10-dodecahydro-1H-indeno[5,4-f]quinolin-2(3H)-one (1.0 g, 3.32 mmol) in a solution of chloroform and benzene (130 mL, 1:1) was added TEA (6.71 g, 66.4 mmol), then followed by a solution of iodine (1.68 g, 6.61 mmol) in benzene (20 mL). After stirring at room temperature for 5 h, the mixture was diluted with chloroform (50 mL), successively washed with HCl (10%, 20 mL), water (20 mL), aqueous Na₂ SO₃ (5%, 20 mL) and water (20 mL), dried over anhydrous sodium sulfate, and then concentrated to give (4aR,4bS,6aS,9aS,9bR)-7-iodo-4a,6a-dimethyl-4,4a,4b,5,6,6a,9,9a,9b,10-decahydro-1H-indeno[5,4-f]quinolin-2(3H)-one (1.2 g, yield 91%) as a white solid. ¹H-NMR (400 MHz, CDCl₃) major characteristic peaks: δ 0.78 (s, 3H), 1.13 (s, 3H), 4.86 (m, 1H), 6.15 (s, 1H), 7.79 (s, 1H). LC-MS (m/z) 398 [M+H]⁺.

### Example 127

### (4aR,4bS,6aS,9aS,9bS)-7-(4-Methoxypyridin-3-yl)-4a,6a-dimethyl-4,4a,4b,5,6,6a,9,9a,9b,10-decahydro-1H-indeno[5,4-f]quinolin-2(3H)-one

Pd(dppf)Cl₂ (37 mg, 0.05 mmol), 4-methoxypyridin-3-ylboronic acid hydrate (131 mg, 0.86 mmol) and K₂ CO₃ (2 M, 1.13 mL, 2.27 mmol) were added consecutively to a stirred solution of ((4aR,4bS,6aS,9aS,9bR)-7-iodo-4a,6a-dimethyl-4,4a,4b,5,6,6a,9,9a,9b,10-decahydro-1H-indeno[5,4-f]quinolin-2(3H)-one (200 mg, 0.50 mmol) in 1,4-dioxane (10 mL). The mixture was heated at reflux under N₂ for 1 h. Then cooled to room temperature and partitioned between ethyl acetate (20 mL) and water (20 mL). The aqueous layer extracted with ethyl acetate (50 mL×3). The combined organic layers were dried over Na₂ SO₄. After filtration, the organic phase was concentrated and purified by prep-TLC (DCM/MeOH, 20/1) to give (4aR,4bS,6aS,9aS,9bS)-7-(4-methoxypyridin-3-yl)-4a,6a-dimethyl-4,4a,4b,5,6,6a,9,9a,9b,10-decahydro-1H-indeno[5,4-f]quinolin-2(3H)-one (19 mg, yield 10%) as a white solid. ¹H NMR (CDCl₃, 400 MHz) major characteristic peaks: δ 0.97 (s, 3H), 1.15 (s, 3H), 3.86 (s, 3H), 4.86 (m, 1H), 5.90 (s, 1H), 6.82 (d, J=5.6 Hz, 1H), 7.45 (s, 1H), 8.24 (s, 1H), 8.40 (d, J=5.2 Hz, 1H). LC-MS (m/z) 379 [M+H]⁺.

### Example 128

### (4aR,4bS,6aS,9aS,9bS)-7-(5-Methoxypyridin-3-yl)-4a,6a-dimethyl-4,4a,4b,5,6,6a,9,9a,9b,10-decahydro-1H-indeno[5,4-f]quinolin-2(3H)-one

Pd(dppf)Cl₂ (37 mg, 0.05 mmol), 5-methoxypyridin-3-ylboronic acid hydrate (131 mg, 0.86 mmol) and K₂ CO₃ (2 M, 1.13 mL, 2.27 mmol) were added consecutively to a stirred solution of (4aR,4bS,6aS,9aS,9bR)-7-iodo-4a,6a-dimethyl-4,4a,4b,5,6,6a,9,9a,9b,10-decahydro-1H-indeno[5,4-f]quinolin-2(3H)-one (200 mg, 0.50 mmol) in 1,4-dioxane (10 mL). The mixture was heated at reflux under N₂ for 1 h. Then cooled to room temperature and partitioned between ethyl acetate (20 mL) and water (20 mL). The aqueous layer extracted with ethyl acetate (50 mL×3). The combined organic layers were dried over Na₂ SO₄. After filtration, the organic phase was concentrated and purified by prep-TLC (DCM/MeOH, 20/1) to give (4aR,4bS,6aS, 9aS,9bS)-7-(5-methoxypyridin-3-yl)-4a,6a-dimethyl-4,4a,4b,5,6,6a,9,9a,9b,10-decahydro-1H-indeno[5,4-f]quinolin-2(3H)-one (35 mg, yield 18%) as a white solid. ¹H NMR (CDCl₃, 400 MHz) major characteristic peaks: δ 1.07 (s, 3H), 1.17 (s, 3H), 3.88 (s, 3H), 4.83 (m, 1H), 6.03 (s, 1H), 7.19 (m, 2H), 8.19 (s, 1H), 8.25 (s, 1H). LC-MS (m/z) 379 [M+H]⁺.

### Example 129

### (4aR,4bS,6aS,9aS,9bS)-7-(6-Methoxypyridin-3-yl)-4a,6a-dimethyl-4,4a,4b,5,6,6a,9,9a,9b,10-decahydro-1H-indeno[5,4-f]quinolin-2(3H)-one

Pd(dppf)Cl₂ (40 mg), 6-methoxypyridin-3-ylboronic acid (131 mg, 0.85 mmol) and K₂ CO₃ (2 M, 1.0 mL, 2.0 mmol) were added consecutively to a stirred solution of (4aR,4bS,6aS,9aS,9bR)-7-iodo-4a,6a-dimethyl-4,4a,4b,5,6,6a,9,9a,9b,10-decahydro-1H-indeno[5,4-f]quinolin-2(3H)-one (200 mg, 0.5 mmol) in 1,4-dioxane (15 mL). The reaction was heated at 100° C. under N₂ for 2 h. Then cooled to room temperature and partitioned between ethyl acetate (100 mL) and water (100 mL). The layers were separated and the aqueous layer extracted with ethyl acetate (25 mL×3). The combined organic layers were dried over Na₂ SO₄. After filtration, the organic phase was concentrated under vacuum and the residue was purified by prep-chromatography to afford (4aR,4bS,6aS,9aS,9bS)-7-(6-methoxypyridin-3-yl)-4a,6a-dimethyl-4,4a,4b,5,6,6a,9,9a,9b,10-decahydro-1H-indeno[5,4-f]quinolin-2(3H)-one (5 mg, yield 3%) as a white solid. ¹H-NMR (CDCl₃, 400 MHz) major characteristic peaks: δ 8.42 (s, 1H), 8.27 (s, 1H), 7.29 (s, 1H), 6.84 (s, 1H), 5.89 (m, 1H), 4.84 (m, 1H), 3.92 (s, 3H), 1.14 (s, 3H), 0.97 (s, 3H). LC-MS (m/z) 379 [M+H]⁺.

### Example 130

### (4aR,4bS,6aS,9aS,9bS)-4a,6a-Dimethyl-7-(pyrazin-2-yl)-4,4a,4b,5,6,6a,9,9a,9b,10-decahydro-1H-indeno[5,4-f]quinolin-2(3H)-one

A mixture of Pd(PPh₃)₄ (35 mg, 0.03 mmol), 2-(tributylstannyl)pyrazine (260 mg, 0.71 mmol) and (4aR,4bS,6aS,9aS,9bR)-7-iodo-4a,6a-dimethyl-4,4a,4b,5,6,6a,9,9a,9b,10-decahydro-1H-indeno[5,4-f]quinolin-2(3H)-one (200 mg, 0.50 mmol) in DMF (15 mL) was heated at 90° C. under N₂ overnight. Then cooled to room temperature and partitioned between ethyl acetate (20 mL) and water (20 mL). The aqueous layer extracted with ethyl acetate (50 mL×3). The combined organic layers were dried over Na₂ SO₄. After filtration, the organic phase was concentrated and purified by prep-HPLC to give (4aR,4bS,6aS,9aS,9bS)-4a,6a-dimethyl-7-(pyrazin-2-yl)-4,4a,4b,5,6,6a,9,9a,9b,10-decahydro-1H-indeno[5,4-f]quinolin-2(3H)-one (12 mg, yield 12%) as a white solid. ¹H-NMR (400 MHz, CDCl₃) major characteristic peaks: δ 0.90 (s, 3H), 1.10 (s, 3H), 4.82 (m, 1H), 6.71 (m, 1H), 7.56 (s, 1H), 8.29 (d, J=2.4 Hz, 1H), 8.42 (s, 1H), 8.65 (s, 1H). LC-MS (m/z) 350 [M+H]⁺.

### Example 131

### (4aR,4bS,6aS,9aS,9bS)-7-(6-methoxypyrazin-2-yl)-4a,6a-dimethyl-4,4a,4b,5,6,6a,9,9a,9b,10-decahydro-1H-indeno[5,4-f]quinolin-2(3H)-one

Using a synthetic procedure and condition similar to Example 130 in the preparation of (4aR,4bS,6aS,9aS,9bS)-4a,6a-dimethyl-7-(pyrazin-2-yl)-4,4a,4b,5,6,6a,9,9a,9b,10-decahydro-1H-indeno[5,4-f]quinolin-2(3H)-one, replacing 2-(tributylstannyl)pyrazine with 2-methoxy-6-(tributylstannyl)pyrazine, (4aR,4bS,6aS,9aS,9bS)-7-(6-methoxypyrazin-2-yl)-4a,6a-dimethyl-4,4a,4b,5,6,6a,9,9a,9b,10-decahydro-1H-indeno[5,4-f]quinolin-2(3H)-one is made.

### Example 132

### (4aR,4bS,6aS,9aS,9bS)-4a,6a-dimethyl-7-(pyrimidin-5-yl)-4,4a,4b,5,6,6a,9,9a,9b,10-decahydro-1H-indeno[5,4-f]quinolin-2(3H)-one

Pd(dppf)Cl₂ (40 mg), pyrimidin-5-ylboronic acid (210 mg, 1.7 mmol) and Cs₂ CO₃ (2 M, 1.0 mL, 2.0 mmol) were added consecutively to a stirred solution of (4aR,4bS,6aS,9aS,9bR)-7-iodo-4a,6a-dimethyl-4,4a,4b,5,6,6a,9,9a,9b,10-decahydro-1H-indeno[5,4-f]quinolin-2(3H)-one (200 mg, 0.5 mmol) in 1,4-dioxane (15 mL). The reaction was heated at 100° C. under N₂ for 2 h. Then cooled to room temperature and partitioned between ethyl acetate (100 mL) and water (100 mL). The layers were separated and the aqueous layer extracted with ethyl acetate (25 mL×3). The combined organic layers were dried over Na₂ SO₄. After filtration, the organic phase was concentrated under vacuum and the residue was purified by prep-chromatogram to afford (4aR,4bS,6aS,9aS,9bS)-4a,6a-dimethyl-7-(pyrimidin-5-yl)-4,4a,4b,5,6,6a,9,9a,9b,10-decahydro-1H-indeno[5,4-f]quinolin-2(3H)-one (5 mg, yield 3%) as a white solid. ¹H-NMR (CDCl₃, 400 MHz) major characteristic peaks: δ 9.08 (s, 1H), 8.74 (s, 2H), 8.13 (s, 1H), 6.12 (m, 1H), 4.92 (m, 1H), 1.16 (s, 3H), 1.07 (s, 3H). LC-MS (m/z) 350 [M+H]⁺.

### Example 133

### (3aS,3bS,9aR,9bS,11aS)-1-(6-Methoxypyridin-3-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 5 in the preparation of (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl 6-methoxypyridin-3-ylboronate as in Example 5C and following the rest of reaction in Example 5, (3aS,3bS,9aR,9bS,11aS)-1-(6-methoxypyridin-3-yl)-9a,11a-dimethyl-3b,4,5,8,9,9a,9b,10,11,11a-decahydro-3H-cyclopenta[i]phenanthridin-7(3aH)-one is made.

### Example 134

### (3aS,3bS,9aR,9bS,11aS)-9a,11a-Dimethyl-1-(pyrazin-2-yl)-3b,4,5,8,9,9a,9b,10,11,11a-decahydro-3H-cyclopenta[i]phenanthridin-7(3aH)-one

### Example 134A

### (3aS,3bS,9aR,9bS,11aS)-1-Iodo-9a,11a-dimethyl-3b,4,5,8,9,9a,9b,10,11,11a-decahydro-3H-cyclopenta[i]phenanthridin-7(3aH)-one

Following the synthetic procedure and condition of Example 127 A and B in the preparation of (4aR,4bS,6aS,9aS,9bR)-7-iodo-4a,6a-dimethyl-4,4a,4b,5,6,6a,9,9a,9b,10-decahydro-1H-indeno[5,4-f]quinolin-2(3H)-one, replacing (4aR,4bS,6aS,9aS,9bR)-4a,6a-dimethyl-4,4a,4b,5,6,6a,9,9a,9b,10-decahydro-1H-indeno[5,4-f]quinoline-2,7(3H,8H)-dione with (3aS,3bR,9aR,9bS,11aS)-9a,11a-dimethyl-3,3a,3b,4,5,8,9,9a,9b,10,11,11a-dodecahydro-1H-cyclopenta[i]phenanthridine-1,7(2H)-dione, (3aS,3bS,9aR,9bS,11aS)-1-iodo-9a,11a-dimethyl-3b,4,5,8,9,9a,9b,10,11,11a-decahydro-3H-cyclopenta[i]phenanthridin-7(3aH)-one is made.

### Example 134

### (3aS,3bS,9aR,9bS,11aS)-9a,11a-Dimethyl-1-(pyrazin-2-yl)-3b,4,5,8,9,9a,9b,10,11,11a-decahydro-3H-cyclopenta[i]phenanthridin-7(3aH)-one

Using a synthetic procedure and condition similar to Example 130 in the preparation of (4aR,4bS,6aS,9aS,9bS)-4a,6a-dimethyl-7-(pyrazin-2-yl)-4,4a,4b,5,6,6a,9,9a,9b,10-decahydro-1H-indeno[5,4-f]quinolin-2(3H)-one, replacing (4aR,4bS,6aS,9aS,9bR)-7-iodo-4a,6a-dimethyl-4,4a,4b,5,6,6a,9,9a,9b,10-decahydro-1H-indeno[5,4-f]quinolin-2(3H)-one with (3aS,3bS,9aR,9bS,11aS)-1-iodo-9a,11a-dimethyl-3b,4,5,8,9,9a,9b,10,11,11a-decahydro-3H-cyclopenta[i]phenanthridin-7(3aH)-one and using 2-(tributylstannyl)pyrazine, (3aS,3bS,9aR,9bS,11aS)-9a,11a-dimethyl-1-(pyrazin-2-yl)-3b,4,5,8,9,9a,9b,10,11,11a-decahydro-3H-cyclopenta[i]phenanthridin-7(3aH)-one is made.

### Example 135

### (3aS,3bS,9aR,9bS,11aS)-1-(6-Methoxypyrazin-2-yl)-9a,11a-dimethyl-3b,4,5,8,9,9a,9b,10,11,11a-decahydro-3H-cyclopenta[i]phenanthridin-7(3aH)-one

Using a synthetic procedure and condition similar to Example 130 in the preparation of (4aR,4bS,6aS,9aS,9bS)-4a,6a-dimethyl-7-(pyrazin-2-yl)-4,4a,4b,5,6,6a,9,9a, 9b,10-decahydro-1H-indeno[5,4-f]quinolin-2(3H)-one, replacing (4aR,4bS,6aS,9aS,9bR)-7-iodo-4a,6a-dimethyl-4,4a,4b,5,6,6a,9,9a,9b,10-decahydro-1H-indeno[5,4-f]quinolin-2(3H)-one with (3aS,3bS,9aR,9bS,11aS)-1-iodo-9a,11a-dimethyl-3b,4,5,8,9,9a,9b,10,11,11a-decahydro-3H-cyclopenta[i]phenanthridin-7(3aH)-one and using 2-methoxy-6-(tributylstannyl)pyrazine instead of 2-(tributylstannyl)pyrazine, (3aS,3bS,9aR,9bS,11aS)-1-(6-methoxypyrazin-2-yl)-9a,11a-dimethyl-3b,4,5,8,9,9a,9b,10,11,11a-decahydro-3H-cyclopenta[i]phenanthridin-7(3aH)-one is made.

### Example 136

### (4aR,4bS,6aS,9aS,9bS)-4a,6a-Dimethyl-7-(oxazol-5-yl)-4,4a,4b,5,6,6a,9,9a,9b,10-decahydro-1H-indeno[5,4-f]quinolin-2(3H)-one

Using a synthetic procedure and condition similar to Example 127 in the preparation of (4aR,4bS,6aS,9aS,9bS)-7-(4-methoxypyridin-3-yl)-4a,6a-dimethyl-4,4a,4b,5,6,6a,9,9a,9b,10-decahydro-1H-indeno[5,4-f]quinolin-2(3H)-one, replacing 4-methoxypyridin-3-ylboronic acid hydrate with oxazol-5-yllboronic acid, (4aR,4bS,6aS,9aS,9bS)-4a,6a-dimethyl-7-(oxazol-5-yl)-4,4a,4b,5,6,6a,9,9a,9b,10-decahydro-1H-indeno[5,4-f]quinolin-2(3H)-one is made.

### Example 137

### (4aR,4bS,6aS,9aS,9bS)-4a,6a-Dimethyl-7-(thiazol-5-yl)-4,4a,4b,5,6,6a,9,9a,9b,10-decahydro-1H-indeno[5,4-f]quinolin-2(3H)-one

Using a synthetic procedure and condition similar to Example 127 in the preparation of (4aR,4bS,6aS,9aS,9bS)-7-(4-methoxypyridin-3-yl)-4a,6a-dimethyl-4,4a,4b,5,6,6a,9,9a,9b,10-decahydro-1H-indeno[5,4-f]quinolin-2(3H)-one, replacing 4-methoxypyridin-3-ylboronic acid hydrate with thiazol-5-yllboronic acid, (4aR,4bS,6aS,9aS,9bS)-4a,6a-dimethyl-7-(thiazol-5-yl)-4,4a,4b,5,6,6a,9,9a,9b,10-decahydro-1H-indeno[5,4-f]quinolin-2(3H)-one is made.

### Example 138

### (4aR,4bS,6aS,9aS,9bS)-7-(Isoxazol-4-yl)-4a,6a-dimethyl-4,4a,4b,5,6,6a,9,9a,9b,10-decahydro-1H-indeno[5,4-f]quinolin-2(3H)-one

Using a synthetic procedure and condition similar to Example 127 in the preparation of (4aR,4bS,6aS,9aS,9bS)-7-(4-methoxypyridin-3-yl)-4a,6a-dimethyl-4,4a,4b,5,6,6a,9,9a,9b,10-decahydro-1H-indeno[5,4-f]quinolin-2(3H)-one, replacing 4-methoxypyridin-3-ylboronic acid hydrate with isoxazol-4-yllboronic acid, (4aR,4bS,6aS,9aS,9bS)-7-(isoxazol-4-yl)-4a,6a-dimethyl-4,4a,4b,5,6,6a,9,9a,9b,10-decahydro-1H-indeno[5,4-f]quinolin-2(3H)-one is made.

### Example 139

### (4aR,4bS,6aS,9aS,9bS)-1,4a,6a-Trimethyl-7-(oxazol-5-yl)-4,4a,4b,5,6,6a,9,9a,9b,10-decahydro-1H-indeno[5,4-f]quinolin-2(3H)-one

Using a synthetic procedure and condition similar to Example 8 in the preparation of (4aR,4bS,6aS,9aS,9bS)-7-(6-methoxypyridin-3-yl)-1,4a,6a-trimethyl-4,4a,4b,5,6,6a,9,9a,9b,10-decahydro-1H-indeno[5,4-f]quinolin-2(3H)-one replacing 6-methoxypyridin-3-ylboronic acid with oxazol-5-ylboronic acid, (4aR,4bS,6aS,9aS,9bS)-1,4a,6a-trimethyl-7-(oxazol-5-yl)-4,4a,4b,5,6,6a,9,9a,9b,10-decahydro-1H-indeno[5,4-f]quinolin-2(3H)-one is made.

### Example 140

### (4aR,4bS,6aS,9aS,9bS)-1,4a,6a-Trimethyl-7-(thiazol-5-yl)-4,4a,4b,5,6,6a,9,9a,9b,10-decahydro-1H-indeno[5,4-f]quinolin-2(3H)-one

Using a synthetic procedure and condition similar to Example 8 in the preparation of (4aR,4bS,6aS,9aS,9bS)-7-(6-methoxypyridin-3-yl)-1,4a,6a-trimethyl-4,4a,4b,5,6,6a,9,9a,9b,10-decahydro-1H-indeno[5,4-f]quinolin-2(3H)-one replacing 6-methoxypyridin-3-ylboronic acid with thiazol-5-ylboronic acid, (4aR,4bS,6aS,9aS,9bS)-1,4a,6a-trimethyl-7-(thiazol-5-yl)-4,4a,4b,5,6,6a,9,9a,9b,10-decahydro-1H-indeno[5,4-f]quinolin-2(3H)-one is made.

### Example 141

### (4aR,4bS,6aS,9aS,9bS)-7-(Isoxazol-4-yl)-1,4a,6a-trimethyl-4,4a,4b,5,6,6a,9,9a,9b,10-decahydro-1H-indeno[5,4-f]quinolin-2(3H)-one

Using a synthetic procedure and condition similar to Example 8 in the preparation of (4aR,4bS,6aS,9aS,9bS)-7-(6-methoxypyridin-3-yl)-1,4a,6a-trimethyl-4,4a,4b,5,6,6a,9,9a,9b,10-decahydro-1H-indeno[5,4-f]quinolin-2(3H)-one replacing 6-methoxypyridin-3-ylboronic acid with isoxazol-4-ylboronic acid, (4aR,4bS,6aS,9aS,9bS)-7-(isoxazol-4-yl)-1,4a,6a-trimethyl-4,4a,4b,5,6,6a,9,9a,9b,10-decahydro-1H-indeno[5,4-f]quinolin-2(3H)-one is made.

### Example 142

### (4aR,4bS,6aS,9aS,9bS)-4a,6a-Dimethyl-7-(oxazol-5-yl)-4,4a,4b,5,6,6a,9,9a,9b,10,11,11a-dodecahydroindeno[5,4-f]chromen-2(3H)-one

Using a synthetic procedure and condition similar to Example 61 in the preparation of (4aR,4bS,6aS,9aS,9bS)-4a,6a-dimethyl-7-(pyridin-3-yl)-4,4a,4b,5,6,6a,9,9a,9b,10,11,11a-dodecahydroindeno[5,4-f]chromen-2(3H)-one, replacing pyridin-3-ylboronic acid with oxazol-5-ylboronic acid in Example 61C followed by reacting the product with NaBH₄ in methanol in similar condition as in Example 61, (4aR,4bS,6aS,9aS,9bS)-4a,6a-dimethyl-7-(oxazol-5-yl)-4,4a,4b,5,6,6a,9,9a,9b,10,11,11a-dodecahydroindeno[5,4-f]chromen-2(3H)-one is made.

### Example 143

### (4aR,4bS,6aS,9aS,9bS)-4a,6a-Dimethyl-7-(thiazol-5-yl)-4,4a,4b,5,6,6a,9,9a,9b,10,11,11a-dodecahydroindeno[5,4-f]chromen-2(3H)-one

Using a synthetic procedure and condition similar to Example 61 in the preparation of (4aR,4bS,6aS,9aS,9bS)-4a,6a-dimethyl-7-(pyridin-3-yl)-4,4a,4b,5,6,6a,9,9a,9b,10,11,11a-dodecahydroindeno[5,4-f]chromen-2(3H)-one, replacing pyridin-3-ylboronic acid with thiazol-5-ylboronic acid in Example 61C followed by reacting the product with NaBH₄ in methanol in similar condition as in Example 61, (4aR,4bS,6aS,9aS,9bS)-4a,6a-dimethyl-7-(thiazol-5-yl)-4,4a,4b,5,6,6a,9,9a,9b,10,11,11a-dodecahydroindeno[5,4-f]chromen-2(3H)-one is made.

### Example 144

### (4aR,4bS,6aS,9aS,9bS)-7-(Isoxazol-4-yl)-4a,6a-dimethyl-4,4a,4b,5,6,6a,9,9a,9b,10,11,11a-dodecahydroindeno[5,4-f]chromen-2(3H)-one

Using a synthetic procedure and condition similar to Example 61 in the preparation of (4aR,4bS,6aS,9aS,9bS)-4a,6a-dimethyl-7-(pyridin-3-yl)-4,4a,4b,5,6,6a,9,9a,9b,10,11,11a-dodecahydroindeno[5,4-f]chromen-2(3H)-one, replacing pyridin-3-ylboronic acid with isoxazol-4-ylboronic acid in Example 61C followed by reacting the product with NaBH₄ in methanol in similar condition as in Example 61, (4aR,4bS,6aS,9aS,9bS)-7-(isoxazol-4-yl)-4a,6a-dimethyl-4,4a,4b,5,6,6a,9,9a,9b,10,11,11a-dodecahydroindeno[5,4-f]chromen-2(3H)-one is made.

### Example 145

### (4aR,4bS,6aS,9aS,9bS)-4a,6a-Dimethyl-7-(oxazol-5-yl)-4,4a,4b,5,6,6a,9,9a,9b,10-decahydroindeno[5,4-f]chromen-2(3H)-one

Using a synthetic procedure and condition similar to Example 62 in the preparation of (4aR,4bS,6aS,9aS,9bS)-4a,6a-dimethyl-7-(pyridin-3-yl)-4,4a,4b,5,6,6a,9,9a,9b,10-decahydroindeno[5,4-f]chromen-2(3H)-one, replacing pyridin-3-ylboronic acid with oxazol-5-ylboronic acid in Example 61C followed by reacting the product with NaOAc in acetic anhydride, in similar experimental condition as in Example 62, (4aR,4bS,6aS,9aS,9bS)-4a,6a-dimethyl-7-(oxazol-5-yl)-4,4a,4b,5,6,6a,9,9a,9b,10-decahydroindeno[5,4-f]chromen-2(3H)-one is made.

### Example 146

### (4aR,4bS,6aS,9aS,9bS)-4a,6a-Dimethyl-7-(thiazol-5-yl)-4,4a,4b,5,6,6a,9,9a,9b,10-decahydroindeno[5,4-f]chromen-2(3H)-one

Using a synthetic procedure and condition similar to Example 62 in the preparation of (4aR,4bS,6aS,9aS,9bS)-4a,6a-dimethyl-7-(pyridin-3-yl)-4,4a,4b,5,6,6a,9,9a,9b,10-decahydroindeno[5,4-f]chromen-2(3H)-one, replacing pyridin-3-ylboronic acid with thiazol-5-ylboronic acid in Example 61C followed by reacting the product with NaOAc in acetic anhydride, in similar experimental condition as in Example 62, (4aR,4bS,6aS,9aS,9bS)-4a,6a-dimethyl-7-(thiazol-5-yl)-4,4a,4b,5,6,6a,9,9a,9b,10-decahydroindeno[5,4-f]chromen-2(3H)-one is made.

### Example 147

### (4aR,4bS,6aS,9aS,9bS)-7-(isoxazol-4-yl)-4a,6a-dimethyl-4,4a,4b,5,6,6a,9,9a,9b,10-decahydroindeno[5,4-f]chromen-2(3H)-one

Using a synthetic procedure and condition similar to Example 62 in the preparation of (4aR,4bS,6aS,9aS,9bS)-4a,6a-dimethyl-7-(pyridin-3-yl)-4,4a,4b,5,6,6a,9,9a,9b,10-decahydroindeno[5,4-f]chromen-2(3H)-one, replacing pyridin-3-ylboronic acid with isoxazol-4-ylboronic acid in Example 61C followed by reacting the product with NaOAc in acetic anhydride, in similar experimental condition as in Example 62, (4aR,4bS,6aS,9aS,9bS)-7-(isoxazol-4-yl)-4a,6a-dimethyl-4,4a,4b,5,6,6a,9,9a,9b,10-decahydroindeno[5,4-f]chromen-2(3H)-one is made.

### Example 148

### (3aS,3bS,9aR,9bS,11aS)-9a,11a-dimethyl-1-(oxazol-5-yl)-3b,4,5,8,9,9a,9b,10,11,11a-decahydro-3H-cyclopenta[i]phenanthridin-7(3aH)-one

Using a synthetic procedure and condition similar to Example 5 in the preparation of (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl oxazol-5-ylboronate as in Example 5C and following the rest of reaction in Example 5, (3aS,3bS,9aR,9bS,11aS)-9a,11a-dimethyl-1-(oxazol-5-yl)-3b,4,5,8,9,9a,9b,10,11,11a-decahydro-3H-cyclopenta[i]phenanthridin-7(3aH)-one is made.

### Example 149

### (3aS,3bS,9aR,9bS,11aS)-9a,11a-dimethyl-1-(thiazol-5-yl)-3b,4,5,8,9,9a,9b,10,11,11a-decahydro-3H-cyclopenta[i]phenanthridin-7(3aH)-one

Using a synthetic procedure and condition similar to Example 5 in the preparation of (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl thiazol-5-ylboronate as in Example 5C and following the rest of reaction in Example 5, (3aS,3bS,9aR,9bS,11aS)-9a,11a-dimethyl-1-(thiazol-5-yl)-3b,4,5,8,9,9a,9b,10,11,11a-decahydro-3H-cyclopenta[i]phenanthridin-7(3aH)-one is made.

### Example 150

### N-[5-(1,4a,6a-Trimethyl-2-oxo-2,3,4,4a,4b,5,6,6a,9,9a,9b,10-dodecahydro-H-indeno[5,4-f]quinolin-7-yl)-pyridin-2-yl]-acetamide

Using a synthetic procedure and condition similar to Example 5 in the preparation of (3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl isoxazol-4-ylboronate as in Example 5C and following the rest of reaction in Example 5, (3aS,3bS,9aR,9bS,11aS)-1-(isoxazol-4-yl)-9a,11a-dimethyl-3b,4,5,8,9,9a,9b,10,11,11a-decahydro-3H-cyclopenta[i]phenanthridin-7(3aH)-one is made.

### Example 151

### (3aS,3bS,9aR,9bS,11aS)-5,9a,11a-trimethyl-1-(oxazol-5-yl)-3b,4,5,8,9,9a,9b,10,11,11a-decahydro-3H-cyclopenta[i]phenanthridin-7(3aH)-one

Using a synthetic procedure and condition similar to Example 4 in the preparation of (3aS,3bS,9aR,9bS,11aS)-5,9a,11a-trimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl oxazol-5-ylboronate, (3aS,3bS,9aR,9bS,11aS)-5,9a,11a-trimethyl-1-(oxazol-5-yl)-3b,4,5,8,9,9a,9b,10,11,11a-decahydro-3H-cyclopenta[i]phenanthridin-7(3aH)-one is made.

### Example 152

### (3aS,3bS,9aR,9bS,11aS)-5,9a,11a-trimethyl-1-(thiazol-5-yl)-3b,4,5,8,9,9a,9b,10,11,11a-decahydro-3H-cyclopenta[i]phenanthridin-7(3aH)-one

Using a synthetic procedure and condition similar to Example 4 in the preparation of (3aS,3bS,9aR,9bS,11aS)-5,9a,11a-trimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl thiazol-5-ylboronate, (3aS,3bS,9aR,9bS,11aS)-5,9a,11a-trimethyl-1-(thiazol-5-yl)-3b,4,5,8,9,9a,9b,10,11,11a-decahydro-3H-cyclopenta[i]phenanthridin-7(3aH)-one is made.

### Example 153

### (3aS,3bS,9aR,9bS,11aS)-1-(isoxazol-4-yl)-5,9a,11a-trimethyl-3b,4,5,8,9,9a,9b,10,11,11a-decahydro-3H-cyclopenta[i]phenanthridin-7(3aH)-one

Using a synthetic procedure and condition similar to Example 4 in the preparation of (3aS,3bS,9aR,9bS,11aS)-5,9a,11a-trimethyl-1-(pyridin-3-yl)-3b,4,5,8,9,9a,9b,10,11,11a-decahydro-3H-cyclopenta[i]phenanthridin-7(3aH)-one, replacing diethyl 3-pyridoborane with diethyl isoxazol-4-ylboronate, (3aS,3bS,9aR,9bS,11aS)-1-(isoxazol-4-yl)-5,9a,11a-trimethyl-3b,4,5,8,9,9a,9b,10,11,11a-decahydro-3H-cyclopenta[i]phenanthridin-7(3aH)-one is made.

### Example 154

### (4aR,4bS,6aS,9aS,9bS)-4a,6a-dimethyl-7-(1H-1,2,3-triazol-4-yl)-4,4a,4b,5,6,6a,9,9a,9b,10-decahydro-1H-indeno[5,4-f]quinolin-2(3H)-one

### Example 154 A

### (4aR,4bS,6aS,9aS,9bS)-4a,6a-dimethyl-7-((trimethylsilyl)ethynyl)-4,4a,4b,5,6,6a,9,9a,9b,10-decahydro-1H-indeno[5,4-f]quinolin-2(3H)-one

A solution of (4aR,4bS,6aS,9aS,9bR)-7-iodo-4a,6a-dimethyl-4,4a,4b,5,6,6a,9,9a,9b,10-decahydro-1H-indeno[5,4-f]quinolin-2(3H)-one (750 mg, 1.89 mmol), ethynyltrimethylsilane (555 mg, 5.67 mmol), triethylamine (30 mL) was purged with nitrogen for 5 min. Pd(PPh₃)₂Cl₂ (70 mg, 0.1 mmol) and CuI (19 mg, 0.1 mmol) were added to the solution and stirred at room temperature for 16 hr under nitrogen. The solution was evaporated to dryness under reduced pressure on a rotary evaporator. The residues were dissolved in DCM (20 mL) and washed with water (3×10 mL). After dried over Na₂ SO₄, the organic phase was concentrated and loaded to a silica gel column. Elution with ethyl acetate in hexane (5-15%) gave (4aR,4bS,6aS,9aS,9bS)-4a,6a-dimethyl-7-((trimethylsilyl)ethynyl)-4,4a,4b,5,6,6a,9,9a,9b,10-decahydro-1H-indeno[5,4-f]quinolin-2(3H)-one as a white solid (455 mg, 66% yield). MS (ESI+) m/e 368.

### Example 154B

### 4aR,4bS,6aS,9aS,9bS)-7-ethynyl-4a,6a-dimethyl-4,4a,4b,5,6,6a,9,9a,9b,10-decahydro-1H-indeno[5,4-f]quinolin-2(3H)-one

To a solution of (4aR,4bS,6aS,9aS,9bS)-4a,6a-dimethyl-7-((trimethylsilyl)ethynyl)-4,4a,4b,5,6,6a,9,9a,9b,10-decahydro-1H-indeno[5,4-f]quinolin-2(3H)-one (455 mg, 1.24 mmol) in MeOH (30 mL) was added K₂ CO₃ (200 mg). It was allowed to stir at room temperature for 3 hr. It was filtered and evaporated to dryness using a rotary evaporator. The residue was then taken up with ethyl acetate (30 mL) and washed with water (3×10 mL). After dried over anhydrous Na₂ SO₄ and solvent removal, it gave (4aR,4bS,6aS,9aS,9bS)-7-ethynyl-4a,6a-dimethyl-4,4a,4b,5,6,6a,9,9a,9b,10-decahydro-1H-indeno[5,4-f]quinolin-2(3H)-one as a white solid (366 mg, 90%).

### Example 154

### (4aR,4bS,6aS,9aS,9bS)-4a,6a-dimethyl-7-(1H-1,2,3-triazol-4-yl)-4,4a,4b,5,6,6a,9,9a,9b,10-decahydro-1H-indeno[5,4-f]quinolin-2(3H)-one

Compound (4aR,4bS,6aS,9aS,9bS)-7-ethynyl-4a,6a-dimethyl-4,4a,4b,5,6,6a,9,9a,9b,10-decahydro-1H-indeno[5,4-f]quinolin-2(3H)-one (300 mg, 1.02 mmol) and TMS-N₃ (350 mg, 3.06 mmol) were dissolved in ^{t}BuOH/H₂O mixture (95:5). To this solution was added CuSO₄ (16 mg, 0.1 mmol) and Na-ascorbate (60 mg, 0.3 mmol). The mixture was stirred at 35 C for 36 hr. Ethyl acetate and water were added to the mixture. The organic phase was separated, washed with brine (3×15 mL), and then dried over sodium sulfate. After removal of solvent by rotary evaporation, the residue was purified with silica gel chromatography using hexane/ethyl acetate as the eluents (90/10). This gave an off-white solid of (4aR,4bS,6aS,9aS,9bS)-4a,6a-dimethyl-7-(1H-1,2,3-triazol-4-yl)-4,4a,4b,5,6,6a,9,9a,9b,10-decahydro-1H-indeno[5,4-f]quinolin-2(3H)-one (50 mg, 15%). MS (ESI+) m/e 339.

### Biological Studies

Human and Murine C_{17,20}-lyase Biochemical Assays:

Recombinant human C_{17,20}-lyase (hLyase) is expressed in baculovirus-infected Sf9 cells and hLyase enriched microsomes are prepared from cultures as described (Barnes H. J.; Jenlins, C. M.; Waterman, M. R. Archives of Biochemistry and Biophysics 1994, 315(2), 489-494). Recombinant murine C_{17,20}-lyase (mLyase) is prepared in a similar manner. hlyase and mLyase preparations are titrated using assay conditions to determine protein concentrations to be used for assays. Both mLyase and hLyase assays are run in an identical manner except that cytochrome b5 is omitted in the murine assay.

Test compound solutions (20 mM in DMSO) are diluted 1:4 with DMSO and put into the top well of a 96-well mother plate. These solutions are then diluted serially in six steps (1:4 each step) with DMSO to obtain 800 M to 51.2 nM concentrations on a mother plate (columns 3-12) for subsequent use in the assay. These compound solutions are further diluted twenty-fold in water to obtain a daughter plate containing compound concentrations ranging from 40 μM to 2.56 nM in 5% DMSO. The first 2 columns (of wells) on each 96-well mother plate are used for the DHEA (dehydroepiandrosterone) standard curve. DHEA standards are serially diluted (in half-logs) in DMSO to obtain 400 μM to 120 nM standards, then diluted (1:19) in water to obtain 20 μM to 6 nM solutions in 5% DMSO on the daughter plate. These 5% DMSO solutions (5 μL each) from the daughter plate are transferred to the SPA assay plate prior to adding the reaction mixture.

To prepare the reaction mixture, clear-bottomed opaque 96-well assay plates are loaded with 50 μL of assay buffer (50 mM Na₃ PO₄, pH 7.5), 5 mL of the diluted compounds (or standards), and 30 mL of substrate solutions (7 mM NADPH, 3.35 μM 17-OH-pregnenolone, 3.35 μg/mL human cytochrome b5 in 50 mM Na₃ PO₄). Reactions are initiated with the addition of hLyase or mLyase in assay buffer (10 μL). Enzymatic reactions are incubated at room temperature for 2 hours with gentle agitation. Reactions are terminated with the addition of 5 μL of 1 mM (50 μM final concentration) YM116, a potent C_{17,20}-lyase inhibitor.

The concentration of DHEA generated by hLyase (or mLyase) is determined by radioimmunoassay (RIA). RIA will utilize a ³H-DHEA (0.08 μCi) tracer in 50 μL of scintillation proximity assay (SPA) buffer (100 mM Tris-HCl, pH 7.5, 50 mM NaCl, 0.5% BSA, 0.2% Tween 20) which is added to each well. DHEA antiserum from rabbit (50 μL) with anti-rabbit SPA beads in SPA buffer is added to all wells. Mixtures are allowed to equilibrate with gentle agitation for 1 hour followed by overnight equilibration with no agitation. H-DHEA bound to the SPA beads is determined by scintillation counting with a Wallac microbeta counter. The concentration of DHEA generated is calculated from raw data (CPM) and the standard curve. The concentration of DHEA formed in the presence of test compounds is then expressed as a percent inhibition compared to the DHEA concentration in the absence of test compounds: [1−(nM DHEA formed in the presence of test compound/nM DHEA formed in the absence of test compounds)]×100. Determination of IC₅₀ for each compound will be performed using the Analyze 5 program.

Human C_{17,20}-lyase Cellular Assay:

Human HEK 293-lyase stable transfectant cells are seeded in a 96-well plate at 10,000 cells/well/100 μL in DMEM plus 10% FBS (supplemented with 1% glutamine, 0.8 mg/mL G418) and allowed to attach overnight. The next day, the media is removed from the cell plate and replaced with 100 μL RPMI without phenol red. Test compounds, DMSO vehicle, or DHEA standards of 5 mL each are added to the cell plate and incubated for 10 min. at room temperature. The reaction is initiated with 10 μL of 5 μM 17-OH-pregnenolone added to all the wells of the cell plate, then incubated for 1 hour at 37° C. Following the incubation, 90 μL of media (containing DHEA product) is removed from the cell plate and transferred to the SPA assay plate. The subsequent SPA procedure for the detection of DHEA product is performed in the same manner as described for the enzyme assay (see above). The mother plate of test compounds is also prepared in the same manner as the enzyme assay.

Reagents (including catalog #) for the SPA assay can be obtained from the following sources: ³H-DHEA: NEN (NET814), Anti-DHEA: Endocrine Sciences (D7-421), Anti-Rabbit SPA Beads: Amersham (RPNQ 0016), 17-OH-pregnenolone: Steraloids (Q4710), NADPH: Sigma (N1630), Cytochrome b5: Panvera (P2252), DHEA (500 μM stock in 100% EtOH), BSA: Sigma (A9647).

Evaluation of a Compound Having the Structure of Formula (I), (II) or (III) as Inhibitors of Testicular Human and Rat 17α-hydroxylase/C_{17,20}-lyase (17α-lyase) In Vitro

The potency as inhibitors of P450₁₇α of the compounds described herein are evaluated in human and rat testicular microsomes.

Human testicular microsomes are prepared from human testes (obtained from untreated prostatic cancer patients undergoing orchidectomy), as described in Li et al., The Prostate, 26:140-150 (1995).

Rat testicular microsomes are prepared from the testes of adult Sprague-Dawley rats, as described by Li et al., J. Med. Chem., 39:4335-4339 (1996).

The microsomes are stored at −70° C. until assayed. Just before use, the thawed microsomes are diluted with 0.1 M phosphate buffer (pH 7.4) to appropriate concentrations.

The protein concentration of the microsomes used in the assay are determined by the method of Lowry et al., J. Biol. Chem., 193:265-275 (1951).

The enzyme reaction (activity) is monitored by determination of the release of C³H₃COOH from [21-³H³]-17α-hydroxypregnenolone during cleavage of the C-21 side-chain in the conversion to dehydroepiandrosterone (DHEA) as described by Njar et al., Steroids, 62:468-473 (1997). This assay measures only the lyase activity of the P450₁₇α enzyme. This assay is comparable to the HPLC assay procedure (which utilizes [7-³H]-pregnenolone as substrate), and measures both the hydroxylase and lyase activities of the enzyme.

IC₅₀ values for inhibitors are calculated from the linear regression line in the plot of logit of lyase activity versus log of inhibitor concentration. Kᵢ values are also determined from assays as described by Njar et al., (1997), supra. Each inhibitor is examined at three concentrations. Data from the various assays are used to obtain Lineweaver-Burk plots and from replots of slopes versus inhibitor concentration, Kᵢ values are obtained and the Kₘ for 17α-hydroxypregnenolone (substrate) is also determined.

Human C_{17,20}-lyase enzymatic assays were conducted in 200 μL volume in Eppendorf tubes, using microsomal fraction from human testis (Celsis Cat #500110) as the enzyme source. Total protein concentration of the microsomal fraction is estimated to be 20 mg/ml. Prior to adding the microsomal fractions, reaction mixtures containing 50 mM NaPO₄ buffer (pH 7.4), 1 mM MgCl₂, 0.1 mM EDTA, 0.1 mM dithiothreitol, 0.5 mM NADPH, 4 μM 17α-hydroxypregnenolone, 1 μL of [21-³H]-17α-hydroxypregnenolone (American Radiolabeled Chemicals, ART #1663, Specific activity=50-60 Ci/mmol), and the appropriate testing compounds were incubated for 5 minutes in a 37° C. shaking water bath (150 rpm). Following the 5-minute pre-incubation, 5 μL of human testis microsome was added to each of the reaction mixtures (except for the Negative Controls, which received 5 μL of H₂O). After 30-minute incubation at 37° C. in shaking water bath (150 rpm), reactions were stopped by addition of 200 μL of cold chloroform and vigorous shaking for 30 minutes. Tubes were centrifuged at 1,500×g for 15 minutes at 4° C., and the aqueous phase was transferred to fresh Eppendorf tubes. Forty microliters (40 μL) of 8.5% charcoal (Sigma Cat #C6241) suspension was added to each tube, mixed well and incubated at 4° C. for 30 minutes. Tubes were centrifuged at 1,500×g for 15 minutes at 4° C., and 100 μL upper layer from each tube was transferred into each well of a 96-well isoplate (PerkinElmer Cat #6005040). Finally, 100 μL of Optiphase supermix scintillation fluid (PerkinElmer Cat #1200430) was added to each well, mixed by pippeting up and down 3 times. Radioactivity was measured with MicroBeta Trilux Counter using tritium program. All testing compounds were dissolved and diluted in methanol. Two microliters (2 μL) of the properly diluted test compound was added to each reaction to reach the desired concentration. In Negative control (no enzyme activity) and Activity control (100% enzyme activity), 2 μL of methanol was added. Each data point was tested in duplicate. Inhibition of human C_{17,20}-lyase activity was calculated either by inhibition rate at 100 nM concentration and the inhibition rate was calculated as following:

$$Inhibition Rate ( \% ) = 100 ⋆ ( 1 - \frac{\begin{matrix}Test compound cpm - \\ Negative control cpm\end{matrix}}{\begin{matrix}Activity control cpm - \\ Negative control cpm\end{matrix}} )$$

or by IC₅₀ value which was generated using Prism software under “non-linear regression analysis”. The inhibition rate expressed in percent for representative compounds of embodiments shown herein are provided as follows: 77.1, 73.8, 74.0, 68.7, 68.6, 72.6, 48.8, 81.3, 81.8, 79.4, 51.8, 78.7 and 82.8.

### In Vivo Antitumor Studies (LAPC-4 Prostate Cancer Xenografts)

All animal studies will be performed according to the guidelines and approval of the Animal Care Committee of the testing facility.

Male sever combined immunodeficient (SCID) mice 4-6 weeks of age are purchased, for example, from the National Cancer Institute-Frederick Cancer Research and Development Center and housed in a pathogen-free environment under controlled conditions of light and humidity and allowed free access to food and water. Tumors are developed from LAPC-4 cells inoculated subcutaneously (s.c.) in the mice. LAPC-4 cells are grown in IMEM with 15% FBS plus 1% PS and nm DHT until 80% confluent. Cells are scraped into DPBS, collected by centrifugation, and resuspended in Matrigel (10 mg/ml) at 3×10⁷ cells/ml. Mice are injected s.c. with 100 μl of the cell suspension at one site on each flank. Tumors will be measured weekly with calipers, and tumor volumes will be calculated by the formula: 4/3Π×r₁²×r₂ (r₁<r₂).

LAPC-4 tumors will be allowed to grow for 8-10 weeks following inoculation. Groups of 5 mice with comparable total tumor volumes will be either castrated under methoxyfluorane anesthesia or treated with a compound having the structure of Formula (I), (II) or (III) (about 0.15 mmol/kg once-daily and 0.15 mmol/kg twice-daily). A compound having the structure of Formula (I), (II) or (III) will be prepared at about 17 mg/ml in about a 0.3% solution of hydroxypropyl cellulose in saline, and mice will receive s.c. injections daily. Control and castrated mice will be treated with vehicle only. Tumors will be measured weekly for the 4 weeks of treatment and tumor volumes will be calculated. At the end of the treatment period, the mice will be sacrificed under halothane anesthesia; the tumors will be excised, weighed and stored at −80° C. The mice will also be weighed weekly and monitored for general health status and signs of possible toxicity due to treatment.

### Human Clinical Trial of the Safety and Efficacy of Compounds of Formulas (I)-(III)

Objective: To evaluate the safety, pharmacokinetics, pharmacodynamics, and anti-tumor activities of an oral CYP17 inhibitor, a compound having the structure of Formula (I), (II) or (III), administered to patients with hormone refractory prostate cancer (HRPC).

Patients: Eligible subjects will be men 18 years and older.

Inclusion criteria for Phase I will include:
Histologically confirmed adenocarcinoma of the prostate;
No prior therapy with chemotherapy for prostate cancer;
Ongoing gonadal androgen deprivation therapy with LHRH analogues or orchiectomy. Patients, who have not had an orchiectomy will be maintained on effective LHRH analogue therapy for the duration of the trial;
Testosterone <50 ng/dL;
Progressive disease after androgen deprivation: PSA evidence for progressive prostate cancer consists of a PSA level of at least 5 ng/ml which has risen on at least 2 successive occasions, at least 2 weeks apart. If the confirmatory PSA value is less than the screening PSA value, then an additional test for rising PSA will be required to document progression;
Antiandrogen Withdrawal Patients who are receiving an antiandrogen as part of primary androgen ablation must demonstrate disease progression following discontinuation of antiandrogen. Disease progression after antiandrogen withdrawal is defined as 2 consecutive rising PSA values, obtained at least 2 weeks apart, or documented osseous or soft tissue progression.
   For patients receiving flutamide, at least one of the PSA values must be obtained 4 weeks or more after flutamide discontinuation;
   For patients receiving bicalutamide or nilutamide, at least one of the PSA values must be obtained 6 weeks or more after antiandrogen discontinuation;
ECOG Performance Status 0-1;
Serum Creatinine ≦1.5×ULN;
K+ more than or equal to 3.5 mmol/L;
Bilirubin ≦1.5×ULN;
AST and ALT ≦2.5×ULN;
Systolic blood pressure <160 mmHg and diastolic blood pressure <110 mmHg documented on at least 3 different days;
Baseline ACTH stimulation test demonstrating a peak cortisol >18 μg/dL; and
Life expectancy of more than or equal to 12 weeks.

Exclusion criteria for Phase I will include:
Therapy with other hormonal therapy, including any dose of megestrol acetate (Megace), finasteride (Proscar), dutasteride (Avodart) any herbal product known to decrease PSA levels (e.g., Saw Palmetto and PC-SPES), or any systemic corticosteroid within 4 weeks prior to first dose of study drug;
Initiation of bisphosphonate therapy within 4 weeks prior to first dose of study drug. Patients on stable doses of bisphosphonates that show subsequent tumor progression may continue on the medication; however, patients will not be allowed to initiate bisphosphonate therapy during the study;
Therapy with supplements or complementary medicines/botanicals within 4 weeks of first dose of study drug, except for any combination of the following:
   conventional multivitamin supplements;
   selenium;
   lycopene;
   soy supplements;
Prior radiation therapy completed <4 weeks prior to enrollment;
Prior chemotherapy for hormone refractory prostate cancer;
Hemoglobin less than or equal to 9.0 g/dL;
ANC less than or equal to 1.5×109/L;
Platelets less than or equal to 100×109/L;
Any “currently active” second malignancy, other than non-melanoma skin cancer. Patients will not considered to have a “currently active” malignancy, if they have completed therapy and are considered by their physician to be at least less than 30% risk of relapse over next 3 months;
Systolic blood pressure more than or equal to 160 mmHg or diastolic blood pressure more than or equal to 110 mmHg measured on at least 2 occasions;
Serum K+<3.5 mmoL/L;
NYHA Class III or IV Congestive Heart Failure;
Myocardial infarction within the 6 months prior to the first dose of study drug;
Serious intercurrent infections or nonmalignant medical illnesses that are uncontrolled;
Active psychiatric illnesses/social situations that would limit compliance with protocol requirements; and
Active or uncontrolled autoimmune disease that may require corticosteroid therapy during study.

Inclusion criteria for Phase II will include the same criteria for Phase I with the following additions:
Neoadjuvant or adjuvant chemotherapy is only allowed if the last dose is >1 year from Cycle 1 Day 1;
Target or Non-Target abnormalities must be present either on screening bone scan, CT or MRI; and
No prior treatment with ketoconazole for the management of androgen independent prostate cancer.

Exclusion criteria for Phase II will include the same criteria as Phase I with the following addition:
Abnormal electrocardiogram, including any finding which would interfere with assessment of intervals (patients with long QT syndrome, bundle branch blocks or hemiblocks will be prohibited).

Study Design: This will be a Phase I/II, non-randomized, open label dose escalation, single group assignment clinical trial of an oral compound of Formulas (I)-(III).

Primary Outcome Measures: Phase I: To determine maximum tolerated dose of a compound having the structure of Formula (I), (II) or (III) administered orally on a continuous once-daily schedule in patients with HRPC. Phase II: To assess proportion of patients achieving a >50% PSA decline during therapy with concurrent prednisone.

Secondary Outcome Measures: Phase I: 1. Safety/tolerability; 2. Pharmacokinetics; 3. Pharmacodynamics; 4. Need for steroids; 5. Preliminary anti-tumor activities. Phase II: 1. To assess safety and tolerability of a compound having the structure of Formula (I), (II) or (III) with concurrent prednisone; 2. Additional parameters for anti-tumor activity and clinical benefits.

Arms: Experimental—Phase I: A compound having the structure of Formula (I), (II) or (III); Phase II: A compound having the structure of Formula (I), (II), or (III) and prednisone.

Assigned Interventions: Drug: A compound having the structure of Formula (I), (II) or (III)—Phase I: Dose escalating; Phase II: 1000 mg of a compound having the structure of Formula (I), (II) or (III) PO daily and 5 mg of prednisone PO bid.

The examples and embodiments described herein are for illustrative purposes only and various modifications or changes suggested to persons skilled in the art are to be included within the spirit and purview of this application and scope of the appended claims.

What is claimed is:

1. A compound having the structure of Formula (I): 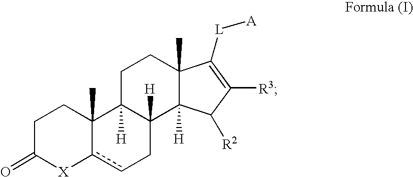
wherein:
X is O or NR¹;
L is a direct bond or 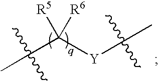
Y is a direct bond, O, C═O, C(O)O, S(O)ᵤ, NR¹ or NR⁷C(O);
q is an integer from 0 to 4;
u is an integer from 0 to 2;
A is a heteroaryl optionally substituted with 1, 2, 3, or 4 R⁸; [symbol]
is a single or double bond;
R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, and haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;
R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or
R_{A} and R_{B} taken together with the nitrogen atom to which they are attached form an optionally substituted 4 to 7 membered heterocyclic ring having one or two heteroatoms;
R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy;
R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl; and
R⁵ and R⁶ are each independently hydrogen, halogen, nitro, cyano, hydroxyl, optionally substituted alkyl, optionally substituted cycloalkyl, perfluoroalkyl, optionally substituted heteroalkyl, optionally substituted heterocycloalkyl, optionally substituted aryl, optionally substituted heteroaryl;
R⁷ is hydrogen or an optionally substituted alkyl;
R⁸ is each independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, or NR_{A}R_{B};
or a pharmaceutically acceptable salt or solvate thereof.

2. A compound having the structure of Formula (II): 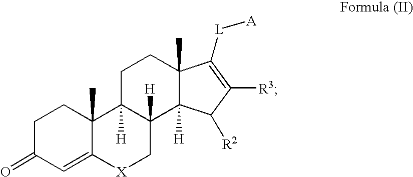
wherein:
X is O or NR¹;
L is a direct bond or 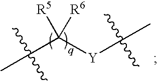
Y is a direct bond, O, C═O, C(O)O, S(O)ᵤ, NR¹, NR⁷C(O);
q is an integer from 0 to 4;
u is an integer from 0 to 2;
A is a heteroaryl optionally substituted with 1, 2, 3, or 4 R⁸;
R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;
R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or
R_{A} and R_{B} taken together with the nitrogen atom to which they are attached form an optionally substituted 4 to 7 membered heterocyclic ring having one or two heteroatoms;
R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy;
R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl; and
R⁵ and R⁶ are each independently hydrogen, halogen, nitro, cyano, hydroxyl, optionally substituted alkyl, optionally substituted cycloalkyl, perfluoroalkyl, optionally substituted heteroalkyl, optionally substituted heterocycloalkyl, optionally substituted aryl, optionally substituted heteroaryl;
R⁷ is hydrogen or an optionally substituted alkyl;
R⁸ is each independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, or NR_{A}R_{B};
or a pharmaceutically acceptable salt or solvate thereof.

3. A compound having the structure of Formula (III): 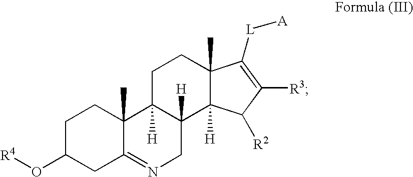
wherein:
L is a direct bond or 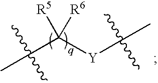
Y is a direct bond, O, C═O, C(O)O, S(O)ᵤ, NR¹, NR⁷C(O);
q is an integer from 0 to 4;
u is an integer from 0 to 2;
A is a heteroaryl optionally substituted with 1, 2, 3, or 4 R⁸;
R¹ is selected from the group consisting of hydrogen, alkyl, cycloalkyl, alkenyl, alkynyl, alkoxyalkyl, haloalkoxyalkyl; wherein the alkyl, cycloalkyl, alkenyl, alkynyl, alkoxyalkyl, haloalkoxyalkyl groups are optionally substituted with 1, 2, or 3 substituents independently selected from the group consisting of halogen, alkenyl, alkoxy, alkoxycarbonyl, hydroxyl, hydroxyalkyl, alkynyl, cyano, haloalkoxy, haloalkyl, nitro, NR_{A}R_{B}, (NR_{A}R_{B})carbonyl;
R_{A} and R_{B} are independently selected from the group consisting of hydrogen, optionally substituted alkyl, halosubstituted alkyl, optionally substituted alkoxyalkyl, optionally substituted cycloalkyl, optionally substituted aryl, optionally substituted arylalkyl, optionally substituted heteroaryl, or optionally substituted heteroarylalkyl; or
R_{A} and R_{B} taken together with the nitrogen atom to which they are attached form an optionally substituted 4 to 7 membered heterocyclic ring having one or two heteroatoms;
R² is selected from the group consisting of hydrogen, halogen, optionally substituted alkyl, optionally substituted cycloalkyl, cyano, nitro, optionally substituted alkoxy, optionally substituted alkoxyalkyl, optionally substituted haloalkoxy, optionally substituted haloalkoxyalkyl, hydroxyl, optionally substituted hydroxyalkyl and optionally substituted alkylcarbonyloxy;
R³ is selected from the group consisting of hydrogen, halogen, optionally substituted alkyl, optionally substituted cycloalkyl, optionally substituted alkynyl, cyano, optionally substituted haloalkoxy, optionally substituted haloalkyl, hydroxyl, optionally substituted hydroxyalkyl, nitro, R_{A}carbonyl, NR_{A}R_{B}, and (NR_{A}R_{B})carbonyl;
R⁴ is selected from the group consisting of hydrogen, optionally substituted alkyl, optionally substituted cycloalkyl, optionally substituted haloalkyl, optionally substituted hydroxyalkyl, R_{A}carbonyl, (NR_{A}R_{B})alkyl, and (NR_{A}R_{B})carbonyl; and
R⁵ and R⁶ are each independently hydrogen, halogen, nitro, cyano, hydroxyl, optionally substituted alkyl, optionally substituted cycloalkyl, perfluoroalkyl, optionally substituted heteroalkyl, optionally substituted heterocycloalkyl, optionally substituted aryl, optionally substituted heteroaryl;
R⁷ is hydrogen or an optionally substituted alkyl;
R⁸ is each independently selected from the group consisting of halogen, cyano, hydroxyl, optionally substituted alkoxy, optionally substituted alkyl, optionally substituted cycloalkyl, optionally substituted heterocycloalkyl, optionally substituted aryl, optionally substituted heteroaryl, COR_{A}, NR_{A}R_{B}carbonyl, or NR_{A}R_{B};
or a pharmaceutically acceptable salt or solvate thereof.

4. The compound of claim 1 having the structure of Formula (IA): 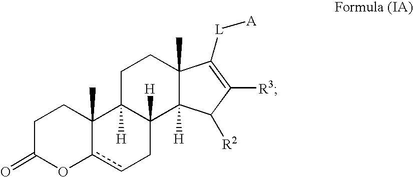
or a pharmaceutically acceptable salt or solvate thereof.

5. The compound of claim 1 having the structure of Formula (IB): 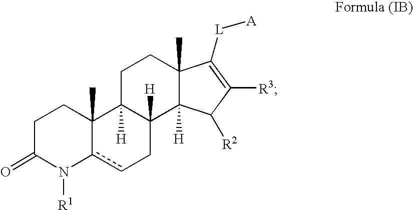
or a pharmaceutically acceptable salt or solvate thereof.

6. The compound of claim 2 having the structure of Formula (IIA): 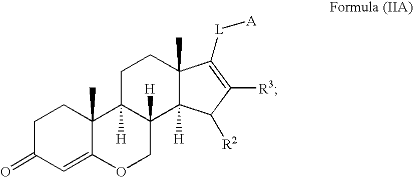
or a pharmaceutically acceptable salt or solvate thereof.

7. The compound of claim 2 having the structure of Formula (IIB): 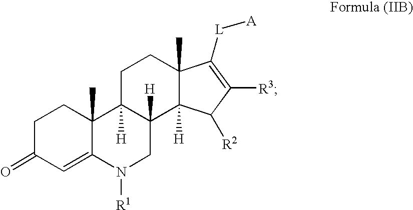
or a pharmaceutically acceptable salt or solvate thereof.

8. The compound of claim 1 wherein A is a heteroaryl selected from the group consisting of pyridine, pyrimidine, pyrazine, pyrazole, oxazole, thiazole, isoxazole, isothiazole, 1,3,4-oxadiazole, pyridazine, 1,3,5-trazine, 1,2,4-triazine, quinoxaline, benzimidazole, benzotriazole, purine, 1H-[1,2,3]triazolo[4,5-d]pyrimidine, triazole, imidazole, thiophene, furan, isobenzofuran, pyrrole, indolizine, isoindole, indole, indazole, isoquinoline, quinoline, phthalazine, naphthyridine, quinazoline, cinnoline, and pteridine.

9. The compound of claim 8 wherein the heteroaryl is selected from pyridine, imidazole, benzimidazole, pyrrole, pyrazole, pyrimidine, pyrazine, pyridazine, oxazole and thiazole.

10. The compound of claim 1 wherein R¹ is hydrogen or C₁-C₆ alkyl; R² is hydrogen or C₁-C₆ alkyl; and R³ is hydrogen or C₁-C₆ alkyl.

11. The compound of claim 3 wherein R⁴ is hydrogen or R_{A}carbonyl.

12. The compound of any of claims 1-3 wherein L is a direct bond.

13. The compound of claim 4 wherein [symbol] is a double bond.

14. The compound of claim 4 wherein [symbol] is a single bond.

15. The compound of claim 5 wherein [symbol] is a double bond.

16. The compound of claim 5 wherein [symbol] is a single bond.

17. A compound selected from the group consisting of:
(4aR,4bS,6aS,9aS,9bS)-1,4a,6a-trimethyl-7-(pyridin-3-yl)-4,4a,4b,5,6,6a,9,9a,9b,10-decahydro-1H-indeno[5,4-f]quinolin-2(3H)-one,
(4aR,4bS,6aS,9aS)-4a,6a-dimethyl-7-(pyridin-3-yl)-4,4a,4b,5,6,6a,9,9a,9b,10-decahydro-1H-indeno[5,4-f]quinolin-2(3H)-one,
(3aS,3bS,9aR,9bS,11aS)-5,9a,11a-trimethyl-1-(pyridin-3-yl)-3b,4,5,8,9,9a,9b,10,11,11a-dodecahydro-3H-cyclopenta[i]phenanthridin-7(3 aH)-one,
(3aS,3bS,9aR,9bS,11aS)-9a,11a-dimethyl-1-(pyridin-3-yl)-3b,4,5,8,9,9a,9b,10,11,11a-decahydro-3H-cyclopenta[i]phenanthridin-7(3 aH)-one,
(4aR,4bS,6aS,9aS,9bS)-7-(1H-benzo[d]imidazol-2-yl)-1,4a,6a-trimethyl-4,4a,5,6,6a,9,9a,9b,10-decahydro-1H-indeno[5,4-f]quinolin-2(3H)-one,
(4aR,4bS,6aS,9aS,9bR)-7-(1H-benzo[d]imidazol-1-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-7-(6-methoxypyridin-3-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-7-(5-methoxypyridin-3-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-1,4a,6a-trimethyl-7-(4-methylpyridin-3-yl)-4,4a,4b,5,6,6a,9,9a,9 b,10-decahydro-1H-indeno[5,4-f]quinolin-2-one,
(4aR,4bS,6aS,9aS,9bS)-1,4a,6a-trimethyl-7-(pyrimidin-5-yl)-4,4a,4b,5,6,6a,9,9a,9b,10-decahydro-1H-indeno[5,4-f]quinolin-2(3H)-one,
(4aR,4bS,6aS,9aS,9bS)-1,4a,6a-trimethyl-7-(pyrazin-2-yl)-4,4a,4b,5,6,6a,9,9a,9b,10-decahydro-1H-indeno[5,4-f]quinolin-2(3H)-one,
(4aR,4bS,6aS,9aS,9bS)-1,4a,6a-trimethyl-7-(quinolin-3-yl)-4,4a,4b,5,6,6a,9,9a,9b,10-decahydro-1H-indeno[5,4-f]quinolin-2(3H)-one,
(4aR,4bS,6aS,9aS,9bS)-7-(5-chloropyridin-3-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-7-(4-chloropyridin-3-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-1-ethyl-4a,6a-dimethyl-7-(pyridin-3-yl)-4,4a,4b,5,6,6a,9,9a,9b,10-decahydro-1H-indeno[5,4-f]quinolin-2(3H)-one,
(4aR,4bS,6aS,9aS,9bS)-1-ethyl-4a,6a-dimethyl-7-(pyrimidin-5-yl)-4,4a,4b,5,6,6a,9,9a,9b,10-decahydro-1H-indeno[5,4-f]quinolin-2(3H)-one,
(4aR,4bS,6aS,9aS,9bS)-1-ethyl-4a,6a-dimethyl-7-(pyrazin-2-yl)-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-4a,6a-dimethyl-7-(pyridin-3-yl)-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-4a,6a-dimethyl-7-(pyrimidin-5-yl)-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-4a,6a-dimethyl-7-(pyrazin-2-yl)-4,4a,4b,5,6,6a,9,9a,9b,10-decahydro-1H-indeno[5,4-f]quinolin-2(3H)-one,
(4aR,4bS,6aS,9aS,9bS)-7-(5-chloropyridin-3-yl)-1-ethyl-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-ethyl-7-(6-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-ethyl-7-(4-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-7-(5-chloropyridin-3-yl)-1-cyclopropyl-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(6-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-7-(5-chloropyridin-3-yl)-4a,6a-dimethyl-1-propyl-4,4a,4b,5,6,6a,9,9a,9b,10-decahydro-1H-indeno[5,4-f]quinolin-2(3H)-one,
(4aR,4bS,6aS,9aS,9bS)-4a,6a-dimethyl-7-(pyridin-3-yl)-4,4a,4b,5,6,6a,9,9a,9b,10,11,11a-dodecahydroindeno[5,4-f]chromen-2(3H)-one,
(4aR,4bS,6aS,9aS,9bR)-4a,6a-dimethyl-7-(pyridin-3-yl)-4,4a,4b,5,6,6a,9,9a,9b,10-decahydroindeno[5,4-f]chromen-2(3H)-one,
(3aS,3bS,9aR,9bS,11aS)-9a,11a-dimethyl-1-(pyridin-3-yl)-3a,3b,4,6,7,8,9,9a,9b,10,11,11a-dodecahydro-3H-cyclopenta[i]phenanthridin-7-ol,
(3aS,3bS,9aR,9bS,11aS)-9a,11a-dimethyl-1-(pyridin-3-yl)-3a,3b,4,6,7,8,9,9a,9b,10,11,11a-dodecahydro-3H-cyclopenta[i]phenanthridin-7-yl acetate,
(3aS,3bS,9aR,9bS,11aS)-1-(5-methoxypyridin-3-yl)-9a,11a-dimethyl-3a,3b,4,6,7,8,9,9a,9b,10,11,11a-dodecahydro-3H-cyclopenta[i]phenanthridin-7-ol,
(3aS,3bS,9aR,9bS,11aS)-1-(5-methoxypyridin-3-yl)-9a,11a-dimethyl-3a,3b,4,6,7,8,9,9a,9b,10,11,11a-dodecahydro-3H-cyclopenta[i]phenanthridin-7-yl acetate,
(3aS,3bS,9aR,9bS,11aS)-9a,11a-dimethyl-1-(pyridin-3-yl)-3,3a,3b,4,9,9a,9b,10,11,11a-decahydroindeno[4,5-c]chromen-7(8H)-one,
(3aS,3bS,9aR,9bS,11aS)-1-(5-methoxypyridin-3-yl)-9a,11a-dimethyl-3,3a,3b,4,9,9a,9b,10,11,11a-decahydroindeno[4,5-c]chromen-7(8H)-one,
(3aS,3bS,9aR,9bS,11aS)-1-(4-methoxypyridin-3-yl)-9a,11a-dimethyl-3,3a,3b,4,9,9a,9b,10,11,11a-decahydroindeno[4,5-c]chromen-7(8H)-one,
(4aR,4bS,6aS,9aS,9bS)-4a,6a-dimethyl-7-(pyrazin-2-yl)-4,4a,4b,5,6,6a,9,9a,9b,10,11,11a-dodecahydroindeno[5,4-f]chromen-2(3H)-one,
(4aR,4bS,6aS,9aS,9bS)-7-(6-methoxypyrazin-2-yl)-4a,6a-dimethyl-4,4a,4b,5,6,6a,9,9a,9b,10,11,11a-dodecahydroindeno[5,4-f]chromen-2(3H)-one,
(4aR,4bS,6aS,9aS,9bS)-4a,6a-dimethyl-7-(pyrazin-2-yl)-4,4a,4b,5,6,6a,9,9a,9b,10-decahydroindeno[5,4-f]chromen-2(3H)-one,
(4aR,4bS,6aS,9aS,9bS)-7-(6-methoxypyrazin-2-yl)-4a,6a-dimethyl-4,4a,4b,5,6,6a,9,9a,9b,10-decahydroindeno[5,4-f]chromen-2(3H)-one,
(4aR,4bS,6aS,9aS,9bS)-4a,6a-dimethyl-7-(pyridin-3-yl)-4,4a,4b,5,6,6a,9,9a,9b,10,11,11a-dodecahydro-1H-indeno[5,4-f]quinolin-2(3H)-one,
(4aR,4bS,6aS,9aS,9bS)-7-(5-methoxypyridin-3-yl)-4a,6a-dimethyl-4,4a,4b,5,6,6a,9,9a,9b,10,11,11a-dodecahydro-1H-indeno[5,4-f]quinolin-2(3H)-one,
(4aR,4bS,6aS,9aS,9bS)-7-(4-methoxypyridin-3-yl)-4a,6a-dimethyl-4,4a,4b,5,6,6a,9,9a,9b,10,11,11a-dodecahydro-1H-indeno[5,4-f]quinolin-2(3H)-one,
(4aR,4bS,6aS,9aS,9bS)-7-(5-fluoropyridin-3-yl)-1,4a,6a-trimethyl-1,3,4,4a,4b,5,6,6a,9,9a,9b,10-dodecahydro-indeno[5,4-f]quinolin-2(3H)-one,
(4aR,4bS,6aS,9aS,9bS)-7-(5-methylpyridin-3-yl)-1,4a,6a-trimethyl-1,3,4,4a,4b,5,6,6a,9,9a,9b,10-dodecahydro-indeno[5,4-f]quinolin-2(3H)-one,
(4aR,4bS,6aS,9aS,9bS)-7-(4-methoxy-pyridin-3-yl)-1,4a,6a-trimethyl-1,3,4,4a,4b,5,6,6a,9,9a,9b,10-dodecahydro-indeno[5,4-f]quinolin-2(3H)-one,
(4aR,4bS,6aS,9aS,9bS)-7-(5-Ethoxy-pyridin-3-yl)-1,4a,6a-trimethyl-1,3,4,4a,4b,5,6,6a,9,9a,9b,10-dodecahydro-indeno[5,4-f]quinolin-2(3H)-one,
(4aR,4bS,6aS,9aS,9bS)-1-ethyl-7-(5-methoxypyridin-3-yl)-4a,6a-dimethyl 4,4a,4b,5,6,6a,9,9a,9b,10-decahydro-1H-indeno[5,4-f]quinolin-2(3H)-one,
(4aR,4bS,6aS,9aS,9bS)-1-ethyl-7-(5-ethoxypyridin-3-yl)-4a,6a-dimethyl 4,4a,4b,5,6,6a,9,9a,9b,10-decahydro-1H-indeno[5,4-f]quinolin-2(3H)-one,
(4aR,4bS,6aS,9aS,9bS)-1-ethyl-7-(5-fluoropyridin-3-yl)-4a,6a-dimethyl 4,4a,4b,5,6,6a,9,9a,9b,10-decahydro-1H-indeno[5,4-f]quinolin-2(3H)-one,
(4aR,4bS,6aS,9aS,9bS)-1-ethyl-7-(5-methylpyridin-3-yl)-4a,6a-dimethyl 4,4a,4b,5,6,6a,9,9a,9b,10-decahydro-1H-indeno[5,4-f]quinolin-2(3H)-one,
(4aR,4bS,6aS,9aS,9bS)-1-ethyl-7-(5-chloropyridin-3-yl)-4a,6a-dimethyl 4,4a,4b,5,6,6a,9,9a,9b,10-decahydro-1H-indeno[5,4-f]quinolin-2(3H)-one,
4aR,4bS,6aS,9aS,9bS)-1-ethyl-7-(5-methylpyridin-3-yl)-4a,6a-dimethyl 4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(5-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(5-ethoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(5-fluoropyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(5-methylpyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(4-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(4-chloropyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(4-methylpyridin-3-yl)-4a,6a-dimethyl-4,4a,4b,5,6,6a,9,9a,9b,10-decahydro-1H-indeno[5,4-f]quinolin-2(3H)-one,
(3aS,3bS,9aR,9bS,11aS)-1-(5-methoxypyridin-3-yl)-5,9a,11a-trimethyl-3b,4,5,8,9,9a,9b,10,11,11a-decahydro-3H-cyclopenta[i]phenanthridin-7(3aH)-one,
(3aS,3bS,9aR,9bS,11aS)-1-(5-ethoxypyridin-3-yl)-5,9a,11a-trimethyl-3b,4,5,8,9,9a,9b,10,11,11a-decahydro-3H-cyclopenta[i]phenanthridin-7(3aH)-one,
(3aS,3bS,9aR,9bS,11aS)-1-(5-fluoropyridin-3-yl)-5,9a,11a-trimethyl-3b,4,5,8,9,9a,9b,10,11,11a-decahydro-3H-cyclopenta[i]phenanthridin-7(3 aH)-one,
(3aS,3bS,9aR,9bS,11aS)-1-(5-chloropyridin-3-yl)-5,9a,11a-trimethyl-3b,4,5,8,9,9a,9b,10,11,11a-decahydro-3H-cyclopenta[i]phenanthridin-7(3 aH)-one,
(3aS,3bS,9aR,9bS,11aS)-1-(5-methylpyridin-3-yl)-5,9a,11a-trimethyl-3b,4,5,8,9,9a,9b,10,11,11a-decahydro-3H-cyclopenta[i]phenanthridin-7(3aH)-one,
(3aS,3bS,9aR,9bS,11aS)-1-(4-methoxypyridin-3-yl)-5,9a,11a-trimethyl-3b,4,5,8,9,9a,9b,10,11,11a-decahydro-3H-cyclopenta[i]phenanthridin-7(3aH)-one,
(3aS,3bS,9aR,9bS,11aS)-1-(4-chloropyridin-3-yl)-5,9a,11a-trimethyl-3b,4,5,8,9,9a,9b,10,11,11a-decahydro-3H-cyclopenta[i]phenanthridin-7(3 aH)-one,
(3aS,3bS,9aR,9bS,11aS)-1-(4-methylpyridin-3-yl)-5,9a,11a-trimethyl-3b,4,5,8,9,9a,9b,10,11,11a-decahydro-3H-cyclopenta[i]phenanthridin-7(3aH)-one,
(3aS,3bS,9aR,9bS,11aS)-1-(5-methoxypyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-1-(5-ethoxypyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-1-(5-fluoropyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-1-(5-chloropyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-1-(5-methylpyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-1-(4-methoxypyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-1-(4-chloropyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,1aS)-1-(4-methylpyridin-3-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-5-ethyl-9a,11a-dimethyl-1-(pyridin-3-yl)-3b,4,5,8,9,9a,9b,10,11,11a-decahydro-3H-cyclopenta[i]phenanthridin-7(3 aH)-one,
(3aS,3bS,9aR,9bS,11aS)-5-ethyl-1-(5-methoxypyridin-3-yl)-9a,11a-dimethyl-3b,4,5,8,9,9a,9b,10,11,11a-decahydro-3H-cyclopenta[i]phenanthridin-7(3aH)-one,
(3aS,3bS,9aR,9bS,11aS)-5-ethyl-1-(5-ethoxypyridin-3-yl)-9a,11a-dimethyl-3b,4,5,8,9,9a,9b,10,11,11a-decahydro-3H-cyclopenta[i]phenanthridin-7(3aH)-one,
(3aS,3bS,9aR,9bS,11aS)-5-ethyl-1-(5-fluoropyridin-3-yl)-9a,11a-dimethyl-3b,4,5,8,9,9a,9b,10,11,11a-decahydro-3H-cyclopenta[i]phenanthridin-7(3aH)-one,
(3aS,3bS,9aR,9bS,11aS)-5-ethyl-1-(5-chloropyridin-3-yl)-9a,11a-dimethyl-3b,4,5,8,9,9a,9b,10,11,11a-decahydro-3H-cyclopenta[i]phenanthridin-7(3aH)-one,
(3aS,3bS,9aR,9bS,11aS)-5-ethyl-1-(5-methylpyridin-3-yl)-9a,11a-dimethyl-3b,4,5,8,9,9a,9b,10,11,11a-decahydro-3H-cyclopenta[i]phenanthridin-7(3aH)-one,
(3aS,3bS,9aR,9bS,11aS)-5-ethyl-1-(4-methoxypyridin-3-yl)-9a,11a-dimethyl-3b,4,5,8,9,9a,9b,10,11,11a-decahydro-3H-cyclopenta[i]phenanthridin-7(3aH)-one,
(3aS,3bS,9aR,9bS,11aS)-5-ethyl-1-(4-chloropyridin-3-yl)-9a,11a-dimethyl-3b,4,5,8,9,9a,9b,10,11,11a-decahydro-3H-cyclopenta[i]phenanthridin-7(3aH)-one,
(3aS,3bS,9aR,9bS,11aS)-5-ethyl-1-(4-methylpyridin-3-yl)-9a,11a-dimethyl-3b,4,5,8,9,9a,9b,10,11,11a-decahydro-3H-cyclopenta[i]phenanthridin-7(3aH)-one,
(4aR,4bS,6aS,9aS,9bS)-7-(6-methoxypyrazin-2-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-1,4a,6a-trimethyl-7-(6-methylpyrazin-2-yl)-4,4a,4b,5,6,6a,9,9a,9b,10-decahydro-1H-indeno[5,4-f]quinolin-2(3H)-one,
(4aR,4bS,6aS,9aS,9bS)-7-(6-ethylpyrazin-2-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-7-(6-ethoxypyrazin-2-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-1-ethyl-7-(6-methoxypyrazin-2-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-ethyl-7-(6-methoxypyrazin-2-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-ethyl-7-(6-ethylpyrazin-2-yl)-4a,6a-dimethyl 4,4a,4b,5,6,6a,9,9a,9b,10-decahydro-1H-indeno[5,4-f]quinolin-2(3H)-one,
(4aR,4bS,6aS,9aS,9bS)-1-ethyl-7-(6-ethoxylpyrazin-2-yl)-4a,6a-dimethyl 4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(6-methoxypyrazin-2-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(6-methylpyrazin-2-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(6-ethylpyrazin-2-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1-cyclopropyl-7-(6-ethoxypyrazin-2-yl)-4a,6a-dimethyl-4,4a,4b,5,6,6a,9,9a,9b,10-decahydro-1H-indeno[5,4-f]quinolin-2(3H)-one,
(3aS,3bS,9aR,9bS,11aS)-5,9a,11a-trimethyl-1-(pyrazin-2-yl)-3b,4,5,8,9,9a,9b,10,11,11a-decahydro-3H-cyclopenta[i]phenanthridin-7(3 aH)-one,
(3aS,3bS,9aR,9bS,11aS)-1-(6-methoxypyrazin-2-yl)-5,9a,11a-trimethyl-3b,4,5,8,9,9a,9b,10,11,11a-decahydro-3H-cyclopenta[i]phenanthridin-7(3aH)-one,
(3aS,3bS,9aR,9bS,11aS)-1-(6-ethoxypyrazin-2-yl)-5,9a,11a-trimethyl-3b,4,5,8,9,9a,9b,10,11,11a-decahydro-3H-cyclopenta[i]phenanthridin-7(3aH)-one,
(3aS,3bS,9aR,9bS,11aS)-1-(6-ethylpyrazin-2-yl)-5,9a,11a-trimethyl-3b,4,5,8,9,9a,9b,10,11,11a-decahydro-3H-cyclopenta[i]phenanthridin-7(3 aH)-one,
(4aR,4bS,6aS,9aS,9bS)-7-(5-methoxypyridin-3-yl)-4a,6a-dimethyl-4,4a,4b,5,6,6a,9,9a,9b,10,11,11a-dodecahydroindeno[5,4-f]chromen-2(3H)-one,
(4aR,4bS,6aS,9aS,9bS)-7-(5-ethoxypyridin-3-yl)-4a,6a-dimethyl-4,4a,4b,5,6,6a,9,9a,9b,10,11,11a-dodecahydroindeno[5,4-f]chromen-2(3H)-one,
(4aR,4bS,6aS,9aS,9bS)-7-(4-methoxypyridin-3-yl)-4a,6a-dimethyl-4,4a,4b,5,6,6a,9,9a,9b,10,11,11a-dodecahydroindeno[5,4-f]chromen-2(3H)-one,
(4aR,4bS,6aS,9aS,9bS)-7-(5-methoxypyridin-3-yl)-4a,6a-dimethyl-4,4a,4b,5,6,6a,9,9a,9b,10-decahydroindeno[5,4-f]chromen-2(3H)-one,
(4aR,4bS,6aS,9aS,9bS)-7-(5-ethoxypyridin-3-yl)-4a,6a-dimethyl-4,4a,4b,5,6,6a,9,9a,9b,10-decahydroindeno[5,4-f]chromen-2(3H)-one,
(4aR,4bS,6aS,9aS,9bS)-7-(4-methoxypyridin-3-yl)-4a,6a-dimethyl-4,4a,4b,5,6,6a,9,9a,9b,10-decahydroindeno[5,4-f]chromen-2(3H)-one,
(4aR,4bS,6aS,9aS,9bS)-7-(4-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-7-(5-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-7-(6-methoxypyridin-3-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-4a,6a-dimethyl-7-(pyrazin-2-yl)-4,4a,4b,5,6,6a,9,9a,9b,10-decahydro-1H-indeno[5,4-f]quinolin-2(3H)-one,
(4aR,4bS,6aS,9aS,9bS)-7-(6-methoxypyrazin-2-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-4a,6a-dimethyl-7-(pyrimidin-5-yl)-4,4a,4b,5,6,6a,9,9a,9b,10-decahydro-1H-indeno[5,4-f]quinolin-2(3H)-one,
(3aS,3bS,9aR,9bS,11aS)-1-iodo-9a,11a-dimethyl-3b,4,5,8,9,9a,9b,10,11,11a-decahydro-3H-cyclopenta[i]phenanthridin-7(3 aH)-one,
(3aS,3bS,9aR,9bS,111aS)-9a,11a-dimethyl-1-(pyrazin-2-yl)-3b,4,5,8,9,9a,9b,10,11,11a-decahydro-3H-cyclopenta[i]phenanthridin-7(3 aH)-one,
(3aS,3bS,9aR,9bS,11aS)-1-(6-methoxypyrazin-2-yl)-9a,11a-dimethyl-3b,4,5,8,9,9a,9b,10,11,11a-decahydro-3H-cyclopenta[i]phenanthridin-7(3 aH)-one,
(4aR,4bS,6aS,9aS,9bS)-4a,6a-dimethyl-7-(oxazol-5-yl)-4,4a,4b,5,6,6a,9,9a,9b,10-decahydro-1H-indeno[5,4-f]quinolin-2(3H)-one,
(4aR,4bS,6aS,9aS,9bS)-4a,6a-dimethyl-7-(thiazol-5-yl)-4,4a,4b,5,6,6a,9,9a,9b,10-decahydro-1H-indeno[5,4-f]quinolin-2(3H)-one,
(4aR,4bS,6aS,9aS,9bS)-7-(isoxazol-4-yl)-4a,6a-dimethyl-4,4a,4b,5,6,6a,9,9a,9b,10-decahydro-1H-indeno[5,4-f]quinolin-2(3H)-one,
(4aR,4bS,6aS,9aS,9bS)-1,4a,6a-trimethyl-7-(oxazol-5-yl)-4,4a,4b,5,6,6a,9,9a,9b,10-decahydro-1H-indeno[5,4-f]quinolin-2(3H)-one,
(4aR,4bS,6aS,9aS,9bS)-1,4a,6a-trimethyl-7-(thiazol-5-yl)-4,4a,4b,5,6,6a,9,9a,9b,10-decahydro-1H-indeno[5,4-f]quinolin-2(3H)-one,
(4aR,4bS,6aS,9aS,9bS)-7-(isoxazol-4-yl)-1,4a,6a-trimethyl-4,4a,4b,5,6,6a,9,9a,9b,10-decahydro-1H-indeno[5,4-f]quinolin-2(3H)-one,
(4aR,4bS,6aS,9aS,9bS)-4a,6a-dimethyl-7-(oxazol-5-yl)-4,4a,4b,5,6,6a,9,9a,9b,10,11,11a-dodecahydroindeno[5,4-f]chromen-2(3H)-one,
(4aR,4bS,6aS,9aS,9bS)-4a,6a-dimethyl-7-(thiazol-5-yl)-4,4a,4b,5,6,6a,9,9a,9b,10,11,11a-dodecahydroindeno[5,4-f]chromen-2(3H)-one,
(4aR,4bS,6aS,9aS,9bS)-7-(isoxazol-4-yl)-4a,6a-dimethyl-4,4a,4b,5,6,6a,9,9a,9b,10,11,11a-dodecahydroindeno[5,4-f]chromen-2(3H)-one,
(4aR,4bS,6aS,9aS,9bS)-4a,6a-dimethyl-7-(oxazol-5-yl)-4,4a,4b,5,6,6a,9,9a,9b,10-decahydroindeno[5,4-f]chromen-2(3H)-one,
(4aR,4bS,6aS,9aS,9bS)-4a,6a-dimethyl-7-(thiazol-5-yl)-4,4a,4b,5,6,6a,9,9a,9b,10-decahydroindeno[5,4-f]chromen-2(3H)-one,
(4aR,4bS,6aS,9aS,9bS)-7-(isoxazol-4-yl)-4a,6a-dimethyl-4,4a,4b,5,6,6a,9,9a,9b,10-decahydroindeno[5,4-f]chromen-2(3H)-one,
(3aS,3bS,9aR,9bS,11aS)-9a,11a-dimethyl-1-(oxazol-5-yl)-3b,4,5,8,9,9a,9b,10,11,11a-decahydro-3H-cyclopenta[i]phenanthridin-7(3 aH)-one,
(3aS,3bS,9aR,9bS,11aS)-9a,11a-dimethyl-1-(thiazol-5-yl)-3b,4,5,8,9,9a,9b,10,11,11a-decahydro-3H-cyclopenta[i]phenanthridin-7(3 aH)-one,
(3aS,3bS,9aR,9bS,11aS)-1-(isoxazol-4-yl)-9a,11a-dimethyl-3b,4,5,8,9,9a,9b,10,11,11a-decahydro-3H-cyclopenta[i]phenanthridin-7(3 aH)-one,
(3aS,3bS,9aR,9bS,11aS)-5,9a,11a-trimethyl-1-(oxazol-5-yl)-3b,4,5,8,9,9a,9b,10,11,11a-decahydro-3H-cyclopenta[i]phenanthridin-7(3 aH)-one,
(3aS,3bS,9aR,9bS,11aS)-5,9a,11a-trimethyl-1-(thiazol-5-yl)-3b,4,5,8,9,9a,9b,10,11,11a-decahydro-3H-cyclopenta[i]phenanthridin-7(3 aH)-one,
(3aS,3bS,9aR,9bS,11aS)-1-(isoxazol-4-yl)-5,9a,11a-trimethyl-3b,4,5,8,9,9a,9b,10,11,11a-decahydro-3H-cyclopenta[i]phenanthridin-7(3 aH)-one,
(4aR,4bS,6aS,9aS,9bS)-4a,6a-dimethyl-7-(1H-1,2,3-triazol-4-yl)-4,4a,4b,5,6,6a,9,9a,9b,10,11,11a-dodecahydroindeno[5,4-f]chromen-2(3H)-one,
(4aR,4bS,6aS,9aS,9bS)-4a,6a-dimethyl-7-(1H-1,2,3-triazol-4-yl)-4,4a,4b,5,6,6a,9,9a,9b,10-decahydroindeno[5,4-f]chromen-2(3H)-one,
(3aS,3bS,9aR,9bS,11aS)-5,9a,11a-trimethyl-1-(1H-1,2,3-triazol-4-yl)-3b,4,5,8,9,9a,9b,10,11,11a-decahydro-3H-cyclopenta[i]phenanthridin-7(3aH)-one,
(3aS,3bS,9aR,9bS,11aS)-9a,11a-dimethyl-1-(1H-1,2,3-triazol-4-yl)-3b,4,5,8,9,9a,9b,10,11,11a-decahydro-3H-cyclopenta[i]phenanthridin-7(3 aH)-one,
(4aR,4bS,6aS,9aS,9bS)-1,4a,6a-trimethyl-7-(1H-1,2,3-triazol-4-yl)-4,4a,4b,5,6,6a,9,9a,9b,10-decahydro-1H-indeno[5,4-f]quinolin-2(3H)-one, and
(4aR,4bS,6aS,9aS,9bS)-4a,6a-dimethyl-7-(1H-1,2,3-triazol-4-yl)-4,4a,4b,5,6,6a,9,9a,9b,10-decahydro-1H-indeno[5,4-f]quinolin-2(3H)-one.

18. A pharmaceutical composition comprising a compound of any of claims 1-3 and a pharmaceutically acceptable carrier, excipient or binder thereof.

19. A method for treating cancer in a subject comprising administering to a subject in need a therapeutically acceptable amount of a compound of any of claims 1-3 or a pharmaceutically acceptable salt or solvate thereof.

20. The method of claim 19 wherein the cancer is selected from the group consisting of bladder cancer, brain cancer, breast cancer, cervical cancer, colorectal cancer, endometrial cancer, gastric cancer, glioblastoma, head and neck cancer, Kaposi's sarcoma, kidney cancer, leiomyosarcoma, leukemia, liver cancer, lung cancer, melanoma, multiple myeloma, Non-Hodgkin lymphoma, ovarian cancer, pancreatic cancer, papillary renal cell carcinoma, prostate cancer, renal cancer, squamous cell cancer, and thoracic cancer.

21. A method of treating an androgen-dependent disorder in a subject comprising administering to a subject in need a therapeutically acceptable amount of a compound of any of claims 1-3 or a pharmaceutically acceptable salt or solvate thereof.

22. The method of claim 21 wherein the androgen-dependent disorder is selected from the group consisting of prostate cancer, benign prostatic hyperplasia, prostatic intraepithelial neoplasia, hirsutism, acne, androgenic alopecia, and polycystic ovary syndrome.
